(19) United States
(12) Patent Application Publication
(10) Pub. No.: US 2003/0212257 A1
(43) Pub. Date: Nov. 13, 2003

(54) Novel human proteins, polynucleotides encoding them and methods of using the same

(76) Inventors: Kimberly A. Spytek, New Haven, CT (US); Li Li, Branford, CT (US); Shlomit R. Edinger, New Haven, CT (US); Karen Ellerman, Branford, CT (US); David J. Stone, Guilford, CT (US); Uriel M. Malyankar, Branford, CT (US); Richard A. Shimkets, Guilford, CT (US); Xiaojia Sasha Guo, Branford, CT (US); David W. Anderson, Branford, CT (US); Meera Patturajan, Branford, CT (US); Constance Berghs, New Haven, CT (US); Valerie Gerlach, Branford, CT (US); Vladimir Y. Gusev, Madison, CT (US); Ramesh Kekuda, Norwalk, CT (US); Linda Gorman, Branford, CT (US); Bryan D. Zerhusen, Branford, CT (US); Jason C. Baumgartner, New Haven, CT (US); Velizar T. Tchernev, Branford, CT (US); Corine A.M. Vernet, Branford, CT (US); Glennda Smithson, Guilford, CT (US); Suresh G. Shenoy, Branford, CT (US); Xiaohong Liu, Lexington, MA (US); John R. MacDoughall, Hamden, CT (US)

Correspondence Address: Ivor R. Elrifi, Mintz, Levin, Cohn, Ferris,, Glovsky and Popeo, P.C., One Financial Center, Boston, MA, 02111, US

(21) Appl. No.: 10/115,482

(22) Filed: Apr. 2, 2002

Related U.S. Application Data
(60) Provisional application No. 60/281,086, filed on Apr. 3, 2001.
(60) Provisional application No. 60/281,136, filed on Apr. 3, 2001.
(60) Provisional application No. 60/281,863, filed on Apr. 5, 2001.
(60) Provisional application No. 60/281,906, filed on Apr. 5, 2001.
(60) Provisional application No. 60/282,934, filed on Apr. 10, 2001.
(60) Provisional application No. 60/283,512, filed on Apr. 12, 2001.
(60) Provisional application No. 60/285,325, filed on Apr. 19, 2001.
(60) Provisional application No. 60/285,890, filed on Apr. 23, 2001.
(60) Provisional application No. 60/286,068, filed on Apr. 24, 2001.
(60) Provisional application No. 60/286,292, filed on Apr. 25, 2001.
(60) Provisional application No. 60/287,213, filed on Apr. 27, 2001.
(60) Provisional application No. 60/288,257, filed on May 2, 2001.
(60) Provisional application No. 60/291,134, filed on May 15, 2001.
(60) Provisional application No. 60/282,020, filed on Apr. 6, 2001.
(60) Provisional application No. 60/291,725, filed on May 17, 2001.
(60) Provisional application No. 60/294,771, filed on May 31, 2001.
(60) Provisional application No. 60/296,965, filed on Jun. 8, 2001.
(60) Provisional application No. 60/299,128, filed on Jun. 18, 2001.
(60) Provisional application No. 60/305,063, filed on Jul. 12, 2001.
(60) Provisional application No. 60/332,780, filed on Nov. 14, 2001.
(60) Provisional application No. 60/345,221, filed on Jan. 4, 2002.

(51) Int. Cl. C07K 1/00; C07K 14/00; C07K 17/00

(52) U.S. Cl. 530/350

(57) ABSTRACT

Disclosed are polypeptides and nucleic acids encoding same. Also disclosed are vectors, host cells, antibodies and recombinant methods for producing the polypeptides and polynucleotides, as well as methods for using same.

### RELATED APPLICATIONS

This application claims priority to provisional patent applications 60/281,086 filed Apr. 4, 2001; No. 60/281,136 filed Apr. 4, 2001; No. 60/281,863 filed Apr. 5, 2001; No. 60/281,906 filed Apr. 5, 2001; No. 60/282,934 filed Apr. 10, 2001; No. 60/283,512 filed Apr. 12, 2001; No. 60/285,325 filed Apr. 19, 2001; No. 60/285,890 filed Apr. 23, 2001; No. 60/286,068, filed Apr. 24, 2001; No. 60/286,292, filed Apr. 25, 2001; No. 60/287,213 filed Apr. 27, 2001; No. 60/288,257 filed May 2, 2001; No. 60/291,134 filed May 12, 2001; No. 60/282,020 filed May 15, 2001; No. 60/291,725 filed May 17, 2001; No. 60/294,771 filed May 31, 2001; No. 60/296,965 filed May 8, 2001; No. 60/299,128 filed Jun. 18, 2001; No. 60/305,063 filed Jul. 12, 2001; No. 60/332,780 filed Nov. 14, 2001; and No. 60/345,221 filed Jan. 4, 2002, each hereby incorporated herein by reference.

### FIELD OF THE INVENTION

The present invention is based in part on nucleic acids encoding proteins that are new members of the following protein families: calcium transport-like proteins, tetratricopeptide repeat-containing proteins, TSG118.1-like proteins, transcription elongation factor-like proteins, DENSIN 180-like proteins, EURL-like proteins, zinc finger protein 106-like proteins, ribosomal-like proteins, intracellular-like proteins, histone deacetylase 4-like proteins, glutaredoxin 3-like proteins, ubiquitin GDX-like proteins, homeodomain-interacting protein kinase-like proteins, mitogen activated kinase-like proteins, Alpha-2 globin-like proteins, enhancer of ZESTE homolog 1-like proteins, pancreatic hormone peptide domain containing protein-like proteins, MAP kinase-activating death domain protein-like proteins, GAR22-like proteins, high sulfur keratin-like proteins, ring finger protein-like proteins, cation transporting ATPase-like proteins, Ig-like proteins, TSP-like proteins, and EGF domain-like proteins.

The invention relates to polynucleotides and the polypeptides encoded by such polynucleotides, as well as vectors, host cells, antibodies and recombinant methods for producing the polypeptides and polynucleotides, as well as methods for using the same.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The present invention is based in part on nucleic acids encoding proteins that are members of the following protein families: calcium transport-like proteins, tetratricopeptide repeat-containing proteins, TSG118.1-like proteins, transcription elongation factor-like proteins, DENSIN 180-like proteins, EURL-like proteins, zinc finger protein 106-like proteins, ribosomal-like proteins, intracellular-like proteins, histone deacetylase 4-like proteins, glutaredoxin 3-like proteins, ubiquitin GDX-like proteins, homeodomain-interacting protein kinase-like proteins, mitogen activated kinase-like proteins, Alpha-2 globin-like proteins, enhancer of ZESTE homolog 1-like proteins, pancreatic hormone peptide domain containing protein-like proteins, MAP kinase-activating death domain protein-like proteins, GAR22-like proteins, high sulfur keratin-like proteins, ring finger protein-like proteins, cation transporting ATPase-like proteins, 1 g-like proteins, TSP-like proteins, and EGF domain-like proteins. The novel polynucleotides and polypeptides are referred to herein as NOV1, NOV2a, NOV2b, NOV3a, NOV3b, NOV4, NOV5, NOV6, NOV7, NOV8a, NOV8b, NOV9, NOV10, NOV11, NOV12, NOV13a, NOV13b, NOV14, NOV15, NOV16, NOV17a, NOV17b, NOV18, NOV19a, NOV19b, NOV20a, NOV20b, NOV21, NOV22, NOV23, NOV24, NOV25, NOV26 and NOV27. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NO:2n-1, wherein n is an integer between 1 and 34. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NO:2n, wherein n is an integer between 1 and 34. The nucleic acid can be, for example, a genomic DNA fragment or a cDNA molecule that includes the nucleic acid sequence of any of SEQ ID NO:2n-1, wherein n is an integer between 1 and 34.

Also included in the invention is an oligonucleotide, e.g., an oligonucleotide which includes at least 6 contiguous nucleotides of a NOVX nucleic acid (e.g., SEQ ID NO:2n-1, wherein n is an integer between 1 and 34) or a complement of said oligonucleotide. Also included in the invention are substantially purified NOVX polypeptides (SEQ ID NO:2n, wherein n is an integer between 1 and 34). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., adrenoleukodystrophy, congenital adrenal hyperplasia, hemophilia, hypercoagulation, idiopathic thrombocytopenic purpura, autoimmune disease, allergies, immunodeficiencies, transplantation, Von Hippel-Lindau (VHL) syndrome, Alzheimer's disease, stroke, tuberous sclerosis, hypercalcemia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, diabetes, renal artery stenosis, interstitial nephritis, glomerulonephritis, polycystic kidney disease, systemic lupus erythematosus, renal tubular acidosis, IgA nephropathy, hypercalcemia, cirrhosis, transplantation, systemic lupus erythematosus, autoimmune disease, asthma, emphysema, scleroderma, allergy, adult respiratory distress syndrome (ARDS), lymphedema, allergies, hemophilia, hypercoagulation, idiopathic thrombocytopenic purpura, autoimmune disease, allergies, immunodeficiencies, transplantation, graft versus host disease (GVHD), lymphedema, fertility, diabetes, pancreatitis, obesity, hemophilia, hypercoagulation, idiopathic thrombocytopenic purpura, immunodeficiencies, graft versus host, hypercalcemia, ulcers, anemia, ataxia-telangiectasia, cancer, trauma, regeneration (in vitro and in vivo), viral infections, bacterial infections, parasitic infections and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

NOVX nucleic acids and polypeptides are further useful in the generation of antibodies that bind immuno-specifically to the novel NOVX substances for use in therapeutic or diagnostic methods. These NOVX antibodies may be generated according to methods known in the art, using prediction from hydrophobicity charts, as described in the “Anti-NOVX Antibodies” section below. The disclosed NOVX proteins have multiple hydrophilic regions, each of which can be used as an immunogen. These NOVX proteins can be used in assay systems for functional analysis of various human disorders, which will help in understanding of pathology of the disease and development of new drug targets for various disorders.

The NOVX nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID NO | | |
|---|---|---|---|---|
| NOVX | Internal | (nucleic | SEQ ID NO | |
| Assignment | Identification | acid) | (polypeptide) | Homology |
| 1 | CG59448-02 | 1 | 2 | CAT-like protein |
| 2a | CG59706-01 | 3 | 4 | small glutamine-rich tetratricopeptide |
| | | | | repeat (TPR)-containing-like protein |
| 2b | CG59706-02 | 5 | 6 | small glutamine-rich tetratricopeptide |
| | | | | repeat (TPR)-containing-like protein |
| 3a | CG59766-01 | 7 | 8 | TSG118.1-like protein |
| 3b | CG59766-02 | 9 | 10 | TSG118.1-like protein |
| 4 | CG59813-01 | 11 | 12 | MRP-S10-like protein |
| 5 | CG59815-01 | 13 | 14 | RIKEN-like protein |
| 6 | CG59817-02 | 15 | 16 | transcription elongation factor S-II -like |
| 7 | CG59849-01 | 17 | 18 | Densin-like protein |
| 8a | CG59958-01 | 19 | 20 | EURL-like protein |
| 8b | CG59958-02 | 21 | 22 | EURL-like protein |
| 9 | CG59961-01 | 23 | 24 | zinc finger-like protein |
| 10 | CG88600-01 | 25 | 26 | cytochrome C-like |
| 11 | CG88655-01 | 27 | 28 | RIKEN-like protein |
| 12 | CG88665-01 | 29 | 30 | MCM2/3/5 family-like protein |
| 13a | CG88685-01 | 31 | 32 | HSPC125-like protein |
| 13b | CG88685-02 | 33 | 34 | HSPC125-like protein |
| 14 | CG88768-01 | 35 | 36 | Histone deacetylase 4-like |
| 15 | CG88856-01 | 37 | 38 | DMR -like protein |
| 16 | CG89958-01 | 39 | 40 | Glutaredoxin-like protein |
| 17a | CG90309-01 | 41 | 42 | Ubiquitin-like protein |
| 17b | CG90309-02 | 43 | 44 | Ubiguitin-like protein |
| 18 | CG90853-01 | 45 | 46 | homeodomain-interacting protein |
| | | | | kinase-like |
| 19a | CG90866-01 | 47 | 48 | KIAA1790-like protein |
| 19b | CG90866-02 | 49 | 50 | KIAA1790-like protein |
| 20a | CG93198-01 | 51 | 52 | Hemoglobin alpha chain-like protein |
| 20b | CG93198-02 | 53 | 54 | Hemoglobin alpha chain-like protein |
| 21 | CG93517-01 | 55 | 56 | zeste homolog 1-like protein |
| 22 | CG93781-01 | 57 | 58 | KIAA1813-like protein |
| 23 | CG93848-02 | 59 | 60 | MAP kinase-activating death domain |
| | | | | protein-like protein |
| 24 | CG94161-01 | 61 | 62 | GAS-2-like protein |
| 25 | CG94346-01 | 63 | 64 | Mucin-like protein |
| 26 | CG94600-01 | 65 | 66 | RET finger protein 2-like protein |
| 27 | CG94820-02 | 67 | 68 | cation-transporting ATPase-like protein |

Table A indicates homology of NOVX nucleic acids to known protein families. Thus, the nucleic acids and polypeptides, antibodies and related compounds according to the invention corresponding to a NOVX as identified in column 1 of Table A will be useful in therapeutic and diagnostic applications implicated in, for example, pathologies and disorders associated with the known protein families identified in column 5 of Table A.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table A, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table A.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g., a variety of cancers.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

In one specific embodiment, the invention includes an isolated polypeptide comprising an amino acid sequence selected from the group consisting of: (a) a mature form of the amino acid sequence selected from the group consisting of SEQ ID NO:2n, wherein n is an integer between 1 and 34; (b) a variant of a mature form of the amino acid sequence selected from the group consisting of SEQ ID NO:2n, wherein n is an integer between 1 and 34, wherein any amino acid in the mature form is changed to a different amino acid, provided that no more than 15% of the amino acid residues in the sequence of the mature form are so changed; (c) an amino acid sequence selected from the group consisting of SEQ ID NO:2n, wherein n is an integer between 1 and 34; (d) a variant of the amino acid sequence selected from the group consisting of SEQ ID NO:2n, wherein n is an integer between 1 and 34, wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO:2n, wherein n is an integer between 1 and 34; (b) a variant of a mature form of the amino acid sequence selected from the group consisting of SEQ ID NO:2n, wherein n is an integer between 1 and 34, wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO:2n, wherein n is an integer between 1 and 34; (d) a variant of the amino acid sequence selected from the group consisting of SEQ ID NO:2n, wherein n is an integer between 1 and 34, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO:2n, wherein n is an integer between 1 and 34, or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO:2n-1, wherein n is an integer between 1 and 34; (b) a nucleotide sequence wherein one or more nucleotides in the nucleotide sequence selected from the group consisting of SEQ ID NO:2n-1, wherein n is an integer between 1 and 34, is changed from that selected from the group consisting of the chosen sequence to a different nucleotide provided that no more than 15% of the nucleotides are so changed; (c) a nucleic acid fragment of the sequence selected from the group consisting of SEQ ID NO:2n-1, wherein n is an integer between 1 and 34; and (d) a nucleic acid fragment wherein one or more nucleotides in the nucleotide sequence selected from the group consisting of SEQ ID NO:2n-1, wherein n is an integer between 1 and 34, is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NO:2n-1, wherein n is an integer between 1 and 34, or a complement of this aforementioned nucleotide sequence, can be isolated using standard molecular biology techniques and the sequence information provided herein. Using all or a portion of the nucleic acid sequence of SEQ ID NO:2n-1, wherein n is an integer between 1 and 34, as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NO:2n-1, wherein n is an integer between 1 and 34, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO:2n-1, wherein n is an integer between 1 and 34, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NO:2n-1, wherein n is an integer between 1 and 34 is one that is sufficiently complementary to the nucleotide sequence shown SEQ ID NO:2n-1, wherein n is an integer between 1 and 34, that it can hydrogen bond with little or no mismatches to the nucleotide sequence shown SEQ ID NO:2n-1, wherein n is an integer between 1 and 34, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n-1, wherein n is an integer between 1 and 34, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NO:2n-1, wherein n is an integer between 1 and 34; or an anti-sense strand nucleotide sequence of SEQ ID NO:2n-1, wherein n is an integer between 1 and 34; or of a naturally occurring mutant of SEQ ID NO:2n-1, wherein n is an integer between 1 and 34.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NO:2n-1, wherein n is an integer between 1 and 34, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NO:2n-1, wherein n is an integer between 1 and 34, due to degeneracy of the genetic code and thus encode the same NOVX proteins as that encoded by the nucleotide sequences shown in SEQ ID NO:2n-1, wherein n is an integer between 1 and 34. In another embodiment, an isolated nucleic acid molecule of the invention has a nucleotide sequence encoding a protein having an amino acid sequence shown in SEQ ID NO:2n, wherein n is an integer between 1 and 34.

In addition to the human NOVX nucleotide sequences shown in SEQ ID NO:2n-1, wherein n is an integer between 1 and 34, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NO:2n-1, wherein n is an integer between 1 and 34, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n-1, wherein n is an integer between 1 and 34. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6× SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2× SSC, 0.01% BSA at 50° C. An isolated nucleic acid molecule of the invention that hybridizes under stringent conditions to the sequences SEQ ID NO:2n-1, wherein n is an integer between 1 and 34, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n-1, wherein n is an integer between 1 and 34, or fragments, analogs or derivatives thereof, under conditions of moderate stringency is provided. A non-limiting example of moderate stringency hybridization conditions are hybridization in 6× SSC, 5× Denhardt's solution, 0.5% SDS and 100 mg/ml denatured salmon sperm DNA at 55° C., followed by one or more washes in 1× SSC, 0.1% SDS at 370C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NO:2n-1, wherein n is an integer between 1 and 34, or fragments, analogs or derivatives thereof, under conditions of low stringency, is provided. A non-limiting example of low stringency hybridization conditions are hybridization in 35% formamide, 5× SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2× SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981.ProcNatlAcadSci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NO:2n-1, wherein n is an integer between 1 and 34, thereby leading to changes in the amino acid sequences of the encoded NOVX proteins, without altering the functional ability of said NOVX proteins. For example, nucleotide 18 A substitutions leading to amino acid substitutions at “non-essential” amino acid residues can be made in the sequence SEQ ID NO:2n, wherein n is an integer between 1 and 34. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO:2n, wherein n is an integer between 1 and 34, yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NO:2n, wherein n is an integer between 1 and 34. Preferably, the protein encoded by the nucleic acid molecule is at least about 60% homologous to SEQ ID NO:2n, wherein n is an integer between 1 and 34; more preferably at least about 70% homologous SEQ ID NO:2n, wherein n is an integer between 1 and 34; still more preferably at least about 80% homologous to SEQ ID NO:2n, wherein n is an integer between 1 and 34; even more preferably at least about 90% homologous to SEQ ID NO:2n, wherein n is an integer between 1 and 34; and most preferably at least about 95% homologous to SEQ ID NO:2n, wherein n is an integer between 1 and 34.

An isolated nucleic acid molecule encoding an NOVX protein homologous to the protein of SEQ ID NO:2n, wherein n is an integer between 1 and 34, can be created by introducing one or more nucleotide substitutions, additions or deletions into the nucleotide sequence of SEQ ID NO:2n-1, wherein n is an integer between 1 and 34, such that one or more amino acid substitutions, additions or deletions are introduced into the encoded protein.

Mutations can be introduced into SEQ ID NO:2n-1, wherein n is an integer between 1 and 34, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis of SEQ ID NO:2n-1, wherein n is an integer between 1 and 34, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NO:2n-1, wherein n is an integer between 1 and 34, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NO:2n, wherein n is an integer between 1 and 34, or antisense nucleic acids complementary to an NOVX nucleic acid sequence of SEQ ID NO:2n-1, wherein n is an integer between 1 and 34, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NO:2n-1, wherein n is an integer between 1 and 34). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660:27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996.supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NO:2n, wherein n is an integer between 1 and 34. The invention also includes a mutant or variant protein any of whose residues may be changed from the corresponding residues shown in SEQ ID NO:2n, wherein n is an integer between 1 and 34, while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NO:2n, wherein n is an integer between 1 and 34) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NO:2n, wherein n is an integer between 1 and 34. In other embodiments, the NOVX protein is substantially homologous to SEQ ID NO:2n, wherein n is an integer between 1 and 34, and retains the functional activity of the protein of SEQ ID NO:2n, wherein n is an integer between 1 and 34, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NO:2n, wherein n is an integer between 1 and 34, and retains the functional activity of the NOVX proteins of SEQ ID NO:2n, wherein n is an integer between 1 and 34.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO:2n-1, wherein n is an integer between 1 and 34.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NO:2n, wherein n is an integer between 1 and 34, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion i protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11:477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′}, and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G. which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fe), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80:2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The TM preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′) 2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immuinol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FclγRI (CD64), FcγR11 (CD32) and FcγRTII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148:2918-2922 (1992). Homodimeric antibodies with enhanced anti-tumor activity can also be prepared using heterobifunctional cross-linkers as described in Wolff et al. Cancer Research, 53:2560-2565 (1993). Alternatively, an antibody can be engineered that has dual Fc regions and can thereby have enhanced complement lysis and ADCC capabilities. See Stevenson et al., Anti-Cancer Drug Design, 3:219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences) It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g. Wada, et al., 1992. Nucl. Acids Res. 20:2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec 1 (Baldari, et al., 1987. EMBO J. 6:229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1:268-277), lymphoid-specific promoters (Calame and Eaton, 1988. Adv. Immunol. 43:235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NO:2n-1, wherein n is an integer between 1 and 34, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and U.S. Pat. No. 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NO:2n-1, wherein n is an integer between 1 and 34), but more preferably, is a non-human homologue of a human NOVX gene. For example, a mouse homologue of human NOVX gene of SEQ ID NO:2n-1, wherein n is an integer between 1 and 34, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test a compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37:2678; Cho, et al., 1993. Science 261: 1303; Carrell, et al., 1994. Angew. Chem. Int. Ed. Engl. 33:2059; Carell, et al., 1994. Angew. Chem. Int. Ed. Engl. 33:2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, Triton® X-114, Thesit®, decanoyl-N-methylglucamide, Triton® X-100, Isotridecypoly(ethylene glycol ether)ₙ, N-dodecyl--N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS(N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72:223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NO:2n-1, wherein n is an integer between 1 and 34, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NO:2n-1, wherein n is an integer between 1 and 34, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO:2n-1, wherein n is an integer between 1 and 34, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method .of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. No. 4,683,195 and U.S. Pat. No. 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7:244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217:286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15:1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86:2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17:2437-2448) or at the extreme 3′-terminus of one primer where, under appropriate conditions, mismatch can prevent, or reduce polymerase extension (see, e.g., Prossner, 1993. Tibtech. 11:238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e. to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderna, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

If In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A. NOVX Clone Information

### Example 1A.

The NOV1 clone was analyzed, and the nucleotide and encoded polypeptide sequences are shown in Table 1A.

TABLE 1A
NOV1 Sequence Analysis

| | SEQ ID NO: 1 | 2782 bp |
|---|---|---|
| NOV1 | GGGACCTCTACAGGGAAGACGGTGGGCCGGCCCTTGGGGGGGCTGATGTGTCCCCAAG | |
| CG59448-02 DNA | GCTGAGTCCCGTCAGGGTCTGGCCTCTGCCTCAGGCCCCCAAGGAGCCGGCCCTACAC | |
| Sequence | CCCATGGGTTTGTCACTGCCCAAGGAGAAAGGGCTAAGACGGGAGTCCTGGGCCCAGA | |
| | GCCGAGATGAGCAGAACCTGCTGCAGCAGAAGAGGATCTGGGAGTCTCCTCTCCTTCT | |
| | AGCTGCCAAAGATAATGATGTCCAGGCCCTGAACAAGTTGCTCAAGTATGAGGATTGC | |
| | AAGGTGCACCAGAGAGGAGCCATGGGGGAAACAGCGCTACACATAGCAGCCCTCTATG | |
| | ACAACCTGGAGGCCGCCATGGTGCTGATGGAGGCTGCCCCGGAGCTGGTCTTTGAGCC | |
| | CATGACATCTGAGCTCTATGAGGGTCAGACTGCGCTGCACATCGCTGTTGTGAACCAG | |
| | AACATGAACCTGGTGCGAGCCCTGCTTGCCCGCAGGGCCAGTGTCTCTGCCAGAGCCA | |
| | CAGGCACTGCCTTCCGCCGTAGTCCCCGCAACCTCATCTACTTTGGGGAGCACCCTTT | |
| | GTCCTTTGCTGCCTGTGTGAACAGTGAGGAGATCGTGCGGCTGCTCATTGAGCATGGA | |
| | GCTGACATCCGGGCCCAGGACTCCCTGGGAAACACAGTGTTACACATCCTCATCCTCC | |
| | AGCCCAACAAAACCTTTGCCTGCCAGATGTACAACCTGTTGCTGTCCTACGACAGACA | |
| | TGGGGACCACCTGCAGCCCCTGGACCTCGTGCCCAATCACCAGGGTCTCACCCCTTTC | |
| | AAGCTGGCTGGAGTGGAGGGTAACACTGTGATGTTTCAGCACCTGATGCAGAAGCGGA | |
| | AGCACACCCAGTGGACGTATGGACCACTGACCTCGACTCTCTATGACCTCACAGAGAT | |
| | CGACTCCTCAGGGGATGAGCAGTCCCTGCTGGAACTTATCATCACCACCAAGAAGCGG | |
| | GAGGCTCGCCAGATCCTGGACCAGACGCCGGTGAAGGAGCTGGTGAGCCTCAAGTGGA | |
| | AGCGGTACGGGCGGCCGTACTTCTGCATGCTGGGTGCCATATATCTGCTGTACATCAT | |
| | CTGCTTCACCATGTGCTGCATCTACCGCCCCCTCAAGCCCAGGACCAATAACCGCACA | |
| | AGCCCCCGGGACAACACCCTCTTACAGCAGAAGCTACTTCAGGAAGCCTACGTGACCC | |
| | CTAAGGACGATATCCGGCTGGTCGGGGAGCTGGTGACTGTCATTGGGGCTATCATCAT | |
| | CCTGCTGGTAGAGGTTCCAGACATCTTCAGAATGGGGGTCACTCGCTTCTTTGGACAG | |
| | ACCATCCTTGGGGGCCCATTCCATGTCCTCATCATCACCTATGCCTTCATGGTGCTGG | |
| | TGACCATGGTGATGCGGCTCATCAGTGCCAGCGGGGAGGTGGTACCCATGTCCTTTGC | |
| | ACTCGTGCTGGGCTGGTGCAACGTCATGTACTTCGCCCGAGGATTCCAGATGCTAGGC | |
| | CCCTTCACCATCATGATTCAGAAGATGATTTTTGGCGACCTGATGCGATTCTGCTGGC | |
| | TGATGGCTGTGGTCATCCTGGGCTTTGCTTCAGCCTTCTATATCATCTTCCAGACAGA | |
| | GGACCCCGAGGAGCTAGGCCACTTCTACGACTACCCCATGGCCCTGTTCAGCACCTTC | |
| | GAGCTGGTCCTTACCATCATCGATGGCCCAGCCAACTACAACGTGGACCTGCCCTTCA | |
| | TGTACAGCATCACCTATGCTGCCTTTGCCATCATCGCCACACTGCTCATGCTCAACCT | |
| | CCTCATTGCCATGATGGGCGACACTCACTGGCGAGTGGCCCATGAGCGGGATGAGCTG | |
| | TGGAGGGCCCAGATTGTGGCCACCACGGTGATGCTGGAGCGGAAGCTGCCTCGCTGCC | |
| | TGTGGCCTCGCTCCGGGATCTGCGGACGGGAGTATGGCCTGGGGGACCGCTGGTTCCT | |
| | GCGGGTGGAAGACAGGCAAGATCTCAACCGGCAGCGGATCCAACGCTACGCACAGGCC | |
| | TTCCACACCCGGGGCTCTGAGGATTTGGACAAAGACTCAGTGGAAAAACTAGAGCTGG | |
| | GCTGTCCCTTCAGCCCCCACCTGTCCCTTCCTACGCCCTCAGTGTCTCGAAGTACCTC | |
| | CCGCAGCAGTGCCAATTGGGAAAGGCTTCGGCAAGGGACCCTGAGGAGAGACCTGCGT | |
| | GGGATAATCAACAGGGGTCTGGAGGACGGGGAGAGCTGGGAATATCAGATCTGACTGC | |
| | GTGTTCTCACTTCGCTTCCTGGAACTTGCTCTCATTTTCCTGGGTGCATCAAACAAAA | |
| | CAAAAACCAAACACCCAGAGGTCTCATCTCCCAGGCCCCAGGGAGAAAGAGGAGTAAGC | |
| | ATGAACGCCAAGGAATGTACGTTGAGAATCACTGCTCCAGGCCTGCATTACTCCTTCA | |
| | GCTCTGGGGCAGAGGAAGCCCAGCCCAAGCACGGGGCTGGCAGGGCGTGAGGAACTCT | |
| | CCTGTGGCCTGCTCATCACCCTTCCGACAGGAGCACTGCATGTCAGAGCACTTTAAAA | |
| | ACAGGCCAGCCTGCTTGGGCCCTCGGTCTCCACCCCAGGGTCATAAGTGGGGAGAGAG | |
| | CCCTTCCCAGGGCACCCAGGCAGGTGCAGGGAAGTGCAGAGCTTGTGGAAAGCGTGTG | |
| | AGTGAGGGAGACAGGAACGGCTCTGGGGGTGGGAAGTGGGGCTAGGTCTTGCCAACTC | |
| | CATCTTCAATAAAGTCGTTTTCGGATCCCTAAAAAAAAAAAAAAAAAAAAAAAAAA | |
| | ORF Start: ATG at 120 | ORF Stop: TGA at 2256 |
| | SEQ ID NO: 2 | 712 aa MW at 81439.8 kD |

| | |
|---|---|
| NOV1 | MGLSLPKEKGLRRESWAQSRDEQNLLQQKRIWESPLLLAAKDNDVQALNKLLKYEDCK |
| CG95448-02 Protein | VHQRGAMGETALHIAALYDNLEAAMVLMEAAPELVFEPMTSELYEGQTALHIAVVNQN |
| Sequence | MNLVRALLARRASVSARATGTAFRRSPRNLIYFGEHPLSFAACVNSEEIVRLLIEHGA |
| | DIRAQDSLGNTVLHILILQPNKTFACQMYNLLLSYDRHGDHLQPLDLVPNHQGLTPFK |
| | LAGVEGNTVMFQHLMQKRKHTQWTYGPLTSTLYDLTEIDSSGDEQSLLELIITTKKRE |
| | ATQILDQTPVKELVSLKWKRYGRPYFCMLGAIYLLYIICFTMCCIYRPLKPRTNNRTS |
| | PRDNTLLQQKLLQEAYVTPKDDIRLVGELVTVIGAIIILLVEVPDIFRMGVTRFFGQT |
| | ILGGPFHVLIITYAFMVLVTMVMRLISASGEVVPMSFALVLGWCNVMYFARGFQMLGP |
| | FTIMIQKMIFGDLMRFCWLMAVVILGFASAFYIIFQTEDPEELGHFYDYPMALFSTFE |
| | LVLTIIDGPANYNVDLPFMYSITYAAFAIIATLLMLNLLIAMMGDTHWRVAHERDELW |
| | RAQIVATTVMLERKLPRCLWPRSGICGREYGLGDRWFLRVEDRQDLNRQRIQRYAQAF |
| | HTRGSEDLDKDSVEKLELGCPFSPHLSLPTPSVSRSTSRSSAVWERLRQGTLRRDLRG |
| | IINRGLEDGESWEYQI |

Further analysis of the NOV1 protein yielded the following properties shown in Table 1.

TABLE 1B
Protein Sequence Properties NOV1

| | |
|---|---|
| PSort | 0.6000 probability located in plasma membrane; 0.4000 probability located in Golgi |
| analysis: | body; 0.3000 probability located in endoplasmic reticulum (membrane); 0.0300 |
| | probability located in mitochondrial inner membrane |
| SignalP | No Known Signal Sequence Predicted |
| analysis: | |

A search of the NOV1 protein against the Geneseq database, a proprietary database that contains sequences published in patents and patent publication, yielded several homologous proteins shown in Table 1C.

TABLE 1C
Geneseq Results for NOV1

| | | NOV1 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Portion | Value |
| AAU00412 | Human calcium ion channel protein | 1 . . . 712 | 708/725 (97%) | 0.0 |
| | VANILREP5 - Homo sapiens , 725 aa. | 1 . . . 725 | 709/725 (97%) | |
| | [WO200114423-A1, 01-MAR-2001] | | | |
| AAG63210 | Amino acid sequence of novel human | 1 . . . 712 | 708/725 (97%) | 0.0 |
| | gene hCCh4 - Homo sapiens , 725 aa. | 1 . . . 725 | 709/725 (97%) | |
| | [WO200153348-A2, 26-JUL-2001] | | | |
| AAG65786 | Human ion channel VR-3 protein | 1 . . . 712 | 707/725 (97%) | 0.0 |
| | sequence - Homo sapiens , 725 aa. | 1 . . . 725 | 708/725 (97%) | |
| | [WO200168857-A2, 20-SEP-2001] | | | |
| AAB31595 | Amino acid sequence of a human | 1 . . . 712 | 706/725 (97%) | 0.0 |
| | calcium-transport protein - Homo | 1 . . . 725 | 708/725 (97%) | |
| | sapiens, 725 aa. [WO200104303-A1, | | | |
| | 18-JAN-2001] | | | |
| AAU00413 | Human calcium ion channel protein | 30 . . . 712 | 679/683 (99%) | 0.0 |
| | VANILREP5 splice variant #1 - Homo | 50 . . . 732 | 680/683 (99%) | |
| | sapiens, 732 aa. [WO200114423-A1, | | | |
| | 01-MAR-2001] | | | |

In a BLAST search of public sequence datbases, the NOV1 protein was found to have homology to the proteins shown in the BLASTP data in Table 1D.

TABLE 1D
Public BLASTP Results for NOV1

| | | NOV1 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9H1D1 | CAT-LIKE A PROTEIN - Homo | 1 . . . 712 | 712/725 (98%) | 0.0 |
| | sapiens (Human), 725 aa. | 1 . . . 725 | 712/725 (98%) | |
| Q9H1D0 | CAT-LIKE B PROTEIN - Homo | 1 . . . 712 | 709/725 (97%) | 0.0 |
| | sapiens (Human), 725 aa. | 1 . . . 725 | 710/725 (97%) | |
| AAL40230 | CALCIUM TRANSPORT | 1 . . . 712 | 708/725 (97%) | 0.0 |
| | PROTEIN CAT1 - Homo sapiens | 1 . . . 725 | 709/725 (97%) | |
| | (Human), 725 aa. | | | |
| CAC93826 | SEQUENCE 1 FROM PATENT | 1 . . . 712 | 707/725 (97%) | 0.0 |
| | WO0168857 - Homo sapiens | 1 . . . 725 | 708/725 (97%) | |
| | (Human), 725 aa. | | | |
| Q9H296 | CALCIUM TRANSPORT | 1 . . . 712 | 706/725 (97%) | 0.0 |
| | PROTEIN CAT1 - Homo sapiens | 1 . . . 725 | 708/725 (97%) | |
| | (Human), 725 aa. | | | |

PFam analysis predicts that the NOV1 protein contains the domains shown in the Table 1E.

TABLE 1E
Domain Analysis of NOV1

| | | Identities/ | |
|---|---|---|---|
| | | Similarities | |
| Pfam Domain | NOV1 Match Region | for the Matched Region | Expect Value |
| ank: domain 1 of 4 | 31 . . . 64 | 9/34 (26%) | 44 |
| | | 21/34 (62%) | |
| ank: domain 2 of 4 | 65 . . . 95 | 11/33 (33%) | 0.042 |
| | | 24/33 (73%) | |
| ank: domain 3 of 4 | 103 . . . 135 | 13/33 (39%) | 4.8e-06 |
| | | 26/33 (79%) | |
| ank: domain 4 of 4 | 149 . . . 181 | 15/33 (45%) | 8.7e-07 |
| | | 26/33 (79%) | |
| ion_trans: domain 1 of 1 | 396 . . . 565 | 34/229 (15%) | 6.9e-16 |
| | | 126/229 (55%) | |

### Example 2.

The NOV2 clone was analyzed, and the nucleotide and encoded polypeptide sequences are shown in Table 2A.

TABLE 2A
NOV2 Sequence Analysis

| | SEQ ID NO:3 | 11051 bp |
|---|---|---|
| NOV2a, | TCAAGGAAAAGTGTTTAAGCTTCTAAAATGTCATCTATCAAGCACCTGGTTTATGCAG | |
| CG59706-01 DNA | TTATTCGTTTCTTACGGGAACAAAGTCAGATGGACACTTACACCTCGGATGAACAAGA | |
| Sequence | AAGTTTGGAAGTTGCAATTCAGTGCTTGGAGACAGTTTTTAAGATCAGCCCAGAAGAT | |
| | ACACACCTAGCAGTTTCACAGCCTTTGACAGAAATGTTTACCAGTTCCGGACGAGACT | |
| | GTATGCCAAAAGGGGCCCAGAGGCCGCGCATCCCACCTATCCAATCGGTCACACAGCA | |
| | AGACTCTGTCTCAAGAGAAAAAAAAAAGACAAAGGGCAATAACCACATGAAAGAAGAA | |
| | AATTATGCTGCTGCAGTGGATTGTTACACACAGGCAATAGAATTGGATCCCAATAATG | |
| | CAGTTTACTATTGCAACAGGAGGGCTGCTGCTCAGAGCAAATTAGGTCACTACACAGA | |
| | TGCGATAAAGGATTGTGAAAAAGCAATAGCAATTGATTCAAAGTACAGCAAGGCCTAT | |
| | GGGAGAATGGGGCTGGCCCTCACTGCCTTGAATAAATTTGAAGAAGCAGTTACAAGTTT | |
| | ATCAAAAGGCATTAGATCTTGACCCTGAAATGATTCCTATAAGTCAAATCTCAAAAT | |
| | AGCAGAACAGAAGTTAAGAGAGGTATCCAGTCCTGTAACAGGAACTGGACTGAGCTTT | |
| | GACATGGCTAGCTTGATAAATAATCCAGCCTTCATTAGTATGGTGAGTATACTTATGC | |
| | AGAACCCTCAAGTTCAACAGCTGAAAAATGGTGTGGCGTCAGGCGCCCATAATCCCAG | |
| | CCACCCTATTCAAACCACATTGCCTCTTTACTACAGGGGACAGCAGTTTGCTCAGCAG | |
| | ATACACCAACAAAATCCTGAACTTATAGAGCAACTGAGAAATCACATCCGGAGCAGAT | |
| | CATTCAGCAGCAGCGCTGAAGAGCATTCCTGATTTAACCAGGGGCTCAAGCCCAAGAT | |
| | ACAATGGTTTATGGCTATGAATGAAGTATTTGTTGTAGATAGTACCCCCTCCCTCCT | |
| | TCAAAAA | |
| | ORF Start: ATG at 28 | ORF Stop: TGA at 958 |
| | SEQ ID NO:4 | 310 aa MW at 34846.8 kD |
| NOV2a. | MSSTKHLVYAVIRFLREQSQMDTYTSDEQESLEVAIQCLETVFKISPEDTHLAVSQPL | |
| CG59706-01 Protein | TEMFTSSGRDCMPKGAQRPRIPPIEWVTEQDSVSREKKKTKGNNHMKEENYAAAVDCY | |
| Sequence | TQAIELDPNNAVYYCNRRAAAQSKLGHYTDAIKDCEKAIAIDSKYSKAYGRMGLALTA | |
| LNKFEEAVTSYQKALDLDPENDSYKSNLKIAEQKLREVSSPVTGTGLSFDMASLINNP | | |
| AFISMVSTLMQNPQVQQLKNGVASGAHNPSHPTQTTLPLYYkGQQFAQQIQQQNPELI | | |
| EQLRNHIRSRSFSSSAEEHS | | |
| | SEQ ID NO:5 | 1009 bp |
| NOV2b, | TCTAAAATGTCATCTATCAAGCACCTGGTTTATGCAGTTATTCGTTTCTTACGGGAAC | |
| CG59706-02 DNA | AAAGTCAGATGGACACTTACACCTCGGATGAACAAGAAAGTTTGGAAGTTGCAATTCA | |
| Sequence | GTGCTTGAGACAGTTTTTAAGATCAGCCCAGAAGATACACACCTAGCAGTTTCACAG | |
| | CCTTTGACAGAAATGTTTACCAGTTCCTTCTGTAAGAATGACGTTCTGCCCCTTTCAA | |
| | ACTCAGTGCCTGAAGATGTGGGAAAAGCTGACCAATTAAAAGATGAAGGCAATAACCA | |
| | CATGAAAGAAGAAAATTATGCTGCTGCAGTGGATTGTTACACACAGGCAATAGAATTG | |
| | GATCCCAATAATGCAGTTTACTATTGCAACAGGGCTGCTGCTCAGAGCAAATTAGGTC | |
| | ACTACACAGATGCGATAAAGGATTGTGAAAAAGCAATAGCAATTGATTCAAAGTACAG | |
| | CAAGGCCTATGGGAGAATGGGGCTGGCCCTCACTGCCTTGAATAAATTTGAAGAAGCA | |
| | GTTACAAGTTATCAAAAGGCATTAGATCTTGACCCTGAAAATGATTCCTATAAGTCAA | |
| | ATCTGAAAATAGCAGAACAGAAGTTAAGAGAGGTATCCAGTCCTACAGGAACTGGACT | |
| | GAGCTTTGACATGGCTAGCTTGATAAATAATCCAGCCTTCATTAGTATGGCGGCAAGT | |
| | TTAATGCAGAACCCTCAAGTTCAACAGCTAATGTCAGGAATCATCACAAATGCCATTG | |
| | GGGGACCTGCTGCTGGAGTTGGGGGCCTAACTGACCTGTCAAGCCTCATCCAAGCGGG | |
| | ACAGCAGTTTGCTCAGCAGATACAGCAACAAAATCCTGAACTTATAGAGCACTGAGA | |
| | AATCACATCCGGAGCAGATCATTCAGCAGCAGCGCTGAAGAGCATTCCTGATTTAACC | |
| | AGGGGCTCAAGCCCAAGATACAAATGGTTTATGGCTATGAATGAAGTATTTGTTGTAG | |
| | ATAGTACCCCCTCCCTCCTTCAA | |
| | ORF Start: ATG at 7 | ORF Stop: TGA at 919 |
| | SEQ ID NO: 6 | 304 aa MW at 33429.1 kD |

| | |
|---|---|
| NOV2b, | MSSIKHLVYAVIRFLREQSQMDTYTSDEQESLEVAIQCLETVFKISPEDTHLAVSQPL |
| CG59706-02 Protein | TEMFTSSFCKNDVLPLSNSVPEDVGKADQLKDEGNNHMKEENYVDCYTQAELDP |
| Sequence | NNAVYYCNRAAQSKLGHYTDAIKDCEKATATDSKYSKAYGRMGLALTALNKFEEAVT |
| | SYQKALDLDPENDSYKSNLKIAEQKLREVSSPTGTGLSFDAASLINNPAFISMAASLM |
| | QNPQVQQLMSGMMTNAIGGPAAGVGGLTDLSSLTQAGQQFAQQTQQQNPELIEQLRNH |
| | IRSRSFSSSAEEHS |

Sequence comparison of the above protein sequences yields the following sequence relationships shown in Table 2B.

TABLE 2B
Comparison of NOV2a against NOV2b.

| Protein | NOV2a Residues/ | Identities/ |
|---|---|---|
| Sequence | Match Residues | Similarities for the Matched Region |
| NOV2b | 1 . . . 310 | 237/316 (75%) |
| | 1 . . . 304 | 248/316 (78%) |

Further analysis of the NOV2a protein yielded the following properties shown in Table 2C.

TABLE 2C
Protein Sequence Properties NOV2a

| | |
|---|---|
| PSort | 0.4961 probability located in mitochondrial matrix space; 0.3000 probability located |
| analysis: | in micro body (peroxisome); 0.2127 probability located in mitochondrial inner |
| | membrane; 0.2127 probability located in mitochondrial intermembrane space |
| SignalP | No Known Signal Sequence Predicted |
| analysis: | |

A search of the NOV2a protein against the Geneseq database, a proprietary database that contains sequences published in patents and patent publication, yielded several homologous proteins shown in Table 2D.

TABLE 2D
Geneseq Results for NOV2a

| | | NOV2a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU69429 | Lung small cell carcinoma antigen #23 | 1 . . . 308 | 181/317 (57%) | 2e-88 |
| | - Homo sapiens , 349 aa. | 37 . . . 346 | 231/317 (72%) | |
| | [WO200177168-A2, 18-OCT-2001] | | | |
| ABG07797 | Novel human diagnostic protein #7788 | 1 . . . 308 | 163/323 (50%) | 8e-71 |
| | - Homo sapiens , 355 aa. | 37 . . . 352 | 217/323 (66%) | |
| | [WO200175067-A2, 11-OCT-2001] | | | |
| ABG07797 | Novel human diagnostic protein #7788 | 1 . . . 308 | 163/323 (50%) | 8e-71 |
| | - Homo sapiens , 355 aa. | 37 . . . 352 | 217/323 (66%) | |
| | [WO200175067-A2, 11-OCT-2001] | | | |
| AAM93168 | Human digestive system antigen SEQ | 180 . . . 310 | 106/135 (78%) | 6e-48 |
| | ID NO: 2517 - Homo sapiens , 144 aa. | 11 . . . 144 | 112/135 (82%) | |
| | [WO200155314-A2, 02-AUG-2001] | | | |
| AAG80155 | SGT domain protein fragment - | 94 . . . 215 | 82/122 (67%) | 7e-40 |
| | Unidentified, 122 aa. [DE10018335- | 2 . . . 122 | 99/122 (80%) | |
| | A1, 04-OCT-2001] | | | |

In a BLAST search of public sequence datbases, the NOV2a protein was found to have homology to the proteins shown in the BLASTP data in Table 2E.

TABLE 2E
Public BLASTP Results for NOV2a

| | | NOV2a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96EQ0 | SIMILAR TO SMALL | 1 . . . 310 | 256/316 (81%) | e-132 |
| | GLUTAMINE-RICH | 1 . . . 304 | 267/316 (84%) | |
| | TETRATRICOPEPTIDE | | | |
| | REPEAT (TPR)-CONTAINING - | | | |
| | Homo sapiens (Human), 304 aa. | | | |
| AAH17611 | HYPOTHETICAL 33.4 KDA | 1 . . . 310 | 247/314 (78%) | e-128 |
| | PROTEIN - Mus musculus (Mouse), | 1 . . . 304 | 264/314 (83%) | |
| | 304 aa. | | | |
| T08782 | hypothetical protein | 1 . . . 308 | 181/317 (57%) | 4e-88 |
| | DKFZp586N1020.1 - human, 349 aa | 37 . . . 346 | 231/317 (72%) | |
| | (fragment). | | | |
| Q9BTZ9 | HYPOTHETICAL 35.6 KDA | 1 . . . 308 | 181/317 (57%) | 4e-88 |
| | PROTEIN - Homo sapiens (Human), | 17 . . . 326 | 231/317 (72%) | |
| | 329 aa (fragment). | | | |
| O43765 | Small glutamine-rich tetratricopeptide | 1 . . . 308 | 181/317 (57%) | 4e-88 |
| | repeat-containing protein - Homo | 1 . . . 310 | 231/317 (72%) | |
| | sapiens (Human), 313 aa. | | | |

PFam analysis predicts that the NOV2a protein contains the domains shown in the Table 2F.

TABLE 2F
Domain Analysis of NOV2a

| | | Identities/ | |
|---|---|---|---|
| | | Similarities | |
| Pfam Domain | NOV2a Match Region | for the Matched Region | Expect Value |
| TPR: domain 1 of 3 | 93 . . . 126 | 14/34 (41%) | 0.00026 |
| | | 27/34 (79%) | |
| TPR: domain 2 of 3 | 128 . . . 161 | 12/34 (35%) | 2.6e-06 |
| | | 28/34 (82%) | |
| TPR: domain 3 of 3 | 162 . . . 195 | 16/34 (47%) | 2.7e-09 |
| | | 30/34 (88%) | |

### Example 3.

The NOV3 clone was analyzed, and the nucleotide and encoded polypeptide sequences are shown in Table 3A.

TABLE 3A
NOV3 Sequence Analysis

| | SEQ ID NO: 7 | 2330 bp |
|---|---|---|
| NOV3a, | AAGGCGGAAAAGCTCTCCGGAGTCCAAGTGGCCAGACAGATGGCAGCGCATGTATGTG | |
| CG59766-01 | CACACAACTAGATGGTGTGGCTGGAACTGGGTAAGTGACCCCAAACACAGGCTTTCCCTC | |
| Sequence | CCGAAGGGGTCATCTGGAGAACAACCTGATGGTACCAAGATTGAGCTATCATCTGA | |
| | TAATAATGAATGATTGAGAATCTCAAAATAAAGAAATCCTGCCAAACAACTGACCTCA | |
| | GAAAGATGAAAGTTTCCAAATATTCTTTGGGAGAACATGTTTTTGGGTTGATTGTTTC | |
| | CGATTTATCTTGAGGACAGGGTGTACGTGAGATCATGGTGAAATGGGCCGGAAAA | |
| | TAGTGCTCTGCACTGAAGCCATGAAAAGCATTTCCTCTACTGGCGCCCGTTCA | |
| | CAGGCTCCTGTTTGACTTTATATCCTTCTGGAGGGAGGCTGCTCAGCTTCATACTC | |
| | CTTGCAGATCTGCTCCCACAGATTCTTGCTTGACGGCTCGGTGACCTGCCACGACGAG | |
| | AGTCTGTAGAGCAGCTGGGCTTGAGGCGCACTATGTGGCTGGGGATTTGCCGCGGTGC | |
| | CGCCATGGCCGCGGTTTCCACCGTAACCGCGTTCGCCCGAAGGCCGCGACCCGGGA | |
| | AGCCGGAACCCGAGAGGGTGGGCCGGCGACTCGAAGTGGACTTCCGGGTCACGGCGGA | |
| | GCTGGCTCTCACGTGGAGGCGGGGAAATTTCGCCCACCGGTGAGATGATCACCGAC | |
| | CCAGAGACTCGATACTCAGTTTTAAACAATGATGATTACTTTGCTGATGTTTCTCCTT | |
| | TAAGAGCTACATCCCCCTCTAAGAGTGTGGCCCATGGGCAGGCACCTGAGATGCCTCT | |
| | AGTGAAGAAAAAGAAGAAGAAAAAGAAGGGTGTCAGCACCCTTTGCGAGGAGCATGTA | |
| | GAACCTGAGACCACGCTGCCTGCTAGACGGACAGAGAAGTCACCCAGCCTCAGGAAGC | |
| | AGGTGTTTGGCCACTTGGAGTTCCTCAGTGGGGAAAAGAAAAATAAGAAGTCACCTCT | |
| | AAACCAGAGTTGGCAAGAAGCTCAAAAAACACAAGAAGGAAAAAAAGGGGGCCCAGG | |
| | GTGTTGGAAAAAAAAGGCAACATGGATGAGCCGCACATAGACCAGGTGAGCCGGG | |
| | CCTTGCAAGAAGAGATCGATCGCGAGTCAGGCAAAACGGAAGCTTCTGAAACCAGG | |
| | GTGGACGGGAACCCAGTTTGGCCAGTGGGATACTGCTGGTTTTGAGAACGAGGACCAA | |
| | AACTGAAATTTCTCAGACTTATGGGTGGCTTCAAAAACCTGTCCCCTTCGTTCAGCC | |
| | GCCCCGCCAGCACGATTGCAAGGCCCAACATGGCCCTCGGCAAGAAGGCGGCTCACAG | |
| | CCTGCAGCAGAATCTGCAGCGGGACTACGACCGGGCCATGAGCTGGAAGTACAGCCGG | |
| | GGAGCCGGCCTCGGCTTCTCCACCGCCCCCAACAAGATCTTTTACATTGACAGGACG | |
| | CTTCCAAGTCAGTCAAGCTGGAAGATTAAACTCTAGAGTTTTGTCCCCCCCTGC | |
| | CACAATTCCTTTGATTATTCCATTTATCCTGGAGATTACAAATTTTTTTTGTGAAAAA | |
| | ATCAGATCTT | |
| | ORF Start: ATG at 671 | ORF Stop: TAA at 2231 |
| | SEQ ID NO: 8 | 520 aa MW at 58132.9 kd |
| NOV3a, | MWLGICRGAAD4AAVSTVTAFAGRPRPGRSRNPRGWAGDSKWTSGSRRSWLSRGGGEIS | |
| CG59766-01 Protein | PTGEMITKTHVDLGLPEKKKKKKVVKEPETRYSVLNNDDYFADVSPLRATSPSKSVA | |
| Sequence | HGQAPEMPLVKKKKKKKKGVSTLCEEHVEPETTLPARRTEKSPSLRKQVFGHLEFLSG | |
| | EKKNKKSPLANSIIASGVKTSPDPRQGEEETRVGKKLKKHKKEKKGAQDPTAFSVQDPW | |
| | FCEAREARDVGDTCSVGKKDEEQAALGQKRKRKSPREHNGKVKKKKKIHQEGDALPGH | |
| | SKPSRSMESSPRKGSKKKPVKVEAPEYIPISDDPKASAKKKMKSKKKVEQPVIEEPAL | |
| | KRKKKKKRKESGVAGDPWKEETDTDLEVVLEKKGNMDEAHIDQVRRLQEEIDRESGI | |
| | KTEASETRKWTGTQFGQWDTAGFENEDQKLKFLRLMGGFKNLSPSFSRPASTIARPNM | |
| | ALGKKAADSLQQNLQRDYDRANSWKYSRGAGLGFSTAPNKIFYIDRNASKSVKLED | |
| | SEQ ID NO: 9 | 2261 bp |
| NOV3b, | AAGGCGGAA3AGCTCTCCGGAGTCCAAGTGGCCAGACAGATGGCAGCGCATGTATGTG | |
| CG59766-02 DNA | CACAACTAGATGGTCTGGCTGGAACTGGGTAAGTGACCCCAAACACAGGCTTTCCCTC | |
| Sequence | CCGAAGGGGTCATCTGGAGAACAACCTGATGGTACCAAGATAATGAGCTATCATCT | |
| | TAATAATGAATGATTGAGAATCTCAAAATAAGAAATCCTGCCCCTGACCTCA | |
| | AAACATTTTTCTTTCTTCGCTTGGTGAAGCAGGCTAGCCATTCCGGGGCAGCAG | |
| | GAAAGATGAAAGTTTCCAATATTCTTTGGGAGAACATGTTTTTGGGTTGATTGTTTC | |
| | TAGTGCTCTGCACTGAAGCCATGAAAAGCATTTCCTCTACTGGCGCCCGTAATCA | |
| | CTTGCAGATCTGCTCCCACAGATTCTTGCTTGACGGCTCGGTGACCTCCACAGAG | |
| | ACAGGCAGCTCGCGGGACGCGAGAGACACGGTGGGCACCGGCGTCCGGGTGCGACG | |
| | AGTCTGTAGAGCAGCTGGGCTTGAGGCGCACTATGTGGCTGGGGATTTGCCGCGGTGC | |
| | CGCCATGGCCGCGGTTTCCACGGTAACCGCOTTCGCCGGAAGGCCGCGACCCGGAAGA | |
| | AGCCGGAACCCGAGAGGGTGGGCCGGCGACTCGAAGTGGACTTCCGGGTCACGGCGGA | |
| | GCTGGCTCTCACGTGGAGGCGGGGAAATTTCGCCCACCGGTGAGATGATCACCGAC | |
| | CCAGAGACTCGATACTCAGTTTTAAACAATGATGATTACTTTGCTGATGTTTCTCCTT | |
| | TAAGAGCTACATCCCCCTCTAAGAGTGTGGCCCATGGGCAGGCACCTGAGATGCCTCT | |
| | GAACCTGAGACCACGCTGCCTGCTAGACGGACAGAGAAGTCACCCAGCCTCAGGAAGC | |
| | AGGTGTTTGGCCACTTGGAGTTCCTCAGTGGGGAAAAGAAAAATAAGAAGTCACCTCT | |
| | AGCCATGTCCCATGCCTCTGGGGTGAAACCTCCCCAGACCCTAGACAGGGTGAGGAG | |
| | GAAACCAGAGTTGGCAAGAAGCTCAAAAAACACAAGAAGGAAAAGGGGGCCCAGG | |
| | ACCCCACAGCCTTCTCGGTCCAGGACCCTTGGTTCTGTGAGGCCAGGGAGGCCAGGGA | |
| | TGTTGGGGACACTTGCTCAGTGGGGAAGAAGGATGAGGCAGGCAGCCTTGGGGCAG | |
| | TCCACCAGGAGGGAGATGCCCTCCCAGGCCACTCCAAGCCCTCCAGGTCCATGGAGAC | |
| | CAGCCCTAGGAAAGGAAGTAAAAAGAAGCCAGTCAAGTTGAGGCTCCGGAATACATC | |
| | CCCATAAGTGATGACCCTAAGGCCTCCGCAAAGAAAAAAGATGAAGTCCAAAAAGAAGG | |
| | TAGAGAGTGGGGTAGCAGGAGACCCTTGGAAGGAGGTGAGGCGGGCCTTGCG | |
| | GAGATCGATCGCGAGTCAGGCAAACGGAACCTTCTGAAACCAGGAAGTGGACGGGAA | |
| | TCTCAGACTTATGGGTGGCTTCAAAAACCTGTCCCCTTCGTTCAGCCGCCCCGCCACC | |
| | ACGATTGCAAGGCCCAACATGGCCCTCGGCAAGAAGGCGGCTGACAGCCTGCAGCAGA | |
| | ATCTGCAGCGGGACAACGACCCGGCCATGAGCTGGAAGTACAGCCGGGGAGCCGGCCT | |
| | CGGCTTCTCCACCGCCCCCAACAAGATCTTTTACATTGACAGGAACGCTTCCAAGTCA | |
| | GTCAAGCTGGAAGATTAAACTCTAGAGTTTTGTCCCCCCAAAACTGCCACTTGCTT | |
| | TGATTATTCCATTTATGCTGCAGATTACAAATTTTTTTTGTGAAAAATCAGATCTT | |
| | ORF Start: ATG at 671 | ORF Stop: TAA at 2162 |
| | SEQ ID NO: 10 | 497 aa MW at 55413.0 kD |

| | |
|---|---|
| NOV3b, | MWLGTCRGAAMAAVSTVTAFAGRPRPGRSRNPRGWAGDSKWTSGSRRSWLSRGGGEIS |
| CG59766-02 Protein | PTGEMTTKTHKVDLGLPEKKKKKKVVKEPETRYSVLNNDDYFADVSPLPATSPSKSVAI |
| Sequence | HGQAPEMPLVKKKKKKKKGVSTLCEEHVEPETTLPARRTEKSPSLRKQVFGHLEFLSG |
| | EKKNKKSPLANSHASGVKTSPDPRQGEEETRVGKKLKKKEKKGAQDPTAFSVQDPW |
| | FCEAREARDVGDTCSVGKKDEEQAALGQKRKRKSPREHNGKVKKKKKIHQEGDALPGH |
| | SKPSRSMESSPRKGSKKKPVKVEAPEYPISDDPKASAKKKMKSKKKVEQPVIEPAL |
| | KRKKKKKRKESGVAGDPWKEVRRKALQEETDRESGKTEASETRKWTGTQFGQWDTAGF |
| | ENEDQKLKFLRLMGGFKNLSPSFSRPASTIARPNMALGKKAADSLQQNLQRDNDPAMS |
| | WKYSRGAGLGFSTAPNKIFYIDRNASKSVKLED |

Sequence comparison of the above protein sequences yields the following sequence relationships shown in Table 3B.

TABLE 3B
Comparison of NOV3a against NOV3b.

| Protein | NOV3a Residues/ | Identities/ |
|---|---|---|
| Sequence | Match Residues | Similarities for the Matched Region |
| NOV3b | 1 . . . 520 | 407/520 (78%) |
| | 1 . . . 497 | 407/520 (78%) |

Further analysis of the NOV3a protein yielded the following properties shown in Table 3C.

TABLE 3C
Protein Sequence Properties NOV3a

| | |
|---|---|
| PSort | 0.9701 probability located in nucleus; 0.7514 probability |
| analysis: | located in mitochondrial matrix space; 0.6015 probability |
| | located in mitochondrial intermembrane space; 0.4307 |
| | probability located in mitochondrial inner membrane |
| SignalP | Cleavage site between residues 22 and 23 |
| analysis: | |

A search of the NOV3a protein against the Geneseq database, a proprietary database that contains sequences published in patents and patent publication, yielded several homologous proteins shown in Table 3D.

TABLE 3D
Geneseq Results for NOV3a

| | | | Identities/ | |
|---|---|---|---|---|
| | | NOV3a | Similarities | |
| | Protein/ | Residues/ | for the | |
| Geneseq | Organism/Length | Match | Matched | Expect |
| Identifier | [Patent #, Date] | Residues | Region | Value |
| AAY60239 | Human | 64 . . . 520 | 454/457 | 0.0 |
| | endometrium | | (99%) | |
| | tumour EST | 1 . . . 456 | 455/457 | |
| | encoded protein | | (99%) | |
| | 299-Homo sapiens, | | | |
| | 456 aa. | | | |
| | [DE19817948-A1, | | | |
| | 21 Oct. 1999] | | | |
| AAB42548 | Human ORFX | 332 . . . 520 | 189/189 | e−106 |
| | ORF2312 | | (100%) | |
| | polypeptide | 1 . . . 189 | 189/189 | |
| | sequence SEQ ID | | (100%) | |
| | NO: 4624-Homo | | | |
| | sapiens, 189 aa. | | | |
| | [WO200058473-A2, | | | |
| | 05 Oct. 2000] | | | |
| AAM78825 | Human protein | 53 . . . 415 | 85/386 | 1e−12 |
| | SEQ ID NO 1487- | | (22%) | |
| | Homo sapiens, | 489 . . . 867 | 147/386 | |
| | 1026 aa. | | (38%) | |
| | [WO200157190-A2, | | | |
| | 09-AUG-2001] | | | |
| AAM79809 | Human protein | 53 . . . 415 | 87/387 | 2e−12 |
| | SEQ ID NO 3455- | | (22%) | |
| | Homo sapiens, | 495 . . . 874 | 146/387 | |
| | 1033 aa. | | (37%) | |
| | [WO200157190-A2, | | | |
| | 09 Aug. 2001] | | | |
| AAM04187 | Peptide #2869 | 53 . . . 415 | 84/386 | 9e−11 |
| | encoded by probe | | (21%) | |
| | for measuring breast | 86 . . . 458 | 148/386 | |
| | gene expression- | | (37%) | |
| | Homo sapiens, | | | |
| | 617 aa. | | | |
| | [WO200157270-A2, | | | |
| | 09 Aug. 2001] | | | |

In a BLAST search of public sequence datbases, the NOV3a protein was found to have homology to the proteins shown in the BLASTP data in Table 3E.

TABLE 3E
Public BLASTP Results for NOV3a

| | | | Identities/ | |
|---|---|---|---|---|
| | | NOV3a | Similarities | |
| Protein | | Residues/ | for the | |
| Accession | Protein/ | Match | Matched | Expect |
| Number | Organism/Length | Residues | Portion | Value |
| Q9Z2Q2 | TSG118.1-Mus | 11 . . . 520 | 326/543 | e−155 |
| | musculus (Mouse), | | (60%) | |
| | 530 aa. | 1 . . . 530 | 377/543 | |
| | | | (69%) | |
| O43328 | HYPOTHETICAL | 332 . . . 520 | 189/189 | e−105 |
| | 21.5 KDA PROTEIN- | | (100%) | |
| | Homo sapiens | 1 . . . 189 | 189/189 | |
| | (Human), 189 aa. | | (100%) | |
| Q9D7H7 | 2310008H09RIK | 288 . . . 520 | 146/268 | 2e−68 |
| | PROTEIN-Mus | | (54%) | |
| | musculus (Mouse), | 53 . . . 318 | 177/268 | |
| | 318 aa. | | (65%) | |
| Q28687 | NEUROFILAMENT- | 74 . . . 415 | 97/366 | 3e−17 |
| | H-Oryctolagus | | (26%) | |
| | cuniculus (Rabbit), | 123 . . . 485 | 148/366 | |
| | 606 aa (fragment). | | (39%) | |
| Q95XW8 | HYPOTHETICAL | 76 . . . 415 | 89/371 | 7e−17 |
| | 77.9 KDA PROTEIN- | | (23%) | |
| | Caenorhabditis | 262 . . . 615 | 151/371 | |
| | elegans, 679 aa. | | (39%) | |

PFam analysis predicts that the NOV3a protein contains the domains shown in the Table 3F.

TABLE 3F
Domain Analysis of NOV3a

| | | | |
|---|---|---|---|
| | | Identities/ | |
| | NOV3a | Similarities | Expect |
| Pfam Domain | Match Region | for the Matched Region | Value |

No Significant Matches Found

### Example 4.

The NOV4 clone was analyzed, and the nucleotide and encoded polypeptide sequences are shown in Table 4A.

TABLE 4A
NOV4 Sequence Analysis

| | SEQ ID NO: 11 | 638 bp |
|---|---|---|
| NOV4, | TTCATAACTGTCCAATACAGCCTTACCATGGCGGCGCGGACGGCGTTTGGGGCCGTG | |
| CG598 13-01 DNA | GCCGGCGCCTCTGGCAGGGATTGGGGAATTTTTCTGTAAACAGTTCTAAGGGCAATA | |
| Sequence | AGCCAAAAATGGTGGCTTTCTTCTCAGTACCAATATGAAGTGGGTACAGTTTTCACI | |
| | CTACACGTTGATGTTCCAAGGATTTCACCAAACCTGTGATAACTCTCTGATGC I | |
| | CAGACACATTATATAAAATTTTAATTCTTATATTGTCACACGGTAAGGCTGTATTGGA | |
| | CAGTTATGAATATTTTGCTGTGCTTGATGCTAAGAACTTGGTATCTCTATTA.GTA | |
| | CACGAACCTCCAAGGAAAATAGAGCGATTTACTCTTCTCATATCAGTCCATATTTATA | |
| | AGAAGCACGGAGTTCAGTATGAAATGAGAACACTTTACAGATGTTTAGAGTTAGAACA | |
| | TCTAACTGGAAGCACAGCAGATGTCTACGTGGAATATATTCAGCGAAACTTACCTGAA | |
| | AGGGTTGCCATGGAGTAACAAGACAOATTAGAACAGTTACCAGAACACATCAAGG | |
| | AGCCAATCTGGGAAACACTATCAGAAGAAAAAGAAGAAAGCAAGTCTTGCCTCAG I | |
| | ORF Start: ATG at 28 | ORF Stop: TAA at 628 |
| | SEQ ID NO: 12 | 200 aa MW at 22937.2 kD |

| | |
|---|---|
| NOV4, | MAARTAFGAVCRRLWQGLGNFSVNSSKGNTAKNGGFLLSTNMKQFSNLHVDVPKDP |
| G59813-01 Protein | TKPVITISDEPDTLYKTLILILSHGVLDSYEYFAVLDAKELGISTKVHEPPRKIER |
| Sequence | FTLLTSVHIYKKHGVQYEMRTLYRCLELEHLTGSTADVYVEYTQRNLPERVAMEVTKT |
| QLEQLPEHIKEPIWETLSEEKEESKS | |

Further analysis of the NOV4 protein yielded the following properties shown in Table 4B.

TABLE 4B
Protein Sequence Properties NOV4

| | |
|---|---|
| PSort | 0.5595 probability located in mitochondrial matrix space; |
| analysis: | 0.2772 probability located in mitochondrial inner membrane; |
| | 0.2772 probability located in mitochondrial intermembrane |
| | space; 0.2772 probability located in mitochondrial outer |
| | membrane |
| SignalP | Cleavage site between residues 12 and 13 |
| analysis: | |

A search of the NOV4 protein against the Geneseq database, a proprietary database that contains sequences published in patents and patent publication, yielded several homologous proteins shown in Table 4C.

TABLE 4C
Geneseq Results for NOV4

| | | | Identities/ | |
|---|---|---|---|---|
| | | NOV4 | Similarities | |
| | Protein/ | Residues/ | for the | |
| Geneseq | Organism/Length | Match | Matched | Expect |
| Identifier | [Patent #, Date] | Residues | Region | Value |
| AAB92952 | Human protein | 1 . . . 200 | 183/201 | e−101 |
| | sequence SEQ ID | | (91%) | |
| | NO: 11633-Homo | 1 . . . 201 | 187/201 | |
| | sapeins, 201 aa. | | (92%) | |
| | [EP1074617-A2, | | | |
| | 07 Feb. 2001] | | | |
| AAB56904 | Human prostate | 2 . . . 200 | 182/200 | e−101 |
| | cancer antigen | | (91%) | |
| | protein sequence | 6 . . . 205 | 187/200 | |
| | SEQ ID NO: 1482- | | (93%) | |
| | Homo sapiens, | | | |
| | 205 aa. | | | |
| | [WO200055174-A1, | | | |
| | 21 Sept. 2000] | | | |
| AAM25553 | Human protein | 1 . . . 200 | 183/208 | e−100 |
| | SEQ ID NO: 1068- | | (87%) | |
| | Homo sapiens, | 8 . . . 215 | 188/208 | |
| | 215 aa. | | (89%) | |
| | [WO200153455-A2, | | | |
| | 26 July 2001] | | | |
| AAM80014 | Human protein | 1 . . . 173 | 156/174 | 2e−85 |
| | SEQ ID NO 3660- | | (89%) | |
| | Homo sapiens, | 8 . . . 181 | 161/174 | |
| | 288 aa. | | (91%) | |
| | [WO200157190-A2, | | | |
| | 09 Aug. 2001] | | | |
| AAM79030 | Human protein | 42 . . . 173 | 118/133 | 4e−62 |
| | SEQ ID NO 1692- | | (88%) | |
| | Homo sapiens, | 1 . . . 133 | 122/133 | |
| | 180 aa. | | (91%) | |
| | [WO200157190-A2, | | | |
| | 09 Aug. 2001] | | | |

In a BLAST search of public sequence datbases, the NOV4 protein was found to have homology to the proteins shown in the BLASTP data in Table 4D.

TABLE 4D
Public BLASTP Results for NOV4

| | | | Identities/ | |
|---|---|---|---|---|
| | | NOV4 | Similarities | |
| Protein | | Residues/ | for the | |
| Accession | Protein/ | Match | Matched | Expect |
| Number | Organism/Length | Residues | Portion | Value |
| P82664 | Mitochondrial 28S | 1 . . . 200 | 183/201 | e−101 |
| | ribosomal protein | | (91%) | |
| | S10 (MRP-S10) | 1 . . . 201 | 188/201 | |
| | (MSTP040)-Homo | | (93%) | |
| | sapiens (Human), | | | |
| | 201 aa. | | | |
| Q9BZS5 | PNAS-122-Homo | 1 . . . 106 | 92/107 | 3e−46 |
| | sapiens (Human), | | (85%) | |
| | 108 aa. | 1 . . . 107 | 96/107 | |
| | | | (88%) | |
| AAL49086 | RE54409P- | 68 . . . 186 | 63/122 | 5e−26 |
| | Drosophila | | (51%) | |
| | melanogaster | 38 . . . 159 | 84/122 | |
| | (Fruit fly), 163 aa. | | (68%) | |
| Q9VFB2 | CG4247 PROTEIN- | 68 . . . 186 | 63/122 | 5e−26 |
| | Drosophila | | (51%) | |
| | melanogaster | 46 . . . 167 | 84/122 | |
| | (Fruit fly), 171 aa. | | (68%) | |
| Q9XWV5 | Y37D8A.18 | 69 . . . 185 | 48/121 | 8e−11 |
| | PROTEIN- | | (39%) | |
| | Caenorhabditis | 37 . . . 155 | 68/121 | |
| | elegans, 156 aa. | | (55%) | |

PFam analysis predicts that the NOV4 protein contains the domains shown in the Table 4E.

TABLE 4E
Domain Analysis of NOV4

| | | | |
|---|---|---|---|
| | | Identities/ | |
| | NOV4 | Similarities | Expect |
| Pfam Domain | Match Region | for the Matched Region | Value |

No Significant Matches Found

### Example 5.

The NOV5 clone was analyzed, and the nucleotide and encoded polypeptide sequences are shown in Table 5A.

TABLE 5A
NOV5 Sequence Analysis

| | SEQ ID NO: 13 | 545 bp |
|---|---|---|
| NOV5, | GGATTTCCTGGGCTATTATGATGGTGACGAATTTCAAGTGGTTGTGGCAGTATCGCTT I | |
| CG59815-01 DNA | CCCGCCCTTTACATTACAGCTGAACGTGGCCACTTGGCAGAAGCAGCTGGCCACCTGG | |
| Sequence | TGTTTGTTGGTTCTGTCCATCTGCTGCCTGCACAGACAGTCAAGCATGATGGTTATGG | |
| | ATGCTCAGGAGATCCTGCTCTTCAGCAACATCAAGCTGTGGAAGCTTCCTGTGGGATC | |
| | AGCAAGTCTAGTTTCCTAATCATGTGGCGGAGGCCAGAAGAATGGGGAAAACTCATCT | |
| | ATCAGTGGGTCTCCAGGAGTGGCCAGAACAACTCCGTACTTAGCCTGTATGAGCTGAC | |
| | CAATGGGGAAGACATAGAGAATGAGGTGTTCCACGGACTAAAGGAGGCCTTCTGTGGG | |
| | CTCTGCAGGCCCTTCAGTAGGAACATAAGGCTGAGATCATCACCATCTCACTCGGAGA | |
| | CCAGTGATGGCTGAGGTGTCAGG | |
| | ORF Start: ATG at 18 | ORF Stop: TGA at 534 |
| | SEQ ID NO: 14 | 172 aa MW at 20203.3 kD |

| | |
|---|---|
| NOV5, | MMVTNFKWLWQYRFPPFTLQLNVATWQKQLATWCLLVLSICCLHRQSSMMVMDAQEIL |
| CG59815-01 Protein | LFSNTKLWKLPVGSIQVVLEELRKNGNLQWLDKSKSSFLINWRRPEEWGKLTYQSR |
| Sequence | SGQNNSVLSLYELTNGEDIENEVFHGLKEAFCGLCRPFSRNTRLRSSPSHSETSDG |

Further analysis of the NOV5 protein yielded the following properties shown in Table 5B.

TABLE 5B
Protein Sequence Properties NOV5

| | |
|---|---|
| PSort | 0.6400 probability located in microbody (peroxisome); |
| analysis: | 0.3600 probability located in mitochondrial matrix space; |
| | 0.3000 probability located in mitochondrial intermembrane |
| | space; 0.1000 probability located in lysosome (lumen) |
| SignalP | Cleavage site between residues 49 and 50 |
| analysis: | |

A search of the NOV5 protein against the Geneseq database, a proprietary database that contains sequences published in patents and patent publication, yielded several homologous proteins shown in Table 5C.

TABLE 5C
Geneseq Results for NOV5

| | | NOV5 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAG93262 | Human protein HP10149 - Homo | 2 . . . 146 | 115/147 (78%) | 9e-61 |
| | sapiens, 176 aa. [WO200142302-A1, | 1 . . . 147 | 125/147 (84%) | |
| | 14 JUN-2001] | | | |
| AAM41667 | Human polypeptide SEQ ID NO | 2 . . . 146 | 115/147 (78%) | 9e-61 |
| | 6598 - Homo sapiens , 226 aa. | 9 . . . 155 | 125/147 (84%) | |
| | [WO200153312-A1, 26-JUL-2001] | | | |
| AAM39881 | Human polypeptide SEQ ID NO 3026 - | 2 . . . 146 | 115/147 (78%) | 9e-61 |
| | Homo sapiens , 176 aa. | 1 . . . 147 | 125/147 (84%) | |
| | [WO200153312-A1, 26-JUL-2001] | | | |
| AAB10244 | Murine adult spleen protein fragment | 2 . . . 146 | 115/147 (78%) | 9e-61 |
| | AE402_li - Mus sp., 176 aa. | 1 . . . 147 | 125/147 (84%) | |
| | [WO200037630-A1, 29-JUN-2000] | | | |
| AAW54437 | Mouse novel secreted protein isolated | 2 . . . 82 | 59/83 (71%) | 1e-24 |
| | from clone AE402_li - Mus sp, 83 aa. | 1 . . . 83 | 66/83 (79%) | |
| | [WO9814470-A2, 09-APR-1998] | | | |

In a BLAST search of public sequence datbases, the NOV5 protein was found to have homology to the proteins shown in the BLASTP data in Table 5D.

TABLE 5C
Public BLASTP Results for NOV5

| | | NOV5 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9BRG1 | SIMILAR TO RIKEN CDNA | 2 . . . 146 | 115/147 (78%) | 2e-60 |
| | 1110020N13 GENE - Homo sapiens | 1 . . . 147 | 125/147 (84%) | |
| | (Human), 176 aa. | | | |
| Q9CQ80 | DNA SEGMENT, CHR 11, WAYNE | 2 . . . 146 | 113/147 (76%) | 2e-59 |
| | STATE UNIVERSITY 68, | 1 . . . 147 | 125/147 (84%) | |
| | EXPRESSED - Mus musculus | | | |
| | (Mouse), 176 aa. | | | |
| Q9D167 | 1110020N13RIK PROTEIN - Mus | 2 . . . 138 | 107/139 (76%) | 7e-56 |
| | musculus (Mouse), 148 aa. | 1 . . . 139 | 119/139 (84%) | |
| Q9U354 | W02A11.2 PROTEIN - Caenorhabditis | 6 . . . 144 | 55/141 (39%) | 3e-23 |
| | elegans, 183 aa. | 11 . . . 151 | 83/141 (58%) | |
| G87978 | protein W02A11.2 [imported] - | 6 . . . 138 | 52/135 (38%) | 3e-21 |
| | Caenorhabditis elegans, 155 aa. | 11 . . . 145 | 79/135 (58%) | |

PFam analysis predicts that the NOV5 protein contains the domains shown in the Table 5E.

TABLE 5E
Domain Analysis of NOV5

| | | | |
|---|---|---|---|
| | | Identities/ | |
| Pfam | | Similarities | Expect |
| Domain | NOV5 Match Region | for the Matched Region | Value |

No Significant Matches Found

### Example 6.

The NOV6 clone was analyzed, and the nucleotide and encoded polypeptide sequences are shown in Table 6A.

TABLE 6A
NOV6 Sequence Analysis

| | SEQ ID NO: 15 | 648 bp |
|---|---|---|
| NOV6, | CGGAGTCCCCTAACAATGGATAAATTCGTCATTCGAACGCCTAGAATCCAGAATAGCCI | |
| CG59817-02 DNA | CTCAGAACAAAGATTCTGGAGGAAAGGTTTACAAGCAGGCCACGATTGAATCTCTGAA | |
| Sequence | CAAACCAAAGAGAATCTTGTTGAAGCCTTACAAGAATTAAAGAAGAAAATACCCTCCA | |
| | GGGAAGTGTTAAAATCAACAAGGATAGGTCACACTGTGAACAAGATGCGTAAACACTC | |
| | AGATTCAGAAGTGGCTTCTCTTGCCAGAGAAGTTTACACTGAGTGGAAAACTTTCACT | |
| | GAAAAACATTCAAATAGACCTTCTATTGAAGTTAGAAGTGATCCCAAAACCGAGTCGT | |
| | TGAGGAAAAATGCTCAGAAATTACTCTCAGAAGCCTTGGAATTAAAGATGGATCACCT | |
| | ACTGGTTGAAAATATTGAACGGGAAACGTTTCATCTCTGCTCCCGCCTCATTAATGGG | |
| | CCGTACCGGCGGACGGTGAGAGCCCTGGTCTTCACATTAAAOCACCGAGCTGAAATCC | |
| | GGGCTCAGGTGAAGAGCGGCTCGCTGCCAGTCGGCACGTTTGTACAGACCCACAAAAA | |
| | GTGACCTGAG | |
| | ORF Start: ATG at 16 | ORF Stop: TGA at 640 |
| | SEQ ID NO: 16 | 208 aa MW at 24149.8 kD |

| | |
|---|---|
| NOV6, | MDKFVTRTPRIQNSPQKKDSGGKVYKQATTESLKRVVVVEDIKRWKTMLELPDQTKEN |
| CG59817-02 Protein | LVEALQELKKKIPSREVLKSTRTGHTVNKMRKHSDSEVASLAREVYTEWKTFTEKHSN |
| Sequence | RPSIEVRSDPKTESLRKNAQKLLSEALELKMDHLLVENIERETFHLCSRLINGPYRRT |
| | VRALVFTLKHRAEIRAQVKSGSLPVGTFVQTHKK |

Further analysis of the NOV6 protein yielded the following properties shown in Table 6B.

TABLE 6B
Protein Sequence Properties NOV6

| | |
|---|---|
| PSort | 0.5336 probability located in nucleus; 0.3000 probability located in microbody |
| analysis: | (peroxisome); 0.1000 probability located in mitochondrial matrix space; 0.1000 |
| | probability located in lysosome (lumen) |
| SignalP | No Known Signal Sequence Predicted |
| analysis: | |

A search of the NOV6 protein against the Geneseq database, a proprietary database that contains sequences published in patents and patent publication, yielded several homologous proteins shown in Table 6C.

TABLE 6C
Geneseq Results for NOV6

| | | NOV6 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB04622 | Human ATP synthase subunit 23 | 1 . . . 208 | 208/208 (100%) | e-115 |
| | protein SEQ ID NO:2 - Homo sapiens, | 1 . . . 208 | 208/208 (100%) | |
| | 208 aa. [CN1307110-A, 08-AUG-2001] | | | |
| AAP93588 | Sequence of transcription factor S-II as | 56 . . . 141 | 35/90 (38%) | 1e-04 |
| | encoded by cDNA from Ehrlich ascites | 18 . . . 99 | 48/90 (52%) | |
| | tumour cells - Homo sapiens, 301 aa. | | | |
| | [EP310030-A, 05-APR-1989] | | | |
| AAB93555 | Human protein sequence SEQ ID | 60 . . . 132 | 25/74 (33%) | 0.004 |
| | NO:12939 - Homo sapiens, 272 aa. | 1 . . . 74 | 39/74 (51%) | |
| | [EP1074617-A2, 07-FEB-2001] | | | |
| AAW93947 | Human regulatory molecule HRM-3 | 57 . . . 108 | 23/53 (43%) | 0.004 |
| | protein - Homo sapiens, 348 aa. | 24 . . . 76 | 34/53 (63%) | |
| | [WO9915658-A2, 01-APR-1999] | | | |
| AAW13852 | Human RNA polymerase transcription | 52 . . . 149 | 29/98 (29%) | 0.005 |
| | factor elongin 110 kDa subunit - Homo | 21 . . . 112 | 48/98 (48%) | |
| | sapiens, 772 aa. [WO9709426-A1, 13- | | | |
| | MAR-1997] | | | |

In a BLAST search of public sequence datbases, the NOV6 protein was found to have homology to the proteins shown in the BLASTP data in Table 6D.

TABLE 6D
Public BLASTP Results for NOV6

| | | NOV6 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96MN5 | CDNA FLJ32112 FIS, CLONE | 1 . . . 208 | 208/208 (100%) | e-115 |
| | OCBBF2001586, WEAKLY SIMILAR | 1 . . . 208 | 208/208 (100%) | |
| | TO TRANSCRIPTION ELONGATION | | | |
| | FACTOR S-II - Homo sapiens | | | |
| | (Human), 208 aa. | | | |
| Q9D7X9 | 2210012G02RIK PROTEIN - Mus | 1 . . . 208 | 189/208 (90%) | e-103 |
| | musculus (Mouse), 207 aa. | 1 . . . 207 | 197/208 (93%) | |
| Q9CZZ2 | 2210012G02RIK PROTEIN - Mus | 1 . . . 207 | 182/207 (87%) | 2e-98 |
| | musculus (Mouse), 228 aa. | 1 . . . 206 | 191/207 (91%) | |
| CAC87121 | AW502783-LIKE PROTEIN - | 1 . . . 81 | 52/81 (64%) | 2e-20 |
| | Tetraodon nigroviridis (Green puffer), | 1 . . . 80 | 65/81 (80%) | |
| | 80 aa (fragment). | | | |
| Q9SG88 | T7M13.10 PROTEIN - Arabidopsis | 28 . . . 131 | 34/105 (32%) | 2e-09 |
| | thaliana (Mouse-ear cress), 416 aa. | 96 . . . 200 | 61/105 (57%) | |

PFam analysis predicts that the NOV6 protein contains the domains shown in the Table 6E.

TABLE 6E
Domain Analysis of NOV6

| | | | |
|---|---|---|---|
| | | Identities/ | |
| Pfam | | Similarities | Expect |
| Domain | NOV6 Match Region | for the Matched Region | Value |

No Significant Matches Found

### Example 7.

The NOV7 clone was analyzed, and the nucleotide and encoded polypeptide sequences are shown in Table 7A.

TABLE 7A
NOV7 Sequence Analysis

| | SEQ ID NO: 17 | 6064 bp |
|---|---|---|
| NOV7, | ATGACCACCAAACGGAAAATCATCGGCCGTCTGGTGCCATCCCGATGTTTCCGAGGTG | |
| CG59849-01 DNA | AAGAAGAAATCATCTCAGTTTTAGATTACTCCCACTGCAGTCTTCAGCAGGTGCCAAA | |
| Sequence | GGAGGTCTTTAACTTCGAACGAACATTAGAGGAGCTTTATCTAGATGCCAATCAAATT | |
| | GAAGAACTACCCAAGCAATTGTTCAACTGTCAAGCTCTACGAAAACTAAGTATTCCTG | |
| | ATAACGACCTTTCAAATCTGCCAACCACTATTGCTAGTTTAGTTAATCTTAAAGAACT | |
| | CGACATCAGTAAAAATGGTGTACAAGAATTTCCAGAAAACATAAAGTGCTGTAAGTGT | |
| | TTAACAATTATTGAAGCCAGTGTCAATCCCATTTCTAAGCTACCTGATCGCTTCACAC | |
| | AGCTCCTAAACCTGACCCAGCTCTACCTGAATGACGCCTTTCTTGAATTTCTTCCAGC | |
| | CAATTTTGGAAGGCTTGTCAAATTGCGGATCTTGGAGTTAAGAGAAAATCACTTGAAA | |
| | ACTCTACCAAAGATGCACAAACTGGCCCAGTTGGAAAGACTTGACCTAGGCAATAATG | |
| | AATTCAGTGAGCTGCCTGAAGTTCTGGATCAAATACAAAATTTGAGGGAGTTATGGAT | |
| | GGATAATAATGCATTACAAGTGTTACCTCGGTCTATAGGGAAGTTAAAGATGTTGGTA | |
| | TACCTGGATATGTCAAAAAACAGAATAGAAACAGTTGACATGGACATTTCTGGATGTG | |
| | AAGCCCTTGAGGACCTCTTATTGTCATCCAATATGTTGCAACAATTGCCTGATTCTAT | |
| | AGGTGGACTTTTGAAAAAACTAACAACTCTAAAAGTAGATGACAATCAACTTACAATG | |
| | CTACCCAATACAATTGGAAGTTTATCTTTATTAGAAGAATTTGACTGTAGCTGTAATG | |
| | AACTGGAGTCACTACCTTCTACTATTGGCTACCTTCATAGTCTTCGGACATTAGCAGT | |
| | GTCATGTCTCTACGCTCCAACAAATTAGAATTTCTTCCTGAAGAGATTGGACAGATGC | |
| | AGAAACTAAGAGTCCTAAATTTGAGTGACAACAGGTTGAAGAATTTACCATTCTCATT | |
| | TACCAAACTTAAAGAGCTTGCAGCTTTGTGGCTTTCTGACAATCAGTCCAAAGCCCTT | |
| | ATCCCTTTACAAACAGAAGCCCATCCAGAAACAAAGCAAAGAGTATTGACTAACTACA | |
| | TGTTTCCCCAGCAGCCTCGTGGTGATGAAGATTTCCAGTCAGACAGTGACAGCTTTAA | |
| | CCCTACACTGTGGGAAGAGCAGAGACAACAACGCATGACTGTTGCCTTTGAATTTGAA | |
| | GACAAAAAAGAAGATGACGAAAATGCTGGGAAAGTTAAGCTCTCCTGCCAAGCCCCCT | |
| | GGGAAAGGGGCCAGCGTGGGATTACTCTCCAACCTGCCAGACTGTCTGGCGATTGCTG | |
| | CACACCATGGGCCAGGTGTGATCAGCAGATCCAAGATATGCCCGTCCCCCAGAATGAC | |
| | CCACAGCTGGCATGGGGTATAAGTATAAGTGGCCTCCAGCAGGGGAGCATGTGTACTC | |
| | CATTGCCAGTTGCAGCACAATCCACCACTCTTCCCTCTCTAAGTGGCAGACAGGTTGA | |
| | TCTGTTCAAAATTTGGTGGGTAAGCCAAGCCATGGAGTGCGTGTTGAGAATTCAAATC | |
| | CAACTGCTAACACGGAGCAAACTGTGAAAGAAAAATATGAACACAAGTGGCCGGTAGC | |
| | GGGGAACTTCACCCTTCATTAGCTGAGACCCCTCTGTACCCACCCAAACTTGTTCTGC | |
| | TAGGGAAGGACAAAAAAGAATCAACTGATGAGTCTGAAGTTGACAAAACTCACTGTCT | |
| | GAATAACAGTGTTTCCTCAGGCACTTACTCAGACTACTCGCCTTCCCAGGCTTCCTCA | |
| | AAAGCCATTACTCAGCCAGCGGGAGGCTGTTCCCCCAGGCAATATACCACAGCGTCCT | |
| | GGATCCTCTAATACCCGGGTTAAAGTGGGGTCCTTGCAGACAACAGCTAAAGATGCAC | |
| | TACATAATTCTTTGTGGGGTAACAGGATTGCACCATCTTTCCCACAGCCTCTTGATTC | |
| | GACCGGCTGCCCATGAGTGATACTTTCACTGACAACTGGACTGATGGCTCGCATTATG | |
| | CTTAAGTTCGAAATCTAGAAGCACATCTTCGCATGGACGCAGGCCTTTGATCAGGCAA | |
| | GACAGGATTGTTGGTGTTCCCCTGGAACTCGAGCAGTCTACACACAGACACACACCAG | |
| | AAACAGAAGTGCCTCCTTCCAATCCTTGGCAGAATTGGACCAGAACCCCTACTCCGTT | |
| | TGAAGACAGGACCGCTTTTCCTTCCAAATTAGAGACAACCCCCACTACCAGCCCATTG | |
| | CCTGAAAGGAAAGAACATATAAAGGAATCTACTGAAATACCTAGTCCTTTTTCTCCAG | |
| | GCGTACCATGGGAGTATCATGATTCCAATCCCAACAGGAGTCTTAGTAATGTCTTTTC | |
| | TCAAATCCATTGCCGCCCGGAATCTTCTAAAGGTGTTATTTCAATTAGCAAAAGCACA | |
| | GAGAGGCTTTCCCCCCTAATGAAAGATATCAAGTCTAATAAATTCAAAAAGTCACAGA | |
| | GTATCGATGAGATTGACATTGGTACATATAAGGTGTATAACATACCATTAGAAAACTA | |
| | TGCTTCTGGGAGTGATCACTTAGGAAGCCACGAACGACCGGATAAGATGCTGGGACCA | |
| | GAGCATGGTATGTCCAGTATGTCTCGAAGCCAGTCAGTCCCAATGCTGGATGATGAGA | |
| | TGCTCACCTACGGAAGTAGTAAGGGGCCACAACAACAAAAAGCTTCTATGACAAAAAA | |
| | AGTCTATCAGTTTGACCAAAGCTTCAATCCTCAAGGATCAGTGGAAGTGAAAGCCGAA | |
| | AAGAGGATACCACCCCCTTTTCAACACAATCCCGAGTACGTGCAACAGGCCACCAAAA | |
| | ACATCGCCAAGGATTTGATTAGTCCTAGAGCTTACAGAGGATACCCACCGATGGAGCA | |
| | AATGTTTTCATTTTCTCAGCCATCTGTGAATGAGGATGCTGTGGTGAATGCCCAGTTC | |
| | GCAAGCCAAGGGGCCAGGGCGGGCTTCCTGAGAAGGGCCGACTCCCTGGTGAGCGCCA | |
| | CAGAAATGGCCATGTTTAGAAGGGTCAATGAGCCTCATGAGCTGCCCCCAACTGATAG | |
| | GAGTCCCAGTTCCTGAAAAGGAATGGCAGGTATGAAGATGAACACCCTTCATATCAAG | |
| | GTCTGCGAGAAGCTACAGTACAGAGAGTTACGGTGCCTCCCAAACCAGGCCAGTTTCA | |
| | GCTAGGCCTACTATGGCAGCTCTTTTGGAAAAAATACCATCTGACTATAACTTCGGTA | |
| | ACTATGGTGACAAGCCATCAGATAACAGTGATTTAAAGACGAGGCCTACTCCTGTGAA | |
| | GGGAGAGGAGAGCTGTGGTAAAATGCCTGCAGACTGGAGACAACAGCTGCTTAGACAT | |
| | ATAGAAGCTAGACGGTTAGACAGGACCCCGTCCCAGCAAAGCAACATTTTAGACAATG | |
| | GACAAGAAGATGTATCTCCTAGTGGCCAATGGAATCCTTATCCACTTGGGAGGCGGGA | |
| | TGTACCTCCGGACACCATTACTAAGAAGGCAGGCAGCCACATCCACACGTTGATGGGG | |
| | TCCCAAAGCCTTCAGCATCGCAGCCGGGAGCAGCAGCCGTATGAAGGAAATATAAACA | |
| | AAGTGACCATCCAGCAATTTCAGTCACCATTGCCTATTCAGATCCCCTCTTCACAGGC | |
| | CACCCGGCGACCTCAGCCTCGACGGTGCTTAATTCAAACTAGGGCAAAGGAGTATG | |
| | GATGGATATCCAGAGCAGTTTTGTGTGAGAATAGAAAAGAATCCTGGCCTTGGATTTA | |
| | GTATCAGTGGTGGAATTAGTGGACAAGGAAATCCATTCAAACCTTCTGACAAGGGTAT | |
| | CTTTGTTACTAGGGTTCAGCCTGATGGGCCAGCATCAAACCTACTGCAGCCTGGTGAT | |
| | AAGATCCTTCAGGCAAATGGACACAGTTTTGTACATATGGCAATGAAAAAGCTGTAT | |
| | TACTACTGAAGAGTTTCCAGAACACAGTAGACCTAGTTATTCAACCTGAGCTTACTGT | |
| | CTAAATATTTTTTATAAATAGTGAAGATACGTCTAGCCAGACCTAATGTTCAAAAATA | |
| | AATTTATACATAGAAACAAATTTTGCCAATTGCTGGACCTGGCAAACATTAGTGCC | |
| | AAATGTATAATACTATATGTTAGCACTGACCATCCTTTGTTAACTCTAT | |
| | TATGATGTTCATGTGGTTATGTATTAGTTTTAATTGTCAGCCTCTGGCTGTGCATTGG | |
| | TGCAGTTTTGTTTCTGTTTTTGTTTTTGTTTTTAATCTGTTTCTTCTCT | |
| | GGATTTCATATAATTTCGGAGCACGGAAGCACACACGCTCTTTATGTTCTGCTC | |
| | TCCATCAGAAACACTGCCTCAAGTTGTATATGCCTTTATATAGTACTATA | |
| | AAGAATTGTAATTCCCATAAAATATTTCTAGCACAAGGTATATGTTGGCATATATACA | |
| | AAAAGAATATAGAGAAAACAATATTTTCATAAACTAAACATCTCAGATAGAGAAAAA | |
| | AATGCCTTATGAAAGTAACTGTACATATGGTATGTGTTTATATTTGGTTCCATAT | |
| | TCATTTGCTAAATTCTCATGACACAGAGTGAAATATTTCATTTAGCCATTTATCT | |
| | CTGGGACCCAAATAAAATAGGATGAACTAATTTGTTCTGCCTTTAGCTTTACA | |
| | ATACATGCAGAGTTTAGAAACAGACTAAAGGTCATTGTAGTTGTCTTTTTCACCAC | |
| | AAATTTAAGCAGTGGATGATGGGTGGCAGGAAAGGTATTGCTTTATTTCTTTCAAGTT | |
| | CATGTTGATTATAACTGTAGCCCCTGTGATTTCTTTACTTGTATGTGGTTTAT | |
| | TTGTGTGTTGCTTAATCTAATTTGCTGCTTTTTATTATTTCGTTTTGG | |
| | ATTGATAAAATTTATCATTACGAAAGACTGCTGTTAGAGTTATGGTAGGTGATTTT | |
| | AAATCCTTGGTATTTAAATATGAAACTTCAAATATAATTTCTCAGAGCTGTGGTCTAC | |
| | CTGTATCATTAATTTCAATGGCTGTTTTTCTGGGCAGTAGATAAAATACTTTTTT | |
| | TCCAAAACAGTTTCAAGGTATGTAATCCTGTGCTTTTTCACTGGAGGA | |
| | CAAGCATGGTTAATGTAGAATTATTTACTTTTCCATTGAACTATTTTCCTGCAT | |
| | TGATCAAAATTTATTTTATAATCCTTTAAAATACTTATCTTTCATATTAGTCATTAAT | |
| | TTAATTACAATATTAATTTGAATTTCCAGGATAATTTCCCGGAGTTGGTTGCATGCAT | |
| | TATCTTTCATTTTTACATAGTTCTTTTGTTATATTGTTTACTTTACATGCTA | |
| | GTGTTTCAAGTATTGTATGAGGATTTTCACAATAGTATCACTGATGATGTCACCAGA | |
| | GCTCTGAGAATAATATTTGTAAGTTAACTGTTTTATGGGGACATTGAATATTGTAT | |
| | TTTTGTAGGGTCTATTAAAATGAGTGTCACTT | |
| | ORF Start: ATG at 1 | ORF Stop: TAA at 4468 |
| | SEQ ID NO: 18 | 1489 aa MW at 167241.9 kD |

| | |
|---|---|
| N0V7, | MTTKRKIIGRLVPCRCFRGEEEIISVLDYSHCSLQQVPKBVFNFERTLEELYLDQI |
| CG59849-01 Protein | EELPKQLFNCQALRKLSIPDNDLSNLPTTTASLLKELDISGVQEFPENIKCCKC |
| Sequence | LTIIEASVNPISKLPDGFTQLLNLTQLYLNDAFLEFLPANFGRLVKLRILELRENHLKI |
| | TLPKMHKLAQLERLDLGNNEFSELPEVLDQIQNLRELWMDNNALQVLPGSIGKLKMLVI |
| | VMSLRSNKLEFLPEEIGQMQKLRVLNLSDNRLKNLPFSFTKLKELAALWLSDNQSKAL |
| | PLQTEAHPETKQRVLTNYMFPQQPPGDEDFQSDSDSFNPTLWEEQRQQRMTVAFEFE |
| | DKKEDDENAGKVKLSCQAPWERGQRGITLQPARLSGDCCTPWARCDQQIQDMPVPQND |
| | PQLAWGCISGLQQERSMCTPLPVAAQSTTLPSLSGRQVEINLKRYPTPYPEDLKNMIKI |
| | SVQNLVGKPSHGVRVENSNPTANTEQTVKEKYEHKWPVAPKEITVEDSFVHPANEMRI |
| | GELHPSLAETFLYPPKLVLLGKDKKESTDESEVDKTHCLNNSVSSGTYSDYSPEQASS |
| | GSSNTRVKVGSLQTTAKDAVHNSLWGNRIAPSFPQPLDSKPLLSQREAVPPGNIPQRP |
| | DRLPMSDTFTDNWTDGSHYDNTGFVAEETTAENANSNPLLSSKSRSTSSHGRRPLIRQ |
| | DRIVGVPLELEQSTHRHTPETEVPPSNPWQNWTRTPSPFEDRTAFPSKLETTPTTSPL |
| | PERKEHIKESTEIPSPFSPGVPWEYHDSNPNRSLSNVFSQIHCRPESSKGVISISK |
| | ERLSPLMKDIKSNKFKKSQSIDEIDIGTYKVYNIPLENYASGSDHLGSHERPDKNLGPI |
| | EBIGMSSMSRSQSVPMLDDEMLTYGSSKGPQQQKASMTKKVYQFDQSFNPQGSVEVKAE |
| | KRIPPPFQHNPEYVQQASKNIAKDLISPAYRGYPPMEQNFSFSQPSEDAWMAQF |
| | ASQGARAGFLRRADSLVSATEMAIAFRRVNEPHELPPTDRYGRPPYRGGLDRQSSVTVT |
| | ESQFLKRNGRYEDEHPSYQEVKAQAGSFPVKNLTQRRPLSARSYSTESYGASQTRPVS |
| | ARPTMAALLEKIPSDYNLGNYGDKPSDNSDLKTRPTPVKGEESCGPADWRQQLLRH |
| | IEARRLDRTPSQQSNILDNGQEDVSPSGQWNPYPLGRRDVPPDTTTKKAGSHIQTLMGI |
| | SQSLQHRSREQQPYEGNINKVTIQQFQSPLPIQTPSSQATRGPQPGRCLIQTKGQRSM |
| | DGYPEQFCVRIEKNPGLGFSISGGISGQGNPFKPSDKGIFVTRVQPDGPASNLLQPGD |
| | KILQANGHSFVHMEHEKAVLLLKSFQNTVDLVIQRELTV |

Further analysis of the NOV7 protein yielded the following properties shown in Table 7B.

TABLE 7B
Protein Sequence Properties NOV7

| | |
|---|---|
| PSort | 0.5192 probability located in mitochondrial matrix space; 0.3000 probability |
| analysis: | located in microbody (peroxisome); 0.2487 probability located in mitochondrial |
| | inner membrane; 0.2487 probability located in mitochondrial intermembrane |
| | space |
| SignalP | No Known Signal Sequence Predicted |
| analysis: | |

A search of the NOV7 protein against the Geneseq database, a proprietary database that contains sequences published in patents and patent publication, yielded several homologous proteins shown in Table 7C.

TABLE 7C
Geneseq Results for NOV7

| | | NOV7 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAM52529 | Human Erbin mutein #5 - Homo | 1 . . . 1488 | 566/1557 (36%) | 0.0 |
| | sapiens, 1371 aa. [FR2807437-A1, | 1 . . . 1370 | 790/1557 (50%) | |
| | 12-OCT-2001] | | | |
| AAM52528 | Human Erbin mutein #4 - Homo | 1 . . . 1488 | 565/1557 (36%) | 0.0 |
| | sapiens, 1371 aa. [FR2807437-A1, | 1 . . . 1370 | 788/1557 (50%) | |
| | 12-OCT-2001] | | | |
| AAM52530 | Human Erbin mutein #6 - Homo | 1 . . . 1488 | 565/1557 (36%) | 0.0 |
| | sapiens, 1371 aa. [FR2807437-A1, | 1 . . . 1370 | 787/1557 (50%) | |
| | 12-OCT-2001] | | | |
| AAM52527 | Human Erbin mutein #3 - Homo | 1 . . . 1488 | 566/1557 (36%) | 0.0 |
| | sapiens, 1371 aa. [FR2807437-A1, | 1 . . . 1370 | 788/1557 (50%) | |
| | 12-OCT-2001] | | | |
| AAM52526 | Human Erbin mutein #2 - Homo | 1 . . . 1488 | 568/1579 (35%) | 0.0 |
| | sapiens, 1419 aa. [FR2807437-A1, | 1 . . . 1418 | 793/1579 (49%) | |
| | 12-OCT-2001] | | | |

In a BLAST search of public sequence datbases, the NOV7 protein was found to have homology to the proteins shown in the BLASTP data in Table 7D.

TABLE 7D
Public BLASTP Results for NOV7

| | | NOV7 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96NW7 | DENSIN-180 - Homo sapiens | 1 . . . 1489 | 1486/1538 (96%) | 0.0 |
| | (Human), 1537 aa. | 1 . . . 1537 | 1487/1538 (96%) | 0.0 |
| P70587 | DENSIN-180 - Rattus norvegicus | 1 . . . 1489 | 1421/1491 (95%) | 0.0 |
| | (Rat), 1495 aa. | 6 . . . 1495 | 1454/1491 (97%) | |
| Q9P212 | KIAA1365 PROTEIN - Homo | 659 . . . 1489 | 829/831 (99%) | 0.0 |
| | sapiens (Human), 831 aa | 1 . . . 831 | 830/831 (99%) | |
| | (fragment). | | | |
| Q96RT1 | DENSIN-180-LIKE PROTEIN - | 1 . . . 1488 | 573/1562 (36%) | 0.0 |
| | Homo sapiens (Human), 1412 aa. | 1 . . . 1411 | 804/1562 (50%) | |
| Q9NR18 | ERBB2-INTERACTING PROTEIN | 1 . . . 1488 | 567/1557 (36%) | 0.0 |
| | ERBIN - Homo sapiens (Human), | 1 . . . 1370 | 789/1557 (50%) | |
| | 1371 aa. | | | |

PFam analysis predicts that the NOV7 protein contains the domains shown in the Table 7E.

TABLE 7E
Domain Analysis of NOV7

| | | Identities/ | |
|---|---|---|---|
| | | Similarities | |
| Pfam Domain | NOV7 Match Region | for the Matched Region | Expect Value |
| LRR: domain 1 of 15 | 47 . . . 69 | 9/25 (36%) | 0.13 |
| | | 19/25 (76%) | |
| LRR: domain 2 of 15 | 70 . . . 92 | 8/25 (32%) | 43 |
| | | 16/25 (64%) | |
| LRR: domain 3 of 15 | 93 . . . 115 | 8/25 (32%) | 0.83 |
| | | 19/25 (76%) | |
| actin: domain 1 of 1 | 87 . . . 117 | 9/31 (29%) | 8.1 |
| | | 21/31 (68%) | |
| LRR: domain 4 of 15 | 116 . . . 138 | 8/25 (32%) | 1e + 02 |
| | | 15/25 (60%) | |
| LRR: domain 5 of 15 | 139 . . . 161 | 10/25 (40%) | 8.1 |
| | | 17/25 (68%) | |
| LRR: domain 6 of 15 | 162 . . . 183 | 8/25 (32%) | 8.1 |
| | | 15/25 (60%) | |
| LRR: domain 7 of 15 | 184 . . . 206 | 10/25 (40%) | 0.048 |
| | | 19/25 (76%) | |
| LRR: domain 8 of 15 | 207 . . . 229 | 12/25 (48%) | 0.041 |
| | | 19/25 (76%) | |
| LRR: domain 9 of 15 | 230 . . . 252 | 5/25 (20%) | 7 |
| | | 17/25 (68%) | |
| LRR: domain 10 of 15 | 253 . . . 275 | 12/25 (48%) | 0.71 |
| | | 18/25 (72%) | |
| LRR: domain 11 of 11 | 277 . . . 299 | 8/25 (32%) | 0.13 |
| | | 21/25 (84%) | |
| LRR: domain 12 of 15 | 300 . . . 322 | 12/25 (48%) | 19 |
| | | 17/25 (68%) | |
| LRR: domain 13 of 15 | 323 . . . 345 | 8/25 (32%) | 30 |
| | | 18/25 (72%) | |
| LRR: domain 14 of 15 | 346 . . . 368 | 8/25 (32%) | 9.2 |
| | | 20/25 (80%) | |
| LRR: domain 15 of 15 | 369 . . . 391 | 11/25 (44%) | 0.00084 |
| | | 20/25 (80%) | |
| ICL: domain 1 of 1 | 1159 . . . 1164 | | 4.7 |
| | | 6/6 (100%) | |
| PDZ: domain 1 of 1 | 1400 . . . 1486 | 34/93 (37%) | 8.5e-19 |
| | | 74/93 (80%) | |

### Example 8.

The NOV8 clone was analyzed, and the nucleotide and encoded polypeptide sequences are shown in Table 8A.

TABLE 8A
NOV8 Sequence Analysis

| | SEQ ID NO: 19 | 982 bp |
|---|---|---|
| NOV8, | TTTCCTTTTCTGTTTCTTAATAGGGGCACTATGAACGAAGAGGAGCAGTTTGTCA | |
| CG59958-01 DNA | TTGATTTGAATGATGACAACATTTGCAGTGTTTGTAAACTGGGAACAGACAAAGAAAC | |
| Sequence | ACTCTCCTTCTGCCACATTTGTTTTGAGCTAAATATTGAGGGTGTACCAAAGTCTGAT | |
| | CTCTTGCACACCAAATCATTAAGGGGCCATAAAGACTGCTTTGAAAAATACCATTTAA | |
| | GATTCAGATTCTGAATGTTCTAAAAACCCCCAGCATCATCTGTTTAATTTCAGGCATA | |
| | AGCCAGAAGAAAAATTACTCCCACAGTTTGACTCCCAAGTACCAAAATATTCTGCAAA | |
| | ATGGATAGATGGAAGTGCAGGTGGCATCTCTAACTGTACAAACGAATTTTGGAGCAG | |
| | AGGGAAAATACAGACTTTGGACTTTCTATCTTACAAGATTCACGTGCCACTTTATGTC | |
| | GTAACAGTGTATTGTGGCCTCATAGTCACAACCAGCCACAGAAAAAAGAAGAGACAAT | |
| | CTCTAGTCCAGAGGCTAATGTCCAGACCCAGCATCCACATTACAGCAGAGAGGAAGTG | |
| | AATTCGATGACTCTTGGTGAGGTAGAGCAACTGAATGCAAAGCTCCTACAGCAAATCC | |
| | AGGAAGTTTTTGAAGAGTTAACTCACCAACTGCAAGAAAAAGATTCTTTGGCCTCACA | |
| | GCTCCATGTCCGCCACGTTGCCATCGAACAGCTTCTGAAGAACTGTTCTAAGTTACCA | |
| | TGTCTGCAAGTAGGGCGAACAGGAATGAAGTCGCACCTACCCATAAACAACTGACCTA | |
| | AACAGACTTACTTCGTATGCCCTGCCCTTTATTGGTCTCCCAGACATGCAAACT | |
| | ORF Start: ATG at 31 | ORF Stop: TGA at 922 |
| | SEQ ID NO: 20 | 297 aa MW at 33933.9 kD |
| NOV8a, | MNEEEQFVNIDLNDDNICSVCKLGTDKETLSFCHICFELNIaGVPKSDLLHTKSLRGH | |
| CG59958-01 Protein | WDCFBKYHLIANQGCPRSKLSKSTYaEVKTTLSKKINWIVQYAQNKDLDSDSECSP | |
| Sequence | QHHLFNFRHKPEEKLLPQFDSQVPKYSAKWIDGSAGGISNCTQRILEQRENTDFGLSM | |
| | LNAKLLQQIQEVFEELTHQVQEKDSLASQLHVRHVAIEQLLKNCSKLPCLQVCRTGM | |
| | LQDSGATLCRNSVLWPHSHNQAQKKEETISSPEANVQTQHPHYSREEVNSMTLGEVEQ | |
| SHLPINN | | |
| | SEQ ID NO: 21 | 981 bp |
| NOV8b, | TTCCTTTTCTGTTTCTTAATAGGGGCACTATGAACGAAGGGGAGCAGTTTGTAAACAT | |
| CG59958-02 DNA | TGATTTGAATGATGACAACATTTGCAGTGTTTGTAACTGGGAACAGACAAGAACA | |
| Sequence | CTCTCCTTCTGCCACATTTGTTTTGAGCTAAATATTGAGGGGGTACCAAAGTCTGATC | |
| | TCTTGCACACCAAATCATTAAGGGGCCATAAAGACTGCTTTGAAAAATACCATTTAAT | |
| | ACCATTTTGAGTAAGAGATAACTGGATTGTGCAGTATGCACTGGATCTGG | |
| | ATTCAGATTCTGAATGTTCTAAAAACCCCCAGCATCATCTGTTTAATTTCAGGCATAA1 | |
| | GCCAGAAGAAAAATTACTCCCACAGTTTGACTCCCAAGTACCAAAATATTCTGCAAAA | |
| | TGGATAGATGGAAGTGCAGGTGGCATCTCTAACTGTACACAAAGAATTTTGGAGCAGA | |
| | IGGGAAAATACAGACTTTGGACTTTCTATGTTACAAGATTCAGGTGCCACTTTATGTCG | |
| | TAACAGTGTATTGTGGCCTCATAGTCACAACCAGGCACAGAAGGAGACTC | |
| | TCTAGTCCAGAGGCTAATGTCCAGACCCAGCATCCACATTACAGCAGAGAGGTTGA | |
| | ATTCGATGACTCTTGCTCAGGTAGAGCAACTGAATGCAAAGCTCCTACAGCTCCA | |
| | GGAAGTTTTTGAAGAGTTAACTCACCAAGTGCAAGAAAAAGATTCTTTGGCCTCACAG | |
| | CTCCATGTCCOCCACGTTGCCATCGAACAGCTTCTGAAGAACTGTTCTAAGTTACCAT1 | |
| | GTCTGCAAGTAGGGCGAACAGGAATGAAGTCGCACCTACCCAAACAACTGACCTAA | |
| | ACAGACTTACTTCGTATGGCCTGCCCTTTATTGGTCTCCCAGACATGCAAACT | |
| | ORF Start: ATG at 30 | ORF Stop: TGA at 921 |
| | SEQ ID NO:22 | 297 aa MW at 33875.9 kD |
| NOV8b, | MNEGEQFVNIDLNDDNICSVCKLGTDKETLSFCHICFELNThGVPKSDLLHTKSLRGH | |
| CG59958-02 Protein | KDCFEKYHLIANQGCPRSKLSKSTYEEVKTILSKKINWIVQYAQNKDLDSDSECSP | |
| Sequence | QHHLFNFRHKPEEKLLPQFDSQVPKYSAKWIDGSAGGISNCTQRILEQRENTDFGLSM | |
| | LQDSGATLCRNSVLWPHSHNQAQKKEETISSPEANVQTQHPHYSPEELNSMTLGEVEQ | |
| | LNAKLLQQIQEVFEELTHQVQEKDSLASQLHVRHVAIEQLLCSKLPCLQVGRTGMK | |
| | SHLPINN | |

Sequence comparison of the above protein sequences yields the following sequence relationships shown in Table 8B.

TABLE 8B
Comparison of NOV8a against NOV8b.

| Protein | NOV8a Residues/ | Identities/ |
|---|---|---|
| Sequence | Match Residues | Similarities for the Matched Region |
| NOV8b | 1 . . . 297 | 295/297 (99%) |
| | | 296/297 (99%) |

Further analysis of the NOV8a protein yielded the following properties shown in Table 8C.

TABLE 8C
Protein Sequence Properties NOV8a

| | |
|---|---|
| PSort | 0.4500 probability located in cytoplasm; 0.3000 probability located in microbody |
| analysis: | (peroxisome); 0.1000 probability located in mitochondrial matrix space; 0.1000 |
| | probability located in lysosome (lumen) |
| SignalP | No Known Signal Sequence Predicted |
| analysis: | |

A search of the NOV8a protein against the Geneseq database, a proprietary database that contains sequences published in patents and patent publication, yielded several homologous proteins shown in Table 8D.

TABLE 8D
Geneseq Results for NOV8a

| | | NOV8a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB43297 | Human ORFX 0RF3061 polypeptide | 1 . . . 221 | 219/221 (99%) | e-131 |
| | sequence SEQ ID NO:6122 - Homo | 1 . . . 221 | 220/221 (99%) | |
| | sapiens, 221 aa. [WO200058473-A2, | | | |
| | 05-OCT-2000] | | | |
| AAM28099 | Peptide #2136 encoded by probe for | 56 . . . 221 | 166/166 (100%) | 2e-97 |
| | measuring placental gene expression - | 1 . . . 166 | 166/166 (100%) | |
| | Homo sapiens, 166 aa. | | | |
| | [WO200157272-A2, 09-AUG-2001] | | | |
| AAM35418 | Peptide #9455 encoded by probe for | 44 . . . 207 | 164/164 (100%) | 2e-95 |
| | measuring placental gene expression - | 1 . . . 164 | 164/164 (100%) | |
| | Homo sapiens, 164 aa. | | | |
| | [WO200157272-A2, 09-AUG-2001] | | | |
| AAM75305 | Human bone marrow expressed probe | 44 . . . 207 | 164/164 (100%) | 2e-95 |
| | encoded protein SEQ ID NO: 35611 - | 1 . . . 164 | 164/164 (100%) | |
| | Homo sapiens, 164 aa. | | | |
| | [WO200157276-A2, 09-AUG-2001] | | | |
| AAM62496 | Human brain expressed single exon | 44 . . . 207 | 164/164 (100%) | 2e-95 |
| | probe encoded protein SEQ ID NO: | 1 . . . 164 | 164/164 (100%) | |
| | 34601 - Homo sapiens, 164 aa. | | | |
| | [WO200157275-A2, 09-AUG-2001] | | | |

In a BLAST search of public sequence datbases, the NOV8a protein was found to have homology to the proteins shown in the BLASTP data in Table 8E.

TABLE 8E
Public BLASTP Results for NOV8a

| | | NOV8a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NYK6 | EURL protein homolog - Homo | 1 . . . 297 | 295/297 (99%) | e-175 |
| | sapiens (Human), 297 aa. | 1 . . . 297 | 296/297 (99%) | |
| Q96BK9 | SIMILAR TO RIKEN CDNA | 1 . . . 297 | 294/297 (98%) | e-174 |
| | 2310009O17 GENE - Homo sapiens | 1 . . . 296 | 296/297 (98%) | |
| | (Human), 296 aa. | | | |
| AAH19957 | RIKEN CDNA 2310009O17 GENE | 1 . . . 297 | 239/297 (80%) | e-138 |
| | - Mus musculus (Mouse), 290 aa. | 1 . . . 290 | 263/297 (88%) | |
| Q9D7G4 | EURL protein homolog - Mus | 1 . . . 297 | 238/297 (80%) | e-137 |
| | musuculus (Mouse), 290 aa. | 1 . . . 290 | 262/297 (88%) | |
| Q9I8W6 | BURL protein - Gallus gallus | 4 . . . 295 | 217/292 (74%) | e-128 |
| | (Chicken), 293 aa. | 3 . . . 293 | 255/292 (87%) | |

PFam analysis predicts that the NOV8a protein contains the domains shown in the Table 8F.

TABLE 8F
Domain Analysis of NOV8a

| | | | |
|---|---|---|---|
| | | Identities/ | |
| Pfam | NOV8a | Similarities | Expect |
| Domain | Match Region | for the Matched Region | Value |

No Significant Matches Found

### Example 9.

The NOV9 clone was analyzed, and the nucleotide and encoded polypeptide sequences are shown in Table 9A.

TABLE 9A
NOV9 Sequence Analysis

| | SEQ ID NO: 23 | 5953 bp |
|---|---|---|
| NOV9, | GATAAGACTTGTAATTTTGGTTATGTGAAGATGAATGTAAGAAGGTACTGAGGAG | |
| CG59961-01 DNA | AGGTTACTAAATGTTACTTCCTCATTGCAGCTGTGACGTTGAGTGCTTCAGATCTGGT | |
| Sequence | CACTATGGTACGAGAACGAAAATGCATATTATGCCACATCGTGTACAGCTCG | |
| | GTAATAATGGAAGAGGGACGAATCTACATGCGGAGCATGTTGCATCACAGGGAACTTG | |
| | AGAACCTCAAGGGCAGGGACATTAGTCATGAGTGCCGAGTGTGCGGGGTCACAGAAGT | |
| | GGGTCTTTCTGCATATGCAGCACATTTCTGGCCAGTTGCACAGATAACGTTGAT | |
| | GCCCAGGAAAGAGAAGATGATGGAAAAGGAGGGGAAGAGGAAGAAGATTATTTTGACA | |
| | AGGAACTCATTCAGTTAATAAACAAAGGAAAGAACAAAGTCGACAAGATGCCTTC | |
| | CAATAGCAACCAAGAAAAAAACTCTGATGACAGACGACCCCAATGGAGACGAGGAC | |
| | CGAATTCCTTACCAAGACAGAGAGAGTTACAGTCAGCCTGCATGGCATCATCGTGGAC | |
| | CTCCACAGCGGGATTGGAAATGGGAAAAAGATGCCTTTAATAATACTAGGAAAAACAGI | |
| | CTTTCCACATTCTTTGAGGAATGGTGGTGGACCAAGAGGACGTTCCGGGTGGCATAG | |
| | GGTGTTGCAGGAGGCTCCTCGACTTGGTTTCACAACCATAGTAATTCTGGAGGTGGTT | |
| | GGCTTTCAATAGTGGAGCAGTAGATTGGATCATAATGGTACAGGAAGGTTCCAG | |
| | TTGGCTTTCTGAAGGAACAGGTGGCTTTTCCAGTTGGCATATGCCAGTCGCA | |
| | AACTGGAAATCCAGTGTACGTAGTACAAATAATTGGAATTACAGTGGCCCTGGAGACA | |
| | AATTTCAACCAGGCAGAACAGAAATTCTAACTGTCTGGGACATGACTATGCT | |
| | ATGGAACAAGAAATCTAATAAGTCAAACAAATACAGTCACGACAGATATTTGGCAG | |
| | CGGCAAGAAAATGACAAACTTGGTACAGTTGCCACATATAGAGGTCCTTCTGGGAT | |
| | TTACAAGTGATAAATTTCCTTCAGAAGGCTTACTCGACTTCAATTTTGAGCAGCTGGA | |
| | AAGCCAACCACTAACAAGCAGACACTGCTACTTCCAGTTAGTGGAGTGGC | |
| | AGTGCGGCAAGGGAAAAGCCTCGTCGCTGGACGCCTTACCCTTCTCAGCACTCTGG | |
| | ATTTACAGTCGCCATTGAAAGACATCACTGCTAACAAGTCAGAAATGATAGAGCC | |
| | TCTCTTTGATTTTAGCTTGATAACTACAGGAATACAGGAGCCCCACTGATGACA | |
| | ATACTTTACGAAACGCCAAAGAGGTGCTACAGTGTCATGAGTCATTGCAATCCACT | |
| | TCTTAGCACTTCTAAAGTACCAGGAACTATGCAAGCAAGTACAATGTACG | |
| | TCTGAAAAAGGGTCTTTGAAAATTGAGTTTCAAGTGCACGCACTAGAAGATGAAAGTG | |
| | ATGGAGAGACATCTGACACGGAAAAGCATGGAACAAAAATTGGAACCCTAGGTTCTGC | |
| | AACTACAGAATTGTTATCTGGCAGCACTCGAACTGCTGATGAGAAAGAGGAGGATGAC | |
| | CGCATCCTGAAGACTTCTAGAGAGCTATCCACTTCCCCATGTAATCCCATAGTTCGC | |
| | AGACTTGAAAACCTCTCTAGAAGATGCACAGGTTGATGACTCTATTAAATCTCATGTA | |
| | CTCACTTCAGAGGGATCTAACCCGGCACATTAGTTTGAAGAGCAAAACTGGAGTACAC | |
| | ATCTCGGAGAAATGTCAACTGGCAACAGGTCATTCAGCAAGTAACCAAGAAAAAGCAA | |
| | GAGCTGGGCAAGGCTTACCCAGGAGGTTTGGCATAGATGGTACCCCTTGTTC | |
| | ATGAACAAGAAGCCTTAGATTTGGATGGGGAACCTGATCTGTCCAGTCTAGGGATT | |
| | CCAGTGGGAAGGTGTTTCCATTTCCTCGTCCCCTGGCTTGGCAAGAAAGCGAAGCCTT | |
| | TCTGAGAGCAGCGTGATCATGGACAGAGCTCCTTCTGTGTATAGCTTCTTCAGTGAGG | |
| | AAGGTACAGGCAAAGAAAATGAGCCCCAGCAGATGGTTTCACCTAGTAACTCATTGAG | |
| | GGCTGGACAGAGCCAGAAAGCAACCATGCACCTCAAACAAGAAGTGACACCTCGGGCT | |
| | GCCTCCCTCCGAACACGTGAAAGGGCTGAAAATGTTGCTACCCAAAGGCGACATAGTG | |
| | CACAATTATCCTCTGACCATATAATACCTTTGATGCATTTGGCAAAGACTTGAACAG | |
| | CCAGGAGAGGTCTATACCACCGTCAGAGTCAGTTCCCAGGAGAGTTGGAGAG | |
| | GGAAACTGTCTGTCATCAAGCGCATCCTCAGCCCTTGCGATCTCCAGTTTAGCGGATG | |
| | CAGCCACAGATAGTAGCTGTACCTCTGGTGCTGAACAAAATGATGGCCAAAGTATTAG | |
| | AAAGAAACGAAGAGCCACTGGAGATGGATCTTCTCCTGAACTCCCAAGTCTTGAGAGA | |
| | AAAAATAAAAGAAGGAAAATTAAAGGAAAAAAGAACGTTCTCAGGTTGACCAGCTGC | |
| | TGAATATTTCTTTAAGGGAGGAGACTTAGTAGTCATTGCAGTGCATGGATC | |
| | TCTTCTGCAAGCCCGTGCAGCCCTTCAGACAGCTTATGTGGAAGTTCAGAGGCTACTT | |
| | ATGCTCAAGCAGCAGATAACTATGGAGATGAGTGCACTGAGGACCCATAGAATACAGA | |
| | TTCTACAGGGATTACAAGAAACATATGAACCTTCTGAGCACCCAGACCAGGTTCCCTG | |
| | TAGCCTCACACGAGAACGAAGGAACAGTAGATCTCAACATCCATTGATGCCGCACTG | |
| | CTGCCCACTCCCTTTTTCCCACTTTTTCTGGAGCCTCCATCTTCCCATGTGTCTCCAT | |
| | CACCCACCGGAGCCTCTCTTCAAATAACCACGTCTCCTACTTTCCAAACCCATGGCAG | |
| | TGTCCCTGCTCCAGACTCATCAGTTCAGATTAAcAGAGCCCATGTCTCCTGC | |
| | GATGAGAATGTGAATGCTGTGCCACCAAGCTCTGCCTGCAATGTGTCCAAGGAATTAC | |
| | TGGAAGCTAATATCAGTGACAGTTGTCCAGTTTATCCAGTCATCACTGCTAGATTGTC | |
| | CTTACCAGAGTCAACAGAAAGTTTCCATGAGCCTAGCCAAGAACTGAAGTTTTCTGTG | |
| | GAGCAAAGAAATACCAGAAACAGAGAAAACTCTCCCTCTTCCCAATCAGCTGGTCTTT | |
| | CTAGCATAAATAAAGAAGGGGAAGAGCCAACCAAAGGCAATAGTGGGTCTGAAGCCTG | |
| | TACCAGTTCTTTTCTAAGATTOTCTTTTGCTTCAGAAACCCCTTTGGAGAAGGAACCC | |
| | CACTCTCCAGCTGACCAGCCTGAACAACACGCAGAATCCACTTTGACATCAGCTGAGA | |
| | CTAGCGGAAGCAAGAAAAAGAAGAAACTCCGGAAGAAGAAAAGTCTACGGGCTCCCCA | |
| | TGTTCCTGAGAATAGTGACACTGAACAGGATGTTTTGACTGTTAACCTGTGGA | |
| | GTAAAGCTGGAAAGTTAATTAAAGGGGGGAAAGTAACAACCTCCACTTGGGAAGACA | |
| | GCAGGACTGGTCGCGAGCAGGAGAGTGTCAGAGATCAGCCAGATAGTGACTCGTCTCT | |
| | GGAAGTCCTAGAAATTCCTAATCCTCAGTTAGAAGTAGTAGCCATTGATTCTTCAG | |
| | TCAGGACAAGAGAAACCAGACAGCCCATCTAAAAAGGATATTTGGAACTCTACAGAGC | |
| | AAAACCCACTAGAAACGTCTCGTTCTGGGTGTGATGAAGTTAGCTCTACCAGTGT | |
| | TGGCACTCGCTATAAAGATGCCATCCCTGTAAGTGTGGCAGCTCAGACTGTGATC | |
| | TCCTCCATAAAAGGATCAAAGAATTCTTCAGAAATATCTTCAGAGCCAGGAGATGATG | |
| | ATGACCCACAGAAGGAAGCTTTGAGGGACACCAAGCTGCCGTTGCAATTCAGAT | |
| | ATTTGGGAACTTGCTATATACCTGTTCAGCAGATAACTGTTCGGGTTTATTCTC | |
| | GTGAGTCGGAATGTATTGGTGTCTTTGAGGGTCATACCTCCAGTTCTGCCTCC | |
| | TGGTTACTCAGACCTCCGGGAAGAATGCTGCCCTTTACACCGGGTCCAGTGACCATAC | |
| | CATCCCCTGCTATAATGTTAAGCAGAGCCGAGAGTGTGTGGAGCAGTTACAGCTGG | |
| | GACCGGGTCCTCTGCCTCCACAGTAGATGGCCAATCCTCTATGCGGGACTGGCTC | |
| | GCACTGTGGTCACCTTCAACATAAAGAACAACAAACGACTTGAGATCTTTCTGCCA | |
| | TCGCCCTCGGGCAGTCAGCTGTCTTGCTACAGCTCAGGAAGGTGCCCGCTGCTG | |
| | GTCGTGGGGTCTTATGACTGCACAATTAGTGTACGCGATGCCCGGTGGACTGCTCC | |
| | TCAGAACTCTGGAGGGCCATAGCAACCATTCTTTGCATGGGTGGTGTGATCT | |
| | CGTCTTCACTGGCTCCAGTGATCAGTCAGTCCATGCTCACCATTCACACTGGTGAG | |
| | CTCGTGCGGATCTATAAGGTCACAATCATGCAGTGACTGTGGTGTATCCTAGG | |
| | KGTGATGCTGACTGCTTCCCTGGATTTTGTTCGTGTCTATGTTACAGGTC | |
| | TCATGATCGATTACAAGTTTATGGAGGACACAAAGACATGATTATGTGTATGACCATC | |
| | CATAAAACCATGATTTACACTGGCTCTTATGATGGCAGTATTCAGGCCGTGAGGCTTA | |
| | ATCTGATCCAGAATTACCCCTGTTGCTGGCATGGTTGCTCTCTGATATTTGGCGTTCT | |
| | AGATCATTTAAAACAACACTTGCTCACCGACCACACTAATCCCAACTTCCAGACTCTG | |
| | AAATGTCGCTCCAAGAACTGCGATGCTTTTTTCACTGCTAGCAAACGATCCCAGG | |
| | ATGCTGCAGGACATATTGAACGACATGCTGAAGATGACAGCAAAATTGATTCATGAG | |
| | TTTTTTGCCTCCCACGTTGGGAAGTCATTAGTTGAACTATTTTCACATTGGCCCCCCA | |
| | CACAGGCCACTCTCTTCCCTTTCTTGGTGAAGTAAGG | |
| | ORF Start: ATG at 121 | ORF Stop: TGA at 5854 |
| | SEQ ID NO:24 | 1911 aa MW at 212465.1 kD |

| | |
|---|---|
| NOV9, | MVRERKCILCHIVYSSKKVIMEEGRIYMRSMLHHRELENLKGRDISHECRVCGVTEVG |
| CG59961-01 Protein | LSAYAKHISGQLHKDNVDAQEREDDGKGGEEEEDYFDKELIQLIKQRKEQSRQDEPSN |
| Sequence | SNQEKNSDDRRFQWRPEDRIPYQDRESYSQPAWMHRGPPQRDWKWEKDCFNNTRKNSF |
| | PHSLRNGGGPRCRSGWHKGVAGGSSTWFHNHSNSGGGWLSNSGAVDWNHNGTGRNSSW |
| | LSEGTGGFSSWHNNNSNGNWKSSVRSTNNWNYSGPGDKFQPGRNRNSNCQMEDMTMLW |
| | NKKSNKSNKYSHDRYNWQRQENDKLGTVATYRGPSEGFTSDKFPSEGLLDFNFEQLES |
| | QTTKQADTATSKVSGKNGSAAREKPRRWTPYPSQKTLDLQSGLKDITGNKSEMIEKPL |
| | FDFSLITTGIQEPQTDETRNSPTQKTQKETHTGSLNHKASSDSAASFEVVRQCPTAEKI |
| | PEQEHTPNKMPSLKSPLLPCPATKSLSQKQDPKNISKNTKTNFFSPGEHSNPSNKPTV |
| | EDNHGPYISKLRSSCPHVLKGNKSTFGSQKQSGDNLNDTLRKAKEVLQCHESLQNPLL |
| | STSKSTRNYAKASRNVEESEKGSLKIEFQVHALEDESDGETSDTEKGTKTCTLCSAT |
| | ILQRDLTRHISLKSTGVLPEPNLNSARRIRNISCNRKSETEKESCLKPTLRQTLNAS |
| | RRNVNWEQVTQQVTKKKQELGKGLPRRFCIEMVPLVQNEQEALDLDGEPDLSSLEGFQ |
| | WEGVSISSSPGLARKRSLSESSVIMDRAPSVYSFFSEEGTGKENEPQQMVSPSNSLRAI |
| | GQSQKATMHLKQEVTPRAASLRTGERAENVATQRRHSAQLSSDHIIPLMHLAKDLNSQ |
| | ERSIPPSENQNSQESNGEGNCLSSSASSALAISSLADAATDSSCTSGAEQNDGQSIRIV |
| | KRRATGDGSSPELPSLERKNKRRKIKGKKERSQVDQLLNISLREEELSKSLQCMDNNL |
| | LQARAALQTAYVEVQRLLMLKQQITMEMSALRTKRIQILQGLQETYEPSEHPDQVPCS |
| | LTRERRNSRSQTSTDAALLPTPFFPLFLEPPSSHVSPSPTGASLQITTSPTPQTHGSV |
| | PAPDSSVQIKQEPMSPEQDENVNAVPPSSACNVSKELLEANISDSCYPVTTARLS |
| | PESTESFHEPSQELKFSVEQRNTRNRENSPSSQSAGLSSTNKEGEEPTKGNSGSEACT |
| | SSFLRLSFASETPLEKEPHSPADQPEQQAESTLTSAETRGSKKKKKLRKKKSLRAAHV |
| | DTEQDVLTVKPVRKVGKLTKGGJTTSTWEDSRTGREQESVRDEPDSDSSLE |
| | PNPQLEVVAIDSSESGEERPDSPSKKDIWNSTEQNPLETSRSGCDEVSSTSEIG |
| | TRYKDGIPVSVAETQTVISSIKGSKNSSEISSEPGDDDEPTEGSFEGHQAAVNAIQIFI |
| | GNLLYTCSADKTVRVYNLVSRKCIGVFEGHTSKVNCLLVTQTSGKNAALYTGSSDHTII |
| | RCYNVKQSRECVEQLQLEDRVLCLHSRWRILYAGLANGTVVTFNIKNNKRLEIPECHG |
| | PRAVSCLATAQEGARKLLVVGSYDCTISVRDARNGLLLRTLEGHSKTILCMKNDLV |
| | FSGSSDQSVHAHNIHTGELVRIYKGHNHAVTVVNILGKVMVTACLDKFVRELQKSHI |
| | DRLQVYCGHKDMIMCMTIHKSMIYTGCYDGSTQAVRLNLMQNYRCWWHGCSLTFGD |
| | HLKQHLLTDHTNPNFQTLKCRWKNCDAFFTARKGSKQDAAGHIERHAEDDSKIDS |

Further analysis of the NOV9 protein yielded the following properties shown in Table 9B.

TABLE 9B
Protein Sequence Properties NOV9

| | |
|---|---|
| PSort | 0.6064 probability located in nucleus; 0.5369 probability located in mitochondrial |
| analysis: | inner membrane; 0.4400 probability located in plasma membrane; 0.3000 |
| | probability located in microbody (peroxisome) |
| SignalP | No Known Signal Sequence Predicted |
| analysis: | |

A search of the NOV9 protein against the Geneseq database, a proprietary database that contains sequences published in patents and patent publication, yielded several homologous proteins shown in Table 9C.

TABLE 9C
Geneseq Results for NOV9

| | | NOV8a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG15238 | Novel human diagnostic protein | 1227 . . . 1911 | 566/687 (82%) | 0.0 |
| | #15229 - Homo sapiens, 938 aa. | 279 . . . 938 | 581/687 (84%) | |
| | [WO200175067-A2, 11-OCT-2001] | | | |
| ABG15238 | Novel human diagnostic protein | 1227 . . . 1911 | 566/687 (82%) | 0.0 |
| | #15229 - Homo sapiens, 938 aa. | 279 . . . 938 | 581/687 (84%) | |
| | [WO200175067-A2, 11-OCT-2001] | | | |
| ABG15239 | Novel human diagnostic protein | 4 . . . 125 | 87/122 (71%) | 1e-37 |
| | #15230 - Homo sapiens, 228 aa. | 3 . . . 98 | 90/122 (73%) | |
| | [WO200175067-A2, 11-OCT-2001] | | | |
| ABG15239 | Novel human diagnostic protein | 4 . . . 125 | 87/122 (71%) | 1e-37 |
| | #15230 - Homo sapiens, 228 aa. | 3 . . . 98 | 90/122 (73%) | |
| | [WO200175067-A2, 11-OCT-2001] | | | |
| ABG15768 | Novel human diagnostic protein | 1654 . . . 1734 | 69/81 (85%) | 1e-32 |
| | #15759 - Homo sapiens, 584 aa. | 379 . . . 459 | 74/81 (91%) | |
| | [WO200175067-A2, 11-OCT-2001] | | | |

In a BLAST search of public sequence datbases, the NOV9 protein was found to have homology to the proteins shown in the BLASTP data in Table 9D.

TABLE 9D
Public BLASTP Results for NOV9

| | | NOV9 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9H2Y7 | ZINC FINGER PROTEIN 106- | 43 . . . 1911 | 1864/1871 (99%) | 0.0 |
| | Homo sapiens (Human), 1883 aa. | 16 . . . 1883 | 1864/1871 (99%) | |
| O88466 | ZINC FINGER PROTEIN 106 - Mus | 1 . . . 1911 | 1476/1917 (76%) | 0.0 |
| | musculus (Mouse), 1888 aa. | 1 . . . 1888 | 1622/1917 (83%) | |
| AAH25424 | HYPOTHETICAL 138.4 KDA | 1 . . . 1259 | 920/1263 (72%) | 0.0 |
| | PROTEIN - Mus musculus (Mouse), | 1 . . . 1243 | 1026/1263 (80%) | |
| | 1245 aa. | | | |
| Q96M37 | CDNA FLJ32848 FIS, CLONE | 283 . . . 1061 | 776/779 (99%) | 0.0 |
| | TEST12003413, MODERATELY | 1 . . . 778 | 778/779 (99%) | |
| | SIMILAR TO ZINC FINGER | | | |
| | PROTEIN 106 - Homo sapiens | | | |
| | (Human), 778 aa (fragment). | | | |
| O55185 | POTENTIAL GRB2 AND FYN- | 245 . . . 848 | 374/607 (61%) | 0.0 |
| | BINDING PROTEIN - Mus musculus | 1 . . . 594 | 439/607 (71%) | |
| | (Mouse), 600 aa | | | |

PFam analysis predicts that the NOV9 protein contains the domains shown in the Table 9E.

TABLE 9E
Domain Analysis of NOV9

| | | Identities/ | |
|---|---|---|---|
| | | Similarities | |
| Pfam Domain | NOV7 Match Region | for the Matched Region | Expect Value |
| zf-C2H2: domain | 47 . . . 71 | 6/26 (23%) | 24 |
| 1 of 2 | | 14/26 (54%) | |
| WD40: domain 1 of 6 | 1549 . . . 1583 | 12/37 (32%) | 0.00024 |
| | | 30/37 (81%) | |
| WD40: domain 2 of 6 | 1589 . . . 1628 | 12/40 (30%) | 0.016 |
| | | 30/40 (75%) | |
| WD40: domain 3 of 6 | 1676 . . . 1713 | 11/39 (28%) | 16 |
| | | 27/39 (69%) | |
| WD40: domain 4 of 6 | 1719 . . . 1753 | 14/37 (38%) | 0.016 |
| | | 29/37 (78%) | |
| WD40: domain 5 of 6 | 1759 . . . 1793 | 10/37 (27%) | 0.045 |
| | | 25/37 (68%) | |
| WD40: domain 6 of 6 | 1800 . . . 1834 | 7/37 (19%) | 0.1 |
| | | 28/37 (76%) | |
| zf-C2H2: domain | 1841 . . . 1866 | 10/26 (38%) | 0.041 |
| 2 of 2 | | 18/26 (69%) | |

### Example 10.

The NOV10 clone was analyzed, and the nucleotide and encoded polypeptide sequences are shown in Table 10A.

TABLE 10A
NOV10 Sequence Analysis

| | SEQ ID NO: 25 | 556 bp |
|---|---|---|
| NOV10, | GCACGGTCCGGGTGAGCCGCGATACTGTCGGCCCCTTGTCGCCTGGAAGTCGTGTCGA | |
| CG88600-01 DNA | TGACCTTGAACAAACTCCTGCTGCTCACCTGCATCTGCCTGACCCTGGCTGCTTGTGG | |
| Sequence | TGGGGTCGACCCCAACTCGCCGTTGGGCAAGCGCCAAGCCGCGTTCAAGGAGATGCTC | |
| | AAGGTCAGCGAAGACCTCGGTGGGATGTTGCGCAATCGTATTCCCTACGACGAAGCCG | |
| | CATTCATCAGCGGCGCAGCCAAGCTCGAGTGTCTGTCGCACGAGCCCTGGCAGCACTT | |
| | TCCACAGGTACGTGACGACGAACGCAGCAAGGCCAATCCCGAGGTCTGGCAGCGCCAG | |
| | GAGCAATTCCAGAAGATGGCGCGTGGTCTGGAGCAGGCCACCGCCGCACTGGTGCAGG | |
| | TGACGACCGCGCCGCCGCTACGCCGCTCCGAGCTGGAGCCGGCAGTGCAGGCCATCGA | |
| | GGACAGTTGCGAGGCCTGCCACAAGGCGTTTCGCGCTTACTGATCGACGCGCGCTTCG | |
| | GCCTCGGCCTGCTCCAGTTCGGCGCGCGCCTCGG | |
| | ORF Start: ATG at 58 | ORF Stop: TGA at 505 |
| | SEQ ID NO: 26 | 149 aa MW at 16625.9 kD |
| NOV10, | MTLKKLLLLTCICLTLAACGGVDPNSPLGKRQAAFKEMLKVSEDLGGMLRNRIPYDEA | |
| CG88600-01 Protein | AFISGAAKLECLSHEPWQHFPQVRDDERSKANPEVWQRQEQFQRD4ARGLEQATAALVQ | |
| Sequence | VTTAPPLRRSELEPAVQAIEDSCEACHKAFRAY | |

Further analysis of the NOV10 protein yielded the following properties shown in Table 10B.

TABLE 10B
Protein Sequence Properties NOV10

| | |
|---|---|
| PSort | 0.8200 probability located in outside; 0.1000 probability located in endoplasmic |
| analysis: | reticulum (membrane); 0.1000 probability located in endoplasmic reticulum |
| | (lumen); 0.1000 probability located in microbody (peroxisome) |
| SignalP | Cleavage site between residues 18 and 19 |
| analysis: | |

A search of the NOV10 protein against the Geneseq database, a proprietary database that contains sequences published in patents and patent publication, yielded several homologous proteins shown in Table 10C.

TABLE 10C
Geneseq Results for NOV10

| | | NOV10 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAY85179 | Cellulose synthase subunit amino acid | 37 . . . 137 | 27/102 (26%) | 0.12 |
| | sequence - Vigna angularis, 1124 aa. | 95 . . . 190 | 50/102 (48%) | |
| | [JP2000060568-A, 29-FEB-2000] | | | |
| AAU21686 | Novel human neoplastic disease | 30 . . . 127 | 21/101 (20%) | 1.0 |
| | associated polypeptide #119 - Homo | 200 . . . 296 | 45/101 (43%) | |
| | sapiens, 354 aa. [W0200155163-A1, | | | |
| | 02-AUG-2001] | | | |
| AAW22779 | Human septin-2 protein clone B3 - | 30 . . . 127 | 21/101 (20%) | 1.0 |
| | Homo sapiens, 401 aa. [WO9727284- | 301 . . . 397 | 45/101 (43%) | |
| | A2, 31-JUL-1997] | | | |
| AAW22776 | Human septin-2 protein - Homo | 30 . . . 127 | 21/101 (20%) | 1.0 |
| | sapiens, 523 aa. [WO9727284-A2, | 423 . . . 519 | 45/101 (43%) | |
| | 31-JUL-1997] | | | |
| AAG14457 | Arabidopsis thaliana protein fragment | 2 . . . 50 | 18/53 (33%) | 1.3 |
| | SEQ ID NO: 14328 - Arabidopsis | 3 . . . 55 | 26/53 (48%) | |
| | thaliana, 542 aa. [EP1033405-A2, 06- | | | |
| | SEP-2000] | | | |

In a BLAST search of public sequence datbases, the NOV10 protein was found to have homology to the proteins shown in the BLASTP data in Table 10D.

TABLE 10D
Public BLASTP Results for NOV10

| | | NOV10 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9I5Z5 | HYPOTHETICAL PROTEIN PA0541 - | 1 . . . 148 | 67/151 (44%) | 7e-3 |
| | Pseudomonas aeruginosa, 152 aa. | 1 . . . 151 | 92/151 (60%) | |
| Q9JZR9 | CYTOCHROME C - Neisseria | 7 . . . 148 | 48/151 (31%) | 1e-08 |
| | meningitidis (serogroup B), 152 aa. | 7 . . . 150 | 70/151 (45%) | |
| Q9JUV4 | PUTATIVE C-TYPE | 7 . . . 148 | 48/151 (31%) | 2e-08 |
| | CYTOCHROME - Neisseria | 7 . . . 150 | 69/151 (44%) | |
| | meningitidis (serogroup A), 152 aa. | | | |
| Q53142 | Cytochrome c-554 precursor (C554) | 45 . . . 147 | 32/107 (29%) | 1e-05 |
| | (High potential cytochrome c) - | 47 . . . 150 | 54/107 (49%) | |
| | Rhodobacter sphaeroides | | | |
| | (Rhodopseudomonas sphaeroides), 153 | | | |
| | aa. | | | |
| P00143 | Cytochrome c’ - Paracoccus sp. (Strain | 23 . . . 148 | 36/131 (27%) | 3e-05 |
| | ATCC 12084), 132 aa. | 2 . . . 131 | 58/131 (43%) | |

PFam analysis predicts that the NOV10 protein contains the domains shown in the Table 10E.

TABLE 10E
Domain Analysis of NOV10

| | | Identities/ | |
|---|---|---|---|
| | | Similarities | |
| | NOV10 Match | for the Matched | Expect |
| Pfam Domain | Region | Region | Value |
| Cytochrome_C_2: domain | 25 . . . 149 | 36/133 (27%) | 5.4e-06 |
| 1 of 1 | | 84/133 (63%) | |

### Example 11.

The NOV11 clone was analyzed, and the nucleotide and encoded polypeptide sequences are shown in Table 11A.

TABLE 11A
NOV11 Sequence Analysis

| | SEQ ID NO: 27 | 1189 bp |
|---|---|---|
| NOV11, | ACCCCGTGGAGCACGCCGATATGGCTGCGCTGACACTGAGGGGTGTCCGGGAGCTGCT | |
| CG88655-01 DNA | GAAGCGTGTGGACCTCGCGACGGTCCCGCGGAGACATCGATATAAGAAGAAATGGGCT | |
| Sequence | GCCACAGAGCCCAAATTCCCTGCTGTTCGACTGGCTTTGCAGAATTTTGACATGACTT | |
| | ACAGTGTGCAGTTTGGAGATCTTTGGCCATCAATCCGTGTCAGTCTCCTCTCAGAGCA | |
| | GAAGTATGGTGCACTGGTCAATAACTTTGCTGCCTGGGATCATGTAAGTGCTAAGCTG | |
| | GAGCAGCTGAGTGCCAAGGATTTTGTGAATGAAGCCATCTCCCACTGGGAACTGCAGT | |
| | CTGAGGGTGGCCAATCTGCAGCCCCATCCCCTGCCTCCTGGGCCTGCAGTCCGAACCT | |
| | TCGATCCTTCACTTTTGACAGAGGGGATATCAGTCGCTTCCCTCCTGCCAGGCCTGGC | |
| | AGCCTGGGTGTCATGGAGTACTACCTGATGGATGCTGCCTCCTTGCTGCCTGTTCTGG | |
| | CCCTCGGCCTGCAGCCTGGGGACATCGTGCTTGACCTATGTGCAGCTCCTGGGGGAAA | |
| | GACACTAGCGTTGCTTCAGACTGGCTGTTGCCGTAATCTTGCTGCCAATGATCTCTCC | |
| | CCGTCCCGAATAGCCAGACTACAGAAGATCCTTCACAGCTATGTGCCTGAAGAGATCA | |
| | GGGATGGAAATCAAGTTCGAGTTACCTCATGGGATGGCAGGAAATGGGGAGAACTGGA | |
| | GGCGGACACCTATGACCGGGTGCTGGTGGATGTGCCCTGTACCACAGACCGCCACTCC | |
| | CTTCATGAGGAGGAGAACAACATCTTTAAGCGGTCAAGGAAGAACGACCGACAGATAT | |
| | TGCCTGTGCTGCAAGTGCAGCTTCTTGCGGCTGGACTCCTTGCCACCAAACCAGGAGG | |
| | CCATGTTGTCTATTCTACCTGCTCACTCTCACACTTACAGAACGAGTATGTGGTGCAA | |
| | GGTGCCATTGAGCTCCTGGCCAATCAATACAGCATCCAGGTACAGGTGGAAGATCTGA | |
| | CTCACTTCCGAAGGGTTTTCATGGACACATTTTGTTTCTTCTCATCCTGTCAGGTTGG | |
| | GGAGCTGGTAATACCAAACCTCATGGCCAATTTTGGCCCCATGTACTTCTGCAAAATG | |
| | CGTAGGCTGACATAGTATCACCCAATCCC | |
| | ORF Start: ATG at 21 | ORF Stop: TAG at 1173 |
| | SEQ ID NO: 28 | 384 aa MW at 43088.1 kD |

| | |
|---|---|
| NOV11, | MAALTLRGVRELLKRVDLATVPRRHRYKKKWAATEPKFPAVRLALQNFDMTYSVQFGD |
| CG88655-01 Protein | LWPSIRVSLLSEQKYGALVNNFAAWDHVSAKLEQLSAKDFVNEAISHWELQSEGGQSA |
| Sequence | APSPASWACSPNLRCFTFDRGDISRFPPARPGSLGVMEYYLMDAASLLPVLALGLQPG |
| | DIVLDLCAAPGGKTLALLQTGCCRNLAANDLSPSRIARLQKILHSYVPEEIRDGNQVR |
| | VTSWDGRKWGELEGDTYDRVLVDVPCTTDRHSLHEEENNIFKRSRKKERQILPVLQVQ |
| | LLAAGLLATKPGGHVVYSTCSLSHLQNEYVVQGAIELLANQYSIQVQVEDLTHFRRVF |
| | MDTFCFFSSCQVGELVIPNLMANFGPMYFCKMRRLT |

Further analysis of the NOV11a protein yielded the following properties shown in Table 11B.

TABLE 10B
Protein Sequence Properties NOV11

| | |
|---|---|
| PSort | 0.5949 probability located in mitochondrial inner membrane; 0.4400 probability |
| analysis: | located in plasma membrane; 0.4200 probability located in nucleus; 0.3797 |
| | probability located in mitochondrial matrix space |
| SignalP | No Known Signal Sequence Predicted |
| analysis: | |

A search of the NOV11 protein against the Geneseq database, a proprietary database that contains sequences published in patents and patent publication, yielded several homologous proteins shown in Table 11C.

TABLE 11C
Geneseq Results for NOV11

| | | NOV11 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB93752 | Human protein sequence SEQ ID | 219 . . . 584 | 164/166 (98%) | 4e-94 |
| | NO:13419 - Homo sapiens, 186 aa. | 21 . . . 186 | 166/166 (99%) | |
| | [EP1074617-A2, 07-FEB-2001] | | | |
| ABG09325 | Novel human diagnostic protein #9316 | 199 . . . 328 | 130/130 (100%) | 1e-70 |
| | - Homo sapiens, 272 aa. | 8 . . . 137 | 130/130 (100%) | |
| | [WO200175067-A2, 11-OCT-2001] | | | |
| ABG09325 | Novel human diagnostic protein #9316 | 199 . . . 328 | 130/130 1000/) | 1e-70 |
| | - Homo sapiens, 272 aa. | 8 . . . 137 | 130/130 (100%) | |
| | [WO200175067-A2, 11-OCT-2001] | | | |
| AAM05754 | Peptide #4436 encoded by probe for | 32 . . . 146 | 115/115 (100%) | 4e-63 |
| | measuring breast gene expression - | 1 . . . 115 | 115/115 (100%) | |
| | Homo sapiens, 115 aa. | | | |
| | [WO200157270-A2, 09-AUG-2001] | | | |
| AAM30628 | Peptide #4665 encoded by probe for | 32 . . . 146 | 115/115 (100%) | 4e-63 |
| | measuring placental gene expression - | 1 . . . 115 | 115/115 (100%) | |
| | Homo sapiens, 115 aa. | | | |
| | [WO200157272-A2, 09-AUG-2001] | | | |

In a BLAST search of public sequence datbases, the NOV11 protein was found to have homology to the proteins shown in the BLASTP data in Table 11D.

TABLE 11D
Public BLASTP Results for NOV11

| | | NOV11 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96CB9 | SIMILAR TO RIKEN CDNA | 1 . . . 384 | 383/384 (99%) | 0.0 |
| | 2810405F18 GENE - Homo sapiens | 1 . . . 384 | 383/384 (99%) | |
| | (Human), 384 aa. | | | |
| Q9CZ57 | 2810405F18RIK PROTEIN - Mus | 1 . . . 383 | 329/383 (85%) | 0.0 |
| | musculus (Mouse), 381 aa. | 1 . . . 380 | 351/383 (90%) | |
| Q9D7F0 | 2310010O12RIK PROTEIN - Mus | 195 . . . 383 | 167/189 (88%) | 1e-96 |
| | musculus (Mouse), 234 aa. | 45 . . . 233 | 180/189 (94%) | |
| Q9HAJ8 | HYPOTHETICAL 21.2 KDA | 219 . . . 384 | 164/166 (98%) | 1e-93 |
| | PROTEIN - Homo sapiens (Human), | 21 . . . 186 | 166/166 (99%) | |
| | 186 aa. | | | |
| Q9VPX3 | CG4749 PROTEIN (LD40271P) - | 100 . . . 382 | 114/287 (39%) | 2e-52 |
| | Drosophila melanogaster (Fruit fly), | 218 . . . 501 | 178/287 (61%) | |
| | 503 aa. | | | |

PFam analysis predicts that the NOV11 protein contains the domains shown in the Table 11E.

TABLE 11E
Domain Analysis of NOV11

| | | Identities/ | |
|---|---|---|---|
| | | Similarities | |
| | NOV11 Match | for the Matched | Expect |
| Pfam Domain | Region | Region | Value |
| Noll_Nop2_Sun: domain | 155 . . . 312 | 48/203 (24%) | 5.8e-13 |
| 1 of 1 | | 112/203 (55%) | |

### Example 12.

The NOV12 clone was analyzed, and the nucleotide and encoded polypeptide sequences are shown in Table 12A.

TABLE 12A
NOV12 Sequence Analysis

| | SEQ ID NO: 29 | 1198 bp |
|---|---|---|
| NOV12, | TTTAGTTACCTAGATTCAAGATGAATAGCGATCAAGTTACACTGGTTGGTCAAGTGTT | |
| CG88665-01 DNA | TGAGTCATATGTTTCCGAATACCATAAGAATGATATTCTTCTAATCTTGAAGGAAAGG | |
| Sequence | GATGAAGATGCTCATTACCCAGTTGTGGTTAATGCCATGACTCTGTTTGAGACCAACA | |
| | TGGAAATCGGGGAATATTTCAACATGTTCCCCAGTGAAGTGCTTACAATTTTTGATAG | |
| | TGCACTGCGAAGGTCAGCCTTGACAATTCTCCAGTCCCTTTCTCAGCCTGAGGCTGTT | |
| | TCCATGAAACAGAATCTTCATGCCAGGATATCAGGTTTGCCTGTCTGTCCTGAGCTGG | |
| | TGAGGGAACACATACCTAAAACCAAGGATGTGGGACACTTTTTATCTGTCACTGGGAC | |
| | AGTGATTCGAACAAGTCTGGTGAAGGTTCTGGAGTTTGAGCGGGATTACATGTGTAAC | |
| | AAATGCAAGCATGTGTTTGTGATCAAGGCTGACTTTGAGCAGTATTACACCTTTTGCC | |
| | GGCCATCCTCGTGTCCCAGCTTGGAGAGCTGTGATTCCTCTAAATTCACTTGCCTCTC | |
| | AGGCTTGTCTTCGTCTCCAACCAGGTGTAGAGATTACCAGGAAATCAAAATTCAGGAA | |
| | CAGGTACAAAGGCTATCTGTTGGAAGTATTCCACGATCTATGAAGGTTATTCTGGAAG | |
| | ATGACTTAGTGGATAGTTGCAAATCTGGTGATGACCTCACTATTTACGGGATTGTAAT | |
| | GCAACGGTGGAAGCCCTTTCAGCAAGATGTGCGCTGTGAAGTGGAGATAGTCCTGAAA | |
| | GCAAATTACATCCAAGTAAATAATGAGCAGTCCTCAGGGATCATCATGGATGAGGAGG | |
| | TCCAAAAGGAATTCGAAGATTTTTGGGAATACTATAAGAGCGATCCCTTTGCAGGTAG | |
| | GAATGTAATATTGGCTAGCTTGTGCCCTCAAGTGTTTGGAATGTATCTAGTAAAGCTT | |
| | GCTGTGGCCATGGTGCTGGCTGGTGGGATTCAAAGGACTGATGCTACAGGAACACGGG | |
| | TCAGAGGTGAATCTCATCTTTTATTGGTTGGGGATCCTGGCACAGGGAAATCTCAGTT | |
| | CCTCAAATATGCAGCAAAGATTACACCAAGATCTGTGCTGACCACAGGAATTGGATCT | |
| | ACTAGTGCAGGTATTGTATGTGACAATTTCAAGTAATT | |
| | ORF Start: ATG at 21 | ORF Stop: TAA at 1194 |
| | SEQ ID NO: 30 | 391 aa MW at 43983.0 kD |

| | |
|---|---|
| NOV12, | MNSDQVTLVGQVFESYVSEYHKNDILLTLKERDEDAHYPVVVNAMTLFETNMEIGEYF |
| CG88665-01 Protein | NMFPSEVLTIFDSALRRSALTILQSLSQPEAVSMKQNLHARISGLPVCPELVREHIPK |
| Sequence | TKDVGHFLSVTGTVIRTSLVKVLEFERDYMCNKCKHVFVIKADFEQYYTFCRPSSCPS |
| | LESCDSSKFTCLSGLSSSPTRCRDYQEIKIQEQVQRLSVGSIPRSMKVILEDDLVDSC |
| | KSGDDLTIYGIVMQRWKPFQQDVRCEVEIVLKANYTQVNNEQSSGIIMDEEVQKEFED |
| | FWEYYKSDPFAGRNVILASLCPQVFGMYLVKLAVANVLAGGIQRTDATGTRVRGESHL |
| | LLVGDPGTGKSQFLKYAAKITPRSVLTTGIGSTSAGIVCDNFK |

Further analysis of the NOV12 protein yielded the following properties shown in Table 12B.

TABLE 12B
Protein Sequence Properties NOV12

| | |
|---|---|
| PSort | 0.8500 probability located in endoplasmic reticulum (membrane); 0.4400 |
| analysis: | probabilty located in plasma membrane; 0.3000 probability located in |
| | microbody (peroxisome); 0.1000 probability located in mitochondrial |
| | inner membrane |
| SignalP | No Known Signal Sequence Predicted |
| analysis: | |

A search of the NOV12 protein against the Geneseq database, a proprietary database that contains sequences published in patents and patent publication, yielded several homologous proteins shown in Table 12C.

TABLE 12C
Geneseq Results for NOV12

| | | NOV12 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAM35524 | Peptide #9561 encoded by probe for | 1 . . . 70 | 70/70 (100%) | 6e-34 |
| | measuring placental gene expression - | 6 . . . 75 | 70/70 (100%) | |
| | Homo sapiens, 75 aa. [WO200157272- | | | |
| | A2, 09-AUG-2001] | | | |
| AAM75412 | Human bone marrow expressed probe | 1 . . . 70 | 70/70 (100%) | 6e-34 |
| | encoded protein SEQ ID NO: 35718 - | 6 . . . 75 | 70/70 (100%) | |
| | Homo sapiens, 75 aa. [WO200157276- | | | |
| | A2, 09-AUG-2001] | | | |
| AAM62602 | Human brain expressed single exon | 1 . . . 70 | 70/70 (100%) | 6e-34 |
| | probe encoded protein SEQ ID NO: | 6 . . . 75 | 70/70 (100%) | |
| | 34707 - Homo sapiens, 75 aa. | | | |
| | [WO200157275-A2, 09-AUG-2001] | | | |
| ABB41728 | Peptide #9234 encoded by human foetal | 1 . . . 70 | 70/70 (100%) | 6e-34 |
| | liver single exon probe - Homo sapiens | 6 . . . 75 | 70/70 (100%) | |
| | 75 aa. [WO200157277-A2, 09-AUG- | | | |
| | 2001] | | | |
| AAM36636 | Peptide #10673 encoded by probe for | 236 . . . 301 | 66/66 (100%) | 4e-33 |
| | measuring placental gene expression - | 1 . . . 66 | 66/66 (100%) | |
| | Homo sapiens, 66 aa. [WO200157272- | | | |
| | A2, 09-AUG-2001] | | | |

In a BLAST search of public sequence datbases, the NOV12 protein was found to have homology to the proteins shown in the BLASTP data in Table 12D.

TABLE 12D
Public BLASTP Results for NOV12

| | | NOV12 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9D344 | 9030408O17RIK PROTEIN - Mus | 1 . . . 386 | 352/386 (91%) | 0.0 |
| | musculus (Mouse), 386 aa. | 1 . . . 386 | 373/386 (96%) | |
| Q9HCV3 | DJ329L24.3 (MEMBER OF MCM2/3/5 | 116 . . . 386 | 271/271 (100%) | e-156 |
| | FAMILY) - Homo sapiens (Human), | 1 . . . 271 | 271/271 (100%) | |
| | 441 aa (fragment). | | | |
| Q9ZPT4 | PUTATIVE DNA REPLICATION | 16 . . . 385 | 160/386 (41%) | 2e-76 |
| | LICENSING FACTOR - Arabidopsis | 17 . . . 398 | 236/386 (60%) | |
| | thaliana (Mouse-ear cress), 610 aa. | | | |
| Q9UXG1 | MINICHROMOSOME | 94 . . . 385 | 93/295 (31%) | 4e-37 |
| | MAINTENANCE (MCM) PROTEIN | 94 . . . 373 | 168/295 (56%) | |
| | (MINICHROMOSOME | | | |
| | MAINTENANCE PROTEIN MCM) - | | | |
| | Sulfolobus solfataricus, 686 aa. | | | |
| AAL63108 | DNA REPLICATION LICENSING | 87 . . . 385 | 97/300 (32%) | 3e-34 |
| | FACTOR (MCM) - Pyrobaculum | 81 . . . 362 | 163/300 (54%) | |
| | aerophilum, 680 aa. | | | |

PFam analysis predicts that the NOV12 protein contains the domains shown in the Table 12E.

TABLE 12E
Domain Analysis of NOV12

| | | Identities/ | |
|---|---|---|---|
| Pfam | NOV12 | Similarities for | Expect |
| Domain | Match Region | the Matched Region | Value |
| MCM: domain 1 of 1 | 106 . . . 391 | 97/623 (16%) | 1.9e-11 |
| | | 212/623 (34%) | |

### Example 13.

The NOV13 clone was analyzed, and the nucleotide and encoded polypeptide sequences are shown in Table 13A.

TABLE 13A
NOV13 Sequence Analysis

| | SEQ ID NO: 31 | 552 bp |
|---|---|---|
| NOV 13a, | TGTTGAGGAGATGGGGGCTGCGGTGACTCGCGGGATCAGGAATTTCAACCTAGAGAAC | |
| CG88685-01DNA | CCAGCGGAACGGGAAATCAGCAAGATGAAGCCCTCTCCCACTCCCGGTTACCCCTCTA | |
| Sequence | CCAACAGCCTCCTGCAAGAGCAGATTAGTCTCTATCCAGAAATTAAGGTAGAGATTGC | |
| | TCGTAAAGATGACAAGATGCTGCCATTTCTAAAAGATGTATATGTTGATTCCAAAGAT | |
| | CCTGTGTCTTCCGTGCAGGTAAAAGCTGCTGAAACACGTCAAGAGCCAGAGGAATTCA | |
| | GATTGCCCAAAGGCTATCACTTTGATATAATAAATATTAAGAGCATTCCCAAAGGCAA | |
| | AATTTCCATTATAGAAGCATTGACTTTTCTCAATAATCATAAACTTTATCAAGAAACA | |
| | TGGACCGCTGAGAAAATAGCGCAAGAATACCATTTAGAACAGAAAGATGTGAGTTCCC | |
| | CTCTTTATTTTGTTACTTTTGAACTCAAAATCTTCCCTCATGAAGACAAGAAAGCAAT | |
| | ACAATCAAAATGAAGAAAATCGCAAAAATT | |
| | ORF Start: ATG at 11 | ORF Stop: TGA at 533 |
| | SEQ ID NO: 32 | 174 aa MW at 20037.7 kD |
| NOV13a, | MGAAVTRGIRNFNLENPAEREISKMKPSPTPGYPSTNSLLQEQISLYPEIKVEIARKD | |
| CG88685-01 Protein | DKMLPFLKDVYVDSKDPVSSVQVKAAETRQEPEEFRLPKGYHFDITNIKSIPKGKISI | |
| Sequence | IEALTFLNNHKLYQETWTAEKTAQEYHLEQKDVSSFLYFVTFELKIFPHEDKKAIQSK | |
| | SEQ ID NO: 33 | 528 bp |
| Nov13b, | ATGGGGGCTGCGGTGACTCGCGGGATCAGGAATTTCAACCTAGAGAACCCACCGGAAA | |
| CG88685-02 DNA | GGGAAATCCGCAACATGAAGCCCTCTCCCACTCCCGGTTACCCCTCTACCAACAGCCT | |
| Sequence | CCTGCAAGAGCAGATTAGTCTCTATCCAGAAATTAAGGGAGAGATTGCTCGTAAAGAT | |
| | GACAAGCTGCTGCCATTTCTAAAAGATGTGTGTGTTGATTCCAAAGATCCTGTGTCTT | |
| | CCGTGCAGCTGAAAGCTGCTGAAACACGTCAAGAGCCAAAGAAATTCAGATTGCCGAA | |
| | AGGCTATCACTTTGATATGATAAATATTAAGAGCATTCCCAAAGGCAAAATTTCCATT | |
| | ATAGAAGCATTGACTTTTCTCAATAATCATAAACTTTATCAAGAAACATGGACCGCTG | |
| | AGAAAATAGCGCAAGAATACCATTTAGAACAGAAACATGTGAATTCCCCTCTTAAATA | |
| | TTTTGTTACTTTTGAACTCAAAATCTTCCCTCATCAAGACAAGAAAGCAATACAATCA | |
| | AAATGA | |
| | ORF Start: ATG at 1 | ORF Stop: TGA at 526 |
| | SEQ ID NO: 34 | 1175 aa MW at 20158.0 kD |

| | |
|---|---|
| NOV13b, | MGAAVTRGIRNFNLENPAEREIRNMKPSPTPGYPSTNSLLQEQISLYPEIKGEIARKD |
| CG88685-02 Protein | DKLLPFLKDVCVDSKDPVSSVQLKAAETRQEPKKFRLPKGYHFDMINIKSTPKGKISI |
| Sequence | IEALTFLNNHKLYQETWTAEKIAQEYHLEQKDVNSPLKYFVTPELKIFPHEDKKATQS |
| | K |

Sequence comparison of the above protein sequences yields the following sequence relationships shown in Table 13B.

TABLE 13B
Comparison of NOV13a against NOV13b.

| Protein | NOV13a Residues/ | Identities/ |
|---|---|---|
| Sequence | Match Residues | Similarities for the Matched Region |
| NOV13b | 1 . . . 174 | 150/175 (85%) |
| | 1 . . . 175 | 156/175 (88%) |

Further analysis of the NOV13a protein yielded the following properties shown in Table 13C.

TABLE 13C
Protein Sequence Properties NOV13a

| | |
|---|---|
| PSort | 0.6500 probability located in cytoplasm; 0.1000 probability located in |
| analysis: | mitochondrial matrix space; 0.1000 probability located in lysosome (lumen); |
| | 0.1000 probability located in plasma membrane |
| SignalP | No Known Signal Sequence Predicted |
| analysis | |

A search of the NOV13 a protein against the Geneseq database, a proprietary database that contains sequences published in patents and patent publication, yielded several homologous proteins shown in Table 13D.

TABLE 13D
Geneseq Results for NOV13a

| | | NOV13a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB43393 | Human ORFX ORF3157 polypeptide | 1 . . . 174 | 143/175 (81%) | 5e-76 |
| | sequence SEQ ID NO:6314 - Homo | 1 . . . 175 | 158/175 (89%) | |
| | sapiens, 175 aa. [WO200058473-A2, | | | |
| | 05-OCT-2000] | | | |
| AAG04027 | Human secreted protein, SEQ ID NO: | 1 . . . 91 | 74/91 (81%) | 4e-35 |
| | 8108 - Homo sapiens, 102 aa. | 1 . . . 91 | 81/91 (88%) | |
| | [EP1033401-A2, 06-SEP-2000] | | | |
| AAM41045 | Human polypeptide SEQ ID NO 5976 - | 7 . . . 79 | 21/73 (28%) | 1.2 |
| | Homo sapiens, 973 aa. [WO200153312- | 165 . . . 232 | 33/73 (44%) | |
| | A1, 26-JUL-2001] | | | |
| AAY53667 | Sequence gi/3328186 from an | 97 . . . 161 | 21/70 (30%) | 2.6 |
| | alignment with protein 608 - | 143 . . . 212 | 37/70 (52%) | |
| | Unidentified, 3117 aa. [WO9960164- | | | |
| | A1, 25-NOV-1999] | | | |
| AAW46822 | Amino acid sequence of FBP encoded | 13 . . . 88 | 22/90 (24%) | 3.4 |
| | by the 5′ region of the gene - | 171 . . . 260 | 35/90 (38%) | |
| | Streptococcus equi, 413 aa. | | | |
| | [WO9801561-A1, 15-JAN-1998] | | | |

In a BLAST search of public sequence datbases, the NOV13a protein was found to have homology to the proteins shown in the BLASTP data in Table 13E.

TABLE 13E
Public BLASTP Results for NOV13a

| | | NOV13a | Identities/ | |
|---|---|---|---|---|
| Protein | | Residues/ | Similarities for | |
| Accession | | Match | the Matched | Expect |
| Number | Protein/Organism/Length | Residues | Region | Value |
| Q9P032 | HSPC125 (MY013 PROTEIN) | 1 . . . 174 | 143/175 (81%) | 1e-75 |
| | (BA22L21.1.1) (HSPC125 PROTEIN, | 1 . . . 175 | 158/175 (89%) | |
| | ISOFORM 1) - Homo sapiens | | | |
| | (Human), 175 aa. | | | |
| Q9NQR8 | HRPAP20 SHORT FORM - Homo | 1 . . . 174 | 119/175 (68%) | 2e-60 |
| | sapiens (Human), 174 aa. | 1 . . . 174 | 142/175 (81%) | |
| Q9D1H6 | 1110007M04RIK PROTEIN - Mus | 1 . . . 741 | 119/175 (68%) | 1e-58 |
| | musculus (Mouse), 173 aa. | 1 . . . 173 | 139/175 (79%) | |
| Q9VH39 | CG11722 PROTEIN - Drosophila | 5 . . . 162 | 57/168 (33%) | 2e-17 |
| | melanogaster (Fruit fly), 203 aa. | 8 . . . 175 | 88/168 (51%) | |
| Q9CTZ6 | 3000003G13RIK PROTEIN - Mus | 1 . . . 45 | 30/45 (66%) | 1e-08 |
| | musculus (Mouse), 120 aa (fragment). | 1 . . . 45 | 33/45 (72%) | |

PFam analysis predicts that the NOV13a protein contains the domains shown in the Table 13F.

TABLE 13F
Domain Analysis of NOV13a

| | | | |
|---|---|---|---|
| | | Identities/ | |
| Pfam | NOV13a | Similarities for | Expect |
| Domain | Match Region | the Matched Region | Value |

No Significant Matches Found

### Example 14.

The NOV14 clone was analyzed, and the nucleotide and encoded polypeptide sequences are shown in Table 14A.

TABLE 14A
NOV14 Sequence Analysis

| | SEQ ID NO: 35 | 3093 bp |
|---|---|---|
| NOV 14, | ATGAGCTCCCAAAGCCATCCAGATGGACTTTCTGGCCGAGACCAGCCAGTGGAGCTGC | |
| CG88768-01 DNA | TGAATCCTGCCCGCGTGAACCACATGCCCAGCACGGTGGATGTGGCCACGCCGCTGCC | |
| Sequence | TCTGCAAGTGGCCCCCTCGGCAGTGCCCATCGACCTGCCCCTGGACCACCAGTTCTCA | |
| | CTGCCTGTGGCAGAGCCGGCCCTGCGGGAGCAGcAGCTGCAGCAGCAGCTCCTGGCGC | |
| | TCAAGCAGAAGCAGCAGATCCAGAGGCAGATCCTCATCGCTGAGTTCCAGAGGCAGCA | |
| | CGAGCAGCTCTCCCGGCAGCACGAGGCGCAGCTCCACGAGCACATCAAGCAACAACAG | |
| | GAGATGCTGGCCATGAAGCACCAGCAGGAGCTGCTGGAACACCAGCGGAAGCTGGAGA | |
| | GGCACCGCCAGGAGCAGGAGCTGGAGAAGCAGCACCGGGAGCAGAAGCTGCAGCAGCT | |
| | CAAGAACAAGGAGAAGGGCAAAGAGAGTGCCGTGGCCAGCACAGAAGTGAAGATGAAG | |
| | TTACAAGAATTTGTCCTCAATAAAAAGAAGGCGCTGGCCCACCGGAATCTGAACCACT | |
| | GCATTTCCAGCGACCCTCGCTACTGGTACGGGAAAACGCAGCACAGTTCCCTTGACCA | |
| | GAGTTCTCCACCCCAGAGCGGAGTGTCGACCTCCTATAACCACCCGGTCCTGGGAATG | |
| | TACGACGCCAAAGATGACTTCCCTCTTAGGAAAACAGCTTCTGAACCGAATCTGAAAT | |
| | TACGGTCCAGGCTAAAGCAGAAAGTGGCCGAAAGACGCAGCAGCCCCCTGTTACGCAG | |
| | GAAAGACGGGCCAGTGGTCACTGCTCTAAAAAAGCGTCCGTTGGATGTCACAGACTCC | |
| | GCGTGCAGCAGCGCCCCAGGCTCCGGACCCAGCTCACCCAACAACAGCTCCGGGAGCG | |
| | TCAGCGCGGAGAACGGTATCGCGCCCGCCGTCCCCAGCATCCCGGCGGAGACGAGTTT | |
| | GGCGCACAGACTTGTGGCACGAGAAGGCTCGGCCGCTCCACTTCCCCTCTACACATCG | |
| | CCATCCTTGCCCAACATCACGCTGGGCCTGCCTGCCACCGGCCCCTCTGCGGGCACGG | |
| | CGGGCCAGCAGGACACCGAGAGACTCACCCTTCCCGCCCTCCAGCAGAGGCTCTCCCT | |
| | TTTCCCCGGCACCCACCTCACTCCCTACCTGAGCACCTCGCCCTTGGAGCGGGACGGA | |
| | GGGGCAGCCCACAGCCCTCTTCTGCAGCACATGGTCTTACTGGAGCAGCCACCGGCAC | |
| | AAGCACCCCTCGTCACAGGCCTGGGAGCACTGCCCCTCCACGCACAGTCCTTGGTTGG | |
| | TGCAGACCGGGTGTCCCCCTCCATCCACAAGCTGCGGCAGCACCGCCCACTGGGGCGG | |
| | ATTCAGTCGGCCCCGCTGCCCCAGAACGCCCAGGCTCTGCAGCACCTGGTCATCCAGC | |
| | AGCAGCATCAGCAGTTTCTGGAGAAACACAAGCAGCAGTTCCAGCAGCAGCAACTGCA | |
| | GATGAACAAGATCATCCCCAAGCCAAGCGAGCCAGCCAGGCAGCCGGAGAGCCACCCG | |
| | GAGGAGACGGAGGAGGAGCTCCGTGAGCAGGAGCTGCTCTTCAGACAGCAAGCCCTCC | |
| | TGCTGGAGCAGCAGCGGATCCACCAGCTGAGGAACTACCAGGCGTCCATGGAGGCCGC | |
| | CGGCATCCCCGTGTCCTTCGGCGGCCACAGGCCTCTGTCCCGGCCGCAGTCCTCACCC | |
| | GCGTCTGCCACCTTCCCCGTGTCTGTGCAGGAGCCCCCCACCAAGCCGAGGTTCACGA | |
| | CAGGCCTCGTGTATGACACGCTGATGCTGAAGCACCAGTGCACCTGCGGGAGTAGCAG | |
| | CAGCCACCCCGAGCACGCCGGGAGGATCCAGAGCATCTGGTCCCGCCTGCAGGAGACG | |
| | GGCCTCCGGGGCAAATGCGAGTGCATCCGCGGACGCAAGGCCACCCTGGAGGAGCTAC | |
| | AGACGGTGCACTCGGAAGCCCACACCCTCCTGTATGGCACGAACCCCCTCAACCGGCA | |
| | GAAACTGGACAGTAAGAAACTTCTAGGCTCGCTCGCCTCCGTGTTCGTCCGGCTCCCT | |
| | TGCGGTGGTGTTGGGGTGGACAGTGACACCATATGGAACGAGGTGCACTCGGCGGGGG | |
| | CAGCCCGCCTGGCTGTGGGCTGCGTGGTAGAGCTGGTCTTCAAGGTGGCCACAGGGGA | |
| | GCTGAAGAATGGCTTTGCTGTGGTCCGCCCCCCTGGACACCATGCGGAGGAGAGCACG | |
| | CCCATGGGCTTTTGCTACTTCAACTCCGTGGCCGTGGCAGCCAAGCTTCTGCAGCAGA | |
| | GGTTGAGCGTCAGCAAGATCCTCATCGTGGACTGGGACGTGCACCATGGAAACGGGAC | |
| | CCAGCAGGCTTTCTACAGCGACCCTAGCGTCCTGTACATGTCCCTCCACCGCTACGAC | |
| | GATGGGAACTTCTTCCCAGGCAGCGGGGCTCCTGATGAGGTGGGCACAGGGCCCGGCG | |
| | TGAGTACTTGGCGGCCTTCACAACGGTGGTCATGCCGATCGCCAGCGAGTTTGCCCCG | |
| | GATGTGGTGCTGGTGTCATCAGGCTTCGATGCCGTGGAGGGCCACCCCACCCCTCTTG | |
| | GGGGCTACAACCTCTCCGCCAGATGCTTCGGGTACCTGACGAAGCAGCTGATGGGCCT | |
| | GGCTGGCGGCCGGATTGTCCTGGCCCTCGAGGGAGGCCACGACCTGACCGCCATTTGC | |
| | GACGCCTCGGAAGCATGTGTTTCTGCCTTGCTGGGAAACGAGCTTGATCCTCTCCCAG | |
| | AAAAGGTTTTACAGCAAAGACCCAATGCAAACGCTGTCCGTTCCATGGAGAAAGTCAT | |
| | GGAGATCCACAGCAAGTACTGGCGCTGCCTGCAGCGCACAACCTCCACAGCGGGGCGT | |
| | TCTCTGATCGAGGCTCAGACTTGCGAGAACGAAGAAGCCGAGACGGTCACCGCCATGG | |
| | CCTCGCTGTCCGTGGGCGTGAAGCCCGCCGAAAAGAGACCAGATGAGGAGCCCATGGA | |
| | AGAGGAGCCGCCCCTGTAG | |
| | ORF Start: ATG at 1 | ORF Stop: TAG at 3091 |
| | SEQ ID NO: 36 | 1030 aa MW at 113012.2 kD |

| | |
|---|---|
| NOV14, | MSSQSHPDCLSGRDQPVELLNPARVNHMPSTVDVATALPLQVAPSAVPMDLRLDHQFS |
| CG88768-01 Protein | LPVAEPALREQQLQQELLALKQKQQIQRQILIAEFQRQHEQLSRQHEAQLHEHIKQQQ |
| Sequence | EMLAMKHQQELLEHQRKLERHRQEQELEKQHREQKLQQLKNKEKGKESAVASTEVKMK |
| | LQEFVLNKKKALAHRNLNHCTSSDPRYWYGKTQHSSLDQSSPPQSGVSTSYNHPVLGM |
| | YDAKDDFPLRKTASEPNLKLRSRLKQKVAERRSSPLLRRKDGPVVTALKKRPLDVTDS |
| | ACSSAPGSGPSSPNNSSGSVSAENGIAPAVPSIPAETSLAHRLVAREGSAAPLPLYTS |
| | PSLPNITLGLPATGPSAGTAGQQDTERLTLPALQQRLSLFPGTHLTPYLSTSPLERDG |
| | GAAHSPLLQHMVLLEQPPAQAPLVTGLGALPLHAQSLVGADRVSPSIHKLRQHRPLGR |
| | IQSAPLPQNAQALQHLVIQQQHQQFLEKHKQQFQQQQLQMNKIIPKPSEPARQPESHP |
| | EETEEELREQELLFRQQALLLEQQRIHQLRNYQASMEAAGTPVSFGGHRPLSRAQSSP |
| | ASATFPVSVQEPPTKPRFTTGLVYDTLMLKHQCTCGSSSSHPEHAGRIQSIWSRLQET |
| | GLRGKCECIRGRKATLEELQTVHSEAHTLLYGTNPLNRQKLDSKKLLGSLASVFVRLP |
| | CGGVGVDSDTTWNEVHSAGAARLAVGCVVELVFKVATGELKNGFAVVRPPGHHAEEST |
| | PMGFCYFNSVAVAAKLLQQRLSVSKILIVDWDVHHGNGTQQAFYSDPSVLYMSLHRYD |
| | DGNFFPGSGAPDEVGTGPGVGFNVNMAFTGGLDPPMGDAEYLAAFRTVVMPIASEFAP |
| | DVVLVSSGFDAVEGHPTPLGGYNLSARCFGYLTKQLMGLAGGRIVLALEGCHDLTATC |
| | DASEACVSALLGNELDPLPEKVLQQRPNANAVRSMEKVMEIHSKYWRCLQRTTSTAGR |
| | SLIEAQTCENEEAETVTAMASLSVGVKPAEKRPDEEPMEEEPPL |

Further analysis of the NOV14 protein yielded the following properties shown in Table 14B.

TABLE 14B
Protein Sequence Properties NOV14

| | |
|---|---|
| PSort | 0.3000 probability located in microbody (peroxisome); 0.3000 probability |
| analysis: | located in nucleus; 0.1580 probability located in lysosome (lumen); 0.1000 |
| | probability located in mitochondrial matrix space |
| SignalP | No Known Signal Sequence Predicted |
| analysis: | |

A search of the NOV14 protein against the Geneseq database, a proprietary database that contains sequences published in patents and patent publication, yielded several homologous proteins shown in Table 14C.

TABLE 14C
Geneseq Results for NOV14

| | | NOV14 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB49957 | Human histone deacetylase HDAC-4 | 118 . . . 1030 | 910/967 (94%) | 0.0 |
| | Homo sapiens, 967 aa. | 1 . . . 967 | 910/967 (94%) | |
| | [WO200071703-A2, 30-NOV-2000] | | | |
| AAB43008 | Human ORFX ORF2772 polypeptide | 8 . . . 1030 | 651/1143 (56%) | 0.0 |
| | sequence SEQ ID NO:5544 - Homo | 27 . . . 1141 | 792/1143 (68%) | |
| | sapiens, 1141 aa. [WO200058473-A2, | | | |
| | 05-OCT-2000] | | | |
| AAY07092 | Colon cancer associated antigen | 177 . . . 1000 | 527/919 (57%) | 0.0 |
| | precursor sequence - Homo sapiens, | 1 . . . 896 | 634/919 (68%) | |
| | 897 aa. [WO9904265-A2, 28-JAN- | | | |
| | 1999] | | | |
| AAM78891 | Human protein SEQ ID NO 1553 - | 100 . . . 1030 | 502/977 (51%) | 0.0 |
| | Homo sapiens, 1008 aa. | 76 . . . 1006 | 627/977 (63%) | |
| | [WO200157190-A2, 09-AUG-2001] | | | |
| AAM79875 | Human protein SEQ ID NO 3521 - | 44 . . . 1030 | 511/1047 (48%) | 0.0 |
| | Homo sapiens, 1020 aa. | 20 . . . 1018 | 650/1047 (61%) | |
| | [WO200157190-A2, 09-AUG-2001] | | | |

In a BLAST search of public sequence datbases, the NOV14 protein was found to have homology to the proteins shown in the BLASTP data in Table 14D.

TABLE 14D
Public BLASTP Results for NOV14

| | | NOV14 | Identities/ | |
|---|---|---|---|---|
| Protein | | Residues/ | Similarities for | |
| Accession | | Match | the Matched | Expect |
| Number | Protein/Organism/Length | Residues | Region | Value |
| P56524 | Histone deacetylase 4 (HD4) | 1 . . . 1030 | 1022/1084 (94%) | 0.0 |
| | (HA6116) - Homo sapiens (Human), | 1 . . . 1084 | 1024/1084 (94%) | |
| | 1084 aa. | | | |
| P83038 | Histone deacetylase 4 (HD4) - Gallus | 1 . . . 1030 | 941/1084 (86%) | 0.0 |
| | gallus (Chicken), 1080 aa. | 1 . . . 1080 | 983/1084 (89%) | |
| Q9UQL6 | Histone deacetylase 5 (HD5) | 1 . . . 1030 | 653/1150 (56%) | 0.0 |
| | (Antigen NY-CO-9) - Homo sapiens | 1 . . . 1122 | 796/1150 (68%) | |
| | (Human), 1122 aa. | | | |
| Q9Z2V6 | Histone deacetylase 5 (HD5) | 1 . . . 1030 | 650/1141 (56%) | 0.0 |
| | (Histone deacetylase mHDA1) - Mus | 1 . . . 1113 | 791/1141 (68%) | |
| | musculus (Mouse), 1113 aa. | | | |
| Q9UKV0 | Histone deacetylase 9 (HD9) | 25 . . . 971 | 579/1016 (56%) | 0.0 |
| | (HD7B) (HD7) - Homo sapiens | 1 . . . 1005 | 718/1016 (69%) | |
| | (Human), 1011 aa. | | | |

PFam analysis predicts that the NOV14 protein contains the domains shown in the Table 14E.

TABLE 14E
Domain Analysis of NOV14

| | NOV14 | Identities/ | |
|---|---|---|---|
| | Match | Similarities for the | Expect |
| Pfam Domain | Region | Matched Region | Value |
| HK: domain 1 of 1 | 453 . . . 462 | 5/10 (50%) | 6.2 |
| | | 10/10 (100%) | |
| REV: domain 1 of 1 | 458 . . . 484 | 11/27 (41%) | 4.1 |
| | | 21/27 (78%) | |
| Hist_deacetyl: | 598 . . . 944 | 134/360 (37%) | 1.4e-109 |
| domain 1 of 1 | | 274/360 (76%) | |
| GATase: domain 1 of 1 | 832 . . . 996 | 36/275 (13%) | 8.5 |
| | | 100/275 (36%) | |

### Example 15.

The NOV15 clone was analyzed, and the nucleotide and encoded polypeptide sequences are shown in Table 15A.

TABLE 15A
NOV15 Sequence Analysis

| | SEQ ID NO: 37 | 1750 bp |
|---|---|---|
| NOV 15a, | CAGGATGAACGCTGCTTTCCAAGATGGCGACGGAGGGAGGAGGGAAGGAGATGAACGA | |
| CG88856-01 DNA | GATTAAGACCCAATTCACCACCCGGGAAGGTCTGTACAAGCTGCTGCCGCACTCGGAG | |
| Sequence | TACAGCCGGCCCAACCGGGTGCCCTTCAACTCGCAGGGATCCAACCCTGTCCGCGTCT | |
| | CCTTCGTAAACCTCAACGACCAGTCTGGCAACGGCGACCGCCTCTGCTTCAATGTGGG | |
| | CCGGGAGCTGTACTTCTATATCTACAAGGGGGTCCGCAAGGCTGCTGACTTGAGTAAA | |
| | CCAATAGATAAAAGGATATACAAAGGAACACAGCCTACTTGTCATGACTTCAACCACC | |
| | TAACAGCCACAGCAGAAAGTGTCTCTCTCCTAGTGGGCTTTTCCGCAGGCCAAGTCCA | |
| | GCTTATAGACCCAATCAAAAAAGAAACTAGCAAACTTTTTAATGAGGAAAGACTAATA | |
| | GACAAGTCACGAGTTACCTGTGTCAAATGGGTTCCCGGTTCGGAAAGCCTTTTCCTAG | |
| | TAGCCCACTCGAGTGGGAACATGTACTTATATAATGTGGAGCACACTTGTGGCACCAC | |
| | AGCCCCCCACTACCAGCTTCTGAAGCAGGGAGAGAGCTTTGCCGTGCACACTTGCAAG | |
| | AGCAAATCCACGAGGAACCCTCTCCTTAAGTGGACGGTGGGCGAGGGGGCCCTCAACG | |
| | AGTTTGCTTTCTCCCCAGATGGCAAGTTCTTAGCGTGCGTGAGCCAGGACGGGTTTCT | |
| | GCGGGTGTTCAACTTTGACTCAGTGGAGCTGCACGGTACGATGAAAAGCTACTTTGGG | |
| | GGCTTGCTGTGTGTGTGCTGGAGCCCGGATGGCAAGTACATCGTGACAGGTGGGGAGG | |
| | ACCACTTGGTGACAGTCTGGTCCTTTGTAGACTGCCGAGTAATAGCCAGAGGCCACGG | |
| | GCACAAGTCCTGGGTCAGTGTTGTAGCGTTTGACCCTTATACCACTAGTGTAGAAGAA | |
| | GGTGACCCTATGGAGTTTAGTGGCAGCGATGAGGACTTCCAAGACCTTCTTCATTTTG | |
| | GCAGAGATCGAGCAAATAGTACACAGTCCAGGCTCTCCAAACGGAACTCTACAGACAG | |
| | CCGCCCCGTAAGTGTCACGTATCGGTTTGGTTCCGTGGGCCAGGACACACACCTCTGT | |
| | TTATGGGACCTTACAGAAGATATCCTTTTCCCTCACCAACCCCTCTCAAGAGCAAGGA | |
| | CACACACAAATGTCATGAATGCCACGAGTCCTCCTGCTGGAAGCAATGGGAACAGTGT | |
| | TACAACACCCGGGAACTCTGTGCCGCCTCCTCTGCCACGGTCCAACAGCCTTCCACAT | |
| | TCAGCAGTCTCAAATGCTGGCAGCAAAAGCAGTGTCATGCACGGGGCCATTGCTTCTG | |
| | GGGTCAGCAAATTTGCAACACTTTCACTACATGACCGGAAGGAGAGGCACCACGAGAA | |
| | AGATCACAAGCGAAATCATAGCATGGGACACATTTCTAGCAAGAGCAGTGACAAACTG | |
| | AATCTAGTTACCAAAACCAAAACGGACCCTGCTAAAACTCTGGGAACCCCCCTCTGTC | |
| | CTCGAATGGAAGATCTTCCCTTGTTAGAGCCGCTGATATGTAAAAACATAGCACATGA | |
| | GAGACTGACTGTACTAATATTTCTTGAAGACTGTATAGTCACTGCTTGTCAGGAGGGA | |
| | TTTATTTGCACATGGGGAAGGCCTGCTAAAGTGGTAAGTTTTAATCCTTAATGCTGCA | |
| | CCAGATCTAG | |
| | ORF Start: ATG at 24 | ORF Stop: TAA at 1731 |
| | SEQ ID NO: 38 | 569 aa MW at 62892.5 kD |

| | |
|---|---|
| NOV15 | MATEGGGKEMNETKTQFTTREGLYKLLPHSEYSRPNRVPFNSQGSNPVRVSFVNLNDQ |
| CG88856-01 Protein | SGNGDRLCFNVGRELYFYIYKGVRKAADLSKPIDKRTYKGTQPTCHDFNHLTATAESV |
| Sequence | SLLVGFSACQVQLIDPIKKETSKLFNEERLIDKSRVTCVKWVPGSESLFLVAHSSCNM |
| | YLYNVEHTCGTTAPHYQLLKQGESFAVHTCKSKSTRNPLLKWTVGEGALNEPAFSPDG |
| | KFLACVSQDGFLRVFNFDSVELHGTMKSYFGGLLCVCWSPDGKYIVTGGEDDLVTVWS |
| | FVDCRVTARGHGHKSWVSVVAFDPYTTSVEEGDPMEFSGSDEDFQDLLHFGRDRANST |
| | QSRLSKRNSTDSRPVSVTYRFGSVGQDTQLCLWDLTEDILFPHQPLSRARTHTNVMNA |
| | TSPPAGSNGNSVTTPGNSVPPPLPRSNSLPHSAVSNAGSKSSVNDGATASGVSKFATL |
| | SLHDRKERHHEKDHKRNHSMGHISSKSSDKLNLVTKTKTDPAKTLGTPLCPRMEDVPL |
| | LEPLICKKIAHERLTVLIFLEDCIVTACQECFICTWGRPGKVVSFNP |

Further analysis of the NOV15 protein yielded the following properties shown in Table 15B.

TABLE 15B
Protein Sequence Properties NOV15

| | |
|---|---|
| Psort | 0.4692 probability located in microbody (peroxisome); 0.4500 |
| analysis: | probability located in cytoplasm; 0.1000 probability located in |
| | mitochondrial matrix space; 0.1000 probability located in |
| | lysosome (lumen) |
| SignalP | No Known Signal Sequence Predicted |
| analysis: | |

A search of the NOV15 protein against the Geneseq database, a proprietary database that contains sequences published in patents and patent publication, yielded several homologous proteins shown in Table 15C.

TABLE 15C
Geneseq Results for NOV15

| | | NOV15 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAG65160 | Human myotonic dystrophy protein | 174 . . . 569 | 396/396 (100%) | 0.0 |
| | kinase 44 - Homo sapiens, 396 aa. | 1 . . . 396 | 396/396 (100%) | 0.0 |
| | [WO200164728-A1, 07-SEP-2001] | | | |
| AAB42704 | Human ORFX ORF2468 polypeptide | 232 . . . 569 | 318/338 (94%) | 0.0 |
| | sequence SEQ ID NO: 4936 - Homo | 1 . . . 337 | 321/338 (94%) | 0.0 |
| | sapiens, 337 aa. [WO200058473-A2, | | | |
| | 05-OCT-2000] | | | |
| AAM40094 | Human polypeptide SEQ ID NO 3239 | 258 . . . 569 | 312/312 (100%) | 0.0 |
| | - Homo sapiens, 312 aa. | 1 . . . 312 | 312/312 (100%) | |
| | [WO200153312-A1, 26-JUL-2001] | | | |
| AAM78352 | Human protein SEQ ID NO 1014- | 12 . . . 563 | 342/634 (53%) | 0.0 |
| | Homo sapiens, 684 aa. | 21 . . . 646 | 405/634 (62%) | |
| | [WO200157190-A2, 09-AUG-2001] | | | |
| AAM79336 | Human protein SEQ ID NO 2982 - | 12 . . . 563 | 339/634 (53%) | e-179 |
| | Homo sapiens, 687 aa. | 21 . . . 646 | 402/634 (62%) | |
| | [WO200157190-A2, 09-AUG-2001] | | | |

In a BLAST search of public sequence datbases, the NOV15 protein was found to have homology to the proteins shown in the BLASTP data in Table 15D.

TABLE 15D
Public BLASTP Results for NOV15

| | | NOV15 | Identities/ | |
|---|---|---|---|---|
| Protein | | Residues/ | Similarities for | |
| Accession | | Match | the Matched | Expect |
| Number | Protein/Organism/Length | Residues | Portion | Value |
| AAL56014 | DMR PROTEIN - Homo sapiens | 10 . . . 568 | 554/559 (99%) | 0.0 |
| | (Human), 572 aa. | 1 . . . 559 | 556/559 (99%) | |
| Q9D5R2 | 4921538B03RIK PROTEIN - Mus | 1 . . . 569 | 526/569 (92%) | 0.0 |
| | musculus (Mouse), 567 aa. | 1 . . . 567 | 540/569 (94%) | |
| Q9D5L0 | 4930427E19RIK PROTEIN - Mus | 174 . . . 569 | 362/396 (91%) | 0.0 |
| | musculus (Mouse), 394 aa. | 1 . . . 394 | 371/396 (93%) | |
| Q9UF86 | HYPOTHETICAL 37.0 KDA | 232 . . . 569 | 337/338 (99%) | 0.0 |
| | PROTEIN - Homo sapiens (Human), | 1 . . . 338 | 337/338 (99%) | |
| | 338 aa (fragment). | | | |
| Q08274 | Dystrophia myotonica-containing WD | 12 . . . 563 | 345/619 (55%) | 0.0 |
| | repeat motif protein (DMR-N9 protein) | 6 . . . 609 | 410/619 (65%) | |
| | - Mus musculus (Mouse), 650 aa. | | | |

PFam analysis predicts that the NOV15 protein contains the domains shown in the Table 15E.

TABLE 15E
Domain Analysis of NOV15

| | | Identities/ | |
|---|---|---|---|
| | NOV15 | Similarities for the | Expect |
| Pfam Domain | Match Region | Matched Region | Value |
| WD40: domain 1 of 7 | 99 . . . 131 | 10/37 (27%) | 8.5e + 02 |
| | | 25/37 (68%) | |
| WD40: domain 2 of 7 | 142 . . . 178 | 7/38 (18%) | 9.3 |
| | | 24/38 (63%) | |
| WD40: domain 3 of 7 | 213 . . . 248 | 14/37 (38%) | 0.025 |
| | | 31/37 (84%) | |
| WD40: domain 4 of 7 | 254 . . . 290 | 10/37 (27%) | 0.0033 |
| | | 28/37 (76%) | |
| WD40: domain 5 of 7 | 296 . . . 328 | 9/37 (24%) | 59 |
| | | 23/37 (62%) | |
| WD40: domain 6 of 7 | 352 . . . 382 | 6/37 (16%) | 7e + 02 |
| | | 22/37 (59%) | |
| WD40: domain 7 of 7 | 526 . . . 559 | 9/37 (24%) | 2.1e + 02 |
| | | 20/37 (54%) | |

### Example 16.

The NOV16 clone was analyzed, and the nucleotide and encoded polypeptide sequences are shown in Table 16A.

TABLE 16A
NOV16 Sequence Analysis

| | SEQ ID NO: 39 | 554 bp |
|---|---|---|
| NOV16, | ACTGGGAAGGCGCAAGCCGTCGTGAAGCAGGCCGGTTACAGTGAGGTCTATTCGCTCG | |
| CG89958-01 DNA | AGGGCGGATTGGCCGCGTGGCAGCAGGCAGGCCTTCCGGGTCGTCAAATAAAGAAACG | |
| Sequence | AGGTTTTGAAGTTATGGCGCACGTGGTTATGTACAGCACCACCGTCTGCCCCTATTGC | |
| | GTGGCAGCGGAACGACTCCTGAAGCAGCGCGGCGTCGAGCAGATCGAAAAGATCCTGA | |
| | TCGACCGCGAACCCGGCAAACGCGAAGAGATGATGACGCGCACGAACCGTCGCACCGT | |
| | GCCGCAGATCTACATCGACGATCGCCACATTGGCGGCTTCGATGATCTCTCTGCGCTG | |
| | GACCGCGAAGGCGGGCTGGTGCCACTGCTGGCGGCCTGAGCGCCACACCAAAACGCCC | |
| | GGCTTTGACCGGGCGTTGCACATTTAGGCCTGCTCTCATGGTGGGCACGATTGCGTCA | |
| | TGTACCATACGCGTCTTGCGCGTGGGACACATCCCCGCCGCGCACTGACCATACATCT | |
| | ATCTGAAGGCGAGTCATGAGCGACCAGCAACA | |
| | ORF Start: ATG at 130 | ORF Stop: TGA at 385 |
| | SEQ ID NO: 40 | 85 aa MW at 9658.1 kD |

| | |
|---|---|
| NOV16, | MAHVVMYSTTVCPYCVAAERLLKQRGVEQIEKILIDREPGKREEMMTRTNRRTVPQIY |
| CG89958-01 Protein | IDDRHIGGFDDLSALDREGGLVPLLAA |
| Sequence | |

Further analysis of the NOV16a protein yielded the following properties shown in Table 16B.

TABLE 16B
Protein Sequence Properties NOV16

| | |
|---|---|
| PSort | 0.4632 probability located in mitochondrial matrix space; |
| analysis: | 0.4500 probability located in cytoplasm; 0.2107 probability |
| | located in lysosome (lumen); 0.1612 probability located in |
| | mitochondrial inner membrane |
| SignalP | Cleavage site between residues 19 and 20 |
| analysis: | |

A search of the NOV16 protein against the Geneseq database, a proprietary database that contains sequences published in patents and patent publication, yielded several homologous proteins shown in Table 16C.

TABLE 16C
Geneseq Results for NOV16

| | | NOV16 | Identities/ | |
|---|---|---|---|---|
| | | Residues/ | Similarities for | |
| Geneseq | Protein/Organism/Length | Match | the Matched | Expect |
| Identifier | Patent [190 , Date] | Residues | Region | Value |
| AAU72998 | Neisseria meningitidis virulence protein | 1 . . . 83 | 39/83 (46%) | 4e-15 |
| | #88 -Neisseria meningitidis, 93 aa. | 9 . . . 91 | 51/83 (60%) | |
| | [WO200185772-A2, 15-NOV-2001] | | | |
| AAG33782 | Arabidopsis thaliana protein fragment | 4 . . . 85 | 34/84 (40%) | 4e-11 |
| | SEQ ID NO: 40996 - Arabidopsis | 46 . . . 129 | 50/84 (59%) | |
| | thaliana, 132 aa. [EP1033405-A2, | | | |
| | 06-SEP-2000] | | | |
| AAG35055 | Arabidopsis thaliana protein fragment | 4 . . . 83 | 34/82 (41%) | 5e-10 |
| | SEQ ID NO: 42764- Arabidopsis | 13 . . . 94 | 47/82 (56%) | |
| | thaliana, 109 aa. [EP1033405-A2, | | | |
| | 06-SEP-2000] | | | |
| AAG35054 | Arabidopsis thaliana protein fragment | 4 . . . 83 | 34/82 (41%) | 5e-10 |
| | SEQ ID NO: 42763 - Arabidopsis | 15 . . . 96 | 47/82 (56%) | |
| | thaliana, 111 aa. [EP1033405-A2, | | | |
| | 06-SEP-2000] | | | |
| AAG45926 | Arabidopsis thaliana protein fragment | 4 . . . 83 | 34/83 (40%) | 6e-10 |
| | SEQ ID NO: 57719- Arabidopsis | 13 . . . 94 | 49/83 (58%) | |
| | thaliana, 109 aa. [EP1033405-A2, | | | |
| | 06-SEP-2000] | | | |

In a BLAST search of public sequence datbases, the NOV16 protein was found to have homology to the proteins shown in the BLASTP data in Table 16D.

TABLE 16D
Public BLASTP Results for NOV16

| | | NOV16 | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD13883 | PROBABLE GLUTAREDOXIN 3 | 1 . . . 85 | 80/85 (94%) | 7e-41 |
| | (GRX3) PROTEIN - Ralstonia | 1 . . . 85 | 82/85 (96%) | |
| | solanacearum (Pseudomonas | | | |
| | solanacearum), 85 aa. | | | |
| CAC88932 | GLUTAREDOXIN - Yersinia pestis, | 1 . . . 83 | 45/83 (54%) | 1e-18 |
| | 82 aa. | 1 . . . 82 | 56/83 (67%) | |
| S47831 | glutaredoxin 3 (grx3) - Escherichia coli, | 1 . . . 83 | 45/83 (54%) | 1e-17 |
| | 83 aa. | 1 . . . 82 | 57/83 (68%) | |
| AAL22561 | GLUTAREDOXIN 3 - Salmonella | 1 . . . 83 | 44/83 (53%) | 2e-17 |
| | typhimurium LT2, 83 aa. | 1 . . . 82 | 58/83 (69%) | |
| Q9PAC3 | GLUTAREDOXIN - Xylella fastidiosa, | 4 . . . 83 | 40/80 (50%) | 3e-17 |
| | 118 aa. | 33 . . . 111 | 55/80 (68%) | |

PFam analysis predicts that the NOV16 protein contains the domains shown in the Table 16E.

TABLE 16E
Domain Analysis of NOV16

| | | Identities/ | |
|---|---|---|---|
| | NOV16 | Similarities for the | Expect |
| Pfam Domain | Match Region | Matched Region | Value |
| glutaredoxin: | 3 . . . 61 | 24/69 (35%) | 1.3e-13 |
| domain 1 of 1 | | 50/69 (72%) | |

### Example 17.

The NOV17 clone was analyzed, and the nucleotide and encoded polypeptide sequences are shown in Table 17A.

TABLE 17A
NOV17 Sequence Analysis

| | SEQ ID NO: 41 | 2267 bp |
|---|---|---|
| NOV17a, | TAGAATTCAGCGGCCGCTGAATTTCTTAACGCTTTAATGGGGCAAATTTGTTCTCTGC | |
| CG90309-01 DNA | ACGGGAAACATGTGGGCCCTTGTCAGGTGCTGCATCAGAGTGAGTTGCCCTCCACCAG | |
| Sequence | CTTCCTAGATCTGGCCGTGTGAGGAGGCAGAAGGAGCCCTCTGAGACTTTGGGGACAT | |
| | CTCCCATGGTGTGGCCCCAATCCTGTCCATCTGATGGTTTGTCCACCACTGAGTCCTC | |
| | CCCTCTAGAGGCTGGGAGTGCCCACATGGCCAAGGAGAAACCTCATCCATCCCCCTGC | |
| | TCCCTCTGCAGAGAAGCCCTGCAGTAAGAGAGTCCACAGGTCTCCCCCACACTGTATG | |
| | TACAGGCGGGTATTCAAGGCATTCTGTGTACCAGGCTTGTGTTGGAAGCGTGGGACAC | |
| | AAAGGCAGAGAAGACCTAGTCCCAGTCTCTGAGAGCTAATGAGGTGTGCTAGATGCAT | |
| | TCACGTAAAGGCAAGATCTTCTTATGTTGTCCAGGCTGGTCTTGGACTCCTCAGCTCA | |
| | AGCTATCTTTCTGCCTTGGTTTCCAAAGTAGCTGGGACTATAGGCATGCATTAACCTG | |
| | AGTTTTGAAGGATGAGTTGGAAGAGTTCAAATAGGACAAGCAAGTAGAGGAAACGCAG | |
| | ACTGGTGTGGCTACGGTGGAGGATGCCAAGTGGGGAGGCACGAGGCTGGGTGACAAGC | |
| | AGAGCCCGGCCAAGGAGTGTCTGCTGCTGGAAAGCCCAATCCAAAAGAGCGTGTAAAG | |
| | TGCTTGACAGAAAGAGGACGCTTGATAAATGCTAGCCAGCTCACTAGTGTGAAAGTGT | |
| | CATGAAGGCAGAAGTCATCCATCTGAATATGACTGTCTCCCCAGGTCCAGGTGCTGGC | |
| | ACAGAGGTGGCACCTAATACACATGTGTTGAAGAAGTCAATAGACATGCTCCTCCCAC | |
| | CACCCTGTCCTTTCCCTCCCTCCCTTTCCCTAGTCTCACTCTCATTTCCCCCAGTCCC | |
| | ACATTTTCTTTCCTAGTGCTCTTTTTCTCCTCTCGTGGAGGAAGGATGCTCTGGGCCC | |
| | AAATACCCCTTTGCTGTCCCAAAAGTTCCACTCTGGAAATGAGCCCCCCCGCAGCATT | |
| | GTGACATCACCGTGCACTAGCCAATGGCTGCCTGCCTAAGCTGGGTCCCTGGTCTCCT | |
| | GGGACTACTAGCCCTTTGTTGATAGGGAGAAGCCAACATCTCCCGCAGGACCCCCTAA | |
| | TCTTCAGGGCAGCTCCCAGAGCATGGATCCCTCCTGATTCCACTCAGCCCGATGTTCC | |
| | TCACAGTCAAGCTGCTCCTGGGCCAGAGATGCAGTCTGAAGGTGTCAGGGCAAGAGAG | |
| | TGTAGCCACGCTGAAGAGACTGGTGTCCAGGCGGCTGAAGGTGCCTGAGGAGCAGCAG | |
| | CACCTGCTTTTCCGTGGCCAGCTCCTGGAGGATGACAAGCACCTCTCTGACTACTGCA | |
| | TTGGGCCCAATGCCTCTATCAATGTCATCATGCAGCCCTTGGAGAAGATGGCGCTAAA | |
| | GGAGGCCCACCAGCCGCAGACCCAGCCCCTGTGGCACCAGCTGGGACTGGTCCTAGCT | |
| | AAACACTTTGAACCACAGGATGCCAAGGCCGTGCTGCAGCTGCTAAGGCAGGAGCACG | |
| | AGGAGCGCCTGCAGAAGATAAGCCTGGAGCACCTGGAGCAGCTGGCCCAGTACCTCCT | |
| | GGCAGAGGAGCCTCACGTGGAGCCAGCTGGAGAGAGGGAGCTTGAGGCGAAGGCACGG | |
| | CCTCAGAGCTCCTGTGACATGGAGGAGAAGGAGGAGGCAGCAGCTGATCAGTAAACGG | |
| | GCCATCCTACCCATTTGCATGCTAAAATTCTCCCGGCCTCATCCTTACGTGTTCCCTG | |
| | GTGACTTTTCCTACTACTTCCTGCTGATGTGGATGCGTCCACACCCCTTTTTGAACCT | |
| | TCCAAGCAGCTGGAGGGTTTTTGGATCCCTGTCCCCTCTTGGGCCTGAGGTCCTCCCT | |
| | CTGAAATGCAGAGTGAACCAACCCTCATCACCATGCTTCCCCTAGAAGGGTTCTGATC | |
| | ACCGGAGGGCAGCCCCAAAGGCCACAGTCCCCTCCTGTGCTGGCAGCTTTGCCCACAC | |
| | ATACCCAGCAGCTCCCCAGGCTGAAAGCAGCCCTGGCCCAGGGTCTCCATGGTTCTAG | |
| | GCAGACCCTCTTTCTCCTTCGGGACAGAAAGACAATGTGAGTTCATTTTCCTCCATCC | |
| | TCAGACCGTGACATCTCCCCTAGGCTCCCCAGCAGCCAAGAGGAGAGGAATGTCAGGT | |
| | AGCTG | |
| | ORF Start: ATG at 1270 | ORF Stop: TAA at 1792 |
| | SEQ ID NO: 42 | 174 aa MW at 19908.7 kD |
| NOV17a, | MFLTVKLLLGQRCSLKVSGQESVATLKRLVSRRLKVPEEQQHLLFRGQLLEDDKHLSD | |
| CG90309-01 Protein | YCIGPNASINVIMQPLEKMALKEAHQPQTQPLWHQLGLVLAKHFEPQDAKAVLQLLRQ | |
| Sequence | EHEERLQKISLEULEQLAQYLLAEEPHVEPAGERELEAKARPQSSCDMEEKEEAAADQ | |
| | SEQ ID NO: 43 | 657 bp |
| NOV17b, | CTTTGTTGATAGGGAGAAGCAACATCTCCCGCAGGACCCCCTAATCTTCAGGGCAGCT | |
| CG90309-02 DNA | CCCAGAGCATGGATCCCTCCTGATTCCACTCAGCCCGATGTTCCTCACAGTCAAGCTG | |
| Sequence | CTCCTGGGCCAGAGATGCAGTCTGAAGGTGTCAGGGCAAGAGAGTGTAGCCACGCTGA | |
| | AGAGACTGGTGTCCAGGCGGCTGAAGGTGCCTGAGGAGCAGCAGCACCTGCTTTTCCG | |
| | TGGCCAGCTCCTGGAGGATGACAAGCACCTCTCTGACTACTGCATTCGGCCCAATGCC | |
| | TCTATCAATGTCATCATGCAGCCCTTGGAGAAGATGGCGCTAAAGGAGGCCCACCAGC | |
| | CGCAGACCCAGCCCCTGTGGCACCAGCTGGGACTGGTCCTAGCTAAACACTTTGAACC | |
| | ACAGGATGCCAAGGCCGTGCTGCAGCTGCTAAGGCAGGAGCACGAGGAGCGCCTGCAG | |
| | AAGATAAGCCTGGAGCACCTGGAGCAGCTGGCCCAGTACCTCCTGGCAGAGGAGCCTC | |
| | ACGTGGAGCCAGCTGGAGAGAGGGAGCTTGAGGCGAAGGCACGGCCTCAGAGCTCCTG | |
| | TGACATGGAGGAGAAGGAGGAGGCAGCAGCTGATCAGTAAACGGGCCATCCTACCCAT | |
| | TTGCATGCTAAAATTCTCC | |
| | ORF Start: ATG at 96 | ORF Stop: TAA at 618 |
| | SEQ ID NO: 44 | 174 aa MW at 19908.7 kD |

| | |
|---|---|
| NOV17b, | MFLTVKLLLGQRCSLKVSGQESVATLKRLVSRRLKVPEEQQHLLFRGQLLEDDKHLSD |
| CG90309-02-Protein | YCIGPNASINVIMQPLEKMALKEAHQPQTQPLWHQLGLVLAKHFEPQDAKAVLQLLRQ |
| Sequence | EHEERLQKISLEHLEQLAQYLLAEEPHVEPAGERELEAKARPQSSCDMEEKEEAAADQ |

Sequence comparison of the above protein sequences yields the following sequence relationships shown in Table 17B.

TABLE 17B
Comparison of NOV17a against NOV17b.

| | NOV17a Residues/ | Identities/Similarities |
|---|---|---|
| Protein Sequence | Match Residues | for the Matched Region |
| NOV17b | 1 . . . 174 | 146/174 (83%) |
| | 1 . . . 174 | 146/174 (83%) |

Further analysis of the NOV17a protein yielded the following properties shown in Table 17C.

TABLE 17C
Protein Sequence Properties NOV17a

| | |
|---|---|
| PSort | 0.4641 probability located in mitochondrial matrix space; |
| analysis: | 0.4500 probability located in cytoplasm; 0.1627 probability |
| | located in mitochondrial inner membrane; 0.1627 probability |
| | located in mitochondrial intermembrane space |
| SignalP | Cleavage site between residues 20 and 21 |
| analysis: | |

A search of the NOV17a protein against the Geneseq database, a proprietary database that contains sequences published in patents and patent publication, yielded several homologous proteins shown in Table 17D.

TABLE 17D
Geneseq Results for NOV17a

| | | NOV17a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAG89144 | Human secreted protein, SEQ ID NO: | 1 . . . 174 | 174/174 (100%) | 7e-96 |
| | 264 - Homo sapiens, 174 aa. | 1 . . . 174 | 174/174 (100%) | |
| | [WO200142451-A2, 14-JUN-2001] | | | |
| AAM95494 | Human reproductive system related | 1 . . . 174 | 171/174 (98%) | 2e-93 |
| | antigen SEQ ID NO: 4152 - Homo | 18 . . . 191 | 171/174 (98%) | |
| | sapiens, 191 aa. [WO200155320-A2, | | | |
| | 02-AUG-2001] | | | |
| AAY12898 | Human 5′ EST secreted protein SEQ | 1 . . . 141 | 141/141 (100%) | 8e-76 |
| | ID NO: 488 - Homo sapiens, 144 aa. | 1 . . . 141 | 141/141 (100%) | |
| | [WO9906549-A2, 11-FEB-1999] | | | |
| AAG41358 | Arabidopsis thaliana protein fragment | 1 . . . 128 | 34/128 (26%) | 5e-07 |
| | SEQ ID NO: 51446 - Arabidopsis | 1 . . . 120 | 67/128 (51%) | |
| | thaliana, 156 aa. [EP1033405-A2, | | | |
| | 06-SEP-2000] | | | |
| AAG41357 | Arabidopsis thaliana protein fragment | 1 . . . 128 | 34/128 (26%) | 5e-07 |
| | SEQ ID NO: 51445 -Arabidopsis | 6 . . . 125 | 67/128 (51%) | |
| | thaliana, 161 aa. [EP1033405-A2, | | | |
| | 06-SEP-2000] | | | |

In a BLAST search of public sequence datbases, the NOV17a protein was found to have homology to the proteins shown in the BLASTP data in Table 17E.

TABLE 17E
Public BLASTP Results for NOV17a

| | | | Identities/ | |
|---|---|---|---|---|
| | | NOV17a | Similarities | |
| Protein | | Residues/ | for the | |
| Accession | Protein/Organism/ | Match | Matched | Expect |
| Number | Length | Residues | Portion | Value |
| Q9CQ84 | 4930522D07RIK | 1 . . . 156 | 112/156 (71%) | 7e-53 |
| | PROTEIN - Mus | 1 . . . 154 | 126/156 (79%) | |
| | musculus (Mouse), | | | |
| | 188 aa. | | | |
| P21126 | Ubiquitin-like | 1 . . . 150 | 172/156 (46%) | 2e-29 |
| | protein GDX | 1 . . . 153 | 105/156 (67%) | |
| | (Ubiquitin-like protein | | | |
| | 4) - Mus musculus | | | |
| | (Mouse), 157 aa. | | | |
| P11441 | Ubiquitin-like | 1 . . . 141 | 68/147 (46%) | 1e-28 |
| | protein GDX | 1 . . . 146 | 97/147 (65%) | |
| | (Ubiquitin-like protein | | | |
| | 4) - Homo sapiens | | | |
| | (Human), 157 aa. | | | |
| Q920U6 | HOUSEKEEPING | 6 . . . 150 | 68/151 (45%) | 1e-27 |
| | PROTEIN DX5254E | 1 . . . 148 | 101/151 (66%) | |
| | - Mus spicilegus | | | |
| | (Steppe mouse), | | | |
| | 152 aa (fragment). | | | |
| Q91F01 | ORF54 UBI - | 1 . . . 72 | 25/72 (34%) | 3e-07 |
| | Cydia pomonella | 1 . . . 72 | 50/72 (68%) | |
| | granulosis virus | | | |
| | (CpGV) (Cydia | | | |
| | pomonella, 94 aa. | | | |

PFam analysis predicts that the NOV17a protein contains the domains shown in the Table 17F.

TABLE 17F
Domain Analysis of NOV17a

| | | Identities/ | |
|---|---|---|---|
| | NOV17a | Similarities for | |
| Pfam Domain | Match Region | the Matched Region | Expect Value |
| ubiquitin: | 1 . . . 74 | 23/83 (28%) | 1.2e-17 |
| domain 1 of 1 | | 58/83 (70%) | |

### Example 18.

The NOV18 clone was analyzed, and the nucleotide and encoded polypeptide sequences are shown in Table 18A.

TABLE 18A
NOV18 Sequence Analysis

| | SEQ ID NO: 45 | 3880 bp |
|---|---|---|
| NOV18, | TTTATCAAGTAAAAGTGTGTGTGTGTGTTTGTGTGTTTTAAATCTAAGCCTTGTATCT | |
| CG90853-01 DNA | TTTATCCTTGTGGTCTAATTCTTCCTTTCTCTCAATATAGGTATGGCATCACAGCTGC | |
| Sequence | AAGTGTTTTCGCCCCCATCAGTGTCGTCGAGTGCCTTCTGCAGTGCGAAGAAACTGAA | |
| | AATAGAGCCCTCTGGCTGGGATGTTTCAGGACAGAGTAGCAACGACAAATATTATACC | |
| | CACAGCAAAACCCTCCCAGCCACACAAGGGCAAGCCAACTCCTCTCACCAGGTAGCAA | |
| | ATTTCAACATCCCTGCTTACGACCAGGGCCTCCTCCTCCCAGCTCCTGCAGTGGAGCA | |
| | TATTGTTGTAACAGCCGCTGATAGCTCGGGCAGTGCTGCTACATCAACCTTCCAAAGC | |
| | AGCCAGACCCTGACTCACAGAAGCAACGTTTCTTTGCTTGAGCCATATCAAAAATGTG | |
| | GATTGAAACGAAAAAGTGAGGAAGTTGACAGCAACGGTAGTGTGCAGATCATAGAAGA | |
| | ACATCCCCCTCTCATGCTGCAAAACAGGACTGTGGTGGGTGCTGCTGCCACAACCACC | |
| | ACTGTGACCACAAAGAGTAGCAGTTCCAGCGGAGAAGGGGATTACCAGCTGGTCCAGC | |
| | ATGAGATCCTTTGCTCTATGACCAATAGCTATGAAGTCTTGGAGTTCCTAGGCCGGGG | |
| | GACATTTGGACAGGTGGCTAAGTGCTGGAAGAGGAGCACCAAGGAAATTGTGGCTATT | |
| | AAAATCTTGAAGAACCACCCCTCCTATGCCAGACAAGGACAGATTGAAGTGAGCATCC | |
| | TTTCCCGCCTAAGCAGTGAAAATGCTGATGAGTATAATTTTGTCCGTTCATACGAGTG | |
| | CTTTCAGCATAAGAATCACACCTGCCTTGTTTTTGAAATGTTGGAGCAGAACTTATAT | |
| | GATTTTCTAAAGCAAAACAAATTTAGCCCACTGCCACTCAAGTACATCAGACCAATCT | |
| | TGCAGCAGGTGGCCACAGCCTTGATGAAGCTCAAGAGTCTTGGTCTGATCCACGCTGA | |
| | CCTTAAGCCTGAAAACATCATGCTGGTTGATCCAGTTCGCCAGCCCTACCGAGTGAAG | |
| | GTCATTGACTTTGGTTCTGCTAGTCACGTTTCCAAAGCTGTGTGCTCAACCTACTTAC | |
| | AGTCACGTTACTACAGGCAGATTCGTTATATTTCACAAACACAAGGCTTGCCAGCTGA | |
| | ATATCTTCTCAGTGCCGGAACAAAAACAACCAGGTTTTTCAACAGAGATCCTAATTTG | |
| | GGGTACCCACTGTGGAGGCTTAAGACACCTGAAGAACATGAACTGGAGACTGGAATAA | |
| | AATCAAAAGAAGCTCGGAAGTACATTTTTAATTGCTTAGATGACATGGCTCAGGTGAA | |
| | TATGTCTACAGACCTGGAGGGAACAGACATGTTGGCAGAGAAGGCAGACCGAAGAGAA | |
| | TACATTGATCTGTTAAAGAAAATGCTCACAATTGATGCAGATAAGAGAATTACCCCTC | |
| | TAAAAACTCTTAACCATCAGTTTGTGACAATGACTCACCTTTTGGATTTTCCACATAG | |
| | CAATGTTAAGTCTTGTTTTCAGAACATGGAGATCTGCAAGCGGAGGGTTCACATGTAT | |
| | GATACAGTGAGTCAGATCAAGAGTCCCTTCACTACACATGTTGCCCCAAATACAAGCA | |
| | CAAATCTAACCATGAGCTTCAGCAATCAGCTCAATACAGTGCACAATCAGGCCAGTGT | |
| | TCTAGCTTCCAGTTCTACTGCAGCAGCTGCTACTCTTTCTCTGGCTAATTCAGATGTC | |
| | TCACTACTAAACTACCAGTCAGCTTTGTACCCATCATCTGCTGCACCAGTTCCTGGAG | |
| | TTGCCCAGCAGGGTGTTTCCTTGCAGCCTGGAACCACCCAGATTTGCACTCAGACAGA | |
| | TCCATTCCAACAGACATTTATAGTATGTCCACCTGCGTTTCAAAGTGGACTACAAGCA | |
| | ACAACAAAGCATTCTGGATTCCCTGTGAGGATGGATAATGCTGTACCGATTGTACCCC | |
| | AGGCACCAGCTGCTCAGCCACAGGGAAGCTGTACACCACTAATGGTAGCAACTCTCCA | |
| | CCCTCAAGTAGCCACCATCACACCGCAGTATGCGGTGCCCTTTACTCTGAGCTGCGCA | |
| | GCCGGCCGGCCGGCGCTGGTTGAACAGACTGCCGCTGTACTGCAGGCGTGGCCTGGAG | |
| | GGACTCAGCAAATTCTCCTGCCTTCAACTTGGCAACAGTTGCCTGGGGTAGCTCTACA | |
| | CAACTCTGTCCAGCCCACAGCAATGATTCCAGAGGCCATGGGGAGTGGACAGCAGCTA | |
| | GCTGACTGGAGGAATGCCCACTCTCATGGCAACCAGTACAGCACTATCATGCAGCAGC | |
| | CATCCTTGCTGACTAACCATGTGACATTGGCCACTGCTCAGCCTCTGAATGTTGGTGT | |
| | TGCCCATGTTGTCAGACAACAACAATCCAGTTCCCTCCCTTCGAAGAAGAATAAGCAG | |
| | TCAGCTCCAGTCTCTTCCAAGTCCTCTCTAGATGTTCTGCCTTCCCAAGTCTATTCTC | |
| | TGGTTGGGAGCAGTCCCCTCCGCACCACATCTTCTTATAATTCCTTGGTCCCTGTCCA | |
| | AGATCAGCATCAGCCCATCATCATTCCAGATACTCCCAGCCCTCCTGTGAGTGTCATC | |
| | ACTATCCGAAGTGACACTGATGAGGAAGAGGACAACAAATACAAGCCCAGTAGCTCTG | |
| | GACTGAAGCCAAGGTCTAATGTCATCAGTTATGTCACTGTCAATGATTCTCCAGACTC | |
| | TGACTCTTCTTTGAGCAGCCCTTATTCCACTGATACCCTGAGTGCTCTCCGAGGCAAT | |
| | AGTGGATCCGTTTTGGAGGGGCCTGGCAGAGTTGTGGCAGATGGCACTGGCACCCGCA | |
| | CTATCATTGTGCCTCCACTGAAAACTCAGCTTGGTGACTGCACTGTAGCAACCCAGGC | |
| | CTCAGGTCTCCTGAGCAATAAGACTAAGCCAGTCGCTTCAGTGAGTGGGCAGTCATCT | |
| | GGATGCTGTATCACCCCCACAGGGTATCGAGCTCAACGCGGGGGGACCAGTGCAGCAC | |
| | AACCACTCAATCTTAGCCAGAACCAGCAGTCATCGGCGGCTCCAACCTCACAGGAGAG | |
| | AAGCAGCAACCCAGCCCCCCGCAGGCAGCAGGCGTTTGTGGCCCCTCTCTCCCAAGCC | |
| | CCCTACACCTTCCAGCATGGCAGCCCGCTACACTCGACAGGGCACCCACACCTTGCCC | |
| | CGGCCCCTGCTCACCTGCCAAGCCAGGCTCATCTGTATACGTATGCTGCCCCGACTTC | |
| | TGCTGCTGCACTGGGCTCAACCAGCTCCATTGCTCATCTTTTCTCCCCACAGGGTTCC | |
| | TCAAGGCATGCTGCAGCCTATACCACTCACCCTAGCACTTTGGTGCACCAGGTCCCTG | |
| | TCAGTGTTGGGCCCAGCCTCCTCACTTCTGCCAGCGTGGCCCCTGCTCAGTACCAACA | |
| | CCAGTTTGCCACCCAATCCTACATTGGGTCTTCCCGAGGCTCAACAATTTACACTGGA | |
| | TACCCGCTGAGTCCTACCAAGATCAGCCAGTATTCCTACTTATAGTTGGTGAGCATGA | |
| | GGGAGGAGGAATCATGGCTACCTTCTCCTGGCCCTGCGTTCTTAATATTGGGCTATGG | |
| | AGAGATCCTCCTTTACCCTCTTGAAATTTCTTAGCCAGCAACTTGTTCTGCAGGGGCC | |
| | CACTGAAGCAGAAGGTTTTTCTCTGGGGGAACCTGTCTCAGTGTTGACTGCATTGTTG | |
| | TAGTCTTCCCAAAGTTTGCCCTATTTTTAAATTCATTATTTTTGTGACAGTAATTTTG | |
| | GTACTTGGAAGAGTTCAGATGCCCATCTTCTGCAGTTACCAAGGAAGAGAGA | |
| | ORF Start: ATG at 101 | ORF Stop: TAG at 3581 |
| | SEQ ID NO: 46 | 1160 aa MW at 125366.9 kD |

| | |
|---|---|
| NOV18, | MASQLQVFSPPSVSSSAFCSAKKLKIEPSGWDVSGQSSNDKYYTHSKTLPATQGQANS |
| CG90853-01 Protein | SHQVANFNIPAYDQGLLLPAPAVEHIVVTAADSSGSAATSTFQSSQTLTHRSNVSLLE |
| Sequence | PYQKCGLKRKSEEVDSNGSVQIIEEHPPLMLQNRTVVGAAATTTTVTTKSSSSSGEGD |
| | YQLVQHEILCSMTNSYEVLEFLGRGTFGQVAKCWKRSTKEIVAIKILKNHPSYARQGQ |
| | IEVSILSRLSSENADEYNFVRSYECFQHKNHTCLVFEMLEQNLYDFLKQNKFSPLPLK |
| | YIRPILQQVATALMKLKSLGLIHADLKPENIMLVDPVRQPYRVKVIDFGSASHVSKAV |
| | CSTYLQSRYYRQIRYISQTQGLPAEYLLSAGTKTTRFFNRDPNLGYPLWRLKTPEEHE |
| | LETGIKSKEARKYIFNCLDDMAQVNMSTDLEGTDMLAEKADRREYIDLLKKMLTIDAD |
| | KRITPLKTLNHQFVTMTHLLDFPHSNVKSCFQNMEICKRRVHMYDTVSQIKSPFTTHV |
| | APNTSTNLTMSFSNQLNTVHNQASVLASSSTAAAATLSLANSDVSLLNYQSALYPSSA |
| | APVPGVAQQGVSLQPGTTQICTQTDPFQQTFIVCPPAFQSGLQATTKHSGFPVRMDNA |
| | VPIVPQAPAAQPQGSCTPLMVATLHPQVATITPQYAVPFTLSCAAGRPALVEQTAAVL |
| | QAWPGGTQQILLPSTWQQLPGVALHNSVQPTAMIPEAMGSGQQLADWRNAHSHGNQYS |
| | TIMQQPSLLTNHVTLATAQPLNVGVAHVVRQQQSSSLPSKKNKQSAPVSSKSSLDVLP |
| | SQVYSLVGSSPLRTTSSYNSLVPVQDQHQPIIIPDTPSPPVSVITIRSDTDEEEDNKY |
| | KPSSSGLKPRSNVISYVTVNDSPDSDSSLSSPYSTDTLSALRGNSGSVLEGPGRVVAD |
| | GTGTRTIIVPPLKTQLGDCTVATQASGLLSNKTKPVASVSGQSSGCCITPTGYRAQRG |
| | GTSAAQPLNLSQNQQSSAAPTSQERSSNPAPRRQQAFVAPLSQAPYTFQHGSPLHSTG |
| | HPHLAPAPAHLPSQAHLYTYAAPTSAAALGSTSSIAHLFSPQGSSRHAAAYTTHPSTL |
| | VHQVPVSVGPSLLTSASVAPAQYQHQFATQSYIGSSRGSTIYTGYPLSPTKISQYSYL |

Further analysis of the NOV18 protein yielded the following properties shown in Table 18B.

TABLE 18B
Protein Sequence Properties NOV18

| | |
|---|---|
| PSort | 0.4974 probability located in mitochondrial matrix space; |
| analysis: | 0.3000 probability located in microbody (peroxisome); 0.2147 |
| | probability located in mitochondrial inner membrane; 0.2147 |
| | probability located in mitochondrial intermembrane space |
| SignalP | No Known Signal Sequence Predicted |
| analysis: | |

A search of the NOV18 protein against the Geneseq database, a proprietary database that contains sequences published in patents and patent publication, yielded several homologous proteins shown in Table 18C.

TABLE 18C
Geneseq Results for NOV18

| | | NOV18 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE11767 | Human kinase (PKIN)-1 protein- | 1 . . . 1160 | 1158/1210 (95%) | 0.0 |
| | Homo sapiens, 1210 aa. | 1 . . . 1210 | 1159/1210 (95%) | |
| | [WO200181555-A2, 01-NOV-2001] | | | |
| AAB65661 | Novel protein kinase, SEQ ID NO: | 1 . . . 1160 | 730/1248 (58%) | 0.0 |
| | 188- Homo sapiens, 1171 aa. | 8 . . . 1171 | 855/1248 (68%) | |
| | [WO200073469-A2, 07-DEC-2000] | | | |
| AAY53013 | Human secreted protein clone | 532 . . . 1160 | 613/654 (93%) | 0.0 |
| | co155_12 protein sequence SEQ ID | 1 . . . 654 | 615/654 (93%) | |
| | NO: 32 - Homo sapiens, 654 aa. | | | |
| | [WO9957132-A1, 11-NOV-1999] | | | |
| AAM25563 | Human protein sequence SEQ ID | 196 . . . 798 | 426/645 (66%) | 0.0 |
| | NO: 1078 - Homo sapiens, 590 aa. | 1 . . . 575 | 473/645 (73%) | |
| | [WO200153455-A2, 26-JUL-2001] | | | |
| AAW00215 | Drug resistance-associated protein | 10 . . . 1133 | 526/1256 (41%) | 0.0 |
| | kinase - Homo sapiens, 1160 aa. | 6 . . . 1160 | 679/1256 (53%) | |
| | [WO9627015-A2, 06-SEP-1996] | | | |

In a BLAST search of public sequence datbases, the NOV18 protein was found to have homology to the proteins shown in the BLASTP data in Table 18D.

TABLE 18D
Public BLASTP Results for NOV18

| | | NOV18 | Identities/ | |
|---|---|---|---|---|
| Protein | | Residues/ | Similarities for | |
| Number | | Match | the Matched | Expect |
| Accession | Protein/Organism/Length | Residues | Portion | Value |
| Q9QUQ8 | NUCLEAR BODY ASSOCIATED | 1 . . . 1160 | 1131/1210 (93%) | 0.0 |
| | KINASE 2B - Mus musculus (Mouse), | 1 . . . 1210 | 1146/1210 (94%) | |
| | 1210 aa. | | | |
| O88904 | HOMEODOMAIN-INTERACTING | 1 . . . 1160 | 1129/1210 (93%) | 0.0 |
| | PROTEIN KINASE 1- Mus musculus | 1 . . . 1209 | 1145/1210 (94%) | |
| | (Mouse), 1209 aa. | | | |
| Q9QZR3 | NUCLEAR BODY ASSOCIATED | 1 . . . 1160 | 1085/1201 (90%) | 0.0 |
| | KINASE 2A - Mus musculus (Mouse), | 1 . . . 1165 | 1102/1201 (91%) | |
| | 1165 aa. | | | |
| Q9QZR5 | Homeodomain-interacting protein kinase | 1 . . . 1160 | 748/1247 (59%) | 0.0 |
| | 2 (EC 2.7.1.-) (Nuclear body associated | 8 . . . 1196 | 878/1247 (69%) | |
| | kinase 1) (Sialophorin tail associated | | | |
| | nuclear serine/threonine kinase) - Mus | | | |
| | musculus (Mouse), 1196 aa. | | | |
| O75125 | KIAA0630 PROTEIN - Homo sapiens | 670 . . . 1160 | 490/491 (99%) | 0.0 |
| | (Human), 490 aa (fragment). | 1 . . . 490 | 490/491 (99%) | |

PFam analysis predicts that the NOV18 protein contains the domains shown in the Table 18E.

TABLE 18E
Domain Analysis of NOV18

| | | Identities/ | |
|---|---|---|---|
| | NOV18 | Similarities for the | Expect |
| Pfam Domain | Match Region | Matched Region | Value |
| pkinase: domain 1 of 2 | 190 . . . 359 | 64/172 (37%) | 1.1e-31 |
| | | 129/172 (75%) | |
| pkinase: domain 2 of 2 | 452 . . . 478 | 13/30 (43%) | 0.013 |
| | | 20/30 (67%) | |

### Example 19.

The NOV19 clone was analyzed, and the nucleotide and encoded polypeptide sequences are shown in Table 19A.

TABLE 19A
NOV19 Sequence Analysis

| | SEQ ID NO: 47 | 3052 bp |
|---|---|---|
| NOV19a, | ACGCAGTTCACTTTCTAAATGAATCAGGAGTCCTTCTTCATTTTCAAGACCCAGCACT | |
| CG90866-01 DNA | GCAGTTAAGTGACTTGTACTTTGTGGAACCCAAGTGGCTTTGTAAAATCATGGCACAG | |
| Sequence | ATTTTGACAGTGAAAGTGGAAGGTTGTCCAAAACACCCTAAGGGCATTATTTCGCGTA | |
| | GAGATGTGGAAAAATTTCTTTCAAAAAAAAGGAAATTTCCAAAGAACTACATGTCACA | |
| | GTATTTTAAGCTCCTAGAAAAATTCCAGATTGCTTTGCCAATAGGAGAAGAATATTTG | |
| | CTGGTTCCAAGCAGTTTGTCTGACCACAGGCCTGTGATAGAGCTTCCCCATTGTGAGA | |
| | ACTCTGAAATTATCATCCGACTATATGAAATGCCTTATTTTCCAATGGGATTTTGGTC | |
| | AAGATTAATCAATCGATTACTTGAGATTTCACCTTACATGCTTTCAGGGAGAGAACGA | |
| | GCACTTCGCCCAAACAGAATGTATTGGCGACAAGGCATTTACTTAAATTGGTCTCCTG | |
| | AAGCTTATTGTCTGGTAGGATCTGAAGTCTTAGACAATCATCCAGAGAGTTTCTTAAA | |
| | AATTACAGTTCCTTCTTGTAGAAAAGGCTGTATTCTTTTGGGCCAAGTTGTGGACCAC | |
| | ATTGATTCTCTCATGGAAGAATGGTTTCCTGGGTTGCTGGAGATTGATATTTGTGGTG | |
| | AAGGAGAAACTCTGTTGAAGAAATGGGCATTATATAGTTTTAATGATGGCGAAGAACA | |
| | TCAAAAAATCTTACTTGATGACTTGATGAAGAAAGCAGAGGAAGGAGATCTCTTAGTA | |
| | AATCCAGATCAACCAAGGCTCACCATTCCAATATCTCAGATTGCCCCTGACTTGATTT | |
| | TGGCTGACCTGCCTAGAAATATTATGTTGAATAATGATGAGTTGGAATTTGAACAAGC | |
| | TCCAGAGTTTCTCCTAGGTGATGGCAGTTTTGGATCAGTTTACCGAGCAGCCTATGAA | |
| | GGAGAAGAAGTGGCTGTGAAGATTTTTAATAAACATACATCACTCAGGCTGTTAAGAC | |
| | AAGAGCTTGTGGTGCTTTGCCACCTCCACCACCCCAGTTTGATATCTTTGCTGGCAGC | |
| | TGGGATTCGTCCCCGGATGTTGGTGATGGAGTTAGCCTCCAAGGGTTCCTTGGATCGC | |
| | CTGCTTCAGCAGGACAAAGCCAGCCTCACTAGAACCCTACAGCACAGGATTGCACTCC | |
| | ACGTAGCTGATGGTTTGAGATACCTCCACTCAGCCATGATTATATACCGAGACCTGAA | |
| | ACCCCACAATGTGCTGCTTTTCACACTGTATCCCAATGCTGCCATCATTGCAAAGATT | |
| | GCTGACTACGGCATTGCTCAGTACTGCTGTAGAATGGGGATAAAAACATCAGAGGGCA | |
| | CACCAGGGTTTCGTGCACCTGAAGTTGCCAGAGGAAATGTCATTTATAACCAACAGGC | |
| | TGATGTTTATTCATTTGGTTTACTACTCTATGACATTTTGACAACTGGAGGTAGAATA | |
| | GTAGAGGGTTTGAAGTTTCCAAATGAGTTTGATGAATTAGAAATACAAGGAAAATTAC | |
| | CTGATCCAGTTAAAGAATATGGTTGTGCCCCATGGCCTATGGTTGAGAAATTAATTAA | |
| | ACAGTGTTTGAAAGAAAATCCTCAAGAAAGGCCTACTTCTGCCCAGGTATTCTCTCAG | |
| | GTCTTTGACATTTTGAATTCAGCTGAATTAGTCTGTCTGACGAGACGCATTTTATTAC | |
| | CTAAAAACGTAATTGTTGAATGCATGGTTGCTACACATCACAACAGCAGGAATGCAAG | |
| | CATTTGGCTGGGCTGTGGGCACACCGACAGAGGACAGCTCTCATTTCTTGACTTAAAT | |
| | ACTGAAGGATACACTTCTGAGGAAGTTGCTGATAGTAGAATATTGTGCTTAGCCTTGG | |
| | TGCATCTTCCTGTTGAAAAGGAAAGCTGGATTGTGTCTGGGACACAGTCTGGTACTCT | |
| | CCTGGTCATCAATACCGAAGATGGGAAAAAGAGACATACCCTAGAAAAGATGACTGAT | |
| | TCTGTCACTTGTTTGTATTGCAATTCCTTTTCCAAGCAAAGCAAACAAAAAAATTTTC | |
| | TTTTGGTTGGAACCGCTGATGGCAAGTTAGCAATTTTTGAAGATAAGACTGTTAAGCT | |
| | TAAAGGAGCTGCTCCTTTGAAGATACTAAATATAGGAAATGTCAGTACTCCATTGATG | |
| | TGTTTGAGTGAATCCACAAATTCAACGGAAAGAAATGTAATGTGGGGAGGATGTGGCA | |
| | CAAAGATTTTCTCCTTTTCTAATGATTTCACCATTCAGAAACTCATTGAGACAAGAAC | |
| | AAGCCAACTGTTTTCTTATGCAGCTTTCAGTGATTCCAACATCATAACAGTGGTGGTA | |
| | GACACTGCTCTCTATATTGCTAAGCAAAATAGCCCTGTTGTGGAAGTGTGGGATAAGA | |
| | AAACTGAAAAACTCTGTGGACTAATAGACTGCGTGCACTTTTTAAGGTTAGTAAAACC | |
| | AAATAGAAAAAAATTATCTAACCTTATGATGTCTTTGGCTTTACATCCTATATGTTTA | |
| | AAATCAAAGTTAAGATGCAGTTCATCCAAAGGAAGATCCCATATTTTGCTTCGTGTAA | |
| | TTTACAACTTTTGTAATTCGGTCAGAGTCATGATGACAGCACAGCTAGGCGGAAGCCT | |
| | TAAAAATGTCATGCTGGTATTGGGCTACAACCGGAAAAATACTGAAGGTACACAAAAG | |
| | CAGAAAGAGATACAATCTTGCTTGACCGTTTGGGACATCAATCTTCCACATGAAGTGC | |
| | AAAATTTAGAAAAACACATTGAAGTGAGAAAAGAATTAGCTGAAAAAATGAGACGAAC | |
| | ATCTGTTGAGTAAGAGAGAAATAGGAATTGTCTTTGGATAGGAAAATTATTCTCTCCT | |
| | CTTGTAAATATTTATTTTAAAAATGTTCACATGGAAAGGGTACTCACATTTTTTGAAA | |
| | TAGCTCGTGTGTATGAAGGAATGTTATTATTTTTAATTTAAATATATGTAAAAATACT | |
| | TACCAGTAAATGTGTATTTTAAAGAACTATTTAAAA | |
| | ORF Start: ATG at 108 | ORF Stop: TAA at 2853 |
| | SEQ ID NO: 48 | 915 aa MW at 103676.4 kD |
| NOV19a, | MAQILTVKVEGCPKHPKGIISRRDVEKFLSKKRKFPKNYMSQYFKLLEKFQIALPIGE | |
| CG90866-01 Protein | EYLLVPSSLSDHRPVIELPHCENSEIIIRLYEMPYFPMGFWSRLINRLLEISPYMLSG | |
| Sequence | RERALRPNRMYWRQGIYLNWSPEAYCLVGSEVLDNHPESFLKITVPSCRKGCILLGQV | |
| | VDHIDSLMEEWFPGLLEIDICGEGETLLKKWALYSFNDGEEHQKILLDDLMKKAEEGD | |
| | LLVNPDQPRLTIPISQIAPDLILADLPRNIMLNNDELEFEQAPEFLLGDGSFGSVYRA | |
| | AYEGEEVAVKIFNKHTSLRLLRQELVVLCHLHHPSLISLLAAGIRPRMLVMELASKGS | |
| | LDRLLQQDKASLTRTLQHRIALHVADGLRYLHSAMIIYRDLKPHNVLLFTLYPNAAII | |
| | AKIADYGIAQYCCRMGIKTSEGTPGFRAPEVARGNVIYNQQADVYSFGLLLYDILTTG | |
| | GRIVEGLKFPNEFDELEIQGKLPDPVKEYGCAPWPMVEKLIKQCLKENPQERPTSAQV | |
| | FSQVFDILNSAELVCLTRRILLPKNVIVECMVATHHNSRNASIWLGCGHTDRGQLSFL | |
| | DLNTEGYTSEEVADSRILCLALVHLPVEKESWIVSGTQSGTLLVINTEDGKKRHTLEK | |
| | MTDSVTCLYCNSFSKQSKQKNFLLVGTADGKLAIFEDKTVKLKGAAPLKILNIGNVST | |
| | PLMCLSESTNSTERNVMWGGCGTKIFSFSNDFTIQKLIETRTSQLFSYAAFSDSNIIT | |
| | VVVDTALYIAKQNSPVVEVWDKKTEKLCGLIDCVHFLRLVKPNRKKLSNLMMSLALHP | |
| | ICLKSKLRCSSSKGRSHILLRVIYNFCNSVRVMMTAQLGGSLKNVMLVLGYNRKNTEG | |
| | TQKQKEIQSCLTVWDINLPHEVQNLEKHIEVRKELAEKMRRTSVE | |
| | SEQ ID NO: 49 | 3040 bp |
| NOV19b, | ACGCAGTTCACTTTCTAAATGAATCAGGAGTCCTTCTTCATTTTCAAGACCCAGCACT | |
| CG90866-02 DNA | GCAGTTAAGTGACTTGTACTTTGTGGAACCCAAGTGGCTTTGTAAAATCATGGCACAG | |
| Sequence | ATTTTGACAGTGAAAGTGGAAGGTTGTCCAAAACACCCTAAGGGCATTATTTCGCGTA | |
| | GAGATGTGGAAAAATTTCTTTCAAAAAAAAGGAAATTTCCAAAGAACTACATGTCACA | |
| | GTATTTTAAGCTCCTAGAAAAATTCCAGATTGCTTTGCCAATAGGAGAAGAATATTTG | |
| | CTGGTTCCAAGCAGTTTGTCTGACCACAGGCCTGTGATAGAGCTTCCCCATTGTGAGA | |
| | ACTCTGAAATTATCATCCGACTATATGAAATGCCTTATTTTCCAATGGGATTTTGGTC | |
| | AAGATTAATCAATCGATTACTTGAGATTTCACCTTACATGCTTTCAGGGAGAGAACGA | |
| | GCACTTCGCCCAAACAGAATGTATTGGCGACAAGGCATTTACTTAAATTGGTCTCCTG | |
| | AAGCTTATTGTCTGGTAGGATCTGAAGTCTTAGACAATCATCCAGAGAGTTTCTTAAA | |
| | AATTACAGTTCCTTCTTGTAGAAAAGGCTGTATTCTTTTGGGCCAAGTTGTGGACCAC | |
| | ATTGATTCTCTCATGGAAGAATGGTTTCCTGGGTTGCTGGAGATTGATATTTGTGGTG | |
| | AAGGAGAAACTCTGTTGAAGAAATGGGCATTATATAGTTTTAATGATGGCGAAGAACA | |
| | TCAAAAAATCTTACTTGATGACTTGATGAAGAAAGCAGAGGAAGGAGATCTCTTAGTA | |
| | AATCCAGATCAACCAAGGCTCACCATTCCAATATCTCAGATTGCCCCTGACTTGATTT | |
| | TGGCTGACCTGCCTAGAAATATTATGTTGAATAATGATGAGTTGGAATTTGAACAAGC | |
| | TCCAGAGTTTCTCCTAGGTGATGGCAGTTTTGGATCAGTTTACCGAGCAGCCTATGAA | |
| | GGAGAAGAAGTGGCTGTGAAGATTTTTAATAAACATACATCACTCAGGCTGTTAAGAC | |
| | AAGAGCTTGTGGTGCTTTGCCACCTCCACCACCCCAGTTTGATATCTTTGCTGGCAGC | |
| | TGGGATTCGTCCCCGGATGTTGGTGATGGAGTTAGCCTCCAAGGGTTCCTTGGATCGC | |
| | CTGCTTCAGCAGGACAAAGCCAGCCTCACTAGAACCCTACAGCACAGGATTGCACTCC | |
| | ACGTAGCTGATGGTTTGAGATACCTCCACTCAGCCATGATTATATACCGAGACCTGAA | |
| | ACCCCACAATGTGCTGCTTTTCACACTGTATCCCAATGCTGCCATCATTGCAAAGATT | |
| | GCTGACTACGGCATTGCTCAGTACTGCTGTAGAATGGGGATAAAAACATCAGAGGGCA | |
| | CACCAGGGTTTCGTGCACCTGAAGTTGCCAGAGGAAATGTCATTTATAACCAACAGGC | |
| | TGATGTTTATTCATTTGGTTTACTACTCTATGACATTTTGACAACTGGAGGTAGAATA | |
| | GTAGAGGGTTTGAAGTTTCCAAATGAGTTTGATGAATTAGAAATACAAGGAAAATTAC | |
| | CTGATCCAGTTAAAGAATATGGTTGTGCCCCATGGCCTATGGTTGAAAAATTAATTAA | |
| | ACAGTGTTTGAAAGAAAATCCTCAAGAAAGGCCTACTTCTGCCCAGGTCTTTGACATT | |
| | TTGAATTCAGCTGAATTAGTCTGTCTGACGAGACGCATTTTATTACCTAAAAACGTAA | |
| | TTGTTGAATGCATGGTTGCTACACATCACAACAGCAGGAATGCAAGCATTTGGCTGGG | |
| | CTGTGGGCACACCGACAGAGGACAGCTCTCATTTCTTGACTTAAATACTGAAGGATAC | |
| | ACTTCTGAGGAAGTTGCTGATAGTAGAATATTGTGCTTAGCCTTGGTGCATCTTCCTG | |
| | TTGAAAAGGAAAGCTGGATTGTGTCTGGGACACAGTCTGGTACTCTCCTGGTCATCAA | |
| | TACCGAAGATGGGAAAAAGAGACATACCCTAGAAAAGATGACTGATTCTGTCACTTGT | |
| | TTGTATTGCAATTCCTTTTCCAAGCAAAGCAAACAAAAAAATTTTCTTTTGGTTGGAA | |
| | CCGCTGATGGCAAGTTAGCAATTTTTGAAGATAAGACTGTTAAGCTTAAAGGAGCTGC | |
| | TCCTTTGAAGATACTAAATATAGGAAATGTCAGTACTCCATTGATGTGTTTGAGTGAA | |
| | TCCACAAATTCAACGGAAAGAAATGTAATGTGGGGAGGATGTGGCACAAAGATTTTCT | |
| | CCTTTTCTAATGATTTCACCATTCAGAAACTCATTGAGACAAGAACAAGCCAACTGTT | |
| | TTCTTATGCAGCTTTCAGTGATTCCAACATCATAACAGTGGTGGTAGACACTGCTCTC | |
| | TATATTGCTAAGCAAAATAGCCCTGTTGTGGAAGTGTGGGATAAGAAAACTGAAAAAC | |
| | TCTGTGGACTAATAGACTGCGTGCACTTTTTAAGGTTAGTAAAACCAAATAGAAAAAA | |
| | ATTATCTAACCTTATGATGTCTTTGGCTTTACATCCTATATGTTTAAAATCAAAGTTA | |
| | AGATGCAGTTCATCCAAAGGAAGATCCCATATTTTGCTTCGTGTAATTTACAACTTTT | |
| | GTAATTCGGTCAGAGTCATGATGACAGCACAGCTAGGCGGAAGCCTTAAAAATGTCAT | |
| | GCTGGTATTGGGCTACAACCGGAAAAATACTGAAGGTACACAAAAGCAGAAAGAGATA | |
| | CAATCTTGCTTGACCGTTTGGGACATCAATCTTCCACATGAAGTGCAAAATTTAGAAA | |
| | AACACATTGAAGTGAGAAAAGAATTAGCTGAAAAAATGAGACGAACATCTGTTGAGTA | |
| | AGAGAGAAATAGGAATTGTCTTTGGATAGGAAAATTATTCTCTCCTCTTGTAAATATT | |
| | TATTTTAAAAATGTTCACATGGAAAGGGTACTCACATTTTTTGAAATAGCTCGTGTGT | |
| | ATGAAGGAATGTTATTATTTTTAATTTAAATATATGTAAAAATACTTACCAGTAAATG | |
| | TGTATTTTAAAGAACTATTTAAAA | |
| | ORF Start: ATG at 108 | ORF Stop: TAA at 2841 |
| | SEQ ID NO: 50 | 911 aa MW at 103214.9 kD |

| | |
|---|---|
| NOV19b, | MAQILTVKVEGCPKHPKGIISRRDVEKFLSKKRKFPKNYMSQYFKLLEKFQIALPIGE |
| CG90866-02 Protein | EYLLVPSSLSDHRPVIELPHCENSEIIIRLYEMPYFPMGFWSRLINRLLEISPYMLSG |
| Sequence | RERALRPNRMYWRQGIYLNWSPEAYCLVGSEVLDNHPESFLKITVPSCRKGCILLGQV |
| | VDHIDSLMEEWFPGLLEIDICGEGETLLKKWALYSFNDGEEHQKILLDDLMKKAEEGD |
| | LLVNPDQPRLTIPISQIAPDLILADLPRNIMLNNDELEFEQAPEFLLGDGSFGSVYRA |
| | AYEGEEVAVKIFNKHTSLRLLRQELVVLCHLHHPSLISLLAAGIRPRMLVMELASKGS |
| | LDRLLQQDKASLTRTLQHRIALHVADGLRYLHSAMIIYRDLKPHNVLLFTLYPNAAII |
| | AKIADYGIAQYCCRMGIKTSEGTPGFRAPEVARGNVIYNQQADVYSFGLLLYDILTTG |
| | GRIVEGLKFPNEFDELEIQGKLPDPVKEYGCAPWPMVEKLIKQCLKENPQERPTSAQV |
| | FDILNSAELVCLTRRILLPKNVIVECMVATHHNSRNASIWLGCGHTDRGQLSFLDLNT |
| | EGYTSEEVADSRILCLALVHLPVEKESWIVSGTQSGTLLVINTEDGKKRHTLEKMTDS |
| | VTCLYCNSFSKQSKQKNFLLVGTADGKLAIFEDKTVKLKGAAPLKILNIGNVSTPLMC |
| | LSESTNSTERNVMWGGCGTKIFSFSNDFTIQKLIETRTSQLFSYAAFSDSNIITVVVD |
| | TALYIAKQNSPVVEVWDKKTEKLCGLIDCVHFLRLVKPNRKKLSNLMMSLALHPICLK |
| | SKLRCSSSKGRSHILLRVIYNFCNSVRVMMTAQLGGSLKNVMLVLGYNRKNTEGTQKQ |
| | KEIQSCLTVWDINLPHEVQNLEKHIEVRKELAEKMRRTSVE |

Sequence comparison of the above protein sequences yields the following sequence relationships shown in Table 19B.

TABLE 19B
Comparison of NOV19a against NOV19b.

| | NOV19a Residues/ | Identities/Similarities |
|---|---|---|
| Protein Sequence | Match Residues | for the Matched Region |
| NOV19b | 1 . . . 915 | 896/915 (97%) |
| | 1 . . . 911 | 896/915 (97%) |

Further analysis of the NOV19a protein yielded the following properties shown in Table 19C.

TABLE 19C
Protein Sequence Properties NOV19a

| | |
|---|---|
| PSort | 0.6000 probability located in nucleus; 0.3000 probability |
| analysis: | located in microbody (peroxisome); 0.1000 probability located |
| | in mitochondrial matrix space; 0.1000 probability located in |
| | lysosome (lumen) |
| SignalP | No Known Signal Sequence Predicted |
| analysis: | |

A search of the NOV19a protein against the Geneseq database, a proprietary database that contains sequences published in patents and patent publication, yielded several homologous proteins shown in Table 19D.

TABLE 19D
Geneseq Results for NOV19a

| | | | Identities/ | |
|---|---|---|---|---|
| | | NOV19a | Similarities | |
| | Protein/ | Residues/ | for the | |
| Geneseq | Organism/Length | Match | Matched | Expect |
| Identifier | [Patent #, Date] | Residues | Region | Value |
| AAU03554 | Human protein | 4 . . . 792 | 735/833 | 0.0 |
| | kinase #54- | | (88%) | |
| | Homo sapiens, | 130 . . . 909 | 735/833 | |
| | 909 aa. [WO- | | (88%) | |
| | 200138503-A2, | | | |
| | 31 May 2001] | | | |
| AAM25477 | Human protein | 309 . . . 492 | 184/184 | e−102 |
| | sequence SEQ ID | | (100%) | |
| | NO: 992-Homo | 1 . . . 184 | 184/184 | |
| | sapiens, 184 aa. | | (100%) | |
| | [WO200153455- | | | |
| | A2, | | | |
| | 26 July 2001] | | | |
| AAG67395 | Amino acid | 18 . . . 528 | 166/588 | 3e−57 |
| | sequence of | | (28%) | |
| | human protein | 985 . . . 1560 | 285/588 | |
| | kinase SGK258- | | (48%) | |
| | Homo sapiens, | | | |
| | 2014 aa. [WO- | | | |
| | 200166594-A2, | | | |
| | 13 Sept. 2001] | | | |
| ABG08051 | Novel human | 181 . . . 673 | 146/539 | 2e−40 |
| | diagnostic protein | | (27%) | |
| | #8042-Homo | 19 . . . 516 | 251/539 | |
| | sapiens, 809 aa. | | (46%) | |
| | [WO200175067- | | | |
| | A2, | | | |
| | 11 Oct. 2001] | | | |
| ABG08051 | Novel human | 181 . . . 673 | 146/539 | 2e−40 |
| | diagnostic protein | | (27%) | |
| | #8042-Homo | 19 . . . 516 | 251/539 | |
| | sapiens, 809 aa. | | (46%) | |
| | [WO200175067- | | | |
| | A2, | | | |
| | 11 Oct. 2001] | | | |

In a BLAST search of public sequence datbases, the NOV19a protein was found to have homology to the proteins shown in the BLASTP data in Table 19E.

TABLE 19E
Public BLASTP Results for NOV19a

| | | | Identities/ | |
|---|---|---|---|---|
| | | NOV19a | Similarities | |
| Protein | | Residues/ | for the | |
| Accession | Protein/ | Match | Matched | Expect |
| Number | Organism/Length | Residues | Portion | Value |
| Q9CQG8 | 4921513O20RIK | 378 . . . 915 | 429/549 | 0.0 |
| | PROTEIN-Mus | | (78%) | |
| | musculus (Mouse), | 18 . . . 561 | 475/549 | |
| | 561 aa. | | (86%) | |
| Q96JN5 | KIAA1790 | 18 . . . 673 | 193/740 | 5e−57 |
| | PROTEIN- | | (26%) | |
| | Homo sapiens | 301 . . . 1009 | 338/740 | |
| | (Human), 1369 aa | | (45%) | |
| | (fragment). | | | |
| T33475 | hypothetical protein | 170 . . . 522 | 131/451 | 2e−30 |
| | T27C10.5- | | (29%) | |
| | Caenorhabditis | 245 . . . 680 | 200/451 | |
| | elegans, 1090 aa. | | (44%) | |
| Q9TZM4 | HYPOTHETICAL | 170 . . . 522 | 131/451 | 2e−30 |
| | 130.7 KDA | | (29%) | |
| | PROTEIN- | 330 . . . 865 | 200/451 | |
| | Caenorhabditis | | (44%) | |
| | elegans, 1175 aa. | | | |
| Q9BI25 | SHK1 PROTEIN- | 270 . . . 530 | 85/276 | 7e−26 |
| | Dictyostelium | | (30%) | |
| | discoideum (Slime | 42 . . . 304 | 149/276 | |
| | mold), 527 aa. | | (53%) | |

PFam analysis predicts that the NOV19a protein contains the domains shown in the Table 19F.

TABLE 19F
Domain Analysis of NOV19a

| | | Identities/ | |
|---|---|---|---|
| | | Similarities | |
| | | for the | |
| Pfam | NOV19a | Matched | Expect |
| Domain | Match Region | Region | Value |
| pkinase: | 279 . . . 528 | 92/288 | 2.6e−38 |
| domain 1 of 1 | | (32%) | |
| | | 169/288 | |
| | | (59%) | |
| WD40: | 587 . . . 626 | 6/41 | 6.5e+02 |
| domain 1 of 2 | | (15%) | |
| | | 26/41 | |
| | | (63%) | |
| WD40: | 632 . . . 674 | 12/43 | 11 |
| domain 2 of 2 | | (28%) | |
| | | 36/43 | |
| | | (84%) | |

### Example 20.

The NOV20 clone was analyzed, and the nucleotide and encoded polypeptide sequences are shown in Table 20A.

TABLE 20A
NOV20 Sequence Analysis

| | SEQ ID NO: 51 | 480 bp |
|---|---|---|
| NOV20a, | CAGAGAGAACCCACCATGGTGCTGTCTCCTGCCGACAAGACCAACGTCAAGGCCGCCT | |
| CG93198-01 DNA | GGGGTAAGGTCGGCGCGCACGCTGGCGAGTATGGTGCGGAGGCCCTGGAGAGGATGTT | |
| Sequence | CCTGTCCTTCCCCACCACCAAGACCTACTTCCCGCACTTCGACCTGAGCCACGGCTCT | |
| | GCCCAGGTTAAGGGCCACGGCAAGAAGGTGGCCGACGCGCTGACCAACGCCGTGGCGC | |
| | ACGTGGACCCGGTCAACTTCAAGCTCCTAAGCCACTGCCTGCTGGTGACCCTGGCCGC | |
| | CCACCTCCCCGCCGAGTTCACCCCTGCGGTGCACGCCTCCCTGGACAAGTTCCTGGCT | |
| | TCTGTGAGCACCGTGCTGACCTCCAAATACCGTTAAGCTGGAGCCTCGGTAGCCGTTC | |
| | CTCCTGCCCGCTGGGCCTCCCAACGGGCCCTCCTCCCCTCCTTGCACCGGCCCTTCCT | |
| | GGTCTTTGAATAAAGT | |
| | ORF Start: ATG at 16 | ORF Stop: TAA at 382 |
| | SEQ ID NO: 52 | 122 aa MW at 13071.9 kD |
| NOV20a, | MVLSPADKTNVKAAWGKVGAHAGEYGAEALERMFLSFPTTKTYFPHFDLSHGSAQVKG | |
| CG93198-01 Protein | HGKKVADALTNAVAHVDPVNFKLLSHCLLVTLAAHLPAEFTPAVHASLDKFLASVSTV | |
| Sequence | LTSKYR | |
| | SEQ ID NO: 53 | 433 bp |
| NOV20b, | CAGACTCAGAGAGAACCCACCATGGTGCTGTCTCCTGCCGACAAGACCAACGTCAAGG | |
| CG93198-02 DNA | CCGCCTGGGGTAAGGTCGGCGCGCACGCTGGCGAGTATGGTGCGGAGGCCCTGGAGAG | |
| Sequence | GATGTTCCTGTCCTTCCCCACCACCAAGACCTACTTCCCGCACTTCGACCTGAGCCAC | |
| | GGCTCTGCCCAGGTTAAGGGCCACGGCAAGAAGGTGGCCGACGCGCTGACCAACGCCG | |
| | TGGCGCACGTGGACGACATGCCCAACGCGCTGTCCGCCCTGAGCGACCTGCACGCCTC | |
| | CCTGGACAAGTTCCTGGCTTCTGTGAGCACCGTGCTGACCTCCAAATACCGTTAAGCT | |
| | GGAGCCTCGGTGGCCATGCTTCTTGCCCCTTGGGCCTCCCCCCAGCCCCTCCTCCCCT | |
| | TCCTGCACCCGTACCCCCGTGGTCTTT | |
| | ORF Start: ATG at 22 | ORF Stop: TAA at 343 |
| | SEQ ID NO: 54 | 107 aa MW at 11415.8 kD |

| | |
|---|---|
| NOV20b, | MVLSPADKTNVKAAWGKVGAHAGEYGAEALERMFLSFPTTKTYFPHFDLSHGSAQVKG |
| CG93198-02 Protein | HGKKVADALTNAVAHVDDMPNALSALSDLHASLDKFLASVSTVLTSKYR |
| Sequence | |

Sequence comparison of the above protein sequences yields the following sequence relationships shown in Table 20B.

TABLE 20B
Comparison of NOV20a against NOV20b.

| Protein | NOV20a Residues/ | Identities/ |
|---|---|---|
| Sequence | Match Residues | Similarities of the Matched Region |
| NOV20b | 1 . . . 122 | 90/122 (73%) |
| | 1 . . . 107 | 91/122 (73%) |

Further analysis of the NOV20a protein yielded the following properties shown in Table 20C.

TABLE 20C
Protein Sequence Properties NOV20a

| | |
|---|---|
| PSort | 0.7480 probability located in microbody (peroxisome); |
| analysis: | 0.2216 probability located in lysosome (lumen); 0.1000 |
| | probability located in mitochondrial matrix space; 0.0000 |
| | probability located in endoplasmic reticulum (membrane) |
| SignalP | No Known Signal Sequence Predicted |
| analysis: | |

A search of the NOV20a protein against the Geneseq database, a proprietary database that contains sequences published in patents and patent publication, yielded several homologous proteins shown in Table 20D.

TABLE 20D
Geneseq Results for NOV20a

| | | | Identities/ | |
|---|---|---|---|---|
| | | NOV20a | Similarities | |
| | Protein/ | Residues/ | for the | |
| Geneseq | Organism/Length | Match | Matched | Expect |
| Identifier | [Patent #, Date] | Residues | Region | Value |
| AAU30056 | Novel human | 1 . . . 122 | 119/123 | 9e−63 |
| | secreted protein | | (96%) | |
| | #547-Homo | 8 . . . 130 | 120/123 | |
| | sapiens, 130 aa. | | (96%) | |
| | [WO200179449-A2, | | | |
| | 25 Oct. 2001] | | | |
| AAU30270 | Novel human | 1 . . . 122 | 122/142 | 1e−62 |
| | secreted protein | | (85%) | |
| | #761-Homo | 8 . . . 149 | 122/142 | |
| | sapiens, 149 aa. | | (85%) | |
| | [WO200179449-A2, | | | |
| | 25 Oct. 2001] | | | |
| AAU27753 | Human full-length | 1 . . . 122 | 122/142 | 1e−62 |
| | polypeptide | | (85%) | |
| | sequence #78-Homo | 258 . . . 399 | 122/142 | |
| | sapiens, 399 aa. | | (85%) | |
| | [WO200164834-A2, | | | |
| | 07 Sept. 2001] | | | |
| AAB66773 | Human hemoglobin | 2 . . . 122 | 121/141 | 4e−62 |
| | adult alpha protein- | | (85%) | |
| | Homo sapiens, | 1 . . . 141 | 121/141 | |
| | 141 aa. | | (85%) | |
| | [US6172039-B1, | | | |
| | 09 Jan. 2001] | | | |
| AAY87793 | Human alpha- | 2 . . . 122 | 121/141 | 4e−62 |
| | hemoglobin protein- | | (85%) | |
| | Homo sapiens, | 1 . . . 141 | 121/141 | |
| | 141 aa. | | (85%) | |
| | [US6054566-A, | | | |
| | 25 Apr. 2000] | | | |

In a BLAST search of public sequence datbases, the NOV20a protein was found to have homology to the proteins shown in the BLASTP data in Table 20E.

TABLE 20E
Public BLASTP Results for NOV20a

| | | | Identities/ | |
|---|---|---|---|---|
| | | NOV20a | Similarities | |
| Protein | | Residues/ | for the | |
| Accession | Protein/ | Match | Matched | Expect |
| Number | Organism/Length | Residues | Portion | Value |
| AAC72839 | ALPHA-2- | 1 . . . 122 | 122/142 | 3e−62 |
| | GLOBIN-Homo | | (85%) | |
| | sapiens (Human), | 1 . . . 142 | 122/142 | |
| | 142 aa. | | (85%) | |
| Q9NYR7 | ALPHA-2- | 1 . . . 122 | 121/142 | 7e−62 |
| | GLOBIN-Homo | | (85%) | |
| | sapiens (Human), | 1 . . . 142 | 122/142 | |
| | 142 aa. | | (85%) | |
| P01922 | Hemoglobin alpha | 2 . . . 122 | 121/141 | 1e−61 |
| | chain-Homo sapiens | | (85%) | |
| | (Human),, 141 aa. | 1 . . . 141 | 121/141 | |
| | | | (85%) | |
| Q96KF1 | HEMOGLOBIN | 1 . . . 122 | 121/142 | 1e−61 |
| | ALPHA-1 | | (85%) | |
| | GLOBIN CHAIN- | 1 . . . 142 | 121/142 | |
| | Homo sapiens | | (85%) | |
| | (Human), 142 aa. | | | |
| P01923 | Hemoglobin alpha | 2 . . . 122 | 120/141 | 3e−61 |
| | chain-Gorilla | | (85%) | |
| | gorilla gorilla | 1 . . . 141 | 121/141 | |
| | (Lowland gorilla), | | (85%) | |
| | 141 aa. | | | |

PFam analysis predicts that the NOV20a protein contains the domains shown in the Table 20F.

TABLE 20F
Domain Analysis of NOV20a

| | | Identities/ | |
|---|---|---|---|
| | | Similarities | |
| | | for the | |
| Pfam | NOV20a | Matched | Expect |
| Domain | Match Region | Region | Value |
| globin: | 2 . . . 72 | 41/79 | 7.2e−26 |
| domain 1 of 2 | | (52%) | |
| | | 60/79 | |
| | | (76%) | |
| globin: | 73 . . . 122 | 28/52 | 8.9e−19 |
| domain 2 of 2 | | (54%) | |
| | | 48/52 | |
| | | (92%) | |

### Example 21.

The NOV21 clone was analyzed, and the nucleotide and encoded polypeptide sequences are shown in Table 21A.

TABLE 21A
NOV21 Sequence Analysis

| | SEQ ID NO:55 | 2522 bp |
|---|---|---|
| NOV21 | GAGGCTGGACACCTGTTCTGCTGTTGTGTCCTGCCATTCTCCTGAAGAACAGAGGCAC | |
| CG93517-01 | ACTGTAAAACCCAACACTTCCCCTTGCATTCTATAAGATTACAGCAAGATGGAAATAC | |
| Sequence | CAAATCCCCCTACCTCCAAATGTATCACTTACTGGAAAAGAAAAGTGAAATCTGAATA | |
| | CATGCGACTTCGACAACTTAAACGGCTTCAGGCAAATATGGGTGCAAAGGCTTTGTAT | |
| | GTGGCAAATTTTGCAAAGGTTCAAGAAAAAACCCAGATCCTCAATGAAGAATCGAAGA | |
| | AGCTTCGTGTCCAACCTGTTCAGTCAATGAAGCCTGTGAGTGGACACCCTTTTCTCAA | |
| | AAAGTGTACCATAGAGAGCATTTTCCCGGGATTTGCAAGCCAACATATGTTAATGAGG | |
| | TCACTGAACACAGTTGCATTGGTTCCCATCATGTATTCCTGGTCCCCTCTCCAACAGA | |
| | ACTTTATGGTAGAAGATGAGACGGTTTTGTGCAATATTCCCTACATGGGAGATGAAGT | |
| | GAAAGAAGAAGATGAGACTTTTATTGAGGAGCTGATCAATAACTATGATGGGAAAGTC | |
| | CATGGTGAAGAAGAGATGATCCCTGGATCCGTTCTGATTAGTGATGCTGTTTTTCTGG | |
| | AGTTGGTCGATGCCCTGAATCAGTACTCAGATGAGGAGGAGCAAGQGCACAATGACAC | |
| | CTCAGATGGAAAGCAGGATGACAGCAAAGAAGATCTGCCAGTAACAAGAAAGAGAAAG | |
| | CGACATGCTATTGAAGGCAACAAAAAGAGTTCCAAGAAACAGTTCCCAAATGACATGA | |
| | TCTTCAGTGCAATTGCCTCAATGTTCCCTGAGAATGGTGTCCCAGATGACATGAAGGA | |
| | GAGGTATCGAGAACTAACAGAGATGTCAGACCCCAATGCACTTCCCCCTCAGTGCACA | |
| | CCCAACATCGATGGCCCCAATGCCAAGTCTGTGCAGCGGGAGCAATCTCTGCACTCCT | |
| | TCCACACACTTTTTTGCCGGCGCTGCTTTAAATACGACTGCTTCCTTCACCCTTTTCA | |
| | TGCCACCCCTAATGTATATAAACGCAAGAATAAAGAAATCAAGATTGAACCAGAACCA | |
| | TGTCCCACACACTGCTTCCTTTTGCTGGAACGAGCAAAGGAGTATGCCATGCTCCACA | |
| | ACCCCCGCTCCAAGTGCTCTGGTCGTCGCCGGAGAAGGCACCACATAGTCAGTGCTTC | |
| | CTGCTCCAATGCCTCAGCCTCTGCTGTGCCTGAGACTAAAGAAGGAGACAGTGACAGG | |
| | GACACACGCAATGACTGGGCCTCCACTTCTTCAGAGGCTAACTCTCOCTGTCAGACTC | |
| | CCACAAAACAGAAGGCTAGTCCAGCCCCACCTCAACTCTGCGTAGTGGAAGCACCCTC | |
| | GGAGCCTGTGGAATGGACTGGGGCTGAAGAATCTCTTTTTCGAGTCTTCCATGGCACC | |
| | TACTTCAACAACTTCTGTTCAATAGCCAGGCTTCTGGGGACCAAGACGTGCAAGCAGG | |
| | TCTTTCAGTTTGCAGTCAAAGAATCACTTATCCTGAAGCTGCCAACAGATGAGCTCAT | |
| | GTACCCCTCACAGAAGAAGAAAAGAAAGCACAGATTGTGGGCTGCACACTGCAGGAAG | |
| | ATTCAGCTGAAGAAAGATAACTCTTCCACACAAGTGTACAACTACCAACCCTGCGACC | |
| | ACCCAGACCGCCCCTGTGACAGCACCTGCCCCTGCATCATCACTCAGAATTTCTGTGA | |
| | GAACTTCTGCCAGTGCAACCCAGACTTGCGACAATGTGACCCTGACCTCTGTCTCACC | |
| | TGTGGGGCCTCACAGCACTGGGACTGCAAGGTGCTTTCCTCTAAAAACTGCACCATCC | |
| | AGCGTGGACTTAAGAAGCACCTGCTGCTGGCCCCCTCTGATGTGGCCGGATGGGGCAC | |
| | CTTCATAAAGGAGTCTGTGCAGAAGAACGAATTCATTTCTGAATACTGTGGTCAGCTC | |
| | ATCTCTCACGATGACCCTGATCGACGCGGAAAGGTCTATGACAAATACATGTCCAGCT | |
| | TCCTCTTCAACCTCAATAATGATTTTGTAGTGGATGCTACTCGGAAAGGAAACAAAAT | |
| | TCGATTTGCAAATCATTCAGTGAATCCCAACTGTTATGCCAAAGTGGTCATGGTGATT | |
| | GGAGACCATCGGATTGGGATCTTTGCCAAGAGGGCAATTCAAGCTGGCGAAGAGCTCT | |
| | TCTTTGATTACAGGTACAGCCAAGCTGATGCTCTCAAGTACGTGGGGATCGAGAGGGA | |
| | GACCGACGTCCTTTAGCCCTCCCAGGCCCCAACGGCAGCACTTATGGTAGCGGCACTG | |
| | TCTTGGCTTTCGTGCTCACACCACTGCTGCTCGAGTCTCCTGCACTGTGTCTCCCACA | |
| | CTGAGAAACCCCCCAACCCACTCCCCCTGTAGTGAGGCCTCTGCCATGTCCAGAGGGC | |
| | ACAAAACTGTCTCAATGAGAGGGGAGACAGAGGCAGCTAGGGCTTGGTCTCCCAGGAC | |
| | AGAGAGTTACAGAAATGGGAGACTGTTT | |
| | ORF Start: ATG AT 107 | ORF Stop: TAG at 2276 |
| | SEQ ID NO:56 | 723 aa MW at 82585.0 kD |

| | |
|---|---|
| NOV21, | MEIPNPPTSKCITYWKRKVKSEYMRLRQLKRLQANMGAKALYVANFAKVQEKTQILNE |
| CG93517-01 Protein | EWKKLRVQPVQSMKPVSGHPFLKKCTIESIFPGPASQMMLMRSLNTVALVPTMYSWSP |
| Sequence | LQQNFMVEDETVLCNIPYMGDEVKEEDETFIEELINNYDGKVHGEEEMIPGSVLISDA |
| | VFLELVDALNOYSDEEEEGHNDTSDGKQDDSKEDLPVTRKRKRHAIEGNKKSSKKQFP |
| | NDMIFSAIASMFPENGVPDDMKERYRELTENSDPNALPPQCTPNIDGPNAKSVQREQS |
| | LHSFHTLFCRRCFKYDCFLHPFHATPNVYKRKNKEIKIEPEPCGTDCFLLLEGAKEYA |
| | MLHNPRSKCSGRRRRRHHIVSASCSNASASAVAETKEGDSDRDTGNDWASSSSEANSR |
| | CQTPTKQKASPAPPQLCVVEAPSEPVEWTGAEESLFRVFHGTYFNNFCSIARLLGTKT |
| | CKQVFQFAVKESLTLKLPTDELMYPSQKKKRKHRLWAAHCRKIQLKKDNSSTQVYNYQ |
| | PCDHPDRPCDSTCPCIMTQNFCEKFCQCNPDLRECDPDLCLTCGASEHWDCKVVSCKN |
| | CSIQRGLKKHLLLAPSDVAGWGTFIKESVQKNEFISEYCGELISQDEADRRGKVYDKY |
| | MSSFLFNLNNDFVVDATRKGNKIRFANHSVNPNCYAKVVMVNGDHRIGIFAKRAIQAG |
| | EELFFDYRYSQADALKYVGTERETDVL |

Further analysis of the NOV21 protein yielded the following properties shown in Table 21B.

TABLE 21B
Protein Sequence Properties NOV21

| | |
|---|---|
| PSort | 0.9600 probability located in nucleus; 0.3000 probability |
| analysis: | located in microbody (peroxisome); 0.1000 probability |
| | located in mitochondrial matrix space; 0.1000 probability |
| | located in lysosome (lumen) |
| SignalP | No Known Signal Sequence Predicted |
| analysis: | |

A search of the NOV21 protein against the Geneseq database, a proprietary database that contains sequences published in patents and patent publication, yielded several homologous proteins shown in Table 21C.

TABLE 21C
Geneseq Results for NOV21

| | | | Identities/ | |
|---|---|---|---|---|
| | | NOV21 | Similarities | |
| | Protein/ | Residues/ | for the | |
| Geneseq | Organism/Length | Match | Matched | Expect |
| Identifier | [Patent #, Date] | Residues | Region | Value |
| AAW05260 | Chromatin regulator | 15 . . . 722 | 463/754 | 0.0 |
| | protein EZH2- | | (61%) | |
| | Homo sapiens, | 15 . . . 745 | 557/754 | |
| | 746 aa. | | (73%) | |
| | [WO9635784-A2, | | | |
| | 14 Nov. 1996] | | | |
| AAB82455 | Arabidopsis seed- | 424 . . . 710 | 123/334 | 2e−52 |
| | specific Polycomb | | (36%) | |
| | group gene MEA 1 | 334 . . . 665 | 173/334 | |
| | product-Arabidopsis | | (50%) | |
| | thaliana, 689 aa. | | | |
| | [WO200138551-A1, | | | |
| | 31 May 2001] | | | |
| AAY57036 | Fertilisation- | 424 . . . 710 | 123/334 | 2e−52 |
| | independent | | (36%) | |
| | endosperm 1 (FIE1) | 334 . . . 665 | 173/334 | |
| | amino acid | | (50%) | |
| | sequence- | | | |
| | Arabidopsis sp, | | | |
| | 689 aa. | | | |
| | [WO9957247-A1, | | | |
| | 11 Nov. 1999] | | | |
| AAB01673 | FIS1 protein | 424 . . . 710 | 123/334 | 2e−52 |
| | sequence- | | (36%) | |
| | Arabidopsis | 334 . . . 665 | 173/334 | |
| | thaliana, 689 aa. | | (50%) | |
| | [WO20016609-A1, | | | |
| | 30 Mar. 2000] | | | |
| AAY42698 | Arabidopsis seed | 424 . . . 710 | 123/334 | 2e−52 |
| | specific regulatory | | (36%) | |
| | protein sequence- | 334 . . . 665 | 173/334 | |
| | Arabidopsis sp, | | (50%) | |
| | 689 aa. | | | |
| | [WO9953083-A1, | | | |
| | 21 Oct. 1999] | | | |

In a BLAST search of public sequence datbases, the NOV21 protein was found to have homology to the proteins shown in the BLASTP data in Table 21D.

TABLE 21D
Public BLASTP Results for NOV21

| | | NOV21 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q92800 | Enhancer of zeste homolog 1 (ENX- | 1 . . . 723 | 721/747 (96%) | 0.0 |
| | 2) - Homo sapiens (Human), 747 aa. | 1 . . . 747 | 722/747 (96%) | |
| Q922L1 | ENHANCER OF ZESTE | 1 . . . 722 | 705/746 (94%) | 0.0 |
| | HOMOLOG 1 (DROSOPHILA) - | 4 . . . 749 | 714/746 (95%) | |
| | Mus musculus (Mouse), 750 aa. | | | |
| P70351 | Enhancer of zeste homolog 1 (ENX- | 1 . . . 722 | 705/746 (94%) | 0.0 |
| | 2) - Mus musculus (Mouse), 747 aa. | 1 . . . 746 | 714/746 (95%) | |
| Q99L74 | ENHANCER OF ZESTE | 15 . . . 722 | 466/754 (61%) | 0.0 |
| | HOMOLOG 2 (DROSOPHILA) - | 15 . . . 745 | 556/754 (72%) | |
| | Mus musculus (Mouse), 746 aa. | | | |
| Q61188 | Enhancer of zeste homolog 2 (ENX- | 15 . . . 722 | 465/754 (61%) | 0.0 |
| | 1) - Mus musculus (Mouse), 746 aa. | 15 . . . 745 | 555/754 (72%) | |

PFam analysis predicts that the NOV21 protein contains the domains shown in the Table 21E.

TABLE 21E
Domain Analysis of NOV21

| | | Identities/ | |
|---|---|---|---|
| | NOV21 | Similarities for | Expect |
| Pfam Domain | Match Region | the Matched Region | Value |
| zf-CCHC: domain | 560 . . . 575 | 6/18 (33%) | 8.9 |
| 1 of 1 | | 8/18 (44%) | |
| SET: domain 1 of 1 | 582 . . . 709 | 65/163 (40%) | 1.8e-60 |
| | | 117/163 (72%) | |

### Example 22.

The NOV22 clone was analyzed, and the nucleotide and encoded polypeptide sequences are shown in Table 22A.

TABLE 22A
NOV22 Sequence Analysis

| | SEQ ID NO:57 | 2010 bp |
|---|---|---|
| NOV22, | ATGGCCATTGTGCAGACTCTGCCAGTGCCACTGGACCCTGCTCCTGAAGCTCCCACTG | |
| CG93781-01 DNA | CCCCACAAGCTCCAGTCATGGGTAGTGTGAGCAGCCTTATCTCAGGCCGGCCCTGTCC | |
| Sequence | CGGGGGGCCAGCTCCTCCCCGCCACCACGGCCCTCCTGGGCCCACCTTCTTCCGCCAG | |
| | CAGGATGGCCTGCTACGGGGTGGCTATGAGGCACAGGAGCCGCTGTGCCCAGCTGTGC | |
| | CCCCTAGGAAGGCTGTCCCTGTCACCAGCTTCACCTACATCAATGAGGACTTCCGGAC | |
| | ACAGTCACCCCCCAGCCCAAGCACTGATGTTGAGGATGCCCGAGAGCAGCGGGCACAC | |
| | AATGCCCACCTCCGCGGCCCACCACCAAAGCTCATCCCTGTCTCTGGAAAGCTGCAGA | |
| | AGAACATGGAGAAGATCCTGATCCGCCCAACAGCCTTCAAGCCAGTGCTGCCCAAACC | |
| | TCGAGGGGCTCCGTCCCTGCCTAGCTTCATGGGTCCTCGGCCCACCCGGCTGTCTGGG | |
| | AGCCAGGGCAGCCTGACGCAGCTGTTTCGGGGCCCTGCCTCCTCCTCCTCCTCTTCCT | |
| | CCTCCTCTTCAGCTGCTGACAAACCCCTGGCATTTAGTGGCTGGGCCAGTGGCTGCCC | |
| | ATCAGGGACGCTATCCGACTCTGGCCGAAACTCACTGTCCAGCCTGCCCACCTACAGC | |
| | ACCGGAGGTGCCGAGCCAACCACCAGCTCCCCAGGCGGGCACCTGCCTTCCCATGGCT | |
| | CTGGGCGAGGGGCACTGCCTGGGCCAGCCCGAGGGGTCCCTACTGGGCCCTCCCACTC | |
| | AGACAGTGGCCGGTCCTCCTCCAGCAAGAGCACAGGCTCCCTAGGGGGCCGTGTGGCT | |
| | GGGGGGCTTTTGGGCAGTGGTACTCGGGCCTCCCCTGACAGCAGCTCCTGTGGGGAGC | |
| | GCTCACCACCACCCCCGCCTCCACCTCCTTCGGATGAGGCCCTGCTGCACTGTGTCCT | |
| | GGAAGGAAAGCTCCGAGACCGGGAGGCAGAGCTTCAGCAGCTGCGGGACAGTCTGGAC | |
| | GAGAATGAGGCTACCATGTGCCAGGCATACGAGGAGCGGCAGCGGCACTGGCAGCCAG | |
| | AGCGTGAGGCCCTGCGAGAGGACTGTGCGGCCCAGGCACAGCGGGCACAGCGGGCCCA | |
| | ACAGCTGCTGCAGCTGCAGGTGTTCCACCTGCAGCAGGAGAAGCGGCAATTGCAGGAC | |
| | GACTTCGCACAGCTGCTGCAGGAGCGCGAACAGCTGGAGCGGCGCTGCGCCACCTTGG | |
| | AGCGGGAGCAGCGGGAGCTCGGGCCGAGGCTTGAGGAGACCAAGTGGGAGGTGTGCCA | |
| | GAAATCAGGCGAGATCTCCCTGCTGAAGCAGCAGCTGAAAGAGTCTCAGGCAGAGCTG | |
| | GTGCAGAAGGGCAGCGAGCTGGTGGCTCTGCGGGTGGCGCTGCGGGAGGCCCGTGCTA | |
| | CGCTGCGGGTCAGTGAGGGCCGTGCGCGGGGTCTACAGGAGGCCGCCCGAGCTCGGGA | |
| | GCTGGAGCTGGAAGCCTGTTCCCAGGAGCTGCAGCGACACCGCCAGGAAGCTGAGCAG | |
| | CTGCGGGAGAAAGCTGGGCAGTTGGATGCTGAGGCGGCCGGACTCCGGGAGCCCCCTG | |
| | TGCCACCTGCCACCGCTGACCCATTCCTCCTGGCAGAGAGTGATGAGGCCAAAGTGCA | |
| | GCGGGCAGCAGCCGGGGTTGGGGGCAGCTTGCGGGCCCAGGTGGAGCGATTGCGGGTG | |
| | GAGCTGCAGCGGGAGCGGCGGCGGGGTGAGGAGCAGCGGGACAGCTTTGAGGGGGAGC | |
| | GGCTGCCCTGGCAGGCAGAGAAGGAGCAGGTGATCCGCTACCAGAAGCAGCTGCAGCA | |
| | CAACTACATCCAGATGTACCGGCGCAACCGGCAGCTAGAGCAGGAGCTGCAGCAGCTC | |
| | AGCCTGGAGCTGGAGGCCCGGGAGCTCGCTGACCTGGGCCTGGCCGAGCAGGCCCCCT | |
| | GCATCTGCCTGGAGGAGATCACTGCTACTGAGATCTAG | |
| | ORF Start: ATG at 1 | ORF Stop: TAG at 2008 |
| | SEQ ID NO:58 | 669 aa MW at 72758.5 kD |

| | |
|---|---|
| NOV22, | MAIVQTLPVPLEPAPEAATAPQAPVMGSVSSLTSGRPCPGGPAPPRHHGPPGPTFFRQ |
| CG93781-01 Protein | QDGLLRGGYEAQEPLCPAVPPRKAVPVTSFTYTNEDFRTESPPSPSSDVEDAREQRAH |
| Sequence | NAHLRGPPPKLIPVSGKLEKNMEKILTRPTAFKPVLPKPRGAPSLPSFMCPRATGLSG |
| | SQGSLTQLFGGPASSSSSSSSSSAADKPLAFSOWASGCPSGTLSDSGRNSLSSLPTYS |
| | TGGAEPTTSSPGGHLPSHGSGRGALPGPARCVPTGPSHSDSGRSSSSKSTGSLGGRVA |
| | GGLLGSGTRASPDSSSCGERSPPPPPPPPSDEALLHCVLEGKLRDREABLQQLRDSLD |
| | ENEATMCQAYEERQRHWQREREALREDCAAQAQRAQRAQQLLQLQVFQLQQEKRQLQD |
| | DFAQLLQEREQLERRCATLEREQRELGPRLEETKWEVCQKSGEISLLKQQLKESQAEL |
| | VQKGSELVALRVALREARATLRVSEGRARGLQEAARARELELEACSQELQRHRQEAEQ |
| | LREKAGQLDABAAGLREPPVPPATADPFLLAESDEAKVQRAAAGVCGSLRAQVERLRV |
| | ELQRERRRGEEQRDSFEGERLAWQAEKEQVIRYQKQLQHNYIQMYRRNRQLEQELQQL |
| | SLELEARELADLGLAEQAPCICLEEITATEI |

Further analysis of the NOV22 protein yielded the following properties shown in Table 22B.

TABLE 22B
Protein Sequence Properties NOV22

| | |
|---|---|
| PSort | 0.4500 probability located in cytoplasm; 0.3000 probability located in |
| analysis: | microbody (peroxisome); 0.1000 probability located in mitochondrial matrix |
| | space; 0.1000 probability located in lysosome (lumen) |
| SignalP | No Known Signal Sequence Predicted |
| analysis: | |

A search of the NOV22 protein against the Geneseq database, a proprietary database that contains sequences published in patents and patent publication, yielded several homologous proteins shown in Table 22C.

TABLE 22C
Geneseq Results for NOV22

| | | NOV22 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB56422 | Human prostate cancer antigen protein | 429 . . . 657 | 228/229 (99%) | e-123 |
| | sequence SEQ ID NO:1000 - Homo | 59 . . . 287 | 228/229 (99%) | |
| | sapiens, 320 aa. [WO200055174-A1, | | | |
| | 21-SEP-2000] | | | |
| AAB42077 | Human ORFX ORF1841 polypeptide | 1 . . . 185 | 184/185 (99%) | e-106 |
| | sequence SEQ ID NO:3682 - Homo | 1 . . . 185 | 185/185 (99%) | |
| | sapiens, 185 aa. [WO200058473-A2, | | | |
| | 05-OCT-2000] | | | |
| AAB08715 | Amino acid sequence of a human FEZ1 | 26 . . . 669 | 243/658 (36%) | 8e-86 |
| | polypeptide - Homo sapiens, 596 aa. | 1 . . . 596 | 320/658 (47%) | |
| | [WO200050565-A2, 31-AUG-2000] | | | |
| AAB08721 | Amino acid sequence of truncated FEZ1 | 26 . . . 669 | 237/659 (35%) | 9e-79 |
| | transcript G3611 - Homo sapiens, 563 | 1 . . . 563 | 311/659 (46%) | |
| | aa. [WO200050565-A2, 31-AUG-2000] | | | |
| AAB08722 | Amino acid sequence of truncated FEZ1 | 26 . . . 669 | 237/658 (36%) | 1e-77 |
| | transcript G3612 - Homo sapiens, 573 | 1 . . . 573 | 306/658 (46%) | |
| | aa. [WO200050565-A2, 31-AUG-2000] | | | |

In a BLAST search of public sequence datbases, the NOV22 protein was found to have homology to the proteins shown in the BLASTP data in Table 22D.

TABLE 22D
Public BLASTP Results for NOV22

| | | NOV22 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96JL2 | KIAA1813 PROTEIN - Homo sapiens | 1 . . . 669 | 669/669 (100%) | 0.0 |
| | (Human), 673 aa (fragment). | 5 . . . 673 | 669/669 (100%) | |
| Q96J79 | LAPSER1 - Homo sapiens (Human), | 26 . . . 669 | 644/644 (100%) | 0.0 |
| | 644 aa. | 1 . . . 644 | 644/644 (100%) | |
| Q9NTP2 | BA108L7.4 (NOVEL PROTEIN | 36 . . . 669 | 634/634 (100%) | 0.0 |
| | SIMILAR TO KIAA0552, KIAA0341 | 1 . . . 634 | 634/634 (100%) | |
| | AND FUGU HYPOTHETICAL | | | |
| | PROTEIN 2) - Homo sapiens (Human), | | | |
| | 634 aa (fragment). | | | |
| Q91YU6 | HYPOTHETICAL 72.6 KDA | 1 . . . 669 | 618/674 (91%) | 0.0 |
| | PROTEIN - Mus musculus | 1 . . . 671 | 634/674 (93%) | |
| | (Mouse), 671 aa. | | | |
| Q9BRK4 | HYPOTHETICAL 36.8 KDA | 354 . . . 669 | 316/316 (100%) | e-175 |
| | PROTEIN - Homo sapiens | 1 . . . 316 | 316/316 (100%) | |
| | (Human), 316 aa. | | | |

PFam analysis predicts that the NOV22 protein contains the domains shown in the Table 22E.

TABLE 22E
Domain Analysis of NOV22

| | | Identities/ | |
|---|---|---|---|
| | NOV22 | Similarities for | Expect |
| Pfam Domain | Match Region | the Matched Region | Value |
| bZIP: domain 1 of 2 | 412 . . . 452 | 14/41 (34%) | 0.19 |
| | | 30/41 (73%) | |
| bZIP: domain 2 of 2 | 514 . . . 539 | 11/26 (42%) | 9.8 |
| | | 20/26 (77%) | |
| DUF164: domain | 382 . . . 591 | 39/243 (16%) | 3.1 |
| 1 of 1 | | 111/243 (46%) | |
| hormone3: domain | 604 . . . 630 | 8/28 (29%) | 8.3 |
| 1 of 1 | | 21/28 (75%) | |

### Example 23.

The NOV23 clone was analyzed, and the nucleotide and encoded polypeptide sequences are shown in Table 23A.

TABLE 23A
NOV23 Sequence Analysis

| | SEQ ID NO:59 | 1590 bp |
|---|---|---|
| NOV23, | ATGGTGCAAAAGAAGAAGTTCTGTCCTCGGTTACTTGACTATCTAGTGATCGTAGGGG | |
| CG93848-02 DNA | CCAGGCACCCGAGCAGTGATAGCGTGGCCCAGACTCCTGAATTGCTACCGCGATACCC | |
| Sequence | CTTGGAGGATCACACTGACTTTCCCCTGCCCCCAGATGTAGTGTTCTTCTGCCAGCCC | |
| | GAGGGCTGCCTGAGCGTGCGGCAGCGGCGCATGAGCCTTCGGGATGATACCTCTTTTG | |
| | TCTTCACCCTCACTGACAAGGACACTGGAGTCACGCGATATGGCATCTGTGTTAACTT | |
| | CTACCGCTCCTTCCAAAAGCGAATCTCTAAGGAGAAGGGGGAAGGTGGGGCAGGGTCC | |
| | CGTGCGAAGGAAGGAACCCATCCCACCTGTGCCTCAGAACAGGCTCGCACTGAGAGCT | |
| | CAGAGAGTGGCTCATCCCTGCAGCCTCTCAGTGCTGACTCTACCCCTGATGTGAACCA | |
| | GTCTCCTCGGGGCAAACGCCGGGCCAAGGCGGGGAGCCGCTCCCGCAACAGTACTCTC | |
| | ACGTCCCTGTGCGTGCTCAGCCACTACCCTTTCTTCTCCACCTTCCGAGAGTGTTTGT | |
| | ATACTCTCAAGCGCCTGGTGGACTGCTGTAGTGAGCGCCTTCTGGGCAAGAAACTGGG | |
| | CATCCCTCGAGGCGTACAAAGGGACACCATGTGGCGGATCTTTACTGCATCGCTGCTG | |
| | GTAGAGGAGAAGTCAAGTGCCCTTCTGCATGACCTTCGAGAGATTGAGGCCTGGATCT | |
| | ATCGATTGCTGCGCTCCCCACTACCCGTCTCTGGGCAGAAGCGAGTAGACATCGAGGT | |
| | CCTACCCCAAGAGCTCCAGCCAGCTCTGACCTTTGCTCTTCCACACCCATCTCGATTC | |
| | ACCCTAGTGGATTTCCCACTGCACCTTCCCTTGGAACTTCTAGGTGTGGACGCCTGTC | |
| | TCCAGGTGCTAACCTGCATTCTGTTAGAGCACAAGGTGGTGCTACAGTCCCGAGACTA | |
| | CAATCCACTCTCCATGTCTGTCATCGCATTCGTGCCAATCATCTACCCACTGCAGTAT | |
| | ATGTTTCCTGTCATCCCGCTGCTACCCACCTGCATGGCATCAGCAGACCAGCTGCTGT | |
| | TGGCTCCAACCCCGTACATCATTGGGGTTCCTGCCAGCTTCTTCCTCTACAAACTGGA | |
| | CTTCAAAATGCCTCATGATGTATGGCTAGTGGATCTGGACAGCAATACGGTGATTGCC | |
| | CCCACCAATGCAGAAGTGCTGCCTATCCTGCCAGAACCAGAATCACTAGAGCTGAAAA | |
| | AGCATTTAAAGCAGGCCTTGGCCAGCATGAGTCTCAACACCCAGCCCATCCTCAATCT | |
| | GGAAGGGATCAACCTCAAATTCATGCACAATCAGGTTTTCATAGAGCTGAATCACATT | |
| | AAAAAGTGCAATACAGTTCGAGGCGTCTTTGTCCTGGAGGAATTTGTTCCTGAAATTA | |
| | AAGAAGTGGTGAGCCACAAGTACAAGACACCAATGCCCCACCAAATCTGCTACTCCGT | |
| | ATTATGTCTCTTCTCGTACGTGGCTGCAGTTCATAGCAGTGAGGAAGATCTCAGAACC | |
| | CCGCCCCGGCCTGTCTCTAGCTGA | |
| | ORF Start: ATG at 1 | ORF Stop: TGA at 1588 |
| | SEQ ID NO: 60 | 529 aa MW at 59525.3 kD |

| | |
|---|---|
| NOV23, | MVQKKKFCPRLLDYLVIVGARHPSSDSVAQTPELLRRYPLEDHTEFPLPPDVVPFCQP |
| CG93848-02 Protein | EGCLSVRQRRMSLRDDTSFVFTLTDKDTGVTRYGICVNFYRSFQKRISKEKGEGGAGS |
| Sequence | RGKEGTHATCASEEGGTESSESGSSLQPLSADSTPDVNQSPRGKRRAKAGSRSRNSTL |
| | TSLCVLSHYPFFSTFRECLYTLKRLVDCCSERLLGKKLGIPRGVQRDTMWRTFTGSLL |
| | VEEKSSALLHDLREIEAWIYRLLRSPVPVSGQKRVDIEVLPQELQPALTFALPDPSRF |
| | TLVDFPLHLPLELLGVDACLQVLTCILLEHKVVLQSRDYNALSMSVIAFVAMIYPLEY |
| | MFPVIPLLPTCMASAEQLLLAPTPYITGVPASFFLYKLDFKMPDDVWLVDLDSNRVIA |
| | PTNAEVLPILPEPESLELKKHLKQALASMSLNTQPILNLEGINLKFMHNQVFIELNHI |
| | KKCNTVRGVFVLEEFVPEIKEVVSHKYKTPMAHEICYSVLCLFSYVAAVHSSEEDLRT |
| | PPRPVSS |

Further analysis of the NOV23 protein yielded the following properties shown in Table 23B.

TABLE 23B
Protein Sequence Properties NOV23

| | |
|---|---|
| PSort | 0.7300 probability located in plasma membrane; 0.6400 probability located in |
| analysis: | endoplasmic reticulum (membrane); 0.1000 probability located in endoplasmic |
| | reticulum (lumen); 0.1000 probability located in outside |
| SignalP | No Known Signal Sequence Predicted |
| analysis: | |

A search of the NOV23 protein against the Geneseq database, a proprietary database that contains sequences published in patents and patent publication, yielded several homologous proteins shown in Table 23C.

TABLE 23C
Geneseq Results for NOV23

| | | NOV23 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Portion | Value |
| AAW35576 | TNF-R1-DD ligand protein clone | 1 . . . 446 | 446/446 (100%) | 0.0 |
| | 57TU4A - Homo sapiens, 1588 aa. | 1 . . . 446 | 446/446 (100%) | |
| | [WO9730084-A1, 21-AUG-1997] | | | |
| AAW64453 | Rat brain Rab3 GEP protein - Rattus sp, | 1 . . . 446 | 430/446 (96%) | 0.0 |
| | 1602 aa. [EP856583-A2, 05-AUG- | 1 . . . 445 | 434/446 (96%) | |
| | 1998] | | | |
| AAM36447 | Peptide #10484 encoded by probe for | 52 . . . 219 | 168/168 (100%) | 1e-94 |
| | measuring placental gene expression - | 1 . . . 168 | 168/168 (100%) | |
| | Homo sapiens, 168 aa. | | | |
| | [WO200157272-A2, 09-AUG-2001] | | | |
| AAM76338 | Human bone marrow expressed probe | 52 . . . 219 | 168/168 (100%) | 1e-94 |
| | encoded protein SEQ ID NO: 36644 - | 1 . . . 168 | 168/168 (100%) | |
| | Homo sapiens, 168 aa. | | | |
| | [WO200157276-A2, 09-AUG-2001] | | | |
| AAM63524 | Human brain expressed single exon | 52 . . . 219 | 168/168 (100%) | 1e-94 |
| | probe encoded protein SEQ ID NO: | 1 . . . 168 | 168/168 (100%) | |
| | 35629 - Homo sapiens, 168 aa. | | | |
| | [WO200157275-A2, 09-AUG-2001] | | | |

In a BLAST search of public sequence datbases, the NOV23 protein was found to have homology to the proteins shown in the BLASTP data in Table 23D.

TABLE 23D
Public BLASTP Results for NOV23

| | | NOV23 | Identities/ | |
|---|---|---|---|---|
| Protein | | Residues/ | Similarities for | |
| Accession | | Match | the Matched | Expect |
| Number | Protein/Organism/Length | Residues | Portion | Value |
| O15293 | MAP KINASE-ACTIVATING | 1 . . . 446 | 446/446 (100%) | 0.0 |
| | DEATH DOMAIN PROTEIN - | 1 . . . 446 | 446/446 (100%) | |
| | Homo sapiens (Human), 1588 aa. | | | |
| O15065 | KIAA0358 PROTEIN - Homo | 1 . . . 446 | 446/446 (100%) | 0.0 |
| | sapiens (Human), 1581 aa. | 1 . . . 446 | 446/446 (100%) | |
| Q15741 | DENN PROTEIN - Homo sapiens | 1 . . . 446 | 446/446 (100%) | 0.0 |
| | (Human), 1587 aa. | 1 . . . 446 | 446/446 (100%) | |
| AAL40268 | INSULINOMA-GLUCAGONOMA | 1 . . . 446 | 443/446 (99%) | 0.0 |
| | PROTEIN 20 SPLICE VARIANT 3- | 1 . . . 446 | 444/446 (99%) | |
| | Homo sapiens (Human), 1545 aa. | | | |
| AAL40267 | INSULINOMA-GLUCAGONOMA | 1 . . . 446 | 443/446 (99%) | 0.0 |
| | PROTEIN 20 SPLICE VARIANT 2 - | 1 . . . 446 | 444/446 (99%) | |
| | Homo sapiens (Human), 1565 aa. | | | |

PFam analysis predicts that the NOV23a protein contains the domains shown in the Table 23E.

TABLE 23E
Domain Analysis of NOV23

| | | Identities/ | |
|---|---|---|---|
| | NOV23 | Similarities for | Expect |
| Pfam Domain | Match Region | the Matched Region | Value |
| DENN: domain 1 of 1 | 254 . . . 402 | 83/154 (54%) | 7e-86 |
| | | 147/154 (95%) | |

### Example 24.

The NOV24 clone was analyzed, and the nucleotide and encoded polypeptide sequences are shown in Table 24A.

TABLE 24A
NOV24 Sequence Analysis

| | SEQ ID NO:61 | 1200 bp |
|---|---|---|
| NOV24, | GAACCTCAGAATCAGGAAGAACCCAGCCGACACCCAGCAGCAGCGGGAGGAAAGAGGC | |
| CG94161-01 DNA | GGCAGTGGGAGAGGGGAGGTGCCCACCTCCTCCCCTGCTGGGGTCCAGCCATGTCCCA | |
| Sequence | GCCTGCGGGAGGCAGGAGGAAGCCCAGGACCCTAGGGCCGCCTGTGTGCAGTATCCGG | |
| | CCTTTCAAGTCGAGTGAGCAGTACCTGGAGGCCATGAAGGAAGACCTGGCTGAGTGGC | |
| | TTCGCGACCTCTATGGGCTGGACATCGACGCAGCCAACTTCCTGCAGGTGCTGGAAAC | |
| | GGGCCTGGTGCTGTGCCAACACGCCAACGTTGTCACTGACGCTGCCCTGGCCTTCCTG | |
| | GCTGAGGCACCTGCCCAAGCCCAGAAGATTCCCATGCCCCGGGTCGGGGTCTCCTGCA | |
| | ATGGGGCCGCCCAGCCAGGTACCTTCCAGGCCAGGGACAATGTCTCTAACTTCATCCA | |
| | GTGGTGTCGAAAGGAGATGGGCATCCCAGAGGTGCTGATGTTCGAGACGGAGGACTTG | |
| | GTGCTGCGCAAGAACGTGAAGAACGTGGTGCTGTGTTTGCTGGAGCTGGGCCGCCGGG | |
| | CGTGGCGCTTTGGTGTTGCGGCGCCCACACTCGTGCAGCTGGACGAGGAGATCGAGGA | |
| | GGAGGTGCGGCGGGAGCTGGCCCTGCCCCCGCCCGACCCCTCGCCGCCAGCGCCCCCC | |
| | AGGCGCCAGCCCTGCCACTTCCGCAACCTGGACCAGATGGTGAGGGCCTCTGCACACG | |
| | CCCTCAGGGCCCCCTTCCCTTTGGTGCAGAGCCTTGTGAGCCACTGCACGTGCCCAGT | |
| | GCAGTTCTCCATGGTCAAAGTGTCTGAGGGGAAGTACCGTGTGGGTGACTCCAACACC | |
| | CTCATCTTCATCCGGGTACAGATCCTCCGGAACCATGTGATGGTACGTGTAGGGGGCG | |
| | GCTGGGACACACTGGGCCATTACCTGGACAAACATGACCCCTGCCGCTGCACATCCCT | |
| | CTGTGAGTCCCCTGAGGGCCCTCTCCCTGTGGGGTTGGTTGAAGAGGCCAGCCCGCGA | |
| | GCTGGTCCAGGAAGAGGGGCTGCCCTCCACCCCGCCCTTAACCTCACCCTTGCCCCCT | |
| | CAGATCCTCCGGAACCATGTCATGGTACGTGTAGGGGGCGGCTCGGACACACTGGGCC | |
| | ATTACCTGGACAAACATGACCCCTGCCGCTGCACATCCCT | |
| | ORF Start: ATG at 109 | ORF Stop: TGA at 1177 |
| | SEQ ID NO:62 | 356 aa MW at 38985.5 kD |

| | |
|---|---|
| NOV24, | MSQPAGGRRKPRTLGPPVCSIRPFKSSEQYLEAMKEDLAEWLRDLYGLDIDAANFLQV |
| CG94161-01 Protein | LETGLVLCQHANVVTDAALAFLAEAPAQAQKIPMPRVGVSCNGAAQPGTFQARDNVSN |
| Sequence | FIQWCRKEMGIPEVLMFETEDLVLRKNVKNVVLCLLELGRRAWRFGVAAPTLVQLEEE |
| | IEEEVRRELALPPPDPSPPAPPRRQPCHFRNLDQMVRGSAHALRAPFPLVQSLVSHCT |
| | CPVQFSMVKVSEGKYRVGDSNTLIFIRVQILRNHVMVRVGGGWDTLGHYLDKHDPCRC |
| | TSLCESPEGPLPVGLVEEASPRAGPGRGAALHPALNLTLAPSDPPEPCDGTCRGRLGH |
| | TGPLPGQT |

TABLE 24B
Protein Sequence Properties NOV24

| | |
|---|---|
| PSort | 0.6000 probability located in nucleus; 0.2252 probability |
| analysis: | located in lysosome (lumen); 0.1000 probability located in |
| | mitochondrial matrix space; 0.0000 probability located in |
| | endoplasmic reticulum (membrane) |
| SignalP | No Known Signal Sequence Predicted |
| analysis: | |

A search of the NOV24 protein against the Geneseq database, a proprietary database that contains sequences published in patents and patent publication, yielded several homologous proteins shown in Table 24C.

TABLE 24C
Geneseq Results for NOV24

| | | NOV24 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU14697 | Novel bone marrow polypeptide #96 - | 228..341 | 47/132 (35%) | 8e-12 |
| | Homo sapiens, 5447 aa. | 5189..5318 | 63/132 (47%) | |
| | [WO200157187-A2, AUG. 09, 2001] | | | |
| AAU14603 | Novel bone marrow polypeptide #2 - | 228..341 | 47/132 (35%) | 8e-12 |
| | Homo sapiens, 5373 aa. | 5115..5244 | 63/132 (47%) | |
| | [WO200157187-A2, AUG. 09, 2001] | | | |
| AAU18529 | Human cytoskeletal element-related | 228..289 | 32/62 (51%) | 2e-10 |
| | polypeptide #22 - Homo sapiens, 1225 | 1160..1219 | 41/62 (65%) | |
| | aa. [WO200155168-A1, AUG. 02, 2001] | | | |
| ABG20425 | Novel human diagnostic protein #20416 - | 228..289 | 31/62 (50%) | 5e-10 |
| | Homo sapiens, 367 aa. | 111..170 | 41/62 (66%) | |
| | [WO200175067-A2, OCT. 11, 2001] | | | |
| ABG20425 | Novel human diagnostic protein #20416 - | 228..289 | 31/62 (50%) | 5e-10 |
| | Homo sapiens, 367 aa. | 111..170 | 41/62 (66%) | |
| | [WO200175067-A2, OCT. 11, 2001] | | | |

In a BLAST search of public sequence datbases, the NOV24 protein was found to have homology to the proteins shown in the BLASTP data in Table 24D.

TABLE 24D
Public BLASTP Results for NOV24

| | | NOV24 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q99501 | GAS-2 related protein on | 1..334 | 185/359 (51%) | 2e-83 |
| | chromosome 22 (GAR22 protein) - | 1..326 | 216/359 (59%) | |
| | Homo sapiens (Human), 329 aa. | | | |
| Q96FE9 | GAS2-RELATED ON | 1..339 | 183/373 (49%) | 9e-82 |
| | CHROMOSOME 22 - Homo sapiens | 1..340 | 214/373 (57%) | |
| | (Human), 681 aa. | | | |
| Q9BUY9 | GAS2-RELATED ON | 1..339 | 183/373 (49%) | 9e-82 |
| | CHROMOSOME 22 - Homo sapiens | 1..340 | 214/373 (57%) | |
| | (Human), 681 aa. | | | |
| Q9D2H3 | 4930500E24RIK PROTEIN - Mus | 1..344 | 173/362 (47%) | 2e-78 |
| | musculus (Mouse), 344 aa. | 1..331 | 210/362 (57%) | |
| P11862 | Growth-arrest-specific protein 2 | 28..289 | 109/262 (41%) | 2e-47 |
| | (GAS-2) - Mus musculus (Mouse), | 31..271 | 155/262 (58%) | |
| | 314aa. | | | |

PFam analysis predicts that the NOV24 protein contains the domains shown in the Table 24E.

TABLE 24E
Domain Analysis of NOV24

| | | Identities/ | |
|---|---|---|---|
| | | Similarities | Expect |
| Pfam Domain | NOV24 Match Region | for the Matched Region | Value |
| GAS2: domain 1 of 1 | 223..292 | 40/77 (52%) | 1e-36 |
| | | 57/77 (74%) | |

### Example 25.

The NOV25 clone was analyzed, and the nucleotide and encoded polypeptide sequences are shown in Table 25A.

TABLE 25A
NOV25 Sequence Analysis

| | SEQ ID NO:63 | 1425 bp |
|---|---|---|
| NOV25, | GTGTGGAGGAAGAACTAAAAGGACATGGAAGCAGGAGGACACCCAGGTGATGGGTGTC | |
| CG94346-01 DNA | GTAAGCCTGGGAAATGTGATGGTAATACAAGCATGTCAAGCCCGCAGATCCTGACACC | |
| Sequence | CTTCAAGGATTGCTGCCATCAGTGTCATCAAACCCAGGGATTCCCTAATGCTAATCCA | |
| | CCAATTAGAGCACGGCTGGTTACTACAGAACCACTGATGAGTTTCAAAAAATGCAGAC | |
| | TTCTGGACCCCACCCTTGGAGATTTTGAATCAGGAGGTCTTAGGGCCAGAACTAGGAA | |
| | GACGAGAAGGCTTTGGGATATGGCTGGCTTCCAGAGACTGAATCAGCAACTCATATTC | |
| | TCAAGACCGAGGTGGGTAGGCCTGACACAGAAGGGTCAGGAGGCTGCTAGAATCTATG | |
| | ACAGGGCAGGAACAGCGCTGAGGCAGCAGAAACTGAGAAAGCAGAAGTCACCAGAAAG | |
| | AGAAAAGAAGTTCAAAGTAGAGGGAAAGACTGAGGAAATAAGCAGGGAGCACTGCACT | |
| | CAGGCTTTGGGTTTTCAGCAGTGGGTGTCCGACTTTAGAGTTGTTTTCCTGGAAGTGC | |
| | TGATACCAAACTTGGCAGAGAAGAATGGTATCGTGTTTCTATATAGCTGCCTGGACAA | |
| | GGGAGTTCGGCCTTTGGGAGATAGCGGGATATGAAGGTCCAACTAAAGAAATATCTCT | |
| | CTATCATATCCTTCTGGGCAAAGGTCCAAGGAACACCACGATGACATCCCGCCTGAAC | |
| | AAGGACCAGAACTGCCTCATGACGGGAACATCTTATCAATATCCTACCGGGCAGCAAG | |
| | CCATACTGCCCAGACCCCTCCCGCCCATACCTATAAATTACCCCAGAGTGTTGTTGGG | |
| | CATGGAGCTGCCAGCTCCGCCCCAGCCAGTCCCCAGCCCTGCCCCTATGCGAACACTG | |
| | CCTATGGCACAAAACTAGGCACAAAAACCAGCAGGCCCACCCCTGCCCTGAGCGGCCA | |
| | ATGCCTCCCATGTGAATGTGCACAGGGGGCACACACAGCCCTGCATCTAGCAGCATCC | |
| | TGCTCCCATGCTAATCCCAACACTGGCACAAACATGTGTACAGTTGCTGGTGAGGGCC | |
| | CCCCAACCTGCCTGAGCCATGCTGCCACTGCTGCTTCTATGAACACCTGCACGAAGGC | |
| | TGGCACTCCGGCATCCACTAGCACCTTGCTGCAGCCAACAAGTGTGCACCCCACTGCA | |
| | CCGCTGCTGCCACTGCGACTGGCACATGCGACTGAGGATGGATCATGTTTCCACAGCC | |
| | CTACAAAGCACTTTGGCTGGCACCATGCCTCAGAGAGTTGTGATCAGAGGTCCAGGAG | |
| | CACCTCAGGCCCCTCCAACATAGCAGGTTCCTAACCTTAAGGAGCCAGAGAACAAGAC | |
| | CGGGGCCTGATACCAGTGCCCCACAGTTATAAC | |
| | ORF Start: ATG at 25 | ORF Stop: TAA at 1366 |
| | SEQ ID NO:64 | 447 aa MW at 48218.9 kD |

| | |
|---|---|
| NOV25, | MEAGGQPGDGCRKPGKCDGNTSMSSPQTLTPFKDCCHQCHQTQGFPNANPPIRARLVT |
| CG94346-01 Protein | TEPLMSFKKCRLLDPTLGDFESGGLRARTRKTRRLWDMAGFQRLNQQLIFSPPRWVGL |
| Sequence | TQKGQEAARIYDRAGTALRQQKLRKQKSPEREKKFKVEGKTEEISREHCTQALGFQQW |
| | VSDFRVVFLEVLIPNLAEKNGIVFLYSCLDKGVRPLGDKAGYEGPTKEISLSYPSGQR |
| | SKEHHDDIPPEQGPELPHDGNILSISYRAASHTAQTPPAHTYKLPQSVVGHGAASSAP |
| | ASPQPCPYANTAYGTKLGTKTSRPTPALSGQCLPCECAQGAHTALHLAASCSHANPNT |
| | GTNMCTVAGEGPPTCLSHAATAASMNTCTKAGTPASTSTLLQPTSVHPTAPLLPLRLA |
| | HATEDGSCFHSPTKHFGWHHASESCDQRSRSTSGPSNIAGS |

Further analysis of the NOV25 protein yielded the following properties shown in Table 25B.

TABLE 25B
Protein Sequence Properties NOV25

| | |
|---|---|
| PSort | 0.4500 probability located in cytoplasm; 0.3000 probability located in microbody |
| analysis: | (peroxisome); 0.1000 probability located in mitochondrial matrix space; 0.1000 |
| | probability located in lysosome (lumen) |
| SignalP | No Known Signal Sequence Predicted |
| analysis: | |

A search of the NOV25 protein against the Geneseq database, a proprietary database that contains sequences published in patents and patent publication, yielded several homologous proteins shown in Table 25C.

TABLE 25C
Geneseq Results for NOV25

| | | NOV25 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG15215 | Novel human diagnostic protein #15206 - | 217..273 | 37/59 (62%) | 7e-12 |
| | Homo sapiens, 368 aa. | 312..368 | 47/59 (78%) | |
| | [WO200175067-A2, OCT. 11, 2001] | | | |
| AA006174 | Human polypeptide SEQ ID NO 20066 - | 217..273 | 37/59 (62%) | 7e-12 |
| | Homo sapiens, 188 aa. [WO200164835- | 132..188 | 47/59 (78%) | |
| | A2, SEP. 01, 2001] | | | |
| ABG15215 | Novel human diagnostic protein #15206 - | 217..273 | 37/59 (62%) | 7e-12 |
| | [Homo sapiens, 368 aa. | 312..368 | 47/59 (78%) | |
| | [WO200175067-A2, OCT. 11, 2001] | | | |
| AAM86251 | Human immune/haematopoietic antigen | 293..423 | 50/136 (36%) | 5e-11 |
| | SEQ ID NO:13844 - Homo sapiens, 130 | 8..130 | 66/136 (47%) | |
| | aa. [WO200157182-A2, AUG. 09, 2001] | | | |
| ABG29412 | Novel human diagnostic protein #29403 - | 232..284 | 29/55 (52%) | 8e-05 |
| | Homo sapiens, 676 aa. | 87.. 137 | 33/55 (59%) | |
| | [WO200175067-A2, OCT. 11, 2001] | | | |

In a BLAST search of public sequence datbases, the NOV25 protein was found to have homology to the proteins shown in the BLASTP data in Table 25D.

TABLE 25D
Public BLASTP Results for NOV25

| | | NOV25 | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAC81810 | MUC1 PROTEiN - Bos taurus | 215..443 | 63/242 (26%) | 3e-04 |
| | (Bovine), 580 aa. | 79..301 | 84/242 (34%) | |
| Q95L89 | MUCIN - Bos taurus (Bovine), 554 aa | 215..443 | 63/242 (26%) | 3e-04 |
| | (fragment). | 79..301 | 84/242 (34%) | |
| O13028 | ANTIFREEZE GLYCOPEPTIDE | 256..408 | 40/158 (25%) | 0.002 |
| | AFGP POLYPROTEIN PRECURSOR - | 268..425 | 55/158 (34%) | |
| | Boreogadus saida, 507 aa. | | | |
| Q95V69 | CELL SURFACE | 286..447 | 51/168 (30%) | 0.002 |
| | IMMOBILIZATION ANTIGEN | 110..265 | 70/168 (41%) | |
| | SERH6 - Tetrahymena thermophila, | | | |
| Q9VYZ5 | DLG1 PROTEIN - Drosophila | 229..403 | 41/179 (22%) | 0.003 |
| | melanogaster (Fruit fly), 960 aa. | 261..437 | 72/179 (39%) | |

PFam analysis predicts that the NOV25 protein contains the domains shown in the Table 25E.

TABLE 25E
Domain Analysis of NOV25

| | | Identities/ | |
|---|---|---|---|
| | | Similarities | Expect |
| Pfam Domain | NOV25 Match Region | for the Matched | Value |
| Keratin_B2: domain 1 of 1 | 252..367 | 27/177 (15%) | 4.3 |
| | | 51/177 (29%) | |

### Example 26.

The NOV26 clone was analyzed, and the nucleotide and encoded polypeptide sequences are shown in Table 26A.

TABLE 26A
NOV26 Sequence Analysis

| | SEQ ID NO:65 | 1485 bp |
|---|---|---|
| NOV26, | TGTGCCTAGTGTGTTGGGCGGGGAGTCCTGGGGGCGCGACGATGGAGGGAGTGGCTTG | |
| CG94600-01 DNA | GGACCTGCACTCATTCCCTCTTGTCCCATACTGGAGTTTGGGGAGCCACTTTCCCGTC | |
| Sequence | CCTCCACTGTGGAGCTGCGTTCCTGTGAGGGAGGAGGCCCTCTGTGGTGGCGAGGAAT | |
| | AAGAATAAAAGATTCTGGAGGAGTTGGAGAAGAGTGTATTCAGCCCCCAAACCACGAG | |
| | ATCAACAAAGAAATGCACAATTTTGAGGAAGAGTTAACTTGTCCCATATGTTATAGTA | |
| | TTTTTGAAGATCCTCGTGTACTGCCATCCTCTCATACATTTTGTAGAAATTGTTTGGA | |
| | AAACATTCTTCAGGCATCTGGTAACTTTTATATATGGAGACCTTTACGAATTCCACTC | |
| | AAGTGCCCTAATTGCAGAAGTATTACTGAAATTGCTCCAACTGGCATTGAATCTTTAC | |
| | CTGTTAATTTTGCACTAAGGGCTATTATTGAAAAGTACCAGCAAGAAGACCATCCAGA | |
| | TATTGTCACCTGCCCTGAACATTACAGGCAACCATTAAATGTTTACTGTCTATTAGAT | |
| | AAAAAATTAGTTTGTGGTCATTGCCTTACCATAGGTCAACATCATGGTCATCCTATAG | |
| | ATGACCTTCAAAGTGCCTATTTGAAAGAAAAGGACACTCCTCAAAAACTGCTTGAACA | |
| | GTTGACTGACACACACTGGACAGATCTTACCCATCTTATTGAAAAGCTGAAAGAACAA | |
| | AAATCTCATTCTGAGAAAATGATCCAAGGCGATAAGGAAGCTGTTCTCCAGTATTTTA | |
| | AGGAGCTTAATGATACATTAGAACAGAAAAAAAAAAGTTTCCTAACGGCTCTCTGTGA | |
| | TGTTGGCAATCTAATTAATCAAGAATATACTCCACAAATTGAAAGAATGAAGGAAATA | |
| | CGAGAGCAGCAGCTTGAATTAATGGCACTGACAATATCTTTACAAGAAGAGTCTCCAC | |
| | TTAAATTTCTTGAAAAAGTTGATGATGTACGCCAGCATGTACAGATCTTGAAACAAAG | |
| | ACCACTTCCTGAGGTTCAACCCGTTGAAATTTATCCTCGAGTAAGCAAAATATTGAAA | |
| | GAAGAATGGAGCAGAACAGAAATTGGACAAATTAAGAACGTTCTCATTCCCAAAATGA | |
| | AAATTTCTCCAAAAAGGATGTCATGTTCCTGGCCTGGTAAGGATGAAAAGGAAGTTGA | |
| | ATTTTTAAAAATTTTAAACATTGTTGTAGTTACATTAATTTCAGTAATACTGATGTCG | |
| | ATACTCTTTTTCAACCAACACATCATAACCTTTTTAAGTGAAATCACTTTAATATGGT | |
| | TTTCTGAAGCCTCTCTATCTGTTTACCAAAGTTTATCTAACAGTCTGCATAAGGTAAA | |
| | GAATATACTGTGTCACATTTTCTATTTGTTGAAGGAATTTGTGTGGAAAATAGTTTCC | |
| | CATTGAAAATGTCAACCTGAATTGTTTAAATGGGC | |
| | ORF Start: ATG at 245 | ORF Stop: TGA at 1454 |
| | SEQ ID NO:66 | 1403 aa MW at 47113.4 kD |

| | |
|---|---|
| NOV 26, | MHNFEEELTCPICYSIFEDPRVLPCSHTFCRNCLENILQASGNFYIWRPLRIPLKCPN |
| CG94600-01 Protein | CRSITEIAPTGIESLPVNFALRAIIEKYQQEDHPDIVTCPEHYRQPLNVYCLLDKKLV |
| Sequence | CGHCLTIGQHHGHPIDDLQSAYLKEKDTPQKLLEQLTDTHWTDLTHLIEKLKEQKSHS |
| | EKMIQGDKEAVLQYFKELNDTLEQKKKSFLTALCDVGNLINQEYTPQIERMKETREQQ |
| | LELMALTISLQEESPLKPLEKVDDVRQHVQILKQRPLPEVQPVEIYPRVSKTLKEEWS |
| | RTETGQIKNVLIPKMKTSFKRMSCSWPGKDEKEVEFLKILNIVVVTLISVTLMSILFF |
| | NQHIITFLSEITLIWFSEASLSVYQSLSNSLHKVKNILCHIFYLLKEFVWKIVSH |

Further analysis of the NOV26 protein yielded the following properties shown in Table 26B.

TABLE 26B
Protein Sequence Properties NOV26

| | |
|---|---|
| PSort | 0.8500 probability located in endoplasmic reticulum (membrane); 0.4400 |
| analysis: | probability located in plasma membrane; 0.1000 probability located in |
| | mitochondrial inner membrane; 0.1000 probability located in Golgi body |
| SignalP | No Known Signal Sequence Predicted |
| analysis: | |

A search of the NOV26 protein against the Geneseq database, a proprietary database that contains sequences published in patents and patent publication, yielded several homologous proteins shown in Table 26C.

TABLE 26C
Geneseq Results for NOV24

| | | NOV24 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG20978 | Novel human diagnostic protein | 1..318 | 313/318 (98%) | 0.0 |
| | #20969 - Homo sapiens, 586 aa. | 159..474 | 313/318 (98%) | |
| | [WO200175067-A2, OCT. 11, 2001] | | | |
| ABG20978 | Novel human diagnostic protein | 1..318 | 313/318 (35%) | 0.0 |
| | #20969 - Homo sapiens, 586 aa. | 159..474 | 313/318 (98%) | |
| | [WO200175067-A2, OCT. 11, 2001] | | | |
| AAU15880 | Human novel secreted protein, Se1q ID | 1..198 | 198/198 (100%) | e-119 |
| | 833 - Homo sapiens, 208 aa. | 11..208 | 198/198 (65%) | |
| | [WO200155322-A2, AUG. 02, 2001] | | | |
| ABB03345 | Human musculoskeletal system related | 1..198 | 198/198 (50%) | e-119 |
| | polypeptide SEQ ID NO 1292 - Homo | 11..208 | 198/198 (66%) | |
| | sapiens, 208 aa. [WO200155367-A1, | | | |
| | AUG. 02, 2001] | | | |
| AAM39361 | Human polypeptide SEQ ID NO 2506 - | 1..304 | 105/306 (34%) | 7e-54 |
| | Homo sapiens, 407 aa. | 1..307 | 174/306 (56%) | |
| | [WO200153312-A1, JUL. 26, 2001] | | | |

In a BLAST search of public sequence datbases, the NOV26 protein was found to have homology to the proteins shown in the BLASTP data in Table 26D.

TABLE 26D
Public BLASTP Results for NOV26

| | | NOV26 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q922Y2 | SIMILAR TO RIKEN CDNA | 1 . . . 402 | 333/402 (82%) | 0.0 |
| | 2310035M22 GENE-Mus musculus | 1 . . . 402 | 363/402 (89%) | |
| | (Mouse), 403 aa. | | | |
| Q9CUD5 | 2310035M22RIK PROTEIN-Mus | 1 . . . 388 | 314/388 (80%) | 0.0 |
| | musculus (Mouse), 389 aa (fragment). | 1 . . . 388 | 348/388 (88%) | |
| Q9CSP2 | 2700022F13RIK PROTEIN-Mus | 1 . . . 196 | 183/196 (93%) | e-111 |
| | musculus (Mouse), 196 aa (fragment). | 1 . . . 196 | 190/196 (96%) | |
| Q9BQ47 | CAR (RET FINGER PROTEIN 2) | 1 . . . 304 | 105/306 (34%) | 2e-53 |
| | (BA34F20.1)-Homo sapiens (Human), | 1 . . . 301 | 174/306 (56%) | |
| | 407aa. | | | |
| O60858 | Ret finger protein 2 (Leukemia associated | 1 . . . 304 | 105/306 (34%) | 2e-53 |
| | protein 5) (B-cell chronic lymphocytic | 1 . . . 301 | 174/306 (56%) | |
| | leukemia tumor suppressor Leu5) | | | |
| | (Putative tumor suppressor RFP2)-Homo | | | |
| | sapiens (Human), 407 aa. | | | |

PFam analysis predicts that the NOV26 protein contains the domains shown in the Table 26E.

TABLE 26E
Domain Analysis of NOV26

| | NOV26 | Identities/ | |
|---|---|---|---|
| Pfam | Match | Similarities | Expect |
| Domain | Region | for the Matched Region | Value |
| zf-C3HC4: | 10 . . . 59 | 19/59 (32%) | 2e-07 |
| domain 1 of 1 | | 35/59 (59%) | |
| zf-B_box: | 92 . . . 134 | 15/49 (31%) | 0.0024 |
| domain 1 of 1 | | 28/49 (57%) | |

### Example 27.

The NOV27 clone was analyzed, and the nucleotide and encoded polypeptide sequences are shown in Table 27A.

TABLE 27A
NOV27 Sequence Analysis

| | SEQ ID NO:67 | 3183 bp |
|---|---|---|
| N0V27, | CCTAGGATGATACCATTCACAATTTTGATTTCTTAAAGGGACTGGATGAAGGTGTTTC | |
| CG94820-02 DNA | TTGTACGTCAATTTATGAAAAGCATAGTGCAGGACTGACAAAGGGGATGCATGCCTAC | |
| Sequence | AGAAAACTGCTTTATGGAGTAAATGAAATTGCTGTAAAAGTGCCTTCTGTTTTTAAGC | |
| | TTCTAATTAAAGAGGTACTCAACCCATTTTACATTTTCCAGCTGTTCAGTGTTATACT | |
| | GTGGAGCACTGATGAATACTATTACTATGCTCTAGCTATTCTGGTTATGTCCATAGTA | |
| | TCAATCGTAAGCTCACTATATTCCATTAGAAAGATCTTTTCTACCGACCTTGTGCCAG | |
| | GAGATGTCATGGTCATTCCATTAAATGGGACAATAATGCCTTGTGATGCTGTGCTTAT | |
| | TAATGGTACCTGCATTGTAAACGAAAGCATGTTAACAGGTAAGGCCACCGCGCCCAGC | |
| | CTAAAACAATTGTTTAAACGAAGAAAAAATTTGAAGGACTCACTTGGATTTAGTACTT | |
| | CCAAAGGACAGCTTGTTCGTTCCATATTGTATCCCAAACCAACTGATTTTAAACTCTA | |
| | CAGAGATGCCTACTTGTTTCTACTATGTCTTGTGGCAGTTGCTGGCATTGGGTTTATC | |
| | TACACTATTATTAATGTACAAGTTGGGGTCAGAATTATCGAGTCCCTTGATATTATCA | |
| | CAATTACTGTGCCCCCTGCACTTCCTGCTGCAATGACTGCTGGTATTGTGTATGCTCA | |
| | GAGAAGACTGAAAAAAATCGGTATTTTCTGTATCAGTCCTCAAAGAATAAATATTTGT | |
| | GGACAGCCCAATCTTGTTTGCTTTGACAAGACTGGAACTCTAACTGAAGATGGTTTAG | |
| | ATCTTTGGGGGATTCAACGAGTGGAAAATGCACGATTTCTTTCACCAGAAGAAAATGT | |
| | GTGCAATGAGATGTTGGTAAAATCCCAGTTTGTTGCTTGTATGGCTACTTGTCATTCA | |
| | CTTACAAAAATTGAAGGAGTGCTCTCTGGTGATCCACTTGATCTGAAZATGTTTGAGG | |
| | CTATTGGATGGATTCTGGAAGAAGCAACTGAAGAAGAAACAGCACTTCATAATCGAAT | |
| | TATGCCCACAGTGGTTCGTCCTCCCAAACAACTGCTTCCTGAATCTACCCCTGCAGGA | |
| | AACCAAGAAATGGAGCTGTTTGAACTTCCAGCTACTTATGAGATAGGAATTGTTCGCC | |
| | AGTTCCCATTTTCTTCTGCTTTGCAACGTATGAGTGTGGTTGCCACGGTGCTGGGGGA | |
| | TAGGAAAATGGACGCCTACATGAAAGGAGCGCCCGAGGCCATTGCCGGTCTCTGTAAA | |
| | CCTGAAACAGTTCCTGTCGATTTTCAAAACGTTTTGGAAGACTTCACTAAACAGGGCT | |
| | TCCGTGTGATTGCTCTTGCACACAGAAAATTGGAGTCAAAACTGACATGGCATAAAGT | |
| | ACAGAATATTAGCAGAGATGCAATTGAGAACAACATGGATTTTATGGGATTAATTATA | |
| | ATGCAGAACAAATTAAAGCAAGAAACCCCTGCAGTACTTGAAGATTTGCATAAAGCCA | |
| | ACATTCGCACCGTCATGGTCACAGGTGACAGTATGTTGACTGCTGTCTCTGTGGCCAG | |
| | AGATTGTGGAATGATTCTACCTCAGGATAAAGTGATTATTGCTGAAGCATTACCTCCA | |
| | AAGGATGGGAAAGTTGCCAAAATAAATTGGCATTATGCAGACTCCCTCACGCAGTGCA | |
| | GTCATCCATCAGCAATTGACCCAGAGGCTATTCCGGTTAAATTGGTCCATGATAGCTT | |
| | AGAGGATCTTCAAATGACTCGTTATCATTTTGCAATGAATGGAAAATCATTCTCAGTG | |
| | ATACTGGAGCATTTTCAAGACCTTGTTCCTAAGTTGATGTTGCATGGCACCGTGTTTG | |
| | CCCGTATGGCACCTGATCAGAAGACACAGTTGATAGAAGCATTGCAAAATGTTGATTA | |
| | TTTTGTTGGGATGTGTGGTGATGGCGCAAATGATTGTGGTGCTTTGAAGAGGGCACAC | |
| | GGAGGCATTTCCTTATCCGAGCTCGAAGCTTCAGTGGCATCTCCCTTTACCTCTAAGA | |
| | CTCCTAGTATTTCCTGTGTGCCAAACCTTATCAGGGAAGGCCGTCCTGCTTTAATAAC | |
| | TTCCTTCTGTGTGTTTAAATTCATGGCATTGTACAGCATTATCCAGTACTTCAGTGTT | |
| | ACTCTGCTGTATTCTATCTTAAGTAACCTAGGAGACTTCCAGTTTCTCTTCATTGATC | |
| | TGGCAATCATTTTGGTAGTGGTATTTACAATGAGTTTAAATCCTGCCTGGAAAGAACT | |
| | TGTGGCACAAAGACCACCTTCGGGTCTTATATCTGGGGCCCTTCTCTTCTCCGTTTTG | |
| | TCTCAGATTATCATCTGCATTGGATTTCAATCTTTGCGTTTTTTTTGGGTCAAACAGC | |
| | AACCTTGGTATGAAGTGTGGCATCCAAAATCAGATGCTTGTAATACAACAGGAAGCGG | |
| | GTTTTGGAATTCTTCACACGTAGACAATGAAACCGAACTTGATGAACATAATATACAA | |
| | AATTATGAAAATACCACAGTATTTTTTATTTCCAGTTTTCAGTACCTCATAGTGGCAA | |
| | TCGCCTTTTCAAAAGGAAAACCCTTCAGGCAACCTTGCTACAAAAATTATTTTTTTGT | |
| | TTTTTCTGTGATTTTTTTATATATTTTTATATTATTCATCATGTTGTATCCAGTTGCC | |
| | TCTGTTGACCAGGTTCTTCAGATAGTGTGTGTACCATATCAGTGGCGTGTAACTATGC | |
| | TCATCATTGTTCTTGTCAATGCCTTTGTGTCTATCACAGTGGAGGAGTCAGTGGATCG | |
| | GTGGGGAAAATGCTGCTTACCCTGGGCCCTGGGCTGTAGAAAGAAGACACCAAAGGCA | |
| | AAGTACATGTATCTGGCGCAGGAGCTCTTGGTTGATCCAGAATGGCCACCAAAACCTC | |
| | AGACAACCACAGAAGCTAAAGCTTTAGTTAAGGAGAATGGATCATGTCAAATCATCAC | |
| | CATAACATAGCAGTGAATCAGTCTCAGTGGTATTGCTGATAGCAGTATTCAGGAATAT | |
| | GTGATTTTAGGAGTTTCTGATCCTGTGTGTCAGAATGGCACTAGTTCAGTTTATGTCC | |
| | CTTCTGATATAGTAGCTTATTTGACAGCTTTGCTCTTCCTTAAAATAAAAA | |
| | ORF Start: ATG at 105 | ORF Stop: TAG at 3024 |
| | SEQ ID NO:68 | 973 aa MW at 109O16.4 kD |

| | |
|---|---|
| NOV27, | MHAYRKLLYGVNEIAVKVPSVPKLLIKEVLNPFYIFQLFSVILWSTDEYYYYALAIVV |
| CG94820-02 Protein | MSIVSIVSSLYSIRKIFSTDLVPGDVMVIPLNGTIMPCDAVLINGTCIVNESMLTGKA |
| Sequence | TAPSLKQLFKRRKNLKDSLGFSTSKGQLVRSILYPKPTDFKLYRDAYLFLLCLVAVAG |
| | IGFIYTIINVQVGVRIIESLDIITITVPPALPAAMTAGIVYAQRRLKKIGIFCISPQR |
| | INTCGQPNLVCFDKTGTLTEDGLDLWGIQRVENARFLSPEENVCNEMLVKSQFVACMA |
| | TCHSLTKIEGVLSGDPLDLKMFEAIGWTLEEATEEETALNRIMPTVVRPPKQLLPESI |
| | TPAGNQEMELFELPATYEIGIVRQFPFSSALQRMSVVARVLGDRKMDAYMKGAPEAIA |
| | GLCKPETVPVDFQNVLEDFTKQGFRVIALAHRKLESKLTWHKVQNISRDAIENNMDFM |
| | GLIIMQNKLKQETPAVLEDLHKANIRTVMVTGDSMLTAVSVARDCGMILPQDKVIIAE |
| | ALPPKDGKVAKINWHYADSLTQCSHPSAIDPEAIPVKLVHDSLEDLQMTRYHFAMNGK |
| | SFSVILEHFQDLVPKLMLHGTVFARMAPDQKTQLIEALQNVDYFVGMCGDGANDCGAL |
| | KRAHGGISLSELEASVASPFTSKTPSTSCVPNLIREGRAALITSFCVFKFMALYSIIQ |
| | YFSVTLLYSILSNLGDFQFLFIDLAIILVVVFTNSLNPAWKELVAQRPPSGLISGALL |
| | FSVLSQIIICIGFQSLGFFWVKQQPWYEVWHPKSDACNTTGSGFWNSSHVDNETELDE |
| | HNIQNYENTTVFFISSFQYLIVAIAFSKGKPFRQPCYKNYFFVFSVIFLYTFILFIML |
| | YPVASVDQVLQIVCVPYQWRVTMLIIVLVNAFVSITVEESVDRWGKCCLPWALGCRKK |
| | TPKAKYMYLAQELLVDPEWPPKPQTTTEAKALVKENGSCQIITIT |

Further analysis of the NOV27 protein yielded the following properties shown in Table 27B.

TABLE 27B
Protein Sequence Properties NOV27

| | |
|---|---|
| PSort | 0.6000 probability located in plasma membrane; 0.4000 |
| analysis: | probability located in Golgi body; 0.3000 probability |
| | located in endoplasmic reticulum (membrane): |
| | 0.3000 probability located in microbody (peroxisome) |
| SignalP | Cleavage site between residues 46 and 47 |
| analysis: | |

A search of the NOV27 protein against the Geneseq database, a proprietary database that contains sequences published in patents and patent publication, yielded several homologous proteins shown in Table 27C.

TABLE 27C
Geneseq Results for NOV27

| | | NOV27 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB40996 | Human ORFX 0RF760 polypeptide | 256 . . . 916 | 661/691 (95%) | 0.0 |
| | sequence SEQ ID NO:1520-Homo | 2 . . . 692 | 661/691 (95%) | |
| | sapiens, 692 aa. [WO200058473-A2, | | | |
| | 05-OCT-2000] | | | |
| AAM93525 | Human polypeptide, SEQ ID NO: 3259- | 469 . . . 973 | 502/505 (99%) | 0.0 |
| | Homo sapiens, 505 aa. [EP1130094- | 1 . . . 505 | 502/505 (99%) | |
| | A2, 05-SEP-2001] | | | |
| AAU23078 | Novel human enzyme polypeptide #164- | 505 . . . 973 | 466/469 (99%) | 0.0 |
| | Homo sapiens, 476 aa. | 8 . . . 476 | 466/469 (99%) | |
| | [WO200155301-A2, 02-AUG-2001] | | | |
| AAM93906 | Human polypeptide, SEQ ID NO: 4053- | 136 . . . 951 | 348/825 (42%) | e-174 |
| | Homo sapiens, 842 aa.[EP1130094- | 61 . . . 837 | 497/825 (60%) | |
| | A2, 05-SEP-2001] | | | |
| AAM79751 | Human protein SEQ ID NO 3397- | 247 . . . 872 | 271/628 (43%) | e-136 |
| | Homo sapiens, 666 aa. [WO200157190- | 1 . . . 585 | 382/628 (60%) | |
| | A2, 09-AUG-2001] | | | |

In a BLAST search of public sequence datbases, the NOV27 protein was found to have homology to the proteins shown in the BLASTP data in Table 27D.

TABLE 27D
Public BLASTP Results for NOV27

| | | NOV27 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9H7F0 | Probable cation-transporting ATPase 3 | 154 . . . 812 | 657/664 (98%) | 0.0 |
| | (EC 3.6.3.-)-Homo sapiens (Human), | 1 . . . 664 | 657/664 (98%) | |
| | 684 aa (fragment). | | | |
| Q96KS1 | HYPOTHETICAL 77.3 KDA | 71 . . . 707 | 600/680 (88%) | 0.0 |
| | PROTEIN-Homo sapiens (Human), | 4 . . . 680 | 612/680 (89%) | |
| | 701aa. | | | |
| Q9NQ11 | Probable cation-transporting ATPase 1 | 5 . . . 951 | 412/1012 (40%) | 0.0 |
| | (EC 3.6.1.-)-Homo sapiens (Human), | 212 . . . 1175 | 585/1012 (57%) | |
| | 1180 aa. | | | |
| Q9N323 | HYPOTHETICAL 126.4 KDA | 3 . . . 912 | 379/975 (38%) | 0.0 |
| | PROTEIN-Caenorhabditis elegans, | 192 . . . 1110 | 557/975 (56%) | |
| | 1127 aa. | | | |
| Q21286 | Probable cation-transporting ATPase | 8 . . . 908 | 386/981 (39%) | e-178 |
| | K07E3.7 in chromosome X (EC 3.6.3.- | 202 . . . 1138 | 549/981 (55%) | |
| | )-Caenorhabditis elegans, 1152 aa. | | | |

PFam analysis predicts that the NOV27 protein contains the domains shown in the Table 27E.

TABLE 27E
Domain Analysis of NOV27

| | | Identities/ | |
|---|---|---|---|
| | NOV27 Match | for the Matched | Expect |
| Pfam Domain | Region | Region | Value |
| E1-E2_ATPase: | 70 . . . 114 | 16/47 (34%) | 3.7e-05 |
| domain 1 of 1 | | 35/47 (74%) | |
| Hydrolase: domain | 239 . . . 651 | 40/423 (9%) | 0.0099 |
| 1 of 1 | | 246/423 (58%) | |
| Hemagglutinin: | 763 . . . 769 | 4/7 (57%) | 8.9 |
| domain 1 of 1 | | 7/7 (100%) | |
| Cation_ATPase_C: | 742 . . . 903 | 27/224 (12%) | 2.1 |
| domain 1 of | | 115/224 (51%) | |

### Example B: Identification of NOVX Clones

The novel NOVX target sequences identified in the present invention may have been subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus.

Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

### Example C. Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune diseases), Panel CNSD.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No.4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1× TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 11° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1× TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl.eff=pl effusion=pleural effusion,

glio=glioma,

astro=astrocytoma, and

neuro=neuroblastoma.

General_Screening_Panel_v1.4 and General_Screening_Panely_v1.5

The plates for Panels 1.4 and 1.5 include 2 control wells (genomic DNA control and chemistry control) and 94 wells containing cDNA from various samples. The samples in Panels 1.4 and 1.5 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panels 1.4 and 1.5 are widely available through the American Type Culture Collection (ATCC), a repository for cultured cell lines, and were cultured using the conditions recommended by the ATCC. The normal tissues found on Panels 1.4 and 1.5 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 1001M non essential amino acids (Gibco), 1 mM sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100CM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 1001M non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_Comprehensive Panel_v1.0

The plates for AI_comprehensive panely_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

Patient 2: Diabetic Hispanic, overweight, not on insulin

Patient 7-9: Nondiabetic Caucasian and obese (BMI>30)

Patient 10: Diabetic Hispanic, overweight, on insulin

Patient 11: Nondiabetic African American and overweight

Patient 12: Diabetic Hispanic on insulin

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

A. CG59448-02: hCaT1

Expression of gene CG59448-02 was assessed using the primer-probe set Ag3440, described in Table AA. Results of the RTQ-PCR runs are shown in Tables AB and AC.

TABLE AA
Probe Name Ag3440

| | | | Start | SEQ |
|---|---|---|---|---|
| Pri- | | | Posi- | ID |
| mers | Sequences | Length | tion | No |
| For- | 5′-gggagagctgggaatatcag-3′ | 20 | 2233 | 70 |
| ward | | | | |
| Probe | TET-5′-atctgactgcgtgttctcac | 26 | 2253 | 71 |
| | ttcgct-3′-TAMRA | | | |
| Re- | 5′-acccaggaaaatgagagcaa-3′ | 20 | 2288 | 72 |
| verse | | | | |

TABLE AB
Panel 1.3D

| | Rel. Exp. (%) Ag3440, |
|---|---|
| Tissue Name | Run 167617401 |
| Liver adenocarcinoma | 1.1 |
| Pancreas | 57.4 |
| Pancreatic ca. CAPAN | 0.3 |
| 2 | |
| Adrenal gland | 2.1 |
| Thyroid | 2.8 |
| Salivary gland | 85.3 |
| Pituitary gland | 0.6 |
| Brain (fetal) | 22.2 |
| Brain (whole) | 40.6 |
| Brain (amygdala) | 8.4 |
| Brain (cerebellum) | 1.2 |
| Brain (hippocampus) | 8.5 |
| Brain (substantia nigra) | 11.1 |
| Brain (thalamus) | 8.5 |
| Cerebral Cortex | 65.1 |
| Spinal cord | 7.5 |
| glio/astro U87-MG | 0.0 |
| glio/astro U-118-MG | 1.0 |
| astrocytoma SW1783 | 0.3 |
| neuro*; met SK-N-AS | 0.3 |
| astrocytoma SF-539 | 0.0 |
| astrocytoma SNB-75 | 0.6 |
| glioma SNB-19 | 0.0 |
| glioma U251 | 0.0 |
| glioma SF-295 | 0.0 |
| Heart (fetal) | 1.6 |
| Heart | 0.0 |
| Skeletal muscle (fetal) | 0.2 |
| Skeletal muscle | 0.0 |
| Bone marrow | 0.0 |
| Thymus | 13.7 |
| Spleen | 0.5 |
| Lymph node | 1.3 |
| Colorectal | 0.6 |
| Stomach | 1.6 |
| Small intestine | 3.3 |
| Colon ca. SW480 | 3.5 |
| Colon ca.* | 23.2 |
| SW620(SW480 met) | |
| Colon ca. HT29 | 2.7 |
| Colon ca. HCT-116 | 0.0 |
| Colon ca. CaCo-2 | 1.0 |
| Colon ca. | 1.7 |
| tissue(ODO3866) | 1.7 |
| Colon ca. HCC-2998 | 0.7 |
| Gastric ca.* (liver met) | 0.9 |
| NCI-N87 | |
| Bladder | 35.1 |
| Trachea | 1.4 |
| Kidney | 7.0 |
| Kidney (fetal) | 23.5 |
| Renal ca. 786-0 | 0.0 |
| Renal ca. A498 | 0.0 |
| Renal ca. RXF 393 | 0.0 |
| Renal ca. ACHN | 0.8 |
| Renal ca. UO-31 | 0.0 |
| Renal ca. TK-10 | 0.0 |
| Liver | 1.9 |
| Liver (fetal) | 0.0 |
| Liver ca. | 1.6 |
| (heptoblast) HepG2 | |
| Lung | 0.7 |
| Lung (fetal) | 3.0 |
| Lung ca. (small cell) | 40.9 |
| LX-1 | |
| Lung ca. (small cell) | 0.0 |
| NCI-H69 | |
| Lung ca. (s.cell var.) | 0.0 |
| SHP-77 | |
| Lung ca. (large | 0.0 |
| cell) NCI-H460 | |
| Lung ca. (non-sm. | 0.0 |
| cell) A549 | |
| Lung ca. (non-s.cell) | 0.0 |
| NCI-H23 | |
| Lung ca. (non-s.cell) | 1.1 |
| HOP-62 | |
| (Lung ca.(non-s.cl) | 1.1 |
| NCI-H522 | |
| Lung ca. (squam.) | 1.1 |
| SW 900 | |
| Lung ca. (squam.) | 0.0 |
| NCI-H596 | |
| Mammary gland | 12.4 |
| Breast ca.* (pl.ef) | 0.0 |
| MCF-7 | |
| Breast ca.* (pl.ef) | 0.0 |
| MDA-MB-231 | |
| Breast ca.* (pl.ef) | 100.0 |
| T47D | |
| Breast ca.BT-549 | 1.2 |
| Breast ca.MDA-N | 0.0 |
| Ovary | 1.2 |
| Ovarian ca. OVCAR-3 | 2.0 |
| Ovarian ca. OVCAR-4 | 0.3 |
| Ovarian ca. OVCAR-5 | 0.1 |
| Ovarian ca. OVCAR-8 | 0.0 |
| Ovarian ca. IGROV-1 | 0.3 |
| Ovarian ca.* (ascites) | 0.0 |
| SK-OV-3 | |
| Uterus | 2.0 |
| Placenta | 85.9 |
| Prostate | 81.2 |
| Prostate ca.* (bone | 1.1 |
| met)PC-3 | |
| Testis | 4.8 |
| Melanoma | 0.0 |
| Hs688(A).T | |
| Melanoma* (met) | 0.4 |
| Hs688(B).T | 0.4 |
| Melanoma UACC-62 | 0.6 |
| Melanoma M14 | 0.0 |
| Melanoma LOX | 0.4 |
| IMVI | |
| Melanoma* (met) | 0.3 |
| SK-MEL-5 | |
| Adipose | 1.8 |

TABLE AC
Panel 5D

| | Rel. Exp. (%) |
|---|---|
| | Ag3440, Run |
| Tissue Name | 168075649 |
| 97457_Patient- | 0.5 |
| 02go_adipose | |
| 97476_Patient- | 0.0 |
| 07sk_skeletal muscle | |
| 97477_Patient- | 0.1 |
| 07ut_uterus | |
| 97478_Patient- | 46.0 |
| 07pl_placenta | |
| 97481_Patient- | 0.1 |
| 08sk_skeletal muscle | |
| 97482_Patient- | 0.0 |
| 08ut_uterus | |
| 97483_Patient- | 31.4 |
| 08pl_placenta | |
| 97486_Patient- | 0.1 |
| 09sk_skeletal muscle | |
| 97487_Patient- | 0.1 |
| 09ut_uterus | |
| 97488_Patient- | 40.3 |
| 09pl_placenta | |
| 97492_Patient- | 0.0 |
| 10ut_uterus | |
| 97493_Patient- | 100.0 |
| 10pl_placenta | |
| 97495_Patient- | 0.7 |
| 11go_adipose | |
| 97496_Patient- | 0.2 |
| 11sk_skeletal muscle | |
| 97497_Patient- | 0.1 |
| 11ut_uterus | |
| 97498_Patient- | 65.5 |
| 11pl_placenta | |
| 97500_Patient- | 0.5 |
| 12go_adipose | |
| 97501_Patient- | 0.4 |
| 12sk_skeletal muscle | |
| 97502_Patient- | 0.3 |
| 12ut_uterus | |
| 97503_Patient- | 32.1 |
| 12pl_placenta | |
| 94721_Donor 2 U- | 0.0 |
| A_Mesenchymal | |
| Stem Cells | |
| 94722_Donor 2 U- | 0.0 |
| B_Mesenchymal | |
| Stem Cells | |
| 94723_Donor 2 U- | 0.0 |
| C_Mesenchymal | |
| Stem Cells | |
| 94709_Donor 2 AM-A_adipose | 0.0 |
| 94710_Donor 2 AM-B_adipose | 0.0 |
| 94711_Donor 2 AM-C_adipose | 0.1 |
| 94712_Donor 2 AD-A_adipose | 0.0 |
| 94713_Donor 2 AD-B_adipose | 0.1 |
| 94714_Donor 2 AD-C_adipose | 0.1 |
| 94742_Donor 3 U- | 0.0 |
| A_Mesenchymal Stem Cells | |
| 94743_Donor 3 U- | 0.0 |
| B_Mesenchymal Stem Cells | |
| 94730_Donor 3 AM-A_adipose | 0.0 |
| 94731_Donor 3 AM-B_adipose | 0.1 |
| 94732_Donor 3 AM-C_adiposet | 0.0 |
| 94733_Donor 3 AD-A_adipose | 0.0 |
| 94734_Donor 3 AD-B_adipose | 0.0 |
| 94735_Donor 3 AD-C_adipose | 0.0 |
| 77138_Liver_HepG2untreated | 0.0 |
| 73556_Heart_Cardiac stromal cells | 0.0 |
| (primary) | |
| 81735_Small Intestine | 1.9 |
| 72409_Kidney_Proximal | 0.2 |
| Convoluted Tubule | |
| 82685_Small intestine_Duodenum | 3.7 |
| 90650_Adrenal_Adrenocortical | 0.0 |
| adenoma | |
| 72410_Kidney_HRCE | 0.0 |
| 72411_Kidney_HRE | 0.2 |
| 73139_Uterus_Uterine smooth | 0.0 |

Panel 1.3D Summary:

Ag3440 Highest expression of the CG59448-02 gene is seen in a breast cancer cell line (CT=29). Moderate levels of expression are also seen in lung and colon cancer cell lines. Thus, expression of this gene could be used to differentiate between the breast cancer cell line and other samples on this panel and as a marker for breast cancer. Furthermore, therapeutic modulation of the expression or function of this gene may be effective in the treatment of breast, lung and colon cancer.

This gene encodes a putative calcium transport protein homologous to hCAT1, which mediates calcium uptake. The CG59448-02 is moderately expressed in a variety of normal tissue samples, including prostate, placenta, salivary gland and pancreas. This expression profile is in agreement with published reports of the expression of hCAT1.

This gene also shows moderate to low levels of expression in the central nervous system, including the amygdala, hippocampus, substantia nigra, thalamus, and cerebral cortex. Inhibition of calcium uptake has been shown to decrease neuronal death in response to cerebral ischemia. Therefore, this gene represents an excellent drug target for the treatment of stroke. Treatment with an antagonist immediately after stroke could decrease total infarct volume and lessen the overall stroke severity (Matsuda T, Arakawa N, Takuma K, Kishida Y, Kawasaki Y, Sakaue M, Takahashi K, Takahashi T, Suzuki T, Ota T, Hamano-Takahashi A, Onishi M, Tanaka Y, Kameo K, Baba A. SEA0400, a novel and selective inhibitor of the Na+−Ca2+ exchanger, attenuates reperfusion injury in the in vitro and in vivo cerebral ischemic models. J Pharmacol Exp Ther 2001 Jul;298(1):249-56; Peng J B, Chen X Z, Berger U V, Weremowicz S, Morton C C, Vassilev P M, Brown E M, Hediger M A. Human calcium transport protein CaT1. Biochem Biophys Res Commun 2000 Nov 19;278(2):326-32).

Panel 5D Summary:

Ag34440 Expression of the CG59448-02 gene is seen primarily in the placenta (CTs=26−28). Moderate to low levels of expression are also seen in the small intestine (CTs=31−32). This expression profile is in agreement with published reports of the expression profile of hCAT1, a protein that mediates calcium uptake in the intestine. hCAT1 has also been identified as the cationic amino acid transporter in human placenta. Thus, the expression of the CG59448-02 gene and its homology to hCAT1 suggest that this gene product is involved in cellular calcium uptake and/or cationic amino acid transfer (Kamath S G, Furesz T C, Way B A, Smith C H. Identification of three cationic amino acid transporters in placental trophoblast: cloning, expression, and characterization of hCAT-1. J Membr Biol 1999 Sep 1;171(1):55-62).

B. CG59706-01 and CG59706-02: Tetratricopeptide Repeat-Containing Protein

Expression of gene CG59706-01 and full length clone CG59706-02 was assessed using the primer-probe set Ag3510, described in Table BA. Results of the RTQ-PCR runs are shown in Tables BB, BC and BD. Please note that 59706-02 represents a fill-length physical clone of the 59706-01 gene, validating the prediction of the gene sequence.

TABLE BA
Probe Name Ag3510

| | | | Start | SEQ |
|---|---|---|---|---|
| | | | Posi- | ID |
| Primers | Sequences | Length | tion | No |
| Forward | 5′-caattcagtgcttggagacagt | 22 | 131 | 73 |
| | -3′ | | | |
| Probe | TET-5′-tcagcccagaagatacaca | 26 | 161 | 74 |
| | cctagca-3′-TAMRA | | | |
| Reverse | 5′-tttctgtcaaaggctgtgaaac | 22 | 187 | 75 |
| | -3′ | | | |

TABLE BB
CNS_neurodegeneration_v1.0

| | Rel. Exp.(%) Ag3510, |
|---|---|
| Tissue Name | Run 210499482 |
| AD 1 Hippo | 3.8 |
| AD 2 Hippo | 18.6 |
| AD 3 Hippo | 1.6 |
| AD 4 Hippo | 2.2 |
| AD 5 hippo | 97.9 |
| AD 6 Hippo | 33.0 |
| Control 2 Hippo | 16.7 |
| Control 4 Hippo | 3.0 |
| Control (Path) 3 | 0.8 |
| Hippo | |
| AD 1 Temporal Ctx | 3.3 |
| AD 2 Temporal Ctx | 19.5 |
| AD 3 Temporal Ctx | 1.6 |
| AD 4 Temporal Ctx | 8.6 |
| AD 5 Inf Temporal | 96.6 |
| Ctx | |
| AD 5 Sup Temporal | 25.7 |
| Ctx | |
| AD 6 Inf Temporal | 38.4 |
| Ctx | |
| AD 6 Sup Temporal | 43.5 |
| Ctx | |
| Control 1 Temporal | 1.4 |
| Ctx | |
| Control 2 Temporal | 42.9 |
| Ctx | |
| Control 3 Temporal | 8.6 |
| Ctx | |
| Control 4 Temporal | 2.5 |
| Ctx | |
| Control (Path) 1 | 60.3 |
| Temporal Ctx | |
| Control (Path) 2 | 41.8 |
| Temporal Ctx | |
| Control (Path) 3 | 1.6 |
| Temporal Ctx | |
| Control (Path) 4 | 31.4 |
| Temporal Ctx | |
| AD 1 Occipital Ctx | 8.5 |
| AD 2 Occipital Ctx | 0.0 |
| (Missing) | |
| AD 3 Occipital Ctx | 1.7 |
| AD 4 Occipital Ctx | 10.7 |
| AD 5 Occipital Ctx | 10.2 |
| AD 6 Occipital Ctx | 41.5 |
| Control 1 Occipital | 1.1 |
| Ctx | |
| Control 2 Occipital | 65.5 |
| Ctx | |
| Control 3 Occipital | 9.3 |
| Ctx | |
| Control 4 Occipital | 2.1 |
| Ctx | |
| Control (Path) 1 | 91.4 |
| Occipital Ctx | |
| Control (Path) 2 | 5.8 |
| Occipital Ctx | |
| Control (Path) 3 | 0.7 |
| Occipital Ctx | |
| Control (Path) 4 | 11.3 |
| Occipital Ctx | |
| Control 1 Parietal | 2.5 |
| Ctx | |
| Control 2 Parietal | 21.9 |
| Ctx | |
| Control 3 Parietal | 14.2 |
| Ctx | |
| Control (Path) 1 | 100.0 |
| Parietal Ctx | |
| Control (Path) 2 | 17.6 |
| Parietal Ctx | |
| Control (Path) 3 | 1.1 |
| Parietal Ctx | |
| Control (Path) 4 | 37.4 |
| Parietal Ctx | |

TABLE BC
General_screening_panel_v1.4

| | Rel Exp. (%) Ag3510, |
|---|---|
| Tissue Name | Run 217240640 |
| Adipose | 5.3 |
| Melanoma* | 52.1 |
| Hs688(A).T | |
| Melanoma* | 71.2 |
| Hs688(B).T | |
| Melanoma* M14 | 39.2 |
| Melanoma* | 32.1 |
| LOXIMVI | |
| Melanoma* SK- | 42.9 |
| MEL-5 | |
| Squamous cell | 3.7 |
| carcinoma SCC-4 | |
| Testis Pool | 2.8 |
| Prostate ca.* (bone | 12.2 |
| met) PC-3 | |
| Prostate Pool | 4.2 |
| Placenta | 1.2 |
| Uterus Pool | 4.4 |
| Ovarian ca. OVCAR-3 | 5.2 |
| Ovarian ca. SK-OV-3 | 55.5 |
| Ovarian ca. OVCAR-4 | 3.3 |
| Ovarian ca. OVCAR-5 | 9.5 |
| Ovarian ca. IGROV-1 | 12.7 |
| Ovarian ca. OVCAR-8 | 19.1 |
| Ovary | 5.6 |
| Breast ca. MCF-7 | 3.6 |
| Breast ca. MDA- | 36.1 |
| MB-231 | |
| Breast ca. BT 549 | 82.9 |
| Breast ca. T47D | 30.6 |
| Breast ca. MDA-N | 30.8 |
| Breast Pool | 11.3 |
| Trachea | 5.8 |
| Lung | 2.7 |
| Fetal Lung | 14.3 |
| Lung ca. NCI-N417 | 3.9 |
| Lung ca. LX-1 | 23.0 |
| Lung ca. NCI-H146 | 41.8 |
| Lung ca. SHP-77 | 44.1 |
| Lung ca. A549 | 29.3 |
| Lung ca. NCI-H526 | 4.6 |
| Lung ca. NCI-H23 | 17.2 |
| Lung ca. NCI-H460 | 42.3 |
| Lung ca. HOP-62 | 5.4 |
| Lung ca. NCI-H522 | 84.7 |
| Liver | 0.3 |
| Fetal Liver | 6.4 |
| Liver ca. HepG2 | 12.9 |
| Kidney Pool | 29.3 |
| Fetal Kidney | 11.6 |
| Renal ca. 786-0 | 15.5 |
| Renal ca. A498 | 7.5 |
| Renal ca. ACHN | 9.5 |
| Renal ca. UO-31 | 12.4 |
| Renal Ca. TK-10 | 28.1 |
| Bladder | 10.1 |
| Gastric ca. (liver met.) | 18.9 |
| NCI-N87 | |
| Gastric ca. KATO III | 13.3 |
| Colon ca. SW-948 | 2.0 |
| Colon ca. SW480 | 46.7 |
| Colon ca.* (SW480 | 28.5 |
| met) SW620 | |
| Colon ca. HT29 | 3.7 |
| Colon ca. HCT-116 | 42.9 |
| Colon ca. CaCo-2 | 20.3 |
| Colon cancer tissue | 11.0 |
| Colon ca. SW1116 | 2.1 |
| Colon ca. Colo-205 | 1.7 |
| Colon ca. SW-48 | 0.0 |
| Colon Pool | 10.7 |
| Small Intestine Pool | 9.3 |
| Stomach Pool | 6.9 |
| Bone Marrow Pool | 4.6 |
| Fetal Heart | 4.7 |
| Heart Pool | 6.1 |
| Lymph Node Pool | 15.9 |
| Fetal Skeletal Muscle | 3.9 |
| Skeletal Muscle Pool | 4.4 |
| Spleen Pool | 6.3 |
| Thymus Pool | 16.2 |
| CNS cancer (glio/astro) | 68.3 |
| U87-MG | |
| CNS cancer (glio/astro) | 37.9 |
| U-118-MG | |
| CNS cancer (neuro;met) | 26.4 |
| SK-N-AS | |
| CNS cancer (astro) SF- | 9.3 |
| 539 | |
| CNS cancer (astro) | 75.8 |
| SNB-75 | |
| CNS cancer (glio) SNB- | 12.9 |
| 19 | |
| CNS cancer (glio) SF- | 71.7 |
| 295 | |
| Brain (Amygdala) Pool | 44.1 |
| Brain (cerebellum) | 30.6 |
| Brain (fetal) | 36.3 |
| Brain (Hippocampus) | 35.1 |
| Pool | |
| Cerebral Cortex Pool | 100.0 |
| Brain (Substantia nigra) | 69.7 |
| Pool | |
| Brain (Thalamus) Pool | 84.1 |
| Brain (whole) | 68.8 |
| Spinal Cord Pool | 23.7 |
| Adrenal Gland | 3.2 |
| Pituitary gland Pool | 4.5 |
| Salivary Gland | 0.4 |
| Thyroid (female) | 2.5 |
| Pancreatic ca. CAPAN2 | 5.1 |
| Pancreas Pool | 11.5 |

TABLE BD
Panel 4D

| | Rel. Exp. (%) | | Rel. Exp. (%) |
|---|---|---|---|
| | Ag3510, Run | | Ag3510, Run |
| Tissue Name | 166407237 | Tissue Name | 166407237 |
| Secondary Th1 act | 12.7 | HUVEC IL-1beta | 13.2 |
| Secondary Th2 act | 9.5 | HUVEC IFN gamma | 13.2 |
| Secondary Tr1 act | 13.5 | HUVEC TNF alpha + IFN | 15.9 |
| Secondary Th1 rest | 21.9 | gamma | |
| Secondary Th2 rest | 14.2 | HUVEC TNF alpha + IL4 | 14.6 |
| Secondary Tr1 rest | 17.2 | HUVEC IL-11 | 7.5 |
| Primary Th1 act | 7.1 | Lung Microvascular EC | 17.0 |
| Primary Th2 act | 10.7 | none | |
| Primary Tr1 act | 19.8 | Lung Microvascular EC | 20.9 |
| Primary Th1 rest | 73.7 | TNFalpha + IL-1beta | |
| Primary Th2 rest | 24.7 | Microvascular Dermal EC | 19.5 |
| Primary Tr1 rest | 23.3 | none | |
| CD45RA CD4 | 26.2 | Microsvasular Dermal EC | 22.5 |
| lymphocyte act | | TNFalpha + IL-1beta | |
| CD45RO CD4 | 24.5 | Bronchial epithelium | 15.7 |
| lymphocyte act | | TNFalpha + IL1beta | |
| CD8 lymphocyte act | 14.6 | Small airway epithelium | 4.8 |
| Secondary CD8 | 23.7 | none | |
| lymphocyte rest | | Small airway epithelium | 34.9 |
| Secondary CD8 | 11.7 | TNFalpha + IL-1beta | |
| lymphocyte act | | Coronery artery SMC rest | 18.3 |
| CD4 lymphocyte none | 40.6 | Coronery artery SMC | 11.6 |
| 2ry Th1/Th2/Tr1_anti- | 31.0 | TNFalpha + IL-1beta | |
| CCD1106 | 8.3 | Astrocytes rest | 23.3 |
| (Keratinocytes) | | | |
| CD95 CH11 | | Astrocytes TNFalpha + IL- | 54.7 |
| LAK cells rest | 21.8 | 1beta | |
| LAK cells IL-2 | 33.9 | KU-812 (Basophil) rest | 1.8 |
| LAK cells IL-2 + IL-12 | 22.5 | KU-812 (Basophil) | 5.6 |
| LAK cells IL-2 + IFN | 34.6 | PMA/ionomycin | |
| gamma | none | | |
| LAK cells IL-2 + IL-18 | 22.8 | CCD1106 (Keratinocytes) | 50.3 |
| LAK cells | 18.7 | TNFalpha + IL-1beta | |
| PMA/ionomycin | Liver cirrhosis | 6.7 | |
| NK Cells IL-2 rest | 11.4 | Lupus kidney | 1.5 |
| Two Way MLR 3 day | 39.2 | NCI-H292 none | 6.6 |
| Two Way MLR 5 day | 22.7 | NCI-H292 IL-4 | 8.5 |
| Two Way MLR 7 day | 16.5 | NCI-H292 IL-9 | 8.5 |
| PBMC rest | 23.3 | NCI-H292 IL-13 | 4.9 |
| PBMC PWM | 22.7 | NCI-H292 IFN gamma | 3.5 |
| PBMC PHA-L | 9.5 | HPAEC none | 6.6 |
| Ramos (B cell) none | 13.1 | HPAEC TNF alpha + IL-1 | 12.9 |
| Ramos (B cell) | 14.5 | beta | |
| ionomycin | | Lung fibroblast none | 24.7 |
| B lymphocytes PWM | 28.7 | Lung fibroblast TNF | 15.0 |
| B lymphocytes CD40L | 29.9 | alpha + IL-1 beta | |
| and IL-4 | | Lung fibroblast IL-4 | 20.3 |
| EOL-1 dbcAMP | 5.8 | Lung fibroblast IL-9 | 14.3 |
| EOL-1 dbcAMP | 10.2 | Lung fibroblast IL-13 | 12.2 |
| PMA/ionomycin | | Lung fibroblast IFN | 24.3 |
| Dendritic cells none | 29.9 | gamma | |
| Dendritic cells LPS | 29.1 | Dermal fibroblast | 69.3 |
| Dendritic cells anti- | 29.1 | CCD1070 rest | |
| CD40 | Dermal fibroblast | 100.0 | |
| Monocytes rest | 36.1 | CCD1070 TNF alpha | |
| Monocytes LPS | 88.9 | Dermal fibroblast | 36.6 |
| Macrophages rest | 90.8 | CCD1070 IL-1 beta | |
| Macrophages LPS | 47.6 | Dermal fibroblast IFN | 7.9 |
| HUVEC none | 17.7 | gamma | |
| HUVEC starved | 28.3 | Dermal fibroblast IL-4 | 19.2 |
| | | IBD Colitis 2 | 3.1 |
| | | IBD Crohn's | 2.8 |
| | | Colon | 22.1 |
| | | Lung | 8.7 |
| | | Thymus | 6.0 |
| | | Kidney | 25.9 |

CNS_Neurodegeneration_v1.0 Summary:

Ag3510 This panel confirms the expression of the CG59706-01 gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_Screening_Panel_v1.4 Summary:

Ag3510 Highest expression of the CG59706-01 gene is seen in cerebral cortex (CT=31). In addition, this gene is expressed at high levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Significant expression of this gene is seen in number of cancer cell lines (CNS, colon, lung, renal, gastric, breast, ovarian, squamous cell carcinoma, prostate and melanoma). Therefore, therapeutic modulation of the activity of the protein encoded by this gene may be beneficial in the treatment of these cancers.

Among tissues with metabolic or endocrine function, this gene is expressed at low levels in pancreas, and the gastrointestinal tract. Therefore, therapeutic modulation of the activity of this gene may prove useful in the treatment of endocrine/metabolically related diseases, such as obesity and diabetes.

Panel 4D Summary:

Ag3510 Highest expression of the CG59706-01 gene is detected in TNF alpha treated dermal fibroblast CCD1070 (CT=31). This gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Interestingly, expression of this gene is decreased in colon samples from patients with IBD colitis and Crohn's disease (CTs=35) relative to normal colon (CT=32). Therefore, therapeutic modulation of the activity of the protein encoded by this gene may be useful in the treatment of inflammatory bowel disease.

C. CG59766-01 and CG59766-02: TSG118.1

Expression of gene CG59766-01 and variant CG59766-02 was assessed using the primer-probe set Ag3579, described in Table CA. Results of the RTQ-PCR runs are shown in Tables CB, CC and CD.

TABLE CA
Probe Name Ag3579

| Primers | Sequences | Length | Start Position | SEQ ID No |
|---|---|---|---|---|
| Forward | 5′-actgggtaagtgaccccaaa-3′ | 20 | 82 | 76 |
| Probe | TET-5′-ctttccctcccgaaggggtcatct-3′-TAMRA | 24 | 108 | 77 |
| Reverse | 5′-tcttggtaccatcaggttgttc-3′ | 22 | 135 | 78 |

TABLE CB
CNS_neurodegeneration_v1.0

| | Rel. Exp. (%) Ag3579, | | Rel. Exp. (%) Ag3579, |
|---|---|---|---|
| Tissue Name | Run 210642349 | Tissue Name | Run 210642349 |
| AD 1 Hippo | 16.8 | Control (Path) 3 | 11.2 |
| AD 2 Hippo | 27.7 | Temporal Ctx | |
| AD 3 Hippo | 26.6 | Control (Path) 4 | 63.7 |
| AD 4 Hippo | 28.3 | Temporal Ctx | |
| AD 5 hippo | 68.8 | AD 1 Occipital Ctx | 30.4 |
| AD 6 Hippo | 41.8 | AD 2 Occipital Ctx | 0.0 |
| Control 2 Hippo | 39.8 | (Missing) | |
| Control 4 Hippo | 34.6 | AD 3 Occipital Ctx | 22.5 |
| Control (Path) 3 | 15.0 | AD 4 Occipital Ctx | 23.3 |
| Hippo | | AD 5 Occipital Ctx | 10.2 |
| AD 1 Temporal Ctx | 32.5 | AD 6 Occipital Ctx | 26.6 |
| AD 2 Temporal Ctx | 32.3 | Control 1 Occipital | 6.6 |
| AD 3 Temporal Ctx | 20.2 | Ctx | |
| AD 4 Temporal Ctx | 39.5 | Control 2 Occipital | 36.3 |
| AD 5 Inf Temporal | 63.3 | Ctx | |
| Ctx | Control 3 Occipital | 25.5 | |
| AD 5 Sup Temporal | 64.2 | Ctx | |
| Ctx | Control 4 Occipital | 23.5 | |
| AD 6 Inf Temporal | 37.1 | Ctx | |
| Ctx | Control (Path) 1 | 100.0 | |
| AD 6 Sup Temporal | 59.5 | Occipital Ctx | |
| Ctx | Control (Path) 2 | 25.5 | |
| Control 1 Temporal | 23.8 | Occipital Ctx | |
| Ctx | | Control (Path) 3 | 15.0 |
| Control 2 Temporal | 18.8 | Occipital Ctx | |
| Ctx | | Control (Path) 4 | 42.0 |
| Control 3 Temporal | 18.7 | Occipital Ctx | |
| Ctx | | Control 1 Parietal | 19.5 |
| Control 4 Temporal | 27.2 | Ctx | |
| Ctx | | Control 2 Parietal | 73.7 |
| Control (Path) 1 | 57.4 | Ctx | |
| Temporal Ctx | | Control 3 Parietal | 14.9 |
| Control (Path) 2 | 52.5 | Ctx | |
| Temporal Ctx | | Control (Path) 1 | 57.0 |
| | | Parietal Ctx | |
| | | Control (Path) 2 | 29.3 |
| | | Parietal Ctx | |
| | | Control (Path) 3 | 8.0 |
| | | Parietal Ctx | |
| | | Control (Path) 4 | 63.3 |
| | | Parietal Ctx | |

TABLE CC
General_screening_panel_v1.4

| | Rel. Exp. (%) Ag3579, | | Rel. Exp. (%) Ag3579, |
|---|---|---|---|
| Tissue Name | Run 217423486 | Tissue Name | Run 217423486 |
| Adipose | 0.6 | Renal ca. TK-10 | 2.2 |
| Melanoma* | 0.4 | Bladder | 3.7 |
| Hs688(A).T | | Gastric ca. (liver met.) | 6.1 |
| Melanoma* | 0.2 | NCI-N87 | |
| Hs688(B).T | | Gastric ca. KATO III | 3.1 |
| Melanoma* M14 | 0.9 | Colon ca. SW-948 | 0.4 |
| Melanoma* | 0.9 | Colon ca. SW480 | 1.8 |
| LOXIMVI | | Colon ca.* (SW480 | 1.4 |
| Melanoma* SK- | 2.3 | met) SW620 | |
| MEL-5 | | Colon ca. HT29 | 2.5 |
| Squamous cell | 0.7 | Colon ca. HCT-116 | 1.7 |
| carcinoma SCC-4 | | Colon ca. CaCo-2 | 1.3 |
| Testis Pool | 1.9 | Colon cancer tissue | 0.6 |
| Prostate ca.* (bone | 1.6 | Colon ca. SW1116 | 0.2 |
| met) PC-3 | | Colon ca. Colo-205 | 0.1 |
| Prostate Pool | 1.4 | Colon ca. SW-48 | 0.2 |
| Placenta | 0.6 | Colon Pool | 2.5 |
| Uterus Pool | 0.7 | Small Intestine Pool | 1.9 |
| Ovarian ca. OVCAR-3 | 3.1 | Stomach Pool | 0.9 |
| Ovarian ca. SK-OV-3 | 2.9 | Bone Marrow Pool | 1.1 |
| Ovarian ca. OVCAR-4 | 0.6 | Fetal Heart | 0.7 |
| Ovarian ca. OVCAR-5 | 5.8 | Heart Pool | 2.2 |
| Ovarian ca. IGROV-1 | 0.9 | Lymph Node Pool | 100.0 |
| Ovarian ca. OVCAR-8 | 0.4 | Fetal Skeletal Muscle | 0.9 |
| Ovary | 1.8 | Skeletal Muscle Pool | 0.4 |
| Breast ca. MCF-7 | 2.1 | Spleen Pool | 0.6 |
| Breast ca. MDA- | 1.6 | Thymus Pool | 0.9 |
| MB-231 | | CNS cancer (glio/astro) | 3.3 |
| Breast ca. BT 549 | 1.9 | U87-MG | |
| Breast ca. T47D | 5.3 | CNS cancer (glio/astro) | 3.4 |
| Breast ca. MDA-N | 0.6 | U-118-MG | |
| Breast Pool | 2.8 | CNS cancer (neuro;met) | 2.7 |
| Trachea | 2.1 | SK-N-AS | |
| Lung | 0.3 | CNS cancer (astro) SF- | 0.7 |
| Fetal Lung | 3.0 | 539 | |
| Lung ca. NCI-N417 | 0.2 | CNS cancer (astro) | 3.4 |
| Lung ca. LX-1 | 2.6 | SNB-75 | |
| Lung ca. NCI-H146 | 1.6 | CNS cancer (glio) SNB- | 0.7 |
| Lung ca. SHP-77 | 2.1 | 19 | |
| Lung ca. A549 | 2.4 | CNS cancer (glio) SF- | 8.1 |
| Lung ca. NCI-H526 | 0.2 | 295 | |
| Lung ca. NCI-H23 | 3.1 | Brain (Amygdala) Pool | 0.6 |
| Lung ca. NCI-H460 | 1.4 | Brain (cerebellum) | 1.7 |
| Lung ca. HOP-62 | 1.2 | Brain (fetal) | 2.3 |
| Lung ca. NCI-H522 | 0.8 | Brain (Hippocampus) | 1.5 |
| Liver | 0.0 | Pool | |
| Fetal Liver | 0.5 | Cerebral Cortex Pool | 1.5 |
| Liver ca. HepG2 | 0.6 | Brain (Substantia nigra) | 1.2 |
| Kidney Pool | 4.1 | Pool | |
| Fetal Kidney | 9.9 | Brain (Thalamus) Pool | 1.5 |
| Renal ca. 786-0 | 1.9 | Brain (whole) | 1.0 |
| Renal ca. A498 | 0.9 | Spinal Cord Pool | 1.3 |
| Renal ca. ACHN | 4.2 | Adrenal Gland | 0.7 |
| Renal ca. UO-31 | 2.7 | Pituitary gland Pool | 1.6 |
| | | Salivary Gland | 0.4 |
| | | Thyroid (female) | 0.5 |
| | | Pancreatic ca. CAPAN2 | 2.5 |
| | | Pancreas Pool | 3.1 |

TABLE CD
Panel 4.1D

| | Rel. Exp. (%) | | Rel. Exp. (%) |
|---|---|---|---|
| | Ag3579, Run | | Ag3579, Run |
| Tissue Name | 169910372 | Tissue Name | 169910372 |
| Secondary Th1 act | 4.5 | HUVEC IL-1beta | 20.7 |
| Secondary Th2 act | 10.5 | HUVEC IFN gamma | 11.7 |
| Secondary Tr1 act | 5.1 | HUVEC TNF alpha + IFN | 12.6 |
| Secondary Th1 rest | 1.4 | gamma | |
| Secondary Th2 rest | 1.3 | HUVEC TNF alpha + IL4 | 9.5 |
| Secondary Tr1 rest | 0.0 | HUVEC IL-11 | 15.6 |
| Primary Th1 act | 7.5 | Lung Microvascular EC | 44.1 |
| Primary Th2 act | 8.4 | none | |
| Primary Tr1 act | 5.8 | Lung Microvascular EC | 92.7 |
| Primary Th1 rest | 0.8 | TNFalpha + IL-1beta | |
| Primary Th2 rest | 0.0 | Microvascular Dermal EC | 22.2 |
| Primary Tr1 rest | 3.4 | none | |
| CD45RA CD4 | 9.5 | Microsvasular Dermal EC | 100.0 |
| lymphocyte act | | TNFalpha + IL-1beta | |
| CD45RO CD4 | 7.4 | Bronchial epithelium | 7.1 |
| lymphocyte act | | TNFalpha + IL1beta | |
| CD8 lymphocyte act | 4.1 | Small airway epithelium | 2.0 |
| Secondary CD8 | 7.2 | none | |
| lymphocyte rest | | Small airway epithelium | 4.1 |
| Secondary CD8 | 3.0 | TNFalpha + IL-1beta | |
| lymphocyte act | | Coronery artery SMC rest | 4.3 |
| CD4 lymphocyte none | 4.7 | Coronery artery SMC | 9.5 |
| 2ry Th1/Th2/Tr1_anti- | 2.6 | TNFalpha + IL-1beta | |
| CD95 CH11 | Astrocytes rest | 6.0 | |
| LAK cells rest | 5.8 | Astrocytes TNFalpha + IL- | 8.6 |
| LAK cells IL-2 | 2.9 | 1beta | |
| LAK cells IL-2 + IL-12 | 6.5 | KU-812 (Basophil) rest | 5.9 |
| LAK cells IL-2 + IFN | 8.8 | KU-812 (Basophil) | 10.8 |
| gamma | | PMA/ionomycin | |
| LAK cells IL-2 + IL-18 | 11.7 | CCD1106 (Keratinocytes) | 13.3 |
| LAK cells | 0.0 | none | |
| PMA/ionomycin | | CCD1106 (Keratinocytes) | 18.7 |
| NK Cells IL-2 rest | 1.6 | TNFalpha + IL-1beta | |
| Two Way MLR 3 day | 10.2 | Liver cirrhosis | 1.9 |
| Two Way MLR 5 day | 2.6 | NCI-H292 none | 28.7 |
| Two Way MLR 7 day | 2.2 | NCI-H292 IL-4 | 25.7 |
| PBMC rest | 3.0 | NCI-H292 IL-9 | 24.1 |
| PBMC PWM | 7.4 | NCI-H292 IL-13 | 19.1 |
| PBMC PHA-L | 6.8 | NCI-H292 IFN gamma | 30.1 |
| Ramos (B cell) none | 2.9 | HPAEC none | 21.2 |
| Ramos (B cell) | 2.7 | HPAEC TNF alpha + IL-1 | 55.9 |
| ionomycin | | beta | |
| B lymphocytes PWM | 5.4 | Lung fibroblast none | 10.3 |
| B lymphocytes CD40L | 0.0 | Lung fibroblast TNF | 4.2 |
| and IL-4 | | alpha + IL-1 beta | |
| EOL-1 dbcAMP | 2.2 | Lung fibroblast IL-4 | 6.6 |
| EOL-1 dbcAMP | 4.8 | Lung fibroblast IL-9 | 8.4 |
| PMA/ionomycin | | Lung fibroblast IL-13 | 7.5 |
| Dendritic cells none | 5.4 | Lung fibroblast IFN | 13.7 |
| Dendritic cells LPS | 3.7 | gamma | |
| Dendritic cells anti- | 10.2 | Dermal fibroblast | 8.5 |
| CD40 | | CCD1070 rest | |
| Monocytes rest | 1.4 | Dermal fibroblast | 17.0 |
| Monocytes LPS | 8.1 | CCD1070 TNF alpha | |
| Macrophages rest | 4.7 | Dermal fibroblast | 6.1 |
| Macrophages LPS | 0.0 | CCD1070 IL-1 beta | |
| HUVEC none | 6.3 | Dermal fibroblast IFN | 6.5 |
| HUVEC starved | 16.7 | gamma | |
| | | Dermal fibroblast IL-4 | 2.9 |
| | | Dermal Fibroblasts rest | 5.1 |
| | | Neutrophils TNFa + LPS | 0.0 |
| | | Neutrophils rest | 0.0 |
| | | Colon | 3.6 |
| | | Lung | 14.3 |
| | | Thymus | 3.3 |
| | | Kidney | 23.8 |

CNS_Neurodegeneration_v1.0 Summary:

Ag3579 This panel confirms the expression of the CG59766-01 gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_Screening_Panel_v1.4 Summary:

Ag3579 Highest expression of the CG59766-01 gene is detected in lymph node (CT=25). Therefore expression of this gene can be used to distinguish this sample from other samples in this panel. In addition, low but significant expression of this gene is associated with number of cancer cell lines (pacreatic, CNS, colon, renal, gastric, lung, breast, ovarian, prostate, squamous cell carcinoma, and melanoma) used in this panel. Therefore, therapeutic modulation of this gene product could be useful in the treatment of these cancers.

Among tissues with metabolic or endocrine function, this gene is expressed at moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

Interestingly, this gene is expressed at much higher levels in fetal (CT=30−33) when compared to adult lung and liver(CT=33−40). This observation suggests that expression of this gene can be used to distinguish fetal from adult lung and liver. In addition, the relative overexpression of this gene in fetal tissue suggests that the protein product may enhance growth or development of lung and liver in the fetus and thus may also act in a regenerative capacity in the adult. Therefore, therapeutic modulation of the protein encoded by this gene could be useful in treatment of lung and liver related diseases.

In addition, this gene is expressed at moderate levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4.1D Summary:

Ag3579 Highest expression of the CG59766-01 gene is detected in TNFalpha+IL-1beta treated microvascular dermal EC cells (CT=31.6). In addition, low to moderate expression of this gene is seen in other endothelial cells, keratinocytes, NCI-H292, lung and kidney. Thus, expression of this gene can be used to distinguish these samples from other samples in this panel. Furthermore, therapeutic modulation of this gene product can be useful in treatment of chronic obstructive pulmonary disease, asthma, allergy, emphysema, psoriasis, and inflammatory disease of kidney including lupus and glomerulonephritis.

D. CG59813-01: Novel Protein

Expression of gene CG59813-01 was assessed using the primer-probe set Ag3593, described in Table DA. Results of the RTQ-PCR runs are shown in Table DB.

TABLE DA
Probe Name Ag3593

| | | | Start | SEQ ID |
|---|---|---|---|---|
| Primers | Sequences | Length | Position | No |
| Forward | 5′-gttccaaaggatttcaccaaa-3′ | 21 | 187 | 79 |
| Probe | TET-5′-cctgtgataacaatctctgatgaacca-3′-TAMRA | 27 | 208 | 80 |
| Reverse | 5′-acagccttaccgtgtgacaa-3′ | 20 | 265 | 81 |

TABLE DB
General_screening_panel_v1.4

| | Rel. Exp. (%) Ag3593, | | Rel. Exp. (%) Ag3593, |
|---|---|---|---|
| Tissue Name | Run 217491551 | Tissue Name | Run 217491551 |
| Adipose | 0.0 | Renal ca. TK-10 | 0.0 |
| Melanoma* | 0.0 | Bladder | 0.0 |
| Hs688(A).T | | Gastric ca. (liver met.) | 3.0 |
| Melanoma* | 0.0 | NCI-N87 | |
| Hs688(B).T | | Gastric ca. KATO III | 6.8 |
| Melanoma* M14 | 0.0 | Colon ca. SW-948 | 0.0 |
| Melanoma* | 0.0 | Colon ca. SW480 | 0.0 |
| LOXIMVI | | Colon ca.* (SW480 | 0.0 |
| Melanoma* SK- | 11.5 | met) SW620 | |
| MEL-5 | | Colon ca. HT29 | 0.0 |
| Squamous cell | 3.6 | Colon ca. HCT-116 | 0.0 |
| carcinoma SCC-4 | | Colon ca. CaCo-2 | 0.0 |
| Testis Pool | 0.0 | Colon cancer tissue | 0.0 |
| Prostate ca.* (bone | 0.0 | Colon ca. SW1116 | 0.0 |
| met) PC-3 | | Colon ca. Colo-205 | 0.0 |
| Prostate Pool | 0.0 | Colon ca. SW-48 | 0.0 |
| Placenta | 0.0 | Colon Pool | 0.0 |
| Uterus Pool | 0.0 | Small Intestine Pool | 0.0 |
| Ovarian ca. OVCAR-3 | 0.0 | Stomach Pool | 0.0 |
| Ovarian ca. SK-OV-3 | 0.0 | Bone Marrow Pool | 0.0 |
| Ovarian ca. OVCAR-4 | 0.0 | Fetal Heart | 0.0 |
| Ovarian ca. OVCAR-5 | 0.0 | Heart Pool | 0.0 |
| Ovarian ca. IGROV-1 | 0.0 | Lymph Node Pool | 0.0 |
| Ovarian ca. OVCAR-8 | 1.6 | Fetal Skeletal Muscle | 0.0 |
| Ovary | 0.0 | Skeletal Muscle Pool | 0.0 |
| Breast ca. MCF-7 | 0.0 | Spleen Pool | 0.0 |
| Breast ca. MDA- | 0.0 | Thymus Pool | 0.0 |
| MB-231 | | CNS cancer (glio/astro) | 0.0 |
| Breast ca. BT 549 | 0.0 | U87-MG | |
| Breast ca. T47D | 0.0 | CNS cancer (glio/astro) | 0.0 |
| Breast ca. MDA-N | 0.0 | U-118-MG | |
| Breast Pool | 0.0 | CNS cancer (neuro;met) | 23.8 |
| Trachea | 0.0 | SK-N-AS | |
| Lung | 0.0 | CNS cancer (astro) SF- | 10.4 |
| Fetal Lung | 0.0 | 539 | |
| Lung ca. NCI-N417 | 100.0 | CNS cancer (astro) | 20.4 |
| Lung ca. LX-1 | 0.0 | SNB-75 | |
| Lung ca. NCI-H146 | 0.0 | CNS cancer (glio) SNB- | 8.5 |
| Lung ca. SHP-77 | 0.0 | 19 | |
| Lung ca. A549 | 0.0 | CNS cancer (glio) SF- | 0.0 |
| Lung ca. NCI-H526 | 0.0 | 295 | |
| Lung ca. NCI-H23 | 0.0 | Brain (Amygdala) Pool | 0.0 |
| Lung ca. NCI-H460 | 4.4 | Brain (cerebellum) | 0.0 |
| Lung ca. HOP-62 | 0.0 | Brain (fetal) | 0.0 |
| Lung ca. NCI-H522 | 3.4 | Brain (Hippocampus) | 0.0 |
| Liver | 0.0 | Pool | |
| Fetal Liver | 0.0 | Cerebral Cortex Pool | 0.0 |
| Liver ca. HepG2 | 0.0 | Brain (Substantia nigra) | 0.0 |
| Kidney Pool | 0.0 | Pool | |
| Fetal Kidney | 0.0 | Brain (Thalamus) Pool | 0.0 |
| Renal ca. 786-0 | 0.0 | Brain (whole) | 0.0 |
| Renal ca. A498 | 0.0 | Spinal Cord Pool | 0.0 |
| Renal ca. ACHN | 0.0 | Adrenal Gland | 0.0 |
| Renal ca. UO-31 | 0.0 | Pituitary gland Pool | 0.0 |
| | | Salivary Gland | 0.0 |
| | | Thyroid (female) | 0.0 |
| | | Pancreatic ca. CAPAN2 | 0.0 |
| | | Pancreas Pool | 0.0 |

CNS_Neurodegeneration_v1.0 Summary:

Ag3593 Expression of the CG59813-01 gene is low/undetectable in all samples on this panel (CTs=40).

General₁₃ Screening_Panel_v1.4 Summary:

Ag3593 Expression of the CG59813-01 gene is restricted to a sample derived from a lung cancer cell line (CT=33.7). Thus, expression of this gene could be used to differentiate between this sample and other samples on this panel and as a marker to detect the presence of lung cancer. Furthermore, therapeutic modulation of the expression or function of this gene may be effective in the treatment of lung cancer.

Panel 4.1D Summary:

Ag3593 Expression of the CG59813-01 gene is low/undetectable in all samples on this panel (CTs40).

E. CG59815-01: Novel Protein.

Expression of gene CG59815-01 was assessed using the primer-probe set Ag3594, described in Table EA. Results of the RTQ-PCR runs are shown in Tables EB.

TABLE EA
Probe Name Ag3594

| | | | Start | SEQ ID |
|---|---|---|---|---|
| Primers | Sequences | Length | Position | No |
| Forward | 5′-ggactaaaggaggccttctgt-3′ | 21 | 441 | 82 |
| Probe | TET-5′-ctctgcaggcccttcagtaggaacat-3′-TAMRA | 26 | 465 | 83 |
| Reverse | 5′-atcactggtctccgagtgaga-3′ | 21 | 510 | 84 |

TABLE EB
General_screening_panel_v1.4

| | Rel. Exp. (%) Ag3594, | | Rel. Exp. (%) Ag3594, |
|---|---|---|---|
| Tissue Name | Run 217494781 | Tissue Name | Run 217494781 |
| Adipose | 4.1 | Renal ca. TK-10 | 0.0 |
| Melanoma* | 0.0 | Bladder | 24.5 |
| Hs688(A).T | | Gastric ca. (liver met.) | 19.6 |
| Melanoma* | 0.0 | NCI-N87 | |
| Hs688(B).T | | Gastric ca. KATO III | 12.8 |
| Melanoma* M14 | 0.0 | Colon ca. SW-948 | 0.0 |
| Melanoma* | 14.8 | Colon ca. SW480 | 8.5 |
| LOXIMVI | Colon ca.* (SW480 | 4.2 | |
| Melanoma* SK- | 4.3 | met) SW620 | |
| MEL-5 | Colon ca. HT29 | 4.4 | |
| Squamous cell | 0.0 | Colon ca. HCT-116 | 100.0 |
| carcinoma SCC-4 | | Colon ca. CaCo-2 | 4.8 |
| Testis Pool | 8.5 | Colon cancer tissue | 3.6 |
| Prostate ca.* (bone | 0.0 | Colon ca. SW1116 | 4.1 |
| met) PC-3 | Colon ca. Colo-205 | 2.9 | |
| Prostate Pool | 0.0 | Colon ca. SW-48 | 0.0 |
| Placenta | 23.7 | Colon Pool | 0.8 |
| Uterus Pool | 1.5 | Small Intestine Pool | 7.2 |
| Ovarian ca. OVCAR-3 | 4.5 | Stomach Pool | 5.8 |
| Ovarian ca. SK-OV-3 | 19.5 | Bone Marrow Pool | 2.1 |
| Ovarian ca. OVCAR-4 | 0.0 | Fetal Heart | 0.0 |
| Ovarian ca. OVCAR-5 | 13.9 | Heart Pool | 9.3 |
| Ovarian ca. IGROV-1 | 14.0 | Lymph Node Pool | 2.7 |
| Ovarian ca. OVCAR-8 | 6.6 | Fetal Skeletal Muscle | 0.3 |
| Ovary | 5.3 | Skeletal Muscle Pool | 0.0 |
| Breast ca. MCF-7 | 15.5 | Spleen Pool | 0.0 |
| Breast ca. MDA- | 8.5 | Thymus Pool | 16.3 |
| MB-231 | | CNS cancer (glio/astro) | 6.2 |
| Breast ca. BT 549 | 33.4 | U87-MG | |
| Breast ca. T47D | 3.6 | CNS cancer (glio/astro) | 5.3 |
| Breast ca. MDA-N | 0.0 | U-118-MG | |
| Breast Pool | 5.8 | CNS cancer (neuro;met) | 17.9 |
| Trachea | 4.6 | SK-N-AS | |
| Lung | 0.0 | CNS cancer (astro) SF- | 0.0 |
| Fetal Lung | 10.2 | 539 | |
| Lung ca. NCI-N417 | 0.0 | CNS cancer (astro) | 0.0 |
| Lung ca. LX-1 | 4.4 | SNB-75 | |
| Lung ca. NCI-H146 | 0.0 | CNS cancer (glio) SNB- | 17.3 |
| Lung ca. SHP-77 | 5.5 | 19 | |
| Lung ca. A549 | 3.6 | CNS cancer (glio) SF- | 2.3 |
| Lung ca. NCI-H526 | 0.0 | 295 | |
| Lung ca. NCI-H23 | 22.5 | Brain (Amygdala) Pool | 1.2 |
| Lung ca. NCI-H460 | 25.7 | Brain (cerebellum) | 2.1 |
| Lung ca. HOP-62 | 31.6 | Brain (fetal) | 0.0 |
| Lung ca. NCI-H522 | 7.2 | Brain (Hippocampus) | 0.7 |
| Liver | 0.0 | Pool | |
| Fetal Liver | 0.0 | Cerebral Cortex Pool | 6.6 |
| Liver ca. HepG2 | 4.5 | Brain (Substantia nigra) | 6.9 |
| Kidney Pool | 8.3 | Pool | |
| Fetal Kidney | 0.0 | Brain (Thalamus) Pool | 4.8 |
| Renal ca. 786-0 | 4.8 | Brain (whole) | 0.0 |
| Renal ca. A498 | 0.0 | Spinal Cord Pool | 8.5 |
| Renal ca. ACHN | 0.0 | Adrenal Gland | 2.9 |
| Renal ca. UO-31 | 4.3 | Pituitary gland Pool | 0.0 |
| | | Salivary Gland | 4.2 |
| | | Thyroid (female) | 2.0 |
| | | Pancreatic ca. CAPAN2 | 5.3 |
| | | Pancreas Pool | 0.0 |

CNS_neurodegeneration_v1.0 Summary:

Ag3594 Expression of the CG59815-01 gene is low/undetectable in all samples on this panel (CTs>35).

General_Screening_Panel v1.4 Summary:

Ag3594 Expression of the CG59815-01 gene is highest in a colon cancer cell line (CT=31.7). Low but significant expression is also seen in other cancer cell lines, including samples derived from breast, lung and ovarian cancer. Thus, expression of this gene could be used to differentiate between the colon cancer and other samples on this panel and as a marker for colon cancer. Furthermore, therapeutic modulation of the expression or function of this gene may be effective in the treatment of colon, breast, lung and ovarian cancers.

Panel 4.1D Summary:

Ag3594 Expression of the CG59815-01 gene is low/undetectable in all samples on this panel (CTs>35).

F. CG59817-02: Novel Transcription Elongation Factor-Like

Expression of gene CG59817-02 was assessed using the primer-probe set Ag3595, described in Table FA. Results of the RTQ-PCR runs are shown in Tables FB, FC and FD.

TABLE FA
Probe Name Ag3595

| | | | Start | SEQ ID |
|---|---|---|---|---|
| Primers | Sequences | Length | Position | No |
| Forward | 5′-aaaatattgaacgggaaacgtt-3′ | 22 | 473 | 85 |
| Probe | TET-5′-tcatctctgctcccgcctcattaatg-3′-TAMRA | 26 | 495 | 86 |
| Reverse | 5′-ctcggtgctttaatgtgaagac-3′ | 22 | 550 | 87 |

TABLE FB
CNS_neurodegeneration_v1.0

| | Rel. Exp. (%) Ag3595, | | Rel. Exp. (%) Ag3595, |
|---|---|---|---|
| Tissue Name | Run 211009917 | Tissue Name | Run 211009917 |
| AD 1 Hippo | 21.8 | Control (Path) 3 | 15.7 |
| AD 2 Hippo | 39.5 | Temporal Ctx | |
| AD 3 Hippo | 13.7 | Control (Path) 4 | 29.9 |
| AD 4 Hippo | 8.4 | Temporal Ctx | |
| AD 5 hippo | 85.9 | AD 1 Occipital Ctx | 18.4 |
| AD 6 Hippo | 54.3 | AD 2 Occipital Ctx | 0.0 |
| Control 2 Hippo | 36.1 | (Missing) | |
| Control 4 Hippo | 20.6 | AD 3 Occipital Ctx | 9.0 |
| Control (Path) 3 | 21.5 | AD 4 Occipital Ctx | 18.3 |
| Hippo | | AD 5 Occipital Ctx | 33.4 |
| AD 1 Temporal Ctx | 38.4 | AD 6 Occipital Ctx | 42.3 |
| AD 2 Temporal Ctx | 40.1 | Control 1 Occipital | 14.2 |
| AD 3 Temporal Ctx | 12.4 | Ctx | |
| AD 4 Temporal Ctx | 22.7 | Control 2 Occipital | 58.2 |
| AD 5 Inf Temporal | 80.7 | Ctx | |
| Ctx | | Control 3 Occipital | 21.0 |
| AD 5 SupTemporal | 44.1 | Ctx | |
| Ctx | | Control 4 Occipital | 18.6 |
| AD 6 Inf Temporal | 54.7 | Ctx | |
| Ctx | | Control (Path) 1 | 100.0 |
| AD 6 Sup Temporal | 44.4 | Occipital Ctx | |
| Ctx | | Control (Path) 2 | 24.0 |
| Control 1 Temporal | 15.9 | Occipital Ctx | |
| Ctx | | Control (Path) 3 | 22.7 |
| Control 2 Temporal | 43.2 | Occipital Ctx | |
| Ctx | | Control (Path) 4 | 22.5 |
| Control 3 Temporal | 19.8 | Occipital Ctx | |
| Ctx | | Control 1 Parietal | 20.9 |
| Control 4 Temporal | 15.4 | Ctx | |
| Ctx | | Control 2 Parietal | 40.9 |
| Control (Path) 1 | 71.7 | Ctx | |
| Temporal Ctx | | Control 3 Parietal | 20.0 |
| Control (Path) 2 | 50.7 | Ctx | |
| Temporal Ctx | | Control (Path) 1 | 65.5 |
| | | Parietal Ctx | |
| | | Control (Path) 2 | 31.0 |
| | | Parietal Ctx | |
| | | Control (Path) 3 | 21.3 |
| | | Parietal Ctx | |
| | | Control (Path) 4 | 38.2 |
| | | Parietal Ctx | |

TABLE FC
General_screening_panel_v1.4

| | Rel. Exp. (%) Ag3595, | | Rel. Exp. (%) Ag3595, |
|---|---|---|---|
| Tissue Name | Run 217499730 | Tissue Name | Run 217499730 |
| Adipose | 4.0 | Renal ca. TK-10 | 55.5 |
| Melanoma* | 12.9 | Bladder | 14.0 |
| Hs688(A).T | | Gastric ca. (liver met.) | 44.8 |
| Melanoma* | 17.2 | NCI-N87 | |
| Hs688(B).T | | Gastric ca. KATO III | 50.0 |
| Melanoma* M14 | 33.4 | Colon ca. SW-948 | 6.1 |
| Melanoma* | 38.4 | Colon ca. SW480 | 41.5 |
| LOXIMVI | | Colon ca.* (SW480 | 17.7 |
| Melanoma* SK- | 30.4 | met) SW620 | |
| MEL-5 | | Colon ca. HT29 | 11.3 |
| Squamous cell | 13.7 | Colon ca. HCT-116 | 26.6 |
| carcinoma SCC-4 | | Colon ca. CaCo-2 | 6.7 |
| Testis Pool | 27.5 | Colon cancer tissue | 7.6 |
| Prostate ca.* (bone | 33.7 | Colon ca. SW1116 | 2.5 |
| met) PC-3 | | Colon ca. Colo-205 | 12.7 |
| Prostate Pool | 7.0 | Colon ca. SW-48 | 3.1 |
| Placenta | 4.9 | Colon Pool | 21.8 |
| Uterus Pool | 3.5 | Small Intestine Pool | 5.7 |
| Ovarian ca. OVCAR-3 | 24.7 | Stomach Pool | 2.9 |
| Ovarian ca. SK-OV-3 | 18.8 | Bone Marrow Pool | 6.7 |
| Ovarian ca. OVCAR-4 | 4.2 | Fetal Heart | 10.2 |
| Ovarian ca. OVCAR-5 | 28.9 | Heart Pool | 6.9 |
| Ovarian ca. IGROV-1 | 5.7 | Lymph Node Pool | 5.1 |
| Ovarian ca. OVCAR-8 | 9.2 | Fetal Skeletal Muscle | 9.7 |
| Ovary | 7.7 | Skeletal Muscle Pool | 11.0 |
| Breast ca. MCF-7 | 75.8 | Spleen Pool | 8.6 |
| Breast ca. MDA- | 38.4 | Thymus Pool | 27.0 |
| MB-231 | | CNS cancer (glio/astro) | 22.2 |
| Breast ca. BT 549 | 39.8 | U87-MG | |
| Breast ca. T47D | 70.7 | CNS cancer (glio/astro) | 94.0 |
| Breast ca. MDA-N | 16.3 | U-118-MG | |
| Breast Pool | 15.9 | CNS cancer (neuro;met) | 48.0 |
| Trachea | 17.4 | SK-N-AS | |
| Lung | 3.2 | CNS cancer (astro) SF- | 31.2 |
| Fetal Lung | 27.5 | 539 | |
| Lung ca. NCI-N417 | 6.7 | CNS cancer (astro) | 62.9 |
| Lung ca. LX-1 | 37.6 | SNB-75 | |
| Lung ca. NCI-H146 | 11.1 | CNS cancer (glio) SNB- | 3.0 |
| Lung ca. SHP-77 | 22.7 | 19 | |
| Lung ca. A549 | 6.1 | CNS cancer (glio) SF- | 42.6 |
| Lung ca. NCI-H526 | 5.4 | 295 | |
| Lung ca. NCI-H23 | 31.6 | Brain (Amygdala) Pool | 6.1 |
| Lung ca. NCI-H460 | 3.3 | Brain (cerebellum) | 11.0 |
| Lung ca. HOP-62 | 19.1 | Brain (fetal) | 4.0 |
| Lung ca. NCI-H522 | 100.0 | Brain (Hippocampus) | 10.6 |
| Liver | 1.1 | Pool | |
| Fetal Liver | 6.9 | Cerebral Cortex Pool | 12.7 |
| Liver ca. HepG2 | 7.5 | Brain (Substantia nigra) | 5.0 |
| Kidney Pool | 9.4 | Pool | |
| Fetal Kidney | 12.9 | Brain (Thalamus) Pool | 12.2 |
| Renal ca. 786-0 | 8.0 | Brain (whole) | 4.9 |
| Renal ca. A498 | 8.1 | Spinal Cord Pool | 8.4 |
| Renal ca. ACHN | 8.7 | Adrenal Gland | 14.0 |
| Renal ca. UO-31 | 6.6 | Pituitary gland Pool | 2.6 |
| | | Salivary Gland | 3.3 |
| | | Thyroid (female) | 4.7 |
| | | Pancreatic ca. CAPAN2 | 11.0 |
| | | Pancreas Pool | 17.1 |

TABLE FD
Panel 4.1D

| | Rel. Exp. (%) | | Rel. Exp. (%) |
|---|---|---|---|
| | Ag3595, Run | | Ag3595, Run |
| Tissue Name | 169910379 | Tissue Name | 169910379 |
| Secondary Th1 act | 49.3 | HUVEC IL-1beta | 39.2 |
| Secondary Th2 act | 100.0 | HUVEC IFN gamma | 26.1 |
| Secondary Tr1 act | 84.1 | HUVEC TNF alpha + IFN | 20.2 |
| Secondary Th1 rest | 26.2 | gamma | |
| Secondary Th2 rest | 44.1 | HUVEC TNF alpha + IL4 | 28.9 |
| Secondary Tr1 rest | 44.8 | HUVEC IL-11 | 18.9 |
| Primary Th1 act | 42.0 | Lung Microvascular EC | 43.8 |
| Primary Th2 act | 62.0 | none | |
| Primary Tr1 act | 47.0 | Lung Microvascular EC | 39.5 |
| Primary Th1 rest | 69.7 | TNFalpha + IL-1beta | |
| Primary Th2 rest | 69.3 | Microvascular Dermal EC | 29.1 |
| Primary Tr1 rest | 69.3 | none | |
| CD45RA CD4 | 43.2 | Microsvasular Dermal EC | 27.0 |
| lymphocyte act | | TNFalpha + IL-1beta | |
| CD45RO CD4 | 64.2 | Bronchial epithelium | 25.5 |
| lymphocyte act | | TNFalpha + IL1beta | |
| CD8 lymphocyte act | 79.0 | Small airway epithelium | 22.1 |
| Secondary CD8 | 64.6 | none | |
| lymphocyte rest | | Small airway epithelium | 19.6 |
| Secondary CD8 | 44.4 | TNFalpha + IL-1beta | |
| lymphocyte act | | Coronery artery SMC rest | 17.9 |
| CD4 lymphocyte none | 21.8 | Coronery artery SMC | 17.9 |
| | | TNFalpha + IL-1beta | |
| 2ry Th1/Th2/Trl_anti- | 41.8 | Astrocytes rest | 11.2 |
| CD95 CH11 | | Astrocytes TNFalpha + IL- | 12.7 |
| LAK cells rest | 40.6 | 1beta | |
| | | KU-812 (Basophil) rest | 49.3 |
| LAK cells IL-2 | 65.5 | KU-812 (Basophil) | 54.7 |
| LAK cells IL-2 + IL-12 | 74.7 | PMA/ionomycin | |
| LAK cells IL-2 + IFN | 90.1 | CCD1106 (Keratinocytes) | 36.6 |
| gamma | | none | |
| LAK cells IL-2 + IL-18 | 83.5 | CCD1106 (Keratinocytes) | 28.7 |
| LAK cells | 5.0 | TNFalpha + IL-1beta | |
| PMA/ionomycin | | Liver cirrhosis | 4.7 |
| NK Cells IL-2 rest | 57.0 | NCI-H292 none | 14.8 |
| Two Way MLR 3 day | 52.5 | NCI-H292 IL-4 | 41.2 |
| Two Way MLR 5 day | 45.1 | NCI-H292 IL-9 | 49.3 |
| | | NCI-H292 IL-13 | 36.6 |
| Two Way MLR 7 day | 33.7 | NCI-H292 IFN gamma | 39.8 |
| PBMC rest | 13.0 | HPAEC none | 21.5 |
| | | HPAEC TNF alpha + IL-1 | 30.4 |
| PBMC PWM | 59.9 beta | | |
| PBMC PHA-L | 57.4 | Lung fibroblast none | 17.1 |
| Ramos (B cell) none | 54.7 | Lung fibroblast TNF | 13.8 |
| Ramos (B cell) | 43.8 | alpha + IL-1 beta | |
| ionomycin | | Lung fibroblast IL-4 | 17.2 |
| B lymphocytes PWM | 59.0 | Lung fibroblast IL-9 | 42.3 |
| | | Lung fibroblast IL-13 | 19.2 |
| B lymphocytes CD40L | 50.7 | Lung fibroblast IFN | 23.0 |
| and IL-4 | | gamma | |
| EOL-1 dbcAMP | 42.3 | Dermal fibroblast | 42.6 |
| | | CCD1070 rest | |
| EOL-1 dbcAMP | 26.6 | Dermal fibroblast | 72.2 |
| PMA/ionomycin | | CCD1070 TNF alpha | |
| Dendritic cells none | 33.2 | Dermal fibroblast | 23.8 |
| Dendritic cells LPS | 35.4 | CCD1070 IL-1 beta | |
| Dendritic cells anti- | 32.3 | Dermal fibroblast IFN | 31.9 |
| CD40 | | gamma | |
| Monocytes rest | 48.0 | Dermal fibroblast IL-4 | 46.0 |
| Monocytes LPS | 23.2 | Dermal fibroblasts rest | 39.0 |
| Macrophages rest | 38.2 | Neutrophils TNFa + LPS | 1.3 |
| Macrophages LPS | 25.5 | Neutrophils rest | 28.5 |
| HUVEC none | 25.2 | Colon | 12.2 |
| HUVEC starved | 23.8 | Lung | 20.9 |
| | | Thymus | 63.7 |
| | | Kidney | 32.1 |

CNS_Neurodegeneration_v1.0 Summary:

Ag3595 This panel confirms the expression of the CG59817-02 gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_Screening_Panel_v1.4 Summary:

Ag3595 Highest expression of the CG59817-02 gene is detected in lung cancer NCI-H522 cell line (CT=26.5). High expressiion of this gene is associated with cluster of cancer cell lines (CNS, colon, gastric, renal, lung, breast, ovarian, prostate, squamous cell carcinoma, and melanoma) used in this panel. Therefore, therapeutic modulation of the activity of this gene or its protein product might be beneficial in the treatment of these cancers.

Among tissues with metabolic or endocrine function, this gene is expressed at high to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

Interestingly, this gene is expressed at much higher levels in fetal (CT=28−30) when compared to adult lung and liver(CT=31−33). This observation suggests that expression of this gene can be used to distinguish fetal from adult lung and liver. In addition, the relative overexpression of this gene in fetal tissue suggests that the protein product may enhance growth or development of these tissues in the fetus and thus may also act in a regenerative capacity in the adult. Therefore, therapeutic modulation of the protein encoded by this gene could be useful in treatment of liver and lung related diseases.

In addition, this gene is expressed at high levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4.1D Summary:

Ag3595 Highest expression of the CG59817-02 gene is detected in activated secondary Th2 cells (CT=29). This gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.5 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Interestingly, expression of this gene is down-regulated in TNF alpha+LPS treated neutrophils as well as PMA/ionomycin treated LAK Cells (CTs=33−35) as compared to the resting cells (CTs=30). Therefore, expression of this gene can be used to distinguish between the resting versus stimulated neutrophils and LAK cells.

G. CG59849-01: DENSIN-180

Expression of gene CG59849-01 was assessed using the primer-probe set Ag3609, described in Table GA. Results of the RTQ-PCR runs are shown in Tables GB, GC and GD.

TABLE GA
Probe Name Ag3609

| | | | Start | SEQ ID |
|---|---|---|---|---|
| Primers | Sequences | Length | Position | No |
| Forward | 5′-acccagagaaattggaagttgt-3′ | 22 | 1011 | 88 |
| Probe | TET-5′-cagtcatgtctctacgctccaacaaa-3′-TAMRA | 26 | 1043 | 89 |
| Reverse | 5′-tgcatctgtccaatctcttca-3′ | 21 | 1083 | 90 |

TABLE GB
CNS_neurodegeneration_v1.0

| | Rel. Exp. (%) Ag3609, | | Rel. Exp. (%) Ag3609, |
|---|---|---|---|
| Tissue Name | Run 210998198 | Tissue Name | Run 210998198 |
| AD 1 Hippo | 10.2 | Control (Path) 3 | 5.5 |
| AD 2 Hippo | 31.4 | Temporal Ctx | |
| AD 3 Hippo | 9.2 | Control (Path) 4 | 39.0 |
| AD 4 Hippo | 9.6 | Temporal Ctx | |
| AD 5 Hippo | 82.9 | AD 1 Occipital Ctx | 16.2 |
| AD 6 Hippo | 56.6 | AD 2 Occipital Ctx | 0.0 |
| Control 2 Hippo | 50.3 | (Missing) | |
| Control 4 Hippo | 5.0 | AD 3 Occipital Ctx | 4.3 |
| Control (Path) 3 | 4.2 | AD 4 Occipital Ctx | 21.3 |
| Hippo | | AD 5 Occipital Ctx | 42.3 |
| AD 1 Temporal Ctx | 17.0 | AD 6 Occipital Ctx | 23.7 |
| AD 2 Temporal Ctx | 33.4 | Control 1 Occipital | 1.7 |
| AD 3 Temporal Ctx | 7.5 | Ctx | |
| AD 4 Temporal Ctx | 24.3 | Control 2 Occipital | 49.7 |
| AD 5 Inf Temporal | 79.6 | Ctx | |
| Ctx | | Control 3 Occipital | 20.0 |
| AD 5 Sup Temporal | 40.9 | Ctx | |
| Ctx | | Control 4 Occipital | 4.6 |
| AD 6 Inf Temporal | 50.0 | Ctx | |
| Ctx | | Control (Path) 1 | 100.0 |
| AD 6 Sup Temporal | 52.9 | Occipital Ctx | |
| Ctx | | Control (Path) 2 | 14.6 |
| Control 1 Temporal | 4.5 | Occipital Ctx | |
| Ctx | | Control (Path) 3 | 1.1 |
| Control 2 Temporal | 35.8 | Occipital Ctx | |
| Ctx | | Control (Path) 4 | 18.9 |
| Control 3 Temporal | 24.1 | Occipital Ctx | |
| Ctx | | Control 1 Parietal | 5.0 |
| Control 3 Temporal | 7.6 | Ctx | |
| Ctx | | Control 2 Parietal | 39.8 |
| Control (Path) 1 | 82.4 | Ctx | |
| Temporal Ctx | | Control 3 Parietal | 13.2 |
| Control (Path) 2 | 50.7 | Ctx | |
| Temporal Ctx | | Control (Path) 1 | 76.3 |
| | | Parietal Ctx | |
| | | Control (Path) 2 | 25.7 |
| | | Parietal Ctx | |
| | | Control (Path) 3 | 3.4 |
| | | Parietal Ctx | |
| | | Control (Path) 4 | 40.9 |
| | | Parietal Ctx | |

TABLE GC
General_screening_panel_v1.4

| | Rel. Exp. (%) Ag3609, | | Rel. Exp. (%) Ag3609, |
|---|---|---|---|
| Tissue Name | Run 217699387 | Tissue Name | Run 217699387 |
| Adipose | 0.1 | Renal ca. TK-10 | 0.0 |
| Melanoma* | 0.0 | Bladder | 0.2 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.0 |
| MEL-5 | | | |
| Squamous cell | 0.0 | Colon ca.* (SW480 | 0.0 |
| carcinoma SCC-4 | | met) SW620 | |
| Testis Pool | 1.1 | Colon ca. HT29 | 0.0 |
| Prostate ca.* (bone | 0.0 | Colon ca. HCT-116 | 0.0 |
| met) PC-3 | | | |
| Prostate Pool | 0.8 | Colon ca. CaCo-2 | 2.5 |
| Placenta | 0.0 | Colon cancer tissue | 0.0 |
| Uterus Pool | 0.1 | Colon ca. SW1116 | 0.0 |
| Ovarian ca. OVCAR- | 0.3 | Colon ca. Colo-205 | 0.0 |
| 3 | | | |
| Ovarian ca. SK-OV- | 0.0 | Colon ca. SW-48 | 0.0 |
| 3 | | | |
| Ovarian ca. OVCAR- | 2.7 | Colon Pool | 0.1 |
| 4 | | | |
| Ovarian ca. OVCAR- | 0.0 | Small Intestine Pool | 0.3 |
| 5 | | | |
| Ovarian ca. IGROV- | 0.1 | Stomach Pool | 1.2 |
| 1 | | | |
| Ovarian ca. OVCAR- | 0.3 | Bone Marrow Pool | 0.2 |
| 8 | | | |
| Ovary | 0.0 | Fetal Heart | 0.4 |
| Breast ca. MCF-7 | 0.0 | Heart Pool | 0.6 |
| Breast ca. MDA- | 0.0 | Lymph Node Pool | 1.2 |
| MB-231 | | | |
| Breast ca. BT 549 | 0.5 | Fetal Skeletal Muscle | 4.9 |
| Breast ca. T47D | 0.0 | Skeletal Muscle Pool | 2.2 |
| Breast ca. MDA-N | 0.0 | Spleen Pool | 0.1 |
| Breast Pool | 0.1 | Thymus Pool | 0.3 |
| Trachea | 0.2 | CNS cancer (glio/astro) | 0.0 |
| | | U87-MG | |
| Lung | 0.1 | CNS cancer (glio/astro) | 0.1 |
| | | U-118-MG | |
| Fetal Lung | 2.1 | CNS cancer (neuro;met) | 6.7 |
| | | SK-N-AS | |
| Lung ca. NCI-N417 | 2.6 | CNS cancer (astro) SF- | 0.1 |
| | | 539 | |
| Lung ca. LX-1 | 0.0 | CNS cancer (astro) | 2.2 |
| | | SNB-75 | |
| Lung ca. NCI-H146 | 0.0 | CNS cancer (glio) SNB- | 0.0 |
| | | 19 | |
| Lung ca. SHP-77 | 0.5 | CNS cancer (glio) SF- | 0.0 |
| | | 295 | |
| Lung ca. A549 | 0.0 | Brain (Amygdala) Pool | 14.2 |
| Lung ca. NCI-H526 | 0.8 | Brain (cerebellum) | 0.3 |
| Lung ca. NCI-H23 | 0.0 | Brain (fetal) | 100.0 |
| Lung ca. NCI-H460 | 0.7 | Brain (Hippocampus) | 22.7 |
| | | Pool | |
| Lung ca. HOP-62 | 2.3 | Cerebral Cortex Pool | 23.7 |
| Lung ca. NCI-H522 | 0.0 | Brain (Substantia nigra) | 13.0 |
| | | Pool | |
| Liver | 0.1 | Brain (Thalamus) Pool | 36.9 |
| Fetal Liver | 1.3 | Brain (whole) | 25.9 |
| Liver ca. HepG2 | 0.0 | Spinal Cord Pool | 2.7 |
| Kidney Pool | 0.7 | Adrenal Gland | 0.3 |
| Fetal Kidney | 6.3 | Pituitary gland Pool | 0.1 |
| Renal ca. 786-0 | 0.0 | Salivary Gland | 0.0 |
| Renal ca. A498 | 0.0 | Thyroid (female) | 0.9 |
| Renal ca. ACHN | 0.0 | Pancreatic ca. CAPAN2 | 0.0 |
| Renal ca. UO-31 | 0.0 | Pancreas Pool | 0.2 |

TABLE GD
Panel 4.1D

| | Rel. Exp. (%) | | Rel. Exp. (%) |
|---|---|---|---|
| | Ag3609, Run | | Ag3609, Run |
| Tissue Name | 169943951 | Tissue Name | 169943951 |
| Secondary Th1 act | 0.0 | HUVEC IL-1beta | 0.0 |
| Secondary Th2 act | 0.0 | HUVEC IFN gamma | 1.2 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 2.2 | Coronery artery SMC | 0.0 |
| lymphocyte act | | TNFalpha + IL-1beta | |
| CD8 lymphocyte act | 0.0 | Astrocytes rest | 100.0 |
| Secondary CD8 | 0.5 | Astrocytes TNFalpha + IL- | 13.3 |
| lymphocyte rest | | 1beta | |
| Secondary CD8 | 0.0 | KU-812 (Basophil) rest | 5.0 |
| lymphocyte act | | | |
| CD4 lymphocyte none | 4.9 | KU-812 (Basophil) | 16.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.3 |
| | | TNFalpha + IL-1beta | |
| LAK cells IL-2 | 2.2 | Liver cirrhosis | 14.9 |
| LAK cells IL-2 + IL-12 | 0.0 | NCI-H292 none | 0.0 |
| LAK cells IL-2 + IFN | 1.1 | NCI-H292 IL-4 | 0.0 |
| gamma | | | |
| LAK cells IL-2 + IL-18 | 0.0 | NCI-H292 IL-9 | 0.0 |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 4.4 | HPAEC none | 0.0 |
| Two Way MLR 5 day | 0.6 | HPAEC TNF alpha + IL-1 | 0.0 |
| | | beta | |
| Two Way MLR 7 day | 0.0 | Lung fibroblast none | 0.0 |
| PBMC rest | 0.9 | Lung fibroblast TNF alpha + | 0.0 |
| | | IL-1 beta | |
| PBMC PWM | 0.0 | Lung fibroblast IL-4 | 1.0 |
| PBMC PHA-L | 0.9 | Lung fibroblast IL-9 | 0.0 |
| Ramos (B cell) none | 0.0 | Lung fibroblast IL-13 | 0.0 |
| Ramos (B cell) | 0.0 | Lung fibroblast IFN | 0.0 |
| ionomycin | | gamma | |
| B lymphocytes PWM | 0.0 | Dermal fibroblast | 0.0 |
| | | CCD1070 rest | |
| B lymphocytes CD40L | 0.0 | Dermal fibroblast | 0.0 |
| and IL-4 | | CCD1070 TNF alpha | |
| EOL-1 dbcAMP | 0.0 | Dermal fibroblast | 0.0 |
| | | CCD1070 IL-1 beta | |
| EOL-1 dbcAMP | 0.0 | Dermal fibroblast IFN | 0.0 |
| PMA/ionomycin | | gamma | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti- | 0.0 | Neutrophils TNFa + LPS | 0.0 |
| CD40 | | | |
| Monocytes rest | 0.0 | Neutrophils rest | 0.0 |
| Monocytes LPS | 0.0 | Colon | 0.2 |
| Macrophages rest | 0.4 | Lung | 0.0 |
| Macrophages LPS | 0.0 | Thymus | 0.0 |
| HUVEC none | 0.0 | Kidney | 20.2 |
| HUVEC starved | 0.0 | | |

CNS_Neurodegeneration_v1.0 Summary:

Ag3609 This panel confirms the expression of the CG59849-01 gene at significant levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_Screening_Panel_v1.4 Summary:

Ag3609 Highest expression of the CG59849-01 gene is detected in fetal brain (CT=26). High expression of this gene is seen exclusivel in in all regions of the central nervous system examined, including amygdala, hippocampus, substantia nigra, thalamus, cerebellum, cerebral cortex, and spinal cord. Therefore, expression of this gene can be used to distinguish between the brain samples from other samples used in this panel. The CG59849-01 gene codes for homolog of rat densin 180 protein, a protein purified from the postsynaptic density fraction of the rat forebrain. Densin 180 is a transmembrane protein that is tightly associated with the postsynaptic density in CNS neurons and involved in specific adhesion between presynaptic and postsynaptic membranes at glutamatergic synapses (Ref. 1, 2). Therefore, therapeutic modulation of densin 180 may be beneficial in the treatment of different neurological disorders such as Alzbeimer's disease, Parkinson's disease, epilepsy, multiple sclerosis, schizophrenia and depression.

Among tissues with metabolic or endocrine function, this gene is expressed at high to low to moderate levels in pancreas, adrenal gland, thyroid, skeletal muscle, heart, liver and the gastrointestinal tract. Therefore, therapeutic modulation of the activity of this gene may prove useful in the treatment of endocrine/metabolically related diseases, such as obesity and diabetes.

Interestingly, this gene is expressed at much higher levels in fetal (CT=32) when compared to adult lung and liver(CT>35). This observation suggests that expression of this gene can be used to distinguish fetal from adult lung and liver. In addition, the relative overexpression of this gene in fetal tissue suggests that the protein product may enhance growth or development of lung and liver in the fetus and thus may also act in a regenerative capacity in the adult. Therefore, therapeutic modulation of the protein encoded by this gene could be useful in treatment of lung and liver related diseases (Apperson M L, Moon I S, Kennedy M B. (1996) Characterization of densin-180, a new brain-specific synaptic protein of the O-sialoglycoprotein family. J Neurosci 16(21):6839-52; Walikonis R S, Oguni A, Khorosheva EM, Jeng C J, Asuncion F J, Kennedy M B. (2001) Densin-180 forms a ternary complex with the (alpha)-subunit of Ca2+/calmodulin-dependent protein kinase II and (alpha)-actinin. J Neurosci 21 (2):423-33).

Panel 4.1D Summary:

Ag3609 Highest expression of the CG59849-01 gene is detected in resting astrocytes (CT=30.4). Interestingly, expression of this gene is down-regulated in TNFalpha+IL-1beta treated astrocytes (CT=33.3). Therefore, expression of this gene can be used to distinguish between the resting and stimulated astrocytes and also to distinguish astrocytes from other samples in the panel. Furthermore, therapeutic modulation of densin 180 encoded by this gene could be important in the treatment of multiple sclerosis or other inflammatory diseases of the CNS.

Moderate expression of this gene is also seen in basophils, liver cirrhosis and kidney. Therefore, therapeutic modulation of this gene product could be beneficial in the treatment of asthma, allergies, hypersensitivity reactions, psoriasis, viral infections, liver cirrhosis and inflammatory or autoimmune diseases that affect the kidney, including lupus and glomerulonephritis.

H. CG59958-01 and CG59958-02: EURL

Expression of gene CG59958-01 and CG59958-02 was assessed using the primer-probe set Ag3638, described in Table HA. Results of the RTQ-PCR runs are shown in Tables HB, and HC. Please note that CG59958-02 represents a full-length physical clone of the CG59958-01 gene, validating the prediction of the gene sequence.

TABLE HA
Probe Name Ag3638

| | | | Start | SEQ ID |
|---|---|---|---|---|
| Primers | Sequences | Length | Position | No |
| Forward | 5′-ccccagcatcatctgtttaa-′ | 20 | 376 | 91 |
| Probe | TET-5′-ttactcccacagtttgactcccaagt-3′-TAMRA | 26 | 421 | 92 |
| Reverse | 5′-tccattttgcagaatattttgg-3′ | 22 | 448 | 93 |

TABLE HB
General_screening_panel_v1.4

| | Rel. Exp. (%) Ag3638, | | Rel. Exp. (%) Ag3638, |
|---|---|---|---|
| Tissue Name | Run 218234120 | Tissue Name | Run 218234120 |
| Adipose | 0.6 | Renal ca. TK-10 | 2.3 |
| Melanoma* | 4.4 | Bladder | 4.6 |
| Hs688(A).T | | | |
| Melanoma* | 3.1 | Gastric ca. (liver met.) | 16.8 |
| Hs688(B).T | | NCI-N87 | |
| Melanoma* M14 | 91.4 | Gastric ca. KATO III | 0.0 |
| Melanoma* | 0.0 | Colon ca. SW-948 | 1.1 |
| LOXIMVI | | | |
| Melanoma* SK- | 100.0 | Colon ca. SW480 | 15.2 |
| MEL-5 | | | |
| Squamous cell | 9.8 | Colon ca.* (SW480 | 6.7 |
| carcinoma SCC-4 | | met) SW620 | |
| Testis Pool | 11.7 | Colon ca. HT29 | 1.1 |
| Prostate ca.* (bone | 10.5 | Colon ca. HCT-116 | 16.4 |
| met) PC-3 | | | |
| Prostate Pool | 1.1 | Colon ca. CaCo-2 | 8.2 |
| Placenta | 2.5 | Colon cancer tissue | 3.6 |
| Uterus Pool | 0.3 | Colon ca. SW1116 | 1.7 |
| Ovarian ca. OVCAR- | 34.9 | Colon ca. Colo-205 | 0.5 |
| 3 | | | |
| Ovarian ca. SK-OV- | 10.6 | Colon ca. SW-48 | 4.6 |
| 3 | | | |
| Ovarian ca. OVCAR- | 4.1 | Colon Pool | 4.0 |
| 4 | | | |
| Ovarian ca. OVCAR- | 0.3 | Small Intestine Pool | 7.5 |
| 5 | | | |
| Ovarian ca. IGROV- | 2.3 | Stomach Pool | 0.2 |
| 1 | | | |
| Ovarian ca. OVCAR- | 5.9 | Bone Marrow Pool | 2.1 |
| 8 | | | |
| Ovary | 2.2 | Fetal Heart | 3.4 |
| Breast ca. MCF-7 | 7.6 | Heart Pool | 1.2 |
| Breast ca. MDA- | 7.4 | Lymph Node Pool | 7.0 |
| MB-231 | | | |
| Breast ca. BT 549 | 0.0 | Fetal Skeletal Muscle | 2.3 |
| Breast ca. T47D | 2.8 | Skeletal Muscle Pool | 2.3 |
| Breast ca. MDA-N | 17.0 | Spleen Pool | 5.1 |
| Breast Pool | 3.3 | Thymus Pool | 7.5 |
| Trachea | 4.1 | CNS cancer (glio/astro) | 0.0 |
| | | U87-MG | |
| Lung | 0.0 | CNS cancer (glio/astro) | 75.8 |
| | | U-118-MG | |
| Fetal Lung | 8.4 | CNS cancer (neuro;met) | 12.7 |
| | | SK-N-AS | |
| Lung ca. NCI-N417 | 5.6 | CNS cancer (astro) SF- | 0.1 |
| | | 539 | |
| Lung ca. LX-1 | 7.7 | CNS cancer (astro) | 41.5 |
| | | SNB-75 | |
| Lung ca. NCI-H146 | 3.1 | CNS cancer (glio) SNB- | 2.2 |
| | | 19 | |
| Lung ca. SHP-77 | 0.0 | CNS cancer (glio) SF- | 8.6 |
| | | 295 | |
| Lung ca. A549 | 6.0 | Brain (Amygdala) Pool | 6.7 |
| Lung ca. NCI-H526 | 1.1 | Brain (cerebellum) | 0.9 |
| Lung ca. NCI-H23 | 9.5 | Brain (fetal) | 2.5 |
| Lung ca. NCI-H460 | 5.3 | Brain (Hippocampus) | 3.5 |
| | | Pool | |
| Lung ca. HOP-62 | 9.8 | Cerebral Cortex Pool | 0.4 |
| Lung ca. NCI-H522 | 0.3 | Brain (Substantia nigra) | 3.1 |
| | | Pool | |
| Liver | 0.2 | Brain (Thalamus) Pool | 2.2 |
| Fetal Liver | 3.1 | Brain (whole) | 6.5 |
| Liver ca. HepG2 | 0.0 | Spinal Cord Pool | 17.7 |
| Kidney Pool | 9.9 | Adrenal Gland | 2.4 |
| Fetal Kidney | 7.5 | Pituitary gland Pool | 1.0 |
| Renal ca. 786-0 | 12.6 | Salivary Gland | 0.7 |
| Renal ca. A498 | 0.0 | Thyroid (female) | 3.5 |
| Renal ca. ACHN | 4.5 | Pancreatic ca. CAPAN2 | 3.4 |
| Renal ca. UO-31 | 0.0 | Pancreas Pool | 8.5 |

TABLE HC
Panel 4.1D

| | Rel. Exp. (%) | | Rel. Exp. (%) |
|---|---|---|---|
| | Ag3638, Run | | Ag3638, Run |
| Tissue Name | 169975057 | Tissue Name | 169975057 |
| Secondary Th1 act | 62.4 | HUVEC IL-1beta | 4.8 |
| Secondary Th2 act | 50.7 | HUVEC IFN gamma | 7.9 |
| Secondary Tr1 act | 48.6 | HUVEC TNF alpha + IFN | 6.4 |
| | | gamma | |
| Secondary Th1 rest | 9.5 | HUVEC TNF alpha + IL4 | 2.7 |
| Secondary Th2 rest | 19.9 | HUVEC IL-11 | 1.7 |
| Secondary Tr1 rest | 13.0 | Lung Microvascular EC | 6.3 |
| | | none | |
| Primary Th1 act | 32.5 | Lung Microvascular EC | 7.5 |
| | | TNFalpha + IL-1beta | |
| Primary Th2 act | 27.0 | Microvascular Dermal EC | 4.5 |
| | | none | |
| Primary Tr1 act | 38.7 | Microsvasular Dermal EC | 4.9 |
| | | TNFalpha + IL-1beta | |
| Primary Th1 rest | 19.6 | Bronchial epithelium | 17.6 |
| | | TNFalpha + IL1beta | |
| Primary Th2 rest | 16.2 | Small airway epithelium | 9.2 |
| | | none | |
| Primary Tr1 rest | 31.2 | Small airway epithelium | 47.6 |
| | | TNFalpha + IL-1beta | |
| CD45RA CD4 | 31.2 | Coronery artery SMC rest | 6.1 |
| lymphocyte act | | | |
| CD45RO CD4 | 66.0 | Coronery artery SMC | 3.6 |
| lymphocyte act | | TNFalpha + IL-1beta | |
| CD8 lymphocyte act | 40.1 | Astrocytes rest | 30.4 |
| Secondary CD8 | 47.3 | Astrocytes TNFalpha + IL- | 21.8 |
| lymphocyte rest | | 1beta | |
| Secondary CD8 | 0.0 | KU-812 (Basophil) rest | 12.8 |
| lymphocyte act | | | |
| CD4 lymphocyte none | 17.2 | KU-812 (Basophil) | 90.1 |
| | | PMA/ionomycin | |
| 2ry Th1/Th2/Tr1_anti- | 15.6 | CCD1106 (Keratinocytes) | 18.9 |
| CD95 CH11 | | none | |
| LAK cells rest | 16.3 | CCD1106 (Keratinocytes) | 27.2 |
| | | TNFalpha + IL-1beta | |
| LAK cells IL-2 | 58.2 | Liver cirrhosis | 2.4 |
| LAK cells IL-2 + IL-12 | 100.0 | NCI-H292 none | 13.9 |
| LAK cells IL-2 + IFN | 84.7 | NCI-H292 IL-4 | 25.2 |
| gamma | | | |
| LAK cells IL-2 + IL-18 | 73.7 | NCI-H292 IL-9 | 31.2 |
| LAK cells | 45.4 | NCI-H292 IL-13 | 20.7 |
| PMA/ionomycin | | | |
| NK Cells IL-2 rest | 38.2 | NCI-H292 IFN gamma | 39.8 |
| Two Way MLR 3 day | 37.4 | HPAEC none | 3.1 |
| Two Way MLR 5 day | 25.0 | HPAEC TNF alpha + IL-1 | 5.6 |
| | | beta | |
| Two Way MLR 7 day | 21.8 | Lung fibroblast none | 5.6 |
| PBMC rest | 11.0 | Lung fibroblast TNF alpha + | 7.2 |
| | | IL-1beta | |
| PBMC PWM | 83.5 | Lung fibroblast IL-4 | 10.2 |
| PBMC PHA-L | 20.4 | Lung fibroblast IL-9 | 13.9 |
| Ramos (B cell) none | 13.7 | Lung fibroblast IL-13 | 10.3 |
| Ramos (B cell) | 15.8 | Lung fibroblast IFN | 20.0 |
| ionomycin | | gamma | |
| B lymphocytes PWM | 20.4 | Dermal fibroblast | 20.3 |
| | | CCD1070 rest | |
| B lymphocytes CD40L | 27.7 | Dermal fibroblast | 10.5 |
| and IL-4 | | CCD1070 TNF alpha | |
| EOL-1 dbcAMP | 43.8 | Dermal fibroblast | 14.3 |
| | | CCD1070 IL-1beta | |
| EOL-1 dbcAMP | 69.7 | Dermal fibroblast IFN | 13.1 |
| PMA/ionomycin | | gamma | |
| Dendritic cells none | 10.6 | Dermal fibroblast IL-4 | 18.2 |
| Dendritic cells LPS | 5.1 | Dermal Fibroblasts rest | 0.0 |
| Dendritic cells anti- | 5.8 | Neutrophils TNFa + LPS | 7.6 |
| CD40 | | | |
| Monocytes rest | 13.8 | Neutrophils rest | 46.0 |
| Monocytes LPS | 22.4 | Colon | 2.6 |
| Macrophages rest | 2.9 | Lung | 20.7 |
| Macrophages LPS | 15.6 | Thymus | 57.8 |
| HUVEC none | 1.4 | Kidney | 10.8 |
| HUVEC starved | 2.0 | | |

CNS_Neurodegeueration_v1.0 Summary:

Ag3638 Results from one experiment with the CG59958-01 gene are not included. The amp plot indicates that there were experimental difficulties with this run.

General_Screening_Panel_v1.4 Summary:

Ag3638 Highest expression of the CG59958-01 gene is seen in melanoma cell lines (CTs=26.8). High levels of expression are also seen in brain cancer cell lines. Thus, expression of this gene could be used to differentiate between these samples and other samples on this panel and as a marker for these cancers. Furthermore, therapeutic modulation of the expression or function of this gene may be effective in the treatment of melanoma and brain cancers.

Among tissues with metabolic function, this gene is expressed at moderate to low levels in pituitary, adipose, adrenal gland, pancreas, thyroid, fetal liver and adult and fetal skeletal muscle, and heart. This widespread expression among these tissues suggests that this gene product may play a role in normal neuroendocrine and metabolic and that disregulated expression of this gene may contribute to neuroendocrine disorders or metabolic diseases, such as obesity and diabetes.

In addition, this gene is expressed at much higher levels in fetal liver tissue (CT=31.6) when compared to expression in the adult counterpart (CT=35.4). Thus, expression of this gene may be used to differentiate between the fetal and adult source of this tissue. In addition, therapeutic modulation of the expression or function of this gene may be useful in the treatment of liver cirrhosis and other diseases that affect the liver.

This gene is also expressed at moderate to low levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 4.1D Summary:

Ag3638 Highest expression of the CG59958-01 gene is seen in IL-2/IL-12 activated LAK cells (CT=27.9). Moderate levels of expression are also seen in a wide variety of samples including a cluster of cytokine activated LAK cells, chronically activated T cells, PBMCs treated with PWM, PMA/ionomycin treated basophils, resting neutrophils and thymus. LAK cells are involved in tumor immunology and cell clearance of virally and bacterial infected cells as well as tumors. The significant expression in a cluster of LAK cells suggests that modulation of the function of the protein encoded by this gene through the application of a small molecule drug or antibody may alter the functions of these cells and lead to improvement of symptoms associated with these conditions. In addition, expression in many samples associated with the immune response also suggests that modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

I. CG59961-01: Zinc Finger Protein 106

Expression of gene CG59961-01 was assessed using the primer-probe sets Ag1070, Ag2252 and Ag914, described in Tables IA, IB and IC. Results of the RTQ-PCR runs are shown in Tables ID, IE and IF.

TABLE IA

Probe Name Ag1070

| | | | Start | SEQ ID |
|---|---|---|---|---|
| Primers | Sequences | Length | Position | No |
| Forward | 5′-taaaatgccatcattgaaatcc-3′ | 22 | 1536 | 94 |
| Probe | TET-5′-tccttccatgtccagccactaaatca-3′-TAMRA | 26 | 1562 | 95 |
| Reverse | 5′-tctttggatcttgcttttgaga-3′ | 22 | 1591 | 96 |

TABLE IB

Probe Name Ag2252

| | | | Start | SEQ ID |
|---|---|---|---|---|
| Primers | Sequences | Length | Position | No |
| Forward | 5′-atgtccagccactaaatcattg-3′ | 22 | 1569 | 97 |
| Probe | TET-5′-tcaaaagcaagatccaaagaatatctca-3′-TAMRA | 28 | 1593 | 98 |
| Reverse | 5′-tgattgggaagagggagagt-3′ | 20 | 1642 | 99 |

TABLE IC

Probe Name Ag914

| | | | Start | SEQ ID |
|---|---|---|---|---|
| Primers | Sequences | Length | Position | No |
| Forward | 5′tgattgggaagagggagagt-3′ | 20 | 4031 | 100 |
| Probe | TET-5′-tgtttctggtatttctttgctccaca-3′-TAMRA | 26 | 3999 | 101 |
| Reverse | 5′-tgagcctagccaagaactga-3′ | 20 | 3972 | 102 |

TABLE ID
Panel 1.3D

| | Rel. Exp. (%) Ag2252, | | Rel. Exp. (%) Ag2252, |
|---|---|---|---|
| Tissue Name | Run 159109785 | Tissue Name | Run 159109785 |
| Liver adenocarcinoma | 8.1 | Kidney (fetal) | 4.9 |
| Pancreas | 0.4 | Renal ca. 786-0 | 2.9 |
| Pancreatic ca. CAPAN | 0.2 | Renal ca. A498 | 3.0 |
| 2 | | | |
| Adrenal gland | 7.9 | Renal ca. RXF 393 | 0.2 |
| Thyroid | 1.2 | Renal ca. ACHN | 0.0 |
| Salivary gland | 7.4 | Renal ca. UO-31 | 0.0 |
| Pituitary gland | 5.5 | Renal ca. TK-10 | 0.0 |
| Brain (fetal) | 7.3 | Liver | 1.4 |
| Brain (whole) | 14.0 | Liver (fetal) | 4.2 |
| Brain (amygdala) | 28.3 | Liver ca. | 0.0 |
| | | (hepatoblast) HepG2 | |
| Brain (cerebellum) | 22.4 | Lung | 13.1 |
| Brain (hippocampus) | 100.0 | Lung (fetal) | 4.7 |
| Brain (substantia nigra) | 2.9 | Lung ca. (small cell) | 6.5 |
| | | LX-1 | |
| Brain (thalamus) | 21.3 | Lung ca. (small cell) | 12.8 |
| | | NCI-H69 | |
| Cerebral Cortex | 80.1 | Lung ca. (s.cell var.) | 6.0 |
| | | SHP-77 | |
| Spinal cord | 1.2 | Lung ca. (large | 0.7 |
| | | cell)NCI-H460 | |
| glio/astro U87-MG | 4.9 | Lung ca. (non-sm. | 0.7 |
| | | cell) A549 | |
| glio/astro U-118-MG | 23.0 | Lung ca. (non-s.cell) | 8.5 |
| | | NCI-H23 | |
| astrocytoma SW1783 | 8.2 | Lung ca. (non-s.cell) | 0.2 |
| | | HOP-62 | |
| neuro*; met SK-N-AS | 49.3 | Lung ca. (non-s.cl) | 0.4 |
| | | NCI-H522 | |
| astrocytoma SF-539 | 11.3 | Lung ca. (squam.) | 3.5 |
| | | SW 900 | |
| astrocytoma SNB-75 | 5.1 | Lung ca. (squam.) | 0.9 |
| | | NCI-H596 | |
| glioma SNB-19 | 4.5 | Mammary gland | 50.0 |
| glioma U251 | 3.7 | Breast ca.* (pl.ef) | 6.2 |
| | | MCF-7 | |
| glioma SF-295 | 0.0 | Breast ca.* (pl.ef) | 27.0 |
| | | MDA-MB-231 | |
| Heart (fetal) | 4.9 | Breast ca.* (pl.ef) | 5.8 |
| | | T47D | |
| Heart | 34.4 | Breast ca. BT-549 | 12.9 |
| Skeletal muscle (fetal) | 16.0 | Breast ca. MDA-N | 26.6 |
| Skeletal muscle | 99.3 | Ovary | 2.1 |
| Bone marrow | 11.8 | Ovarian ca. OVCAR- | 3.0 |
| | | 3 | |
| Thymus | 0.2 | Ovarian ca, OVCAR- | 0.0 |
| | | 4 | |
| Spleen | 2.5 | Ovarian ca. OVCAR- | 0.0 |
| | | 5 | |
| Lymph node | 1.8 | Ovarian ca. OVCAR- | 1.2 |
| | | 8 | |
| Colorectal | 2.5 | Ovarian ca. IGROV-1 | 0.0 |
| Stomach | 1.1 | Ovarian ca.* (ascites) | 1.7 |
| | | SK-OV-3 | |
| Small intestine | 3.1 | Uterus | 2.0 |
| Colon ca. SW480 | 0.0 | Placenta | 5.1 |
| Colon ca.* | 0.5 | Prostate | 2.0 |
| SW620(SW480 met) | | | |
| Colon ca. HT29 | 0.3 | Prostate ca.* (bone | 2.1 |
| | | met)PC-3 | |
| Colon ca. HCT-116 | 1.7 | Testis | 0.8 |
| Colon ca. CaCo-2 | 6.9 | Melanoma | 6.9 |
| | | Hs688(A).T | |
| Colon ca. | 3.8 | Melanoma* (met) | 2.5 |
| tissue(ODO3866) | | Hs688(B).T | |
| Colon ca. HCC-2998 | 10.9 | Melanoma UACC-62 | 14.0 |
| Gastric ca.* (liver met) | 11.7 | Melanoma M14 | 18.0 |
| NCI-N87 | | | |
| Bladder | 8.1 | Melanoma LOX | 6.3 |
| | | IMVI | |
| Trachea | 3.6 | Melanoma* (met) | 22.7 |
| | | SK-MEL-5 | |
| Kidney | 1.5 | Adipose | 19.5 |

TABLE IE
Panel 2D

| | Rel. Exp. (%) | | Rel. Exp. (%) |
|---|---|---|---|
| | Ag2252, Run | | Ag2252, Run |
| Tissue Name | 159109181 | Tissue Name | 159109181 |
| Normal Colon | 86.5 | Kidney Margin | 0.6 |
| | | 8120608 | |
| CC Well to Mod Diff | 9.3 | Kidney Cancer | 0.5 |
| (ODO3866) | | 8120613 | |
| CC Margin (ODO3866) | 13.3 | Kidney Margin | 0.0 |
| | | 8120614 | |
| CC Gr.2 rectosigmoid | 6.5 | Kidney Cancer | 0.0 |
| (ODO3868) | | 9010320 | |
| CC Margin (ODO3868) | 9.5 | Kidney Margin | 0.6 |
| | | 9010321 | |
| CC Mod Diff (ODO3920) | 22.5 | Normal Uterus | 7.1 |
| CC Margin (ODO3920) | 19.2 | Uterus Cancer 064011 | 10.2 |
| CC Gr.2 ascend colon | 38.7 | Normal Thyroid | 7.1 |
| (ODO3921) | | | |
| CC Margin (ODO3921) | 17.2 | Thyroid Cancer | 4.0 |
| | | 064010 | |
| CC from Partial | 18.0 | Thyroid Cancer | 7.3 |
| Hepatectomy (ODO4309) | | A302152 | |
| Mets | | | |
| Liver Margin (ODO4309) | 10.4 | Thyroid Margin | 0.0 |
| | | A302153 | |
| Colon mets to lung | 1.9 | Normal Breast | 4.5 |
| (OD04451-01) | | | |
| Lung Margin (OD04451- | 8.4 | Breast Cancer | 2.3 |
| 02) | | (OD04566) | |
| Normal Prostate 6546-1 | 3.8 | Breast Cancer | 4.9 |
| | | (OD04590-01) | |
| Prostate Cancer | 45.7 | Breast Cancer Mets | 12.6 |
| (OD04410) | | (OD04590-03) | |
| Prostate Margin | 35.6 | Breast Cancer | 12.8 |
| (OD04410) | | Metastasis (OD04655- | |
| | | 05) | |
| Prostate Cancer | 26.1 | Breast Cancer 064006 | 8.5 |
| (OD04720-01) | | | |
| Prostate Margin | 38.2 | Breast Cancer 1024 | 0.5 |
| (OD04720-02) | | | |
| Normal Lung 061010 | 39.2 | Breast Cancer | 8.7 |
| | | 9100266 | |
| Lung Met to Muscle | 9.1 | Breast Margin | 4.1 |
| (ODO4286) | | 9100265 | |
| Muscle Margin | 28.5 | Breast Cancer | 11.7 |
| (ODO4286) | | A209073 | |
| Lung Malignant Cancer | 8.2 | Breast Margin | 14.4 |
| (OD03126) | | A209073 | |
| Lung Margin (OD03126) | 9.2 | Normal Liver | 4.5 |
| Lung Cancer (OD04404) | 1.7 | Liver Cancer 064003 | 8.9 |
| Lung Margin (OD04404) | 6.8 | Liver Cancer 1025 | 1.1 |
| Lung Cancer (OD04565) | 3.0 | Liver Cancer 1026 | 0.4 |
| Lung Margin (OD04565) | 6.9 | Liver Cancer 6004-T | 0.7 |
| Lung Cancer (OD04237- | 15.7 | Liver Tissue 6004-N | 2.2 |
| 01) | | | |
| Lung Margin (OD04237- | 14.8 | Liver Cancer 6005-T | 0.8 |
| 02) | | | |
| Ocular Mel Met to Liver | 100.0 | Liver Tissue 6005-N | 0.9 |
| (ODO4310) | | | |
| Liver Margin (ODO4310) | 4.8 | Normal Bladder | 24.5 |
| Melanoma Mets to Lung | 20.2 | Bladder Cancer 1023 | 3.1 |
| (OD04321) | | | |
| Lung Margin (OD04321) | 17.6 | Bladder Cancer | 16.8 |
| | | A302173 | |
| Normal Kidney | 13.1 | Bladder Cancer | 13.0 |
| | | (OD04718-01) | |
| Kidney Ca, Nuclear grade | 2.8 | Bladder Normal | 22.4 |
| 2 (OD04338) | | Adjacent (OD04718- | |
| | | 03) | |
| Kidney Margin | 3.9 | Normal Ovary | 2.1 |
| (OD04338) | | | |
| Kidney Ca Nuclear grade | 4.0 | Ovarian Cancer | 13.2 |
| 1/2 (OD04339) | | 064008 | |
| Kidney Margin | 5.5 | Ovarian Cancer | 17.6 |
| (OD04339) | | (OD04768-07) | |
| Kidney Ca, Clear cell type | 7.7 | Ovary Margin | 8.0 |
| (OD04340) | | (OD04768-08) | |
| Kidney Margin | 9.6 | Normal Stomach | 19.9 |
| (OD04340) | | | |
| Kidney Ca, Nuclear grade | 3.6 | Gastric Cancer | 6.4 |
| 3 (OD04348) | | 9060358 | |
| Kidney Margin | 12.5 | Stomach Margin | 14.0 |
| (OD04348) | | 9060359 | |
| Kidney Cancer | 4.5 | Gastric Cancer | 30.4 |
| (OD04622-01) | | 9060395 | |
| Kidney Margin | 0.4 | Stomach Margin | 23.7 |
| (OD04622-03) | | 9060394 | |
| Kidney Cancer | 4.6 | Gastric Cancer | 20.7 |
| (OD04450-01) | | 9060397 | |
| Kidney Margin | 4.4 | Stomach Margin | 2.1 |
| (OD04450-03) | | 9060396 | |
| Kidney Cancer 8120607 | 0.7 | Gastric Cancer 064005 | 71.2 |

TABLE IF
Panel 4D

| | Rel. Exp. (%) | | Rel. Exp. (%) |
|---|---|---|---|
| | Ag2252, Run | | Ag2252, Run |
| Tissue Name | 159112027 | Tissue Name | 159112027 |
| Secondary Th1 act | 79.6 | HUVEC IL-1beta | 5.0 |
| Secondary Th2 act | 73.7 | HUVEC IFN gamma | 7.4 |
| Secondary Tr1 act | 84.1 | HUVEC TNF alpha + IFN | 8.3 |
| | | gamma | |
| Secondary Th1 rest | 27.2 | HUVEC TNF alpha + IL4 | 29.7 |
| Secondary Th2 rest | 20.9 | HUVEC IL-11 | 10.5 |
| Secondary Tr1 rest | 27.7 | Lung Microvascular EC | 21.2 |
| | | none | |
| Primary Th1 act | 77.4 | Lung Microvascular EC | 22.5 |
| | | TNFalpha + IL-1beta | |
| Primary Th2 act | 77.9 | Microvascular Dermal EC | 28.7 |
| | | none | |
| Primary Tr1 act | 80.1 | Microsvasular Dermal EC | 21.0 |
| | | TNFalpha + IL-1beta | |
| Primary Th1 rest | 96.6 | Bronchial epithelium | 29.5 |
| | | TNFalpha + IL1beta | |
| Primary Th2 rest | 56.6 | Small airway epithelium | 8.7 |
| | | none | |
| Primary Tr1 rest | 23.7 | Small airway epithelium | 35.6 |
| | | TNFalpha + IL-1beta | |
| CD45RA CD4 | 29.3 | Coronery artery SMC rest | 9.9 |
| lymphocyte act | | | |
| CD45RO CD4 | 66.4 | Coronery artery SMC | 7.0 |
| lymphocyte act | | TNFalpha + IL-1beta | |
| CD8 lymphocyte act | 22.1 | Astrocytes rest | 11.5 |
| Secondary CD8 | 37.4 | Astrocytes TNFalpha + IL- | 13.3 |
| lymphocyte rest | | 1beta | |
| Secondary CD8 | 27.5 | KU-812 (Basophil) rest | 18.0 |
| lymphocyte act | | | |
| CD4 lymphocyte none | 11.0 | KU-812 (Basophil) | 49.0 |
| | | PMA/ionomycin | |
| 2ry Th1/Th2/Tr1_anti- | 24.1 | CCD1106 (Keratinocytes) | 17.8 |
| CD95 CH11 | | none | |
| LAK cells rest | 48.3 | CCD1106 (Keratinocytes) | 12.2 |
| | | TNFalpha + IL-1beta | |
| LAK cells IL-2 | 31.0 | Liver cirrhosis | 3.6 |
| LAK cells IL-2 + IL-12 | 21.0 | Lupus kidney | 4.8 |
| LAK cells IL-2 + IFN | 36.6 | NCI-H292 none | 59.5 |
| gamma | | | |
| LAK cells IL-2 + IL-18 | 23.5 | NCI-H292 IL-4 | 39.2 |
| LAK cells | 10.4 | NCI-H292 IL-9 | 37.1 |
| PMA/ionomycin | | | |
| NK Cells IL-2 rest | 18.2 | NCI-H292 IL-13 | 8.1 |
| Two Way MLR 3 day | 30.1 | NCI-H292 IFN gamma | 22.1 |
| Two Way MLR 5 day | 22.8 | HPAEC none | 23.0 |
| Two Way MLR 7 day | 15.2 | HPAEC TNF alpha + IL-1 | 27.7 |
| | | beta | |
| PBMC rest | 17.4 | Lung fibroblast none | 45.4 |
| PBMC PWM | 100.0 | Lung fibroblast TNF alpha + | 15.6 |
| | | IL-1 beta | |
| PBMC PHA-L | 37.9 | Lung fibroblast IL-4 | 81.8 |
| Ramos (B cell) none | 26.1 | Lung fibroblast IL-9 | 64.6 |
| Ramos (B cell) | 75.3 | Lung fibroblast IL-13 | 45.7 |
| ionomycin | | | |
| B lymphocytes PWM | 67.8 | Lung fibroblast IFN | 85.3 |
| | | gamma | |
| B lymphocytes CD40L | 10.4 | Dermal fibroblast | 40.9 |
| and IL-4 | | CCD1070 rest | |
| EOL-1 dbcAMP | 17.3 | Dermal fibroblast | 87.7 |
| | | CCD1070 TNF alpha | |
| EOL-1 dbcAMP | 20.6 | Dermal fibroblast | 15.1 |
| PMA/ionomycin | | CCD1070 IL-1 beta | |
| Dendritic cells none | 27.5 | Dermal fibroblast IFN | 17.8 |
| | | gamma | |
| Dendritic cells LPS | 23.5 | Dermal fibroblast IL-4 | 44.4 |
| Dendritic cells anti- | 54.0 | IBD Colitis 2 | 7.0 |
| CD40 | | | |
| Monocytes rest | 68.3 | IBD Crohn's | 14.5 |
| Monocytes LPS | 15.1 | Colon | 59.5 |
| Macrophages rest | 46.7 | Lung | 53.2 |
| Macrophages LPS | 29.5 | Thymus | 90.1 |
| HUVEC none | 19.6 | Kidney | 69.7 |
| HUVEC starved | 25.3 | | |

CNS_Neurodegeneration_v1.0 Summary:

Ag2252 Expression of the CG59961-01 gene is low/undetectable (Ct values>35) in all samples in Panel CNS_neurodegeneration_v1.0.

Panel 1.3D Summary:

Ag2252 The CG59961-01 gene encodes a homolog of Zfp 106 and is expressed at moderate levels in the brain. Highest expression is seen in the hippocampus (CT=31) and cerebral cortex, regions that show marked neurodegeneration in Alzheimer's disease. In addition, the gene product shows homology to a 600 amino acid sequence implicated in the insulin receptor-signalling pathway. This insulin receptor has also been implicated in the pathogenesis of Alzheimer's disease, possibly through glucose metabolism by neurons. This fact, coupled with the localization of the expression of this gene to the hippocampus and cortex, make the protein product an excellent drug target for the treatment of Alzheimer's disease. Thus, therapeutic upregulation of this gene or its protein product may be beneficial in slowing the neurodegeneration associated with Alzheimer's.

Among tissues with metabolic function, this gene is expressed at low but significant levels in adipose, the adrenal gland, adult heart, and adult and fetal skeletal muscle. Since this gene is expressed at higher levels in tissue from adult heart (CT=32.5) and skeletal muscle (CT=31) than in fetal heart (CT=35.3) and skeletal muscle (CT=33.6), expression of the gene could potentially be used to differentiate between the sources of heat and skeletal muscle tissue.

This gene is also expressed in cell lines derived from breast, brain cancer and melanoma. Moreover, therapeutic modulation of the expression of this gene or this gene product, through the use of small molecule drugs, antibodies or protein therapeutics could be of use in the treatment of brain cancer, breast cancer or melanoma (Zuberi A R, Christianson G J, Mendoza L M, Shastri N, Roopenian D C. (1998) Positional cloning and molecular characterization of an immunodominant cytotoxic determinant of the mouse H3 minor histocompatibility complex. Immunity. 9:687-98; Frolich L, Blum-Degen D, Riederer P, Hoyer S. (1999) A disturbance in the neuronal insulin receptor signal transduction in sporadic Alzheimer's disease. Ann N Y Acad. Sci. 893:290-3; Frolich L, Blum-Degen D, Bernstein H G, Engelsberger S, Humrich J, Laufer S, Muschner D, Thalheimer A, Turk A, Hoyer S, Zochling R, Boissl K W, Jellinger K, Riederer P. (1998) Brain insulin and insulin receptors in aging and sporadic Alzheimer's disease. J Neural Transm. 105(4-5):423-38).

Panel 2D Summary:

Ag2252 Highest expression of the CG59961-01 gene in this panel is seen in a metastatic ocular melanoma (CT=30.9). Significant expression is also seen in gastric cancer. Thus, the expression of this gene could be used to distinguish between the ocular melanoma metastasis and the gastric cancer samples and the other samples in the panel. Moreover, therapeutic modulation of the expression of this gene or this gene product, through the use of small molecule drugs, antibodies or protein therapeutics could be of use in the treatment of ocular melanoma or gastric cancer.

Panel 4D Summary:

Ag2252 The CG59961-01 gene is expressed ubiquitously in this panel, with highest expression in PWM treated mononuclear cells (CT=31). This gene encodes a ZFP106 like molecule with potential involvement in a signaling pathway based on its homology to ZFP106 (Ref. 1). It may be important in insulin receptor signaling pathway and in minor histocompatability antigen signaling. Therefore, treatments designed with the protein encoded for by the CG59961-01 gene may be effective both in the enhancement of immunosurveillance mechanisms and in the treatment of graft versus host disease.

J. CG88655-01: Novel Protein

Expression of gene CG88655-01 was assessed using the primer-probe set Ag3651, described in Table JA. Results of the RTQ-PCR runs are shown in Tables JB, JC and JD.

TABLE JA
Probe Name Ag3651

| | | | Start | SEQ ID |
|---|---|---|---|---|
| Primers | Sequences | Length | Position | No |
| Forward | 5′-taatcttgctgccaatgatctc-3′ | 23 | 614 | 103 |
| Probe | TET-5′-ccgtcccgaatagccagactacagaa-3′-TAMRA | 26 | 639 | 104 |
| Reverse | 5′-gatttccatccctgatctcttc-3′ | 22 | 687 | 105 |

TABLE JB
CNS_neurodegeneration_v1.0

| | Rel. Exp. (%) Ag3651, | | Rel. Exp. (%) Ag3651, |
|---|---|---|---|
| Tissue Name | Run 211019101 | Tissue Name | Run 211019101 |
| AD 1 Hippo | 11.8 | Control (Path) 3 | 3.8 |
| | | Temporal Ctx | |
| AD 2 Hippo | 12.4 | Control (Path) 4 | 31.9 |
| | | Temporal Ctx | |
| AD 3 Hippo | 5.9 | AD 1 Occipital Ctx | 14.8 |
| AD 4 Hippo | 4.8 | AD 2 Occipital Ctx | 0.0 |
| | | (Missing) | |
| AD 5 hippo | 75.8 | AD 3 Occipital Ctx | 4.2 |
| AD 6 Hippo | 62.4 | AD 4 Occipital Ctx | 15.4 |
| Control 2 Hippo | 25.5 | AD 5 Occipital Ctx | 20.3 |
| Control 4 Hippo | 10.7 | AD 6 Occipital Ctx | 54.7 |
| Control (Path) 3 | 9.7 | Control 1 Occipital | 3.1 |
| Hippo | | Ctx | |
| AD 1 Temporal Ctx | 15.8 | Control 2 Occipital | 51.8 |
| | | Ctx | |
| AD 2 Temporal Ctx | 24.3 | Control 3 Occipital | 8.6 |
| | | Ctx | |
| AD 3 Temporal Ctx | 4.6 | Control 4 Occipital | 6.1 |
| | | Ctx | |
| AD 4 Temporal Ctx | 17.3 | Control (Path) 1 | 74.2 |
| | | Occipital Ctx | |
| AD 5 Inf Temporal | 100.0 | Control (Path) 2 | 9.3 |
| Ctx | | Occipital Ctx | |
| AD 5 SupTemporal | 18.4 | Control (Path) 3 | 2.5 |
| Ctx | | Occipital Ctx | |
| AD 6 Inf Temporal | 64.6 | Control (Path) 4 | 18.4 |
| Ctx | | Occipital Ctx | |
| AD 6 Sup Temporal | 62.0 | Control 1 Parietal | 5.5 |
| Ctx | | Ctx | |
| Control 1 Temporal | 5.5 | Control 2 Parietal | 28.7 |
| Ctx | | Ctx | |
| Control 2 Temporal | 36.1 | Control 3 Parietal | 11.6 |
| Ctx | | Ctx | |
| Control 3 Temporal | 9.2 | Control (Path) 1 | 62.4 |
| Ctx | | Parietal Ctx | |
| Control 4 Temporal | 5.4 | Control (Path) 2 | 24.5 |
| Ctx | | Parietal Ctx | |
| Control (Path) 1 | 48.3 | Control (Path) 3 | 2.3 |
| Temporal Ctx | | Parietal Ctx | |
| Control (Path) 2 | 21.3 | Control (Path) 4 | 7.5 |
| Temporal Ctx | | Parietal Ctx | |

TABLE JC
General_screening_panel_v1.4

| | Rel. Exp. (%) Ag3651, | | Rel. Exp. (%) Ag3651, |
|---|---|---|---|
| Tissue Name | Run 218952683 | Tissue Name | Run 218952683 |
| Adipose | 7.2 | Renal ca. TK-10 | 77.4 |
| Melanoma* | 19.6 | Bladder | 13.8 |
| Hs688(A).T | | | |
| Melanoma* | 21.5 | Gastric ca. (liver met.) | 79.0 |
| Hs688(B).T | | NCI-N87 | |
| Melanoma* M14 | 70.7 | Gastric ca. KATO III | 55.1 |
| Melanoma* | 34.4 | Colon ca. SW-948 | 14.9 |
| LOXIMVI | | | |
| Melanoma* SK- | 27.4 | Colon ca. SW480 | 37.1 |
| MEL-5 | | | |
| Squamous cell | 16.3 | Colon ca.* (SW480 | 47.3 |
| carcinoma SCC-4 | | met) SW620 | |
| Testis Pool | 36.1 | Colon ca. HT29 | 11.2 |
| Prostate ca.* (bone | 35.1 | Colon ca. HCT-116 | 64.6 |
| met) PC-3 | | | |
| Prostate Pool | 7.1 | Colon ca. CaCo-2 | 22.7 |
| Placenta | 6.1 | Colon cancer tissue | 10.5 |
| Uterus Pool | 4.8 | Colon ca. SW1116 | 10.2 |
| Ovarian ca. OVCAR- | 28.1 | Colon ca. Colo-205 | 14.4 |
| 3 | | | |
| Ovarian ca. SK-OV- | 85.9 | Colon ca. SW-48 | 11.1 |
| 3 | | | |
| Ovarian ca. OVCAR- | 18.0 | Colon Pool | 13.3 |
| 4 | | | |
| Ovarian ca. OVCAR- | 39.5 | Small Intestine Pool | 13.2 |
| 5 | | | |
| Ovarian ca. IGROV- | 37.9 | Stomach Pool | 6.8 |
| 1 | | | |
| Ovarian ca. OVCAR- | 20.2 | Bone Marrow Pool | 6.6 |
| 8 | | | |
| Ovary | 8.8 | Fetal Heart | 6.6 |
| Breast ca. MCF-7 | 37.1 | Heart Pool | 4.3 |
| Breast ca. MDA- | 24.3 | Lymph Node Pool | 12.9 |
| MB-231 | | | |
| Breast ca. BT 549 | 100.0 | Fetal Skeletal Muscle | 6.2 |
| Breast ca. T47D | 86.5 | Skeletal Muscle Pool | 11.1 |
| Breast ca. MDA-N | 24.8 | Spleen Pool | 9.5 |
| Breast Pool | 12.2 | Thymus Pool | 16.7 |
| Trachea | 14.5 | CNS cancer (glio/astro) | 27.0 |
| | | U87-MG | |
| Lung | 3.5 | CNS cancer (glio/astro) | 44.1 |
| | | U-118-MG | |
| Fetal Lung | 27.7 | CNS cancer (neuro;met) | 28.3 |
| | | SK-N-AS | |
| Lung ca. NCI-N417 | 8.8 | CNS cancer (astro) SF- | 20.0 |
| | | 539 | |
| Lung ca. LX-1 | 52.5 | CNS cancer (astro) | 64.6 |
| | | SNB-75 | |
| Lung ca. NCI-H146 | 3.3 | CNS cancer (glio) SNB- | 44.8 |
| | | 19 | |
| Lung ca. SHP-77 | 28.7 | CNS cancer (glio) SF- | 54.7 |
| | | 295 | |
| Lung ca. A549 | 21.6 | Brain (Amygdala) Pool | 6.9 |
| Lung ca. NCI-H526 | 8.4 | Brain (cerebellum) | 16.4 |
| Lung ca. NCI-H23 | 42.6 | Brain (fetal) | 12.9 |
| Lung ca. NCI-H460 | 31.2 | Brain (Hippocampus) | 6.1 |
| | | Pool | |
| Lung ca. HOP-62 | 11.7 | Cerebral Cortex Pool | 9.6 |
| Lung ca. NCI-H522 | 29.9 | Brain (Substantia nigra) | 7.7 |
| | | Pool | |
| Liver | 1.5 | Brain (Thalamus) Pool | 12.7 |
| Fetal Liver | 11.9 | Brain (whole) | 13.5 |
| Liver ca. HepG2 | 18.9 | Spinal Cord Pool | 6.9 |
| Kidney Pool | 16.7 | Adrenal Gland | 29.7 |
| Fetal Kidney | 20.2 | Pituitary gland Pool | 4.2 |
| Renal ca. 786-0 | 30.1 | Salivary Gland | 6.9 |
| Renal ca. A498 | 10.4 | Thyroid (female) | 6.5 |
| Renal ca. ACHN | 27.4 | Pancreatic ca. CAPAN2 | 14.5 |
| Renal ca. UO-31 | 24.3 | Pancreas Pool | 17.1 |

TABLE JD
Panel 4.1D

| | Rel. Exp. (%) | | Rel. Exp. (%) |
|---|---|---|---|
| | Ag3651, Run | | Ag3651, Run |
| Tissue Name | 169975803 | Tissue Name | 169975803 |
| Secondary Th1 act | 55.1 | HUVEC IL-1beta | 34.2 |
| Secondary Th2 act | 97.9 | HUVEC IFN gamma | 24.3 |
| Secondary Tr1 act | 83.5 | HUVEC TNF alpha + IFN | 32.3 |
| | | gamma | |
| Secondary Th1 rest | 15.2 | HUVEC TNF alpha + IL4 | 36.9 |
| Secondary Th2 rest | 31.2 | HUVEC IL-11 | 7.1 |
| Secondary Tr1 rest | 14.7 | Lung Microvascular EC | 45.1 |
| | | none | |
| Primary Th1 act | 85.3 | Lung Microvascular EC | 52.1 |
| | | TNFalpha + IL-1beta | |
| Primary Th2 act | 90.1 | Microvascular Dermal EC | 15.1 |
| | | none | |
| Primary Tr1 act | 74.2 | Microvasular Dermal EC | 21.9 |
| | | TNFalpha + IL-1beta | |
| Primary Th1 rest | 25.0 | Bronchial epithelium | 24.0 |
| | | TNFalpha + IL1beta | |
| Primary Th2 rest | 18.9 | Small airway epithelium | 13.2 |
| | | none | |
| Primary Tr1 rest | 37.6 | Small airway epithelium | 22.1 |
| | | TNFalpha + IL-1beta | |
| CD45RA CD4 | 58.6 | Coronery artery SMC rest | 11.1 |
| lymphocyte act | | | |
| CD45RO CD4 | 83.5 | Coronery artery SMC | 12.3 |
| lymphocyte act | | TNFalpha + IL-1beta | |
| CD8 lymphocyte act | 79.6 | Astrocytes rest | 17.1 |
| Secondary CD8 | 66.4 | Astrocytes TNFalpha + IL- | 11.8 |
| lymphocyte rest | | 1beta | |
| Secondary CD8 | 39.5 | KU-812 (Basophil) rest | 62.4 |
| lymphocyte act | | | |
| CD4 lymphocyte none | 9.5 | KU-812 (Basophil) | 84.1 |
| | | PMA/ionomycin | |
| 2ry Th1/Th2/Tr1_anti- | 18.8 | CCD1106 (Keratinocytes) | 30.1 |
| CD95 CH11 | | none | |
| LAK cells rest | 27.0 | CCD1106 (Keratinocytes) | 23.2 |
| | | TNFalpha + IL-1beta | |
| LAK cells IL-2 | 41.5 | Liver cirrhosis | 3.4 |
| LAK cells IL-2 + IL-12 | 47.6 | NCI-H292 none | 24.3 |
| LAK cells IL-2 + IFN | 76.3 | NCI-H292 IL-4 | 32.8 |
| gamma | | | |
| LAK cells IL-2 + IL-18 | 66.0 | NCI-H292 IL-9 | 57.4 |
| LAK cells | 46.3 | NCI-H292 IL-13 | 38.7 |
| PMA/ionomycin | | | |
| NK Cells IL-2 rest | 37.1 | NCI-H292 IFN gamma | 56.6 |
| Two Way MLR 3 day | 42.3 | HPAEC none | 23.3 |
| Two Way MLR 5 day | 35.4 | HPAEC TNF alpha + IL-1 | 44.1 |
| | | beta | |
| Two Way MLR 7 day | 23.2 | Lung fibroblast none | 20.0 |
| PBMC rest | 9.6 | Lung fibroblast TNF alpha + | 16.2 |
| | | IL-1 beta | |
| PBMC PWM | 78.5 | Lung fibroblast IL-4 | 24.7 |
| PBMC PHA-L | 37.6 | Lung fibroblast IL-9 | 28.7 |
| Ramos (B cell) none | 76.3 | Lung fibroblast IL-13 | 20.4 |
| Ramos (B cell) | 100.0 | Lung fibroblast IFN | 34.2 |
| ionomycin | | gamma | |
| B lymphocytes PWM | 52.1 | Dermal fibroblast | 36.9 |
| | | CCD1070 rest | |
| B lymphocytes CD40L | 88.9 | Dermal fibroblast | 50.0 |
| and IL-4 | | CCD1070 TNF alpha | |
| EOL-1 dbcAMP | 47.3 | Dermal fibroblast | 25.3 |
| | | CCD1070 IL-1 beta | |
| EOL-1 dbcAMP | 39.5 | Dermal fibroblast IFN | 12.3 |
| PMA/ionomycin | | gamma | |
| Dendritic cells none | 25.3 | Dermal fibroblast IL-4 | 37.9 |
| Dendritic cells LPS | 18.0 | Dermal Fibroblasts rest | 13.4 |
| Dendritic cells anti- | 27.2 | Neutrophils TNFa + LPS | 7.6 |
| CD40 | | | |
| Monocytes rest | 29.9 | Neutrophils rest | 11.6 |
| Monocytes LPS | 34.4 | Colon | 7.1 |
| Macrophages rest | 25.3 | Lung | 23.5 |
| Macrophages LPS | 13.2 | Thymus | 22.5 |
| HUVEC none | 12.5 | Kidney | 24.0 |
| HUVEC starved | 28.9 | | |

CNS_Neurodegeneration_v1.0 Summary:

Ag3651 This panel does not show differential expression of the CG88655-01 gene in Alzheimer's disease. However, this expression profile confirms the presence of this gene in the brain. Please see Panel 1.4 for discussion of utility of this gene in the central nervous system.

General_Screening_Panel_v1.4 Summary:

Ag3651 The CG88655-01 gene is widely expressed in this panel, with expression higher in the cancer cell lines than in the normal tissue samples. Highest expression is seen in a breast cancer cell line (CT=29). Moderate levels of expression are seen in samples derived from melanoma, ovarian, breast, lung, gastric, colon, renal and brain cancer cell lines. Thus, expression of this gene could be used as a marker for cancer and modulation of its activity may be useful in the treatment of these cancers.

Among tissues with metabolic function, this gene is expressed at moderate to low levels in pituitary, adipose, adrenal gland, pancreas, thyroid, fetal liver and adult and fetal skeletal muscle, and heart. This widespread expression among these tissues suggests that this gene product may play a role in normal neuroendocrine and metabolic and that disregulated expression of this gene may contribute to neuroendocrine disorders or metabolic diseases, such as obesity and diabetes.

This gene is also expressed at moderate to low levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 4.1D Summary:

Ag3651 The CG88655-01 gene is ubiquitously expressed in this panel, with highest expression in the ionomycin treated B cell line Ramos. (CT=31). Expression in activated T cells appears to be slightly upregulated when compared to expression in resting T cells. In addition, this gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

K. CG88665-01: Novel Protein

Expression of gene CG88665-01 was assessed using the primer-probe set Ag3652, described in Table KA. Results of the RTQ-PCR runs are shown in Tables KB, KC and KD.

TABLE KA
Probe Name Ag3652

| | | | Start | SEQ ID |
|---|---|---|---|---|
| Primers | Sequences | Length | Position | No |
| Forward | 5′-gatcctggcacagggaaat-3′ | 19 | 1077 | 106 |
| Probe | TET-5′-tcagttcctcaaatatgcagcaaaga-3′-TAMRA | 26 | 1097 | 107 |
| Reverse | 5′-ttcctgtggtcagcacagat-3′ | 20 | 1133 | 108 |

TABLE KB
CNS_neurodegeneration_v1.0

| | Rel. Exp. | | Rel. Exp. |
|---|---|---|---|
| | (%) | | (%) |
| | Ag3652, | | Ag3652, |
| | Run | | Run |
| Tissue Name | 224079117 | Tissue Name | 224079117 |
| AD 1 Hippo | 0.6 | Control (Path) 3 | 0.3 |
| AD 2 Hippo | 0.6 | Temporal Ctx | |
| AD 3 Hippo | 14.1 | Control (Path) 4 | 0.8 |
| AD 4 Hippo | 0.9 | Temporal Ctx | |
| AD 5 Hippo | 2.5 | AD 1 Occipital Ctx | 100.0 |
| AD 6 Hippo | 3.5 | AD 2 Occipital Ctx | 0.0 |
| Control 2 Hippo | 0.9 | (Missing) | |
| Control 4 Hippo | 0.5 | AD 3 Occipital Ctx | 3.2 |
| Control (Path) 3 | 0.5 | AD 4 Occipital Ctx | 0.3 |
| Hippo | | AD 5 Occipital Ctx | 1.7 |
| AD 1 Temporal Ctx | 7.8 | AD 6 Occipital Ctx | 0.7 |
| AD 2 Temporal Ctx | 1.0 | Control 1 Occipital | 0.7 |
| AD 3 Temporal Ctx | 14.5 | Ctx | |
| AD 4 Temporal Ctx | 0.9 | Control 2 Occipital | 1.2 |
| AD 5 Inf Temporal | 3.0 | Ctx | |
| Ctx | | Control 3 Occipital | 0.7 |
| AD 5 Sup Temporal | 2.0 | Ctx | |
| Ctx | | Control 4 Occipital | 0.5 |
| AD 6 Inf Temporal | 2.6 | Ctx | |
| Ctx | | Control (Path) 1 | 2.4 |
| AD 6 Sup Temporal | 2.1 | Occipital Ctx | |
| Ctx | | Control (Path) 2 | 0.6 |
| Control 1 Temporal | 0.5 | Occipital Ctx | |
| Ctx | | Control (Path) 3 | 0.3 |
| Control 2 Temporal | 1.2 | Occipital Ctx | |
| Ctx | | Control (Path) 4 | 0.7 |
| Control 3 Temporal | 0.7 | Occipital Ctx | |
| Ctx | | Control 1 Parietal | 0.4 |
| Control 3 Temporal | 0.5 | Ctx | |
| Ctx | | Control 2 Parietal | 2.0 |
| Control (Path) 1 | 1.7 | Ctx | |
| Temporal Ctx | | Control 3 Parietal | 0.6 |
| Control (Path) 2 | 0.6 | Ctx | |
| Temporal Ctx | | Control (Path) 1 | 1.4 |
| | | Parietal Ctx | |
| | | Control (Path) 2 | 0.5 |
| | | Parietal Ctx | |
| | | Control (Path) 3 | 0.3 |
| | | Parietal Ctx | |
| | | Control (Path) 4 | 0.7 |
| | | Parietal Ctx | |

TABLE KC
General_screening_panel_v1.4

| | Rel. Exp. | | Rel. Exp. |
|---|---|---|---|
| | (%) | | (%) |
| | Ag3652, | | Ag3652, |
| | Run | | Run |
| Tissue Name | 218951380 | Tissue Name | 218951380 |
| Adipose | 6.2 | Renal ca. TK-10 | 26.6 |
| Melanoma* | 8.3 | Bladder | 25.7 |
| Hs688(A).T | | Gastric ca. (liver met.) | 27.0 |
| Melanoma* | 7.0 | NCI-N87 | |
| Hs688(B).T | | Gastric ca. KATO III | 100.0 |
| Melanoma* M14 | 18.3 | Colon ca. SW-948 | 8.6 |
| Melanoma* | 12.9 | Colon ca. SW480 | 25.7 |
| LOXIMVI | | Colon ca.* (SW480 | 29.5 |
| Melanoma* | 29.5 | met) SW620 | |
| SK-MEL-5 | | Colon ca. HT29 | 10.4 |
| Squamous cell | 18.8 | Colon ca. HCT-116 | 42.0 |
| carcinoma SCC-4 | | Colon ca. CaCo-2 | 23.5 |
| Testis Pool | 9.9 | Colon cancer tissue | 11.3 |
| Prostate ca.* | 12.8 | Colon ca. SW1116 | 8.2 |
| (bone met) PC-3 | | Colon ca. Colo-205 | 6.2 |
| Prostate Pool | 8.0 | Colon ca. SW-48 | 8.5 |
| Placenta | 8.4 | Colon Pool | 16.5 |
| Uterus Pool | 7.1 | Small Intestine Pool | 18.4 |
| Ovarian ca. | 25.7 | Stomach Pool | 12.6 |
| OVCAR-3 | | Bone Marrow Pool | 9.0 |
| Ovarian ca. | 59.0 | Fetal Heart | 8.7 |
| SK-OV-3 | | Heart Pool | 7.3 |
| Ovarian ca. | 7.4 | Lymph Node Pool | 19.9 |
| OVCAR-4 | | Fetal Skeletal Muscle | 6.6 |
| Ovarian ca. | 43.8 | Skeletal Muscle Pool | 6.4 |
| OVCAR-5 | | Spleen Pool | 13.0 |
| Ovarian ca. | 20.6 | Thymus Pool | 21.8 |
| IGROV-1 | | CNS cancer (glio/ | 25.9 |
| Ovarian ca. | 6.8 | astro) U87-MG | |
| OVCAR-8 | | CNS cancer (glio/ | 37.6 |
| Ovary | 8.1 | astro) U-118-MG | |
| Breast ca. MCF-7 | 36.6 | CNS cancer (neuro; | 10.9 |
| Breast ca. MDA- | 25.3 | met) SK-N-AS | |
| MB-231 | | CNS cancer (astro) | 10.6 |
| Breast ca. BT 549 | 36.9 | SF-539 | |
| Breast ca. T47D | 71.7 | CNS cancer (astro) | 33.0 |
| Breast ca. MDA-N | 16.5 | SNB-75 | |
| Breast Pool | 20.0 | CNS cancer (glio) | 21.2 |
| Trachea | 13.3 | SNB-19 | |
| Lung | 2.8 | CNS cancer (glio) | 51.4 |
| Fetal Lung | 29.1 | SF-295 | |
| Lung ca. NCI-N417 | 5.5 | Brain (Amygdala) | 4.0 |
| Lung ca. LX-1 | 35.1 | Pool | |
| Lung ca. NCI-H146 | 8.9 | Brain (cerebellum) | 2.9 |
| Lung ca. SHP-77 | 18.3 | Brain (fetal) | 6.3 |
| Lung ca. A549 | 36.1 | Brain (Hippocampus) | 3.9 |
| Lung ca. NCI-H526 | 6.9 | Pool | |
| Lung ca. NCI-H23 | 32.5 | Cerebral Cortex Pool | 4.6 |
| Lung ca. NCI-H460 | 15.2 | Brain (Substantia | 3.0 |
| Lung ca. HOP-62 | 13.1 | nigra) Pool | |
| Lung ca. NCI-H522 | 17.9 | Brain (Thalamus) Pool | 6.5 |
| Liver | 1.1 | Brain (whole) | 5.3 |
| Fetal Liver | 24.7 | Spinal Cord Pool | 7.6 |
| Liver ca. HepG2 | 18.8 | Adrenal Gland | 7.4 |
| Kidney Pool | 29.3 | Pituitary gland Pool | 3.2 |
| Fetal Kidney | 30.4 | Salivary Gland | 4.6 |
| Renal ca. 786-0 | 25.2 | Thyroid (female) | 4.7 |
| Renal ca. A498 | 5.1 | Pancreatic ca. | 24.3 |
| Renal ca. ACHN | 19.6 | CAPAN2 | |
| Renal ca. UO-31 | 20.7 | Pancreas Pool | 21.3 |

TABLE KD
Panel 4.1D

| | Rel. Exp. | | Rel. Exp. |
|---|---|---|---|
| | (%) | | (%) |
| | Ag3652, | | Ag3652, |
| | Run | | Run |
| Tissue Name | 169975808 | Tissue Name | 169975808 |
| Secondary Th1 act | 69.3 | HUVEC IL-1beta | 28.3 |
| Secondary Th2 act | 80.7 | HUVEC IFN gamma | 31.4 |
| Secondary Tr1 act | 100.0 | HUVEC TNF alpha + | 14.9 |
| Secondary Th1 rest | 21.9 | IFN gamma | |
| Secondary Th2 rest | 28.1 | HUVEC TNF alpha + | 15.9 |
| Secondary Tr1 rest | 24.5 | IL4 | |
| Primary Th1 act | 58.2 | HUVEC IL-11 | 14.7 |
| Primary Th2 act | 63.3 | Lung Microvascular | 40.1 |
| Primary Tr1 act | 64.6 | EC none | |
| Primary Th1 rest | 31.9 | Lung Microvascular | 29.7 |
| Primary Th2 rest | 29.5 | EC TNFalpha + | |
| Primary Tr1 rest | 48.3 | IL-1beta | |
| CD45RA CD4 | 39.0 | Microvascular Dermal | 17.4 |
| lymphocyte act | | EC none | |
| CD45RO CD4 | 71.2 | Microvascular Dermal | 20.6 |
| lymphocyte act | | EC TNFalpha + | |
| CD8 lymphocyte act | 67.8 | IL-1beta | |
| Secondary CD8 | 64.6 | Bronchical epithelium | 13.3 |
| lymphocyte rest | | TNFalpha + IL1beta | |
| Secondary CD8 | 41.8 | Small airway | 9.2 |
| lymphocyte act | | epithelium none | |
| CD4 lymphocyte | 24.7 | Small airway | 20.4 |
| none | | epithelium | |
| 2ry Th1/Th2/Tr1_{—} | 39.8 | TNFalpha + IL-1beta | |
| anti CD95 CH11 | | Coronery artery | 9.4 |
| LAK cells rest | 34.2 | SMC rest | |
| LAK cells IL-2 | 75.3 | Coronery artery SMC | 9.7 |
| LAK cells IL-2 + | 44.8 | TNFalpha + IL-1beta | |
| IL-12 | | Astrocytes rest | 11.3 |
| LAK cells IL-2 + | 53.2 | Astrocytes | 8.1 |
| IFN gamma | | TNFalpha + IL-1beta | |
| LAK cells IL-2 + | 61.6 | KU-812 (Basophil) | 52.1 |
| IL-18 | | rest | |
| LAK cells | 26.8 | KU-812 (Basophil) | 85.3 |
| PMA/ionomycin | | PMA/ionomycin | |
| NK Cells IL-2 rest | 51.4 | CCD1106 | 23.7 |
| Two Way MLR 3 | 61.6 | (Keratinocytes) none | |
| day | | CCD1106 | 18.7 |
| Two Way MLR 5 | 42.9 | (Keratinocytes) | |
| day | | TNFalpha + IL-1beta | |
| Two Way MLR 7 | 30.6 | Liver cirrhosis | 8.6 |
| day | | NCI-H292 none | 29.3 |
| PBMC rest | 25.3 | NCI-H292 IL-4 | 57.4 |
| PBMC PWM | 48.0 | NCI-H292 IL-9 | 67.8 |
| PBMC PHA-L | 32.8 | NCI-H292 IL-13 | 57.8 |
| Ramos (B cell) none | 51.4 | NCI-H292 IFN gamma | 57.0 |
| Ramos (B cell) | 34.2 | HPAEC none | 15.3 |
| ionomycin | | HPAEC TNF alpha + | 28.1 |
| B lymphocytes | 41.2 | IL-1 beta | |
| PWM | | Lung fibroblast none | 15.1 |
| B lymphocytes | 51.1 | Lung fibroblast | 10.0 |
| CD40L and IL-4 | | TNF alpha + IL-1 beta | |
| EOL-1 dbcAMP | 54.0 | Lung fibroblast IL-4 | 18.4 |
| EOL-1 dbcAMP | 56.6 | Lung fibroblast IL-9 | 19.1 |
| PMA/ionomycin | | Lung fibroblast IL-13 | 22.7 |
| Dendritic cells none | 34.9 | Lung fibroblast IFN | 18.6 |
| Dendritic cells LPS | 30.1 | gamma | |
| Dendritic cells | 32.3 | Dermal fibroblast | 22.4 |
| anti-CD40 | | CCD1070 rest | |
| Monocytes rest | 50.3 | Dermal fibroblast | 51.4 |
| Monocytes LPS | 37.1 | CCD1070 TNF alpha | |
| Macrophages rest | 45.4 | Dermal fibroblast | 16.2 |
| Macrophages LPS | 18.4 | CCD1070 IL-1 beta | |
| HUVEC none | 16.8 | Dermal fibroblast | 12.9 |
| HUVEC starved | 21.9 | IFN gamma | |
| | | Dermal fibroblast IL-4 | 16.0 |
| | | Dermal fibroblast rest | 11.3 |
| | | Neutrophilis TNFa + | 6.8 |
| | | LPS | |
| | | Neutrophils rest | 33.0 |
| | | Colon | 12.2 |
| | | Lung | 19.1 |
| | | Thymus | 84.7 |
| | | Kidney | 35.6 |

CNS_Neurodegeneration_v1.0 Summary:

Ag3652 The CG388665-01 gene appears to be slightly upregulated in the temporal cortex of Alzheimer's disease patients. Therefore, blockade of this receptor may decrease neuronal death and be of use in the treatment of this disease.

General_Screening_Panel_v1.4 Summary:

Ag3652 Highest expression of the CGS8665-01 gene is seen in a gastric cancer cell line (CT=27.6). Expression in breast and ovarian cancer cell lines appears to be higher than in the normal tissue samples. The CG88665-01 gene codes for a novel protein belonging to minichromosome maintenance (MCM) protein family. Recently, MCM proteins have been considered as pre-cancer markers (ref. 1). Thus, expression of this gene may be used as a diagnostic markers for these cancers. Therapeutic modulation of this gene product may also be useful in the treatment of these cancers.

Among tissues with metabolic function, this gene is expressed at moderate to low levels in pituitary, adipose, adrenal gland, pancreas, thyroid, and adult and fetal skeletal muscle, heart and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

In addition, this gene is expressed at much higher levels in fetal lung and (CTs=29−30) when compared to expression in the adult counterpart (CTs=33−34). Thus, expression of this gene may be used to differentiate between the fetal and adult source of these tissues.

This gene is also expressed at moderate to low levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Overall, the ubiquitous expression of the gene in this panel suggests a broader role for this gene product in cell growth and proliferation. (Alison M R, Hunt T, Forbes S J. (2002) Minichromosome maintenance (MCM) proteins may be pre-cancer markers Gut. 2002 50(3):290-1).

Panel 4.1D Summary:

Ag3652 Highest expression of the CG88665-01 gene is seen in chronically activated Tr1 cells (CT=29.5). Expression of this gene also appears to be slightly upregulated in activated T cells when compared to expression in resting T cells. This gene also is expressed at moderate to low levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in 110 agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

L. CG88856-01: Novel Protein

Expression of gene CG88856-01 was assessed using the primer-probe sets Ag3597 and Ag3679, described in Tables LA and LB. Results of the RTQ-PCR runs are shown in Tables LC and LD.

TABLE LA

Probe Name Ag3597

| | | | Start | SEQ ID |
|---|---|---|---|---|
| Primers | Sequences | Length | Position | No |
| Forward | 5′-aaggaacacagcctacttgtca-3′ | 22 | 313 | 109 |
| Probe | TET-5′-cttcaaccacctaacagccacagcag-3′-TAMRA | 26 | 338 | 110 |
| Reverse | 5′-aaagcccactaggagagagaca-3′ | 22 | 368 | 111 |

TABLE LB

Probe Name Ag3679

| | | | Start | SEQ ID |
|---|---|---|---|---|
| Primers | Sequences | Length | Position | No |
| Forward | 5′-acaaaggaacacagcctacttg-3′ | 22 | 310 | 112 |
| Probe | TET-5′-cttcaaccacctaacagccacagcag-3′-TAMRA | 26 | 338 | 113 |
| Reverse | 5′-gcccactaggagagagacactt-3′ | 22 | 365 | 114 |

TABLE LC
CNS_neurodegeneration_v1.0

| | Rel. Exp. | | Rel. Exp. |
|---|---|---|---|
| | (%) | | (%) |
| | Ag3597, | | Ag3597, |
| | Run | | Run |
| Tissue Name | 211010103 | Tissue Name | 211010103 |
| AD 1 Hippo | 18.2 | Control (Path) 3 | 11.0 |
| AD 2 Hippo | 24.0 | Temporal Ctx | |
| AD 3 Hippo | 13.8 | Control (Path) 4 | 28.7 |
| AD 4 Hippo | 7.1 | Temporal Ctx | |
| AD 5 hippo | 72.7 | AD 1 Occipital Ctx | 21.0 |
| AD 6 Hippo | 47.6 | AD 2 Occipital Ctx | 0.0 |
| Control 2 Hippo | 19.5 | (Missing) | |
| Control 4 Hippo | 9.8 | AD 3 Occipital Ctx | 11.3 |
| Control (Path) 3 | 11.3 | AD 4 Occipital Ctx | 18.0 |
| Hippo | | AD 5 Occipital Ctx | 32.3 |
| AD 1 Temporal Ctx | 26.6 | AD 6 Occipital Ctx | 30.8 |
| AD 2 Temporal Ctx | 32.3 | Control 1 Occipital | 7.9 |
| AD 3 Temporal Ctx | 7.0 | Ctx | |
| AD 4 Temporal Ctx | 29.1 | Control 2 Occipital | 33.0 |
| AD 5 Inf Temporal | 100.0 | Ctx | |
| Ctx | | Control 3 Occipital | 18.7 |
| AD 5 Sup Temporal | 49.7 | Ctx | |
| Ctx | | Control 4 Occipital | 8.8 |
| AD 6 Inf Temporal | 47.0 | Ctx | |
| Ctx | | Control (Path) 1 | 55.9 |
| AD 6 Sup Temporal | 42.9 | Occipital Ctx | |
| Ctx | | Control (Path) 2 | 12.5 |
| Control 1 Temporal | 10.0 | Occipital Ctx | |
| Ctx | | Control (Path) 3 | 11.3 |
| Control 2 Temporal | 25.2 | Occipital Ctx | |
| Ctx | | Control (Path) 4 | 14.6 |
| Control 3 Temporal | 17.1 | Occipital Ctx | |
| Ctx | | Control 1 Parietal | 13.1 |
| Control 4 Temporal | 12.7 | Ctx | |
| Ctx | | Control 2 Parietal | 54.0 |
| Control (Path) 1 | 37.9 | Ctx | |
| Temporal Ctx | | Control 3 Parietal | 15.9 |
| Control (Path) 2 | 27.9 | Ctx | |
| Temporal Ctx | | Control (Path) 1 | 43.2 |
| | | Parietal Ctx | |
| | | Control (Path) 2 | 22.4 |
| | | Parietal Ctx | |
| | | Control (Path) 3 | 10.7 |
| | | Parietal Ctx | |
| | | Control (Path) 4 | 28.3 |
| | | Parietal Ctx | |

TABLE LC
General_screening_panel_v1.4

| | Rel. Exp. (%) | Rel. Exp. (%) | | Rel. Exp. (%) | Rel. Exp. (%) |
|---|---|---|---|---|---|
| | Ag3597, Run | Ag3679, Run | | Ag3597, Run | Ag3679, Run |
| Tissue Name | 218307127 | 218941309 | Tissue Name | 218307127 | 218941309 |
| Adipose | 17.7 | 4.6 | Renal ca. TK-10 | 26.8 | 25.0 |
| Melanoma* | 22.2 | 22.5 | Bladder | 23.0 | 27.7 |
| Hs688(A).T | | | | | |
| Melanoma* | 22.1 | 23.5 | Gastric ca. (liver | 36.9 | 37.1 |
| Hs688(B).T | | | met.) NCI-N87 | | |
| Melanoma* | 19.9 | 21.3 | Gastric ca. KATO | 45.7 | 51.8 |
| M14 | | | III | | |
| Melanoma* | 23.3 | 21.5 | Colon ca. SW- | 7.2 | 10.7 |
| LOXIMVI | | | 948 | | |
| Melanoma* | 27.4 | 38.2 | Colon ca. SW480 | 26.8 | 46.0 |
| SK-MEL-5 | | | | | |
| Squamous cell | 22.8 | 32.3 | Colon ca.* | 21.5 | 19.3 |
| carcinoma | | | (SW480 met) | | |
| SCC-4 | | | SW620 | | |
| Testis Pool | 31.0 | 26.1 | Colon ca. HT29 | 11.4 | 10.5 |
| Prostate ca.* | 42.3 | 43.5 | Colon ca. HCT- | 32.1 | 34.9 |
| (bone met) | | | 116 | | |
| PC-3 | | | | | |
| Prostate Pool | 12.4 | 13.1 | Colon ca. CaCo-2 | 27.7 | 33.9 |
| Placenta | 20.3 | 21.0 | Colon cancer | 15.6 | 12.8 |
| | | | tissue | | |
| Uterus Pool | 13.1 | 12.8 | Colon ca. | 11.0 | 12.5 |
| | | | SW1116 | | |
| Ovarian ca. | 33.2 | 26.8 | Colon ca. Colo- | 2.9 | 5.1 |
| OVCAR-3 | | | 205 | | |
| Ovarian ca. | 36.6 | 25.5 | Colon ca. SW-48 | 3.8 | 6.5 |
| SK-OV-3 | | | | | |
| Ovarian ca. | 16.4 | 17.8 | Colon Pool | 19.2 | 24.3 |
| OVCAR-4 | | | | | |
| Ovarian ca. | 33.2 | 58.2 | Small Intestine | 31.4 | 33.7 |
| OVCAR-5 | | | Pool | | |
| Ovarian ca. | 15.7 | 18.6 | Stomach Pool | 11.9 | 14.8 |
| IGROV-1 | | | | | |
| Ovarian ca. | 5.8 | 9.7 | Bone Marrow | 11.2 | 10.6 |
| OVCAR-8 | | | Pool | | |
| Ovary | 13.4 | 12.1 | Fetal Heart | 22.7 | 24.3 |
| Breast ca. | 47.0 | 57.8 | Heart Pool | 10.5 | 12.8 |
| MCF-7 | | | | | |
| Breast ca. | 40.9 | 48.3 | Lymph Node | 27.0 | 24.7 |
| MDA-MB- | | | Pool | | |
| 231 | | | | | |
| Breast ca. BT | 52.1 | 50.0 | Fetal Skeletal | 17.3 | 18.3 |
| 549 | | | Muscle | | |
| Breast ca. | 100.0 | 100.0 | Skeletal Muscle | 30.8 | 28.5 |
| T47D | | | Pool | | |
| Breast ca. | 13.6 | 21.8 | Spleen Pool | 17.1 | 19.9 |
| MDA-N | | | | | |
| Breast Pool | 22.2 | 20.7 | Thymus Pool | 19.8 | 19.3 |
| Trachea | 21.5 | 21.2 | CNS cancer | 31.9 | 45.4 |
| | | | (glio/astro) U87- | | |
| | | | MG | | |
| Lung | 5.6 | 5.3 | CNS cancer | 46.3 | 56.3 |
| | | | (glio/astro)U- | | |
| | | | 118-MG | | |
| Fetal Lung | 36.9 | 35.6 | CNS cancer | 29.3 | 27.4 |
| | | | (neuro;met) SK- | | |
| | | | N-AS | | |
| Lung ca. NCI- | 4.0 | 7.3 | CNS cancer | 10.4 | 12.5 |
| N417 | | | (astro) SF-539 | | |
| Lung ca. LX-1 | 36.1 | 34.6 | CNS cancer | 40.1 | 51.4 |
| | | | (astro) SNB-75 | | |
| Lung ca. NCI- | 5.3 | 6.3 | CNS cancer (glio) | 14.2 | 19.9 |
| H146 | | | SNB-19 | | |
| Lung ca. SHP-77 | 13.5 | 24.5 | CNS cancer (glio) | 49.7 | 44.4 |
| 77 | | | SF-295 | | |
| Lung ca. A549 | 22.5 | 27.7 | Brain (Amygdala) | 22.5 | 20.7 |
| | | | Pool | | |
| Lung ca. NCI- | 8.4 | 12.5 | Brain | 77.9 | 79.0 |
| H526 | | | (cerebellum) | | |
| Lung ca. NCI- | 24.0 | 35.1 | Brain (fetal) | 36.9 | 37.1 |
| H23 | | | | | |
| Lung ca. NCI- | 9.9 | 15.5 | Brain | 18.8 | 19.9 |
| H460 | | | (Hippocampus) | | |
| | | | Pool | | |
| Lung ca. | 9.6 | 12.8 | Cerebral Cortex | 19.9 | 21.3 |
| HOP-62 | | | Pool | | |
| Lung ca. NCI- | 24.0 | 23.8 | Brain (Substantia | 18.9 | 19.6 |
| H522 | | | nigra) Pool | | |
| Liver | 5.1 | 5.2 | Brain (Thalamus) | 30.1 | 31.0 |
| | | | Pool | | |
| Fetal Liver | 14.0 | 24.1 | Brain (whole) | 23.8 | 25.5 |
| Liver ca. | 17.0 | 18.8 | Spinal Cord Pool | 25.0 | 27.7 |
| HepG2 | | | | | |
| Kidney Pool | 30.8 | 43.2 | Adrenal Gland | 36.1 | 34.6 |
| Fetal Kidney | 24.7 | 28.5 | Pituitary gland | 5.8 | 8.2 |
| | | | Pool | | |
| Renal ca. 786-0 | 21.8 | 21.6 | Salivary Gland | 15.6 | 15.7 |
| Renal ca. | 4.2 | 4.6 | Thyroid (female) | 13.4 | 13.2 |
| A498 | | | | | |
| Renal ca. | 17.6 | 18.8 | Pancreatic ca. | 27.7 | 27.5 |
| ACHN | | | CAPAN2 | | |
| Renal ca. UO-31 | 24.7 | 22.1 | Pancreas Pool | 29.7 | 27.9 |

TABLE LD
Panel 4.1D

| | Rel. | Rel. | | Rel. | Rel. |
|---|---|---|---|---|---|
| | Exp. (%) | Exp. (%) | | Exp. (%) | Exp. (%) |
| | Ag3597, | Ag3679, | | Ag3597, | Ag3679, |
| | Run | Run | | Run | Run |
| Tissue Name | 169910426 | 169988037 | Tissue Name | 169910426 | 169988037 |
| Secondary Th1 act | 63.7 | 64.9 | HUVEC IL-1 beta | 25.9 | 18.8 |
| Secondary Th2 act | 64.2 | 95.3 | HUVEC IFN | 34.4 | 33.0 |
| | | | gamma | | |
| Secondary Tr1 act | 82.4 | 87.7 | HUVEC TNF | 25.3 | 27.5 |
| | | | alpha + IFN | | |
| | | | gamma | | |
| Secondary Th1 rest | 26.8 | 41.8 | HUVEC TNF | 27.2 | 30.4 |
| | | | alpha + IL4 | | |
| Secondary Th2 rest | 42.3 | 60.7 | HUVEC IL-11 | 13.1 | 21.6 |
| Secondary Tr1 rest | 36.6 | 46.0 | Lung | 44.4 | 52.1 |
| | | | Microvascular EC | | |
| | | | none | | |
| Primary Th1 act | 43.5 | 54.0 | Lung | 48.3 | 48.6 |
| | | | Microvascular EC | | |
| | | | TNF alpha + IL- | | |
| | | | 1 beta | | |
| Primary Th2 act | 55.5 | 63.3 | Microvascular | 24.3 | 35.1 |
| | | | Dermal EC none | | |
| Primary Tr1 act | 51.1 | 73.7 | Microsvasular | 25.9 | 24.8 |
| | | | Dermal EC | | |
| | | | TNF alpha + IL- | | |
| | | | 1 beta | | |
| Primary Th1 rest | 48.6 | 56.3 | Bronchial | 35.4 | 31.9 |
| | | | epithelium | | |
| | | | TNF alpha + | | |
| | | | IL1 beta | | |
| Primary Th2 rest | 46.7 | 57.4 | Small airway | 17.2 | 18.7 |
| | | | epithelium none | | |
| Primary Tr1 rest | 49.7 | 69.3 | Small airway | 38.2 | 46.3 |
| | | | epithelium | | |
| | | | TNF alpha + IL- | | |
| | | | 1 beta | | |
| CD45RA CD4 | 51.4 | 63.3 | Coronery artery | 24.0 | 36.6 |
| lymphocyte act | | | SMC rest | | |
| CD45RO CD4 | 66.9 | 95.3 | Coronery artery | 33.0 | 32.5 |
| lymphocyte act | | | SMC TNF alpha + | | |
| | | | IL-1 beta | | |
| CD8 lymphocyte | 58.6 | 75.8 | Astrocytes rest | 19.8 | 26.6 |
| act | | | | | |
| Secondary CD8 | 51.1 | 69.3 | Astrocytes | 17.2 | 26.6 |
| lymphocyte rest | | | TNF alpha + IL- | | |
| | | | 1 beta | | |
| Secondary CD8 | 38.7 | 37.9 | KU-812 (Basophil) | 37.1 | 50.7 |
| lymphocyte act | | | rest | | |
| CD4 lymphocyte | 42.0 | 58.6 | KU-812 (Basophil) | 72.7 | 68.3 |
| none | | | PMA/ionomycin | | |
| 2ry | 41.5 | 56.6 | CCD1106 | 65.1 | 64.2 |
| Th1/Th2/Tr1_anti- | | | (Keratinocytes) | | |
| CD95 CH11 | | | none | | |
| LAK cells rest | 61.1 | 71.7 | CCD1106 | 48.3 | 58.6 |
| | | | (Keratinocytes) | | |
| | | | TNF alpha + IL- | | |
| | | | 1 beta | | |
| LAK cells IL-2 | 61.1 | 72.7 | Liver cirrhosis | 14.9 | 20.4 |
| LAK cells IL-2 + | 100.0 | 62.0 | NCI-H292 none | 22.1 | 30.6 |
| IL-12 | | | | | |
| LAK cells IL- | 85.3 | 65.1 | NCI-H292 IL-4 | 36.9 | 42.0 |
| 2 + IFN gamma | | | | | |
| LAK cells IL 2 + | 73.7 | 100.0 | NCI-H292 IL-9 | 62.9 | 70.2 |
| IL-18 | | | | | |
| LAK cells | 58.6 | 83.5 | NCI-H292 IL-13 | 42.0 | 37.4 |
| PMA/ionomycin | | | | | |
| NK Cells IL-2 rest | 59.9 | 98.6 | NCI-H292 IFN | 46.0 | 48.3 |
| | | | gamma | | |
| Two Way MLR 3 | 72.7 | 65.5 | HPAEC none | 27.4 | 26.2 |
| day | | | | | |
| Two Way MLR 5 | 43.8 | 56.3 | HPAEC TNF | 37.1 | 48.3 |
| day | | | alpha + IL-1 beta | | |
| Two Way MLR 7 | 29.3 | 40.1 | Lung fibroblast | 27.0 | 29.5 |
| day | | | none | | |
| PBMC rest | 44.1 | 58.6 | Lung fibroblast | 17.2 | 24.7 |
| | | | TNF alpha + IL-1 | | |
| | | | beta | | |
| PBMC PWM | 48.3 | 60.7 | Lung fibroblast IL-4 | 25.3 | 31.6 |
| PBMC PHA-L | 31.9 | 52.5 | Lung fibroblast IL-9 | 45.4 | 43.2 |
| Ramos (B cell) | 65.5 | 87.1 | Lung fibroblast IL- | 30.1 | 25.0 |
| none | | | 13 | | |
| Ramos (B cell) | 71.2 | 87.1 | Lung fibroblast | 31.4 | 32.1 |
| ionomycin | | | IFN gamma | | |
| B lymphocytes | 33.2 | 52.9 | Dermal fibroblast | 45.4 | 51.1 |
| PWM | | | CCD1070 rest | | |
| B lymphocytes | 58.2 | 78.5 | Dermal fibroblast | 74.2 | 98.6 |
| CD40L and IL-4 | | | CCD1070 TNF | | |
| | | | alpha | | |
| EOL-1 dbcAMP | 40.1 | 60.3 | Dermal fibroblast | 32.5 | 34.9 |
| | | | CCD1070 IL-1 | | |
| | | | beta | | |
| EOL-1 dbcAMP | 50.7 | 75.8 | Dermal fibroblast | 20.3 | 27.9 |
| PMA/ionomycin | | | IFN gamma | | |
| Dendritic cells none | 41.5 | 52.9 | Dermal fibroblasts | 41.2 | 41.2 |
| | | | IL-4 | | |
| Dendritic cells LPS | 28.1 | 42.0 | Dermal Fibroblasts | 24.8 | 29.7 |
| | | | rest | | |
| Dendritic cells anti- | 36.9 | 40.9 | Neutrophils | 15.6 | 29.5 |
| CD40 | | | TNFa + LPS | | |
| Monocytes rest | 55.1 | 60.3 | Neutrophils rest | 84.1 | 76.8 |
| Monocytes LPS | 57.4 | 82.4 | Colon | 34.9 | 34.4 |
| Macrophages rest | 40.1 | 54.0 | Lung | 31.0 | 29.3 |
| Macrophages LPS | 22.5 | 31.4 | Thymus | 90.1 | 85.3 |
| HUVEC none | 15.0 | 24.0 | Kidney | 49.7 | 52.5 |
| HUVEC starved | 28.1 | 29.7 | | | |

CNS_Neurodegeneration_v1.0 Summary: Ag3597 This panel does not show differential expression of the CG88856-01 gene in Alzheimer's disease. However, this expression profile confirms the presence of this gene in the brain. Please see Panel 1.4 for discussion of utility of this gene in the central nervous system. Results from a second experiment with the probe primer Ag3679 are not included. The amp plot indicates there were experimental difficulties with this run.

General_Screening_Panel_v1.4 Summary: Ag3597/Ag3679 Two experiments with the same probe and primer produce results that are in excellent agreement. Highest expression of the CG88856-01 gene is seen in a breast cancer cell line. Higher levels of expression are also seen in breast, prostate, ovarian and lung tissues when compared to expression in normal tissue. Thus, expression of this gene could be used as a marker of these cancers and therapeutic modulation of the activity of this gene may be effective in their treatment.

Among tissues with metabolic function, this gene is expressed at high to moderate levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

This gene is also expressed at high to moderate levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

The CG88856-01 gene codes for variant of DMR protein and a homologue of mouse dystrophia myotonica-containing WD repeat motif protein (DMR-N9 protein). DMR-N9 has been implicated in myotonic dystrophy (MD) (Ref. 1). Therefore, therapeutic modulation of this gene could be useful in the treatment of MD. (Groenen P, Wieringa B.(1998)Expanding complexity in myotonic dystrophy. Bioessays 20(11):901-12).

Panel 4.1D Summary:

Ag3597/Ag3679 Two experiments with the same probe and primer produce results that are in excellent agreement. Highest expression of the CG88856-01 gene is seen in cytokine activated LAK cells. In addition, this gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screeningpanel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

M. CG90853-01: Homeodomain-Interacting Protein Kinase

Expression of gene CG90853-01 was assessed using the primer-probe set Ag3768, described in Table MA. Results of the RTQ-PCR runs are shown in Tables MB, MC and MD.

TABLE MA
Probe Name Ag3768

| | | | Start | SEQ ID |
|---|---|---|---|---|
| Primers | Sequences | Length | Position | No |
| Forward | 5′-ccagatttgcactcagacaga-3′ | 21 | 1894 | 116 |
| Probe | TET-5′-tccaacagacatttatagtatgtccacctg-3′-TAMRA | 30 | 1920 | 117 |
| Reverse | 5′-gcttgtagtccactttgaaacg-3′ | 22 | 1950 | 118 |

TABLE MB
CNS_neurodegeneration_v1.0

| | Rel. Exp. | | Rel. Exp. |
|---|---|---|---|
| | (%) | | (%) |
| | Ag3768, | | Ag3768, |
| | Run | | Run |
| Tissue Name | 211176319 | Tissue Name | 211176319 |
| AD 1 Hippo | 20.2 | Control (Path) 3 | 16.2 |
| AD 2 Hippo | 32.5 | Temporal Ctx | |
| AD 3 Hippo | 19.5 | Control (Path) 4 | 28.5 |
| AD 4 Hippo | 7.9 | Temporal Ctx | |
| AD 5 Hippo | 100.0 | AD 1 Occipital Ctx | 27.4 |
| AD 6 Hippo | 76.3 | AD 2 Occipital Ctx | 0.0 |
| Control 2 Hippo | 24.0 | (Missing) | |
| Control 4 Hippo | 26.1 | AD 3 Occipital Ctx | 15.4 |
| Control (Path) 3 | 15.0 | AD 4 Occipital Ctx | 15.5 |
| Hippo | | AD 5 Occipital Ctx | 36.1 |
| AD 1 Temporal Ctx | 35.4 | AD 6 Occipital Ctx | 28.7 |
| AD 2 Temporal Ctx | 22.5 | Control 1 Occipital | 9.5 |
| AD 3 Temporal Ctx | 9.4 | Ctx | |
| AD 4 Temporal Ctx | 28.1 | Control 2 Occipital | 46.3 |
| AD 5 Inf Temporal | 73.2 | Ctx | |
| Ctx | | Control 3 Occipital | 24.7 |
| AD 5 Sup Temporal | 63.3 | Ctx | |
| Ctx | | Control 4 Occipital | 11.3 |
| AD 6 Inf Temporal | 64.2 | Ctx | |
| Ctx | | Control (Path) 1 | 71.7 |
| AD 6 Sup Temporal | 64.2 | Occipital Ctx | |
| Ctx | | Control (Path) 2 | 17.1 |
| Control 1 Temporal | 10.3 | Occipital Ctx | |
| Ctx | | Control (Path) 3 | 13.0 |
| Control 2 Temporal | 30.6 | Occipital Ctx | |
| Ctx | | Control (Path) 4 | 7.9 |
| Control 3 Temporal | 20.0 | Occipital Ctx | |
| Ctx | | Control 1 Parietal | 15.7 |
| Control 3 Temporal | 5.4 | Ctx | |
| Ctx | | Control 2 Parietal | 49.7 |
| Control (Path) 1 | 57.4 | Ctx | |
| Temporal Ctx | | Control 3 Parietal | 16.8 |
| Control (Path) 2 | 39.2 | Ctx | |
| Temporal Ctx | | Control (Path) 1 | 11.6 |
| | | Parietal Ctx | |
| | | Control (Path) 2 | 19.2 |
| | | Parietal Ctx | |
| | | Control (Path) 3 | 12.9 |
| | | Parietal Ctx | |
| | | Control (Path) 4 | 16.6 |
| | | Parietal Ctx | |

TABLE MC
General_screening_panel_v1.4

| | Rel. Exp. | | Rel. Exp. |
|---|---|---|---|
| | (%) | | (%) |
| | Ag3768, | | Ag3768, |
| | Run | | Run |
| Tissue Name | 218981616 | Tissue Name | 218981616 |
| Adipose | 6.8 | Renal ca. TK-10 | 26.6 |
| Melanoma* | 17.6 | Bladder | 14.1 |
| Hs688(A).T | | Gastric ca. (liver met.) | 36.6 |
| Melanoma* | 15.6 | NCI-N87 | |
| Hs688(B).T | | Gastric ca. KATO III | 26.8 |
| Melanoma* M14 | 20.0 | Colon ca. SW-948 | 5.7 |
| Melanoma* | 14.7 | Colon ca. SW480 | 20.6 |
| LOXIMVI | | Colon ca.* (SW480 | 14.9 |
| Melanoma* | 11.3 | met) SW620 | |
| SK-MEL-5 | | Colon ca. HT29 | 11.1 |
| Squamous cell | 14.7 | Colon ca. HCT-116 | 23.5 |
| carcinoma SCC-4 | | Colon ca. CaCo-2 | 19.3 |
| Testis Pool | 26.1 | Colon cancer tissue | 18.7 |
| Prostate ca.* | 20.7 | Colon ca. SW1116 | 4.2 |
| (bone met) PC-3 | | Colon ca. Colo-205 | 7.7 |
| Prostate Pool | 4.1 | Colon ca. SW-48 | 7.1 |
| Placenta | 8.2 | Colon Pool | 16.6 |
| Uterus Pool | 3.7 | Small Intestine Pool | 10.4 |
| Ovarian ca. | 12.0 | Stomach Pool | 12.7 |
| OVCAR-3 | | Bone Marrow Pool | 5.2 |
| Ovarian ca. | 66.0 | Fetal Heart | 13.7 |
| SK-OV-3 | | Heart Pool | 6.1 |
| Ovarian ca. | 8.3 | Lymph Node Pool | 16.2 |
| OVCAR-4 | | Fetal Skeletal Muscle | 7.2 |
| Ovarian ca. | 28.1 | Skeletal Muscle Pool | 8.5 |
| OVCAR-5 | | Spleen Pool | 10.1 |
| Ovarian ca. | 14.8 | Thymus Pool | 20.6 |
| IGROV-1 | | CNS cancer (glio/ | 28.1 |
| Ovarian ca. | 17.3 | astro) U87-MG | |
| OVCAR-8 | | CNS cancer (glio/ | 36.9 |
| Ovary | 9.4 | astro) U-118-MG | |
| Breast ca. MCF-7 | 100.0 | CNS cancer (neuro; | 18.0 |
| Breast ca. MDA- | 25.5 | met) SK-N-AS | |
| MB-231 | | CNS cancer (astro) | 23.2 |
| Breast ca. BT 549 | 39.2 | SF-539 | |
| Breast ca. T47D | 47.3 | CNS cancer (astro) | 43.8 |
| Breast ca. MDA-N | 6.1 | SNB-75 | |
| Breast Pool | 18.0 | CNS cancer (glio) | 14.4 |
| Trachea | 20.4 | SNB-19 | |
| Lung | 4.6 | CNS cancer (glio) | 37.4 |
| Fetal Lung | 51.1 | SF-295 | |
| Lung ca. NCI-N417 | 6.8 | Brain (Amygdala) | 8.1 |
| Lung ca. LX-1 | 14.2 | Pool | |
| Lung ca. NCI-H146 | 4.1 | Brain (cerebellum) | 37.6 |
| Lung ca. SHP-77 | 14.0 | Brain (fetal) | 13.5 |
| Lung ca. A549 | 15.4 | Brain (Hippocampus) | 11.3 |
| Lung ca. NCI-H526 | 9.5 | Pool | |
| Lung ca. NCI-H23 | 33.0 | Cerebral Cortex Pool | 13.6 |
| Lung ca. NCI-H460 | 12.3 | Brain (Substantia | 12.0 |
| Lung ca. HOP-62 | 7.4 | nigra) Pool | |
| Lung ca. NCI-H522 | 16.8 | Brain (Thalamus) Pool | 15.9 |
| Liver | 1.6 | Brain (whole) | 29.1 |
| Fetal Liver | 34.4 | Spinal Cord Pool | 17.9 |
| Liver ca. HepG2 | 8.5 | Adrenal Gland | 21.5 |
| Kidney Pool | 18.6 | Pituitary gland Pool | 7.1 |
| Fetal Kidney | 7.0 | Salivary Gland | 5.7 |
| Renal ca. 786-0 | 18.9 | Thyroid (female) | 6.0 |
| Renal ca. A498 | 7.7 | Pancreatic ca. | 10.7 |
| Renal ca. ACHN | 9.1 | CAPAN2 | |
| Renal ca. UO-31 | 15.7 | Pancreas Pool | 16.3 |

TABLE MD
Panel 4.1D

| | Rel. Exp. | | Rel. Exp. |
|---|---|---|---|
| | (%) | | (%) |
| | Ag3768, | | Ag3768, |
| | Run | | Run |
| Tissue Name | 170069115 | Tissue Name | 170069115 |
| Secondary Th1 act | 30.8 | HUVEC IL-1beta | 22.4 |
| Secondary Th2 act | 44.1 | HUVEC IFN gamma | 11.8 |
| Secondary Tr1 act | 51.1 | HUVEC TNF alpha + | 15.9 |
| Secondary Th1 rest | 13.3 | IFN gamma | |
| Secondary Th2 rest | 16.5 | HUVEC TNF alpha + | 17.7 |
| Secondary Tr1 rest | 19.6 | IL4 | |
| Primary Th1 act | 16.7 | HUVEC IL-11 | 12.3 |
| Primary Th2 act | 32.5 | Lung Microvascular | 22.7 |
| Primary Tr1 act | 26.6 | EC none | |
| Primary Th1 rest | 20.3 | Lung Microvascular | 19.1 |
| Primary Th2 rest | 14.8 | EC TNFalpha + | |
| Primary Tr1 rest | 19.8 | IL-1beta | |
| CD45RA CD4 | 21.5 | Microvascular Dermal | 16.7 |
| lymphocyte act | | EC none | |
| CD45RO CD4 | 25.9 | Microvascular Dermal | 19.3 |
| lymphocyte act | | EC TNFalpha + | |
| CD8 lymphocyte act | 31.6 | IL-1beta | |
| Secondary CD8 | 32.3 | Bronchical epithelium | 12.3 |
| lymphocyte rest | | TNFalpha + IL1beta | |
| Secondary CD8 | 25.5 | Small airway | 4.1 |
| lymphocyte act | | epithelium none | |
| CD4 lymphocyte | 14.9 | Small airway | 14.6 |
| none | | epithelium | |
| 2ry Th1/Th2/Tr1_{—} | 22.1 | TNFalpha + IL-1beta | |
| anti CD95 CH11 | | Coronery artery | 8.7 |
| LAK cells rest | 35.6 | SMC rest | |
| LAK cells IL-2 | 26.4 | Coronery artery SMC | 8.8 |
| LAK cells IL-2 + | 30.8 | TNFalpha + IL-1beta | |
| IL-12 | | Astrocytes rest | 11.5 |
| LAK cells IL-2 + | 31.4 | Astrocytes | 6.8 |
| IFN gamma | | TNFalpha + IL-1beta | |
| LAK cells IL-2 + | 31.2 | KU-812 (Basophil) | 30.8 |
| IL-18 | | rest | |
| LAK cells | 19.1 | KU-812 (Basophil) | 56.3 |
| PMA/ionomycin | | PMA/ionomycin | |
| NK Cells IL-2 rest | 63.3 | CCD1106 | 8.4 |
| Two Way MLR 3 | 46.3 | (Keratinocytes) none | |
| day | | CCD1106 | 15.2 |
| Two Way MLR 5 | 26.8 | (Keratinocytes) | |
| day | | TNFalpha + IL-1beta | |
| Two Way MLR 7 | 25.9 | Liver cirrhosis | 6.6 |
| day | | NCI-H292 none | 7.8 |
| PBMC rest | 27.7 | NCI-H292 IL-4 | 16.5 |
| PBMC PWM | 33.2 | NCI-H292 IL-9 | 19.6 |
| PBMC PHA-L | 19.2 | NCI-H292 IL-13 | 11.0 |
| Ramos (B cell) none | 34.4 | NCI-H292 IFN gamma | 17.9 |
| Ramos (B cell) | 31.0 | HPAEC none | 12.9 |
| ionomycin | | HPAEC TNF alpha + | 28.9 |
| B lymphocytes | 21.9 | IL-1 beta | |
| PWM | | Lung fibroblast none | 7.0 |
| B lymphocytes | 41.5 | Lung fibroblast | 7.4 |
| CD40L and IL-4 | | TNF alpha + IL-1 beta | |
| EOL-1 dbcAMP | 17.1 | Lung fibroblast IL-4 | 17.1 |
| EOL-1 dbcAMP | 17.0 | Lung fibroblast IL-9 | 12.9 |
| PMA/ionomycin | | Lung fibroblast IL-13 | 9.6 |
| Dendritic cells none | 26.8 | Lung fibroblast IFN | 15.3 |
| Dendritic cells LPS | 18.9 | gamma | |
| Dendritic cells | 22.4 | Dermal fibroblast | 17.2 |
| anti-CD40 | | CCD1070 rest | |
| Monocytes rest | 34.6 | Dermal fibroblast | 48.6 |
| Monocytes LPS | 48.0 | CCD1070 TNF alpha | |
| Macrophages rest | 22.7 | Dermal fibroblast | 9.2 |
| Macrophages LPS | 18.0 | CCD1070 IL-1 beta | |
| HUVEC none | 15.8 | Dermal fibroblast | 7.9 |
| HUVEC starved | 16.2 | IFN gamma | |
| | | Dermal fibroblast IL-4 | 15.7 |
| | | Dermal fibroblast rest | 6.0 |
| | | Neutrophilis TNFa + | 7.1 |
| | | LPS | |
| | | Neutrophils rest | 35.4 |
| | | Colon | 10.5 |
| | | Lung | 18.3 |
| | | Thymus | 100.0 |
| | | Kidney | 15.5 |

CNS_Neurodegeneration_v1.0 Summary:

Ag3768 The CG90853-01 gene appears to be slightly upregulated in the temporal cortex of Alzheimer's disease patients and also in pateint not demented but showing severe AD-like pathology as compared to non-demented patient with no neuropathology. The temporal cortex is a region that shows degeneration at the mid-stages of this disease. These results suggest that this gene may be a marker of Alzheimer's-like neurodegeneration, and may also be involved in the process of neurodegeneration.

General_Screening_Panel_v1.4 Summary:

Ag3768 Expression of the CG90853-01 gene is ubiquitous in this panel, with highest expression in a breast cancer MCF-7 cell line (CT=28.6). Significant expression is also seen in a cluster of breast and ovarian cancer cell lines. Thus, therapeutic modulation of the expression or function of this gene may be effective in the treatment of these cancers.

In addition, this gene is expressed at much higher levels in fetal lung and liver tissue (CTs=30) when compared to expression in the adult counterpart (CTs=33−34). Thus, expression of this gene may be used to differentiate between the fetal and adult source of these tissues.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

This gene is also expressed at moderate levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 4.1D Summary:

Ag3678 Expression of the CG90853-01 gene is ubiquitous in this panel, with highest expression in the thymus (CT=29.6). This gene also is expressed at moderate to low levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

N. CG90866-01 and CG90866-02: Protein Kinase

Expression of gene CG90866-01 and CG90866-01 was assessed using the primer-probe sets Ag1088, Ag941 and Ag3771, described in Tables NA, NB and NC. Results of the RTQ-PCR runs are shown in Tables ND, NE, NF and NG.

TABLE NA

Probe Name Ag1088

| | | | | |
|---|---|---|---|---|
| | | | Start | |
| Primers | Sequences | Length | Position | SEQ ID No |

| | | | | |
|---|---|---|---|---|
| Forward | 5′-cttgatgaagaaagcagaggaa-3′ | 22 | 776 | 119 |
| Probe | TET-5′-atccagatcaaccaaggctcaccatt-3′- | 26 | 814 | 120 |
| | TAMRA | | | |
| Reverse | 5′-agtcaggggcaatctgagatat-3′ | 22 | 843 | 121 |

TABLE NB

Probe Name Ag941

| | | | | |
|---|---|---|---|---|
| | | | Start | |
| Primers | Sequences | Length | Position | SEQ ID No |

| | | | | |
|---|---|---|---|---|
| Forward | 5′-cctccactcagccatgatta-3′ | 20 | 1241 | 122 |
| Probe | TET-5′-ataccgagacctgaaaccccacaatg-3′- | 26 | 1262 | 123 |
| | TAMRA | | | |
| Reverse | 5′-gcagcattgggatacagtgt-3′ | 20 | 1299 | 124 |

TABLE NC

Probe Name Ag3771

| | | | | |
|---|---|---|---|---|
| | | | Start | SEQ ID |
| Primers | Sequences | Length | Position | No |

| | | | | |
|---|---|---|---|---|
| Forward | 5′-ggcacaaagattttctcctttt-3′ | 22 | 2259 | 125 |
| Probe | TET-5′-tgatttcaccattcagaaactcattga-3′- | 27 | 2285 | 126 |
| | TAMRA | | | |
| Reverse | 5′-gaaaacagttggcttgttcttg-3′ | 22 | 2314 | 127 |

TABLE ND
CNS_neurodegeneration_v1.0

| | Rel. Exp. | | Rel. Exp. |
|---|---|---|---|
| | (%) | | (%) |
| | Ag3771, | | Ag3771, |
| | Run | | Run |
| Tissue Name | 211175148 | Tissue Name | 211175148 |
| AD 1 Hippo | 6.9 | Control (Path) 3 | 9.1 |
| AD 2 Hippo | 21.5 | Temporal Ctx | |
| AD 3 Hippo | 8.8 | Control (Path) 4 | 48.3 |
| AD 4 Hippo | 8.9 | Temporal Ctx | |
| AD 5 hippo | 100.0 | AD 1 Occipital Ctx | 27.4 |
| AD 6 Hippo | 45.7 | AD 2 Occipital Ctx | 0.0 |
| Control 2 Hippo | 23.2 | (Missing) | |
| Control 4 Hippo | 11.9 | AD 3 Occipital Ctx | 6.2 |
| Control (Path) 3 | 10.8 | AD 4 Occipital Ctx | 21.6 |
| Hippo | | AD 5 Occipital Ctx | 48.0 |
| AD 1 Temporal Ctx | 13.7 | AD 6 Occipital Ctx | 52.9 |
| AD 2 Temporal Ctx | 25.3 | Control 1 Occipital | 4.9 |
| AD 3 Temporal Ctx | 5.6 | Ctx | |
| AD 4 Temporal Ctx | 19.9 | Control 2 Occipital | 66.0 |
| AD 5 Inf Temporal | 77.9 | Ctx | |
| Ctx | | Control 3 Occipital | 28.3 |
| AD 5 Sup Temporal | 40.3 | Ctx | |
| Ctx | | Control 4 Occipital | 11.4 |
| AD 6 Inf Temporal | 62.4 | Ctx | |
| Ctx | | Control (Path) 1 | 97.3 |
| AD 6 Sup Temporal | 73.2 | Occipital Ctx | |
| Ctx | | Control (Path) 2 | 28.1 |
| Control 1 Temporal | 10.4 | Occipital Ctx | |
| Ctx | | Control (Path) 3 | 3.6 |
| Control 2 Temporal | 34.9 | Occipital Ctx | |
| Ctx | | Control (Path) 4 | 39.5 |
| Control 3 Temporal | 21.5 | Occipital Ctx | |
| Ctx | | Control 1 Parietal | 7.1 |
| Control 4 Temporal | 12.6 | Ctx | |
| Ctx | | Control 2 Parietal | 44.8 |
| Control (Path) 1 | 66.0 | Ctx | |
| Temporal Ctx | | Control 3 Parietal | 18.6 |
| Control (Path) 2 | 55.9 | Ctx | |
| Temporal Ctx | | Control (Path) 1 | 86.5 |
| | | Parietal Ctx | |
| | | Control (Path) 2 | 34.9 |
| | | Parietal Ctx | |
| | | Control (Path) 3 | 7.1 |
| | | Parietal Ctx | |
| | | Control (Path) 4 | 54.0 |
| | | Parietal Ctx | |

TABLE NE
General_screening_panel_v1.4

| | Rel. Exp. | | Rel. Exp. |
|---|---|---|---|
| | (%) | | (%) |
| | Ag3771, | | Ag3771, |
| | Run | | Run |
| Tissue Name | 218982528 | Tissue Name | 218982528 |
| Adipose | 11.7 | Renal ca. TK-10 | 5.6 |
| Melanoma* | 2.3 | Bladder | 8.0 |
| Hs688(A).T | | Gastric ca. (liver met.) | 0.0 |
| Melanoma* | 0.9 | NCI-N87 | |
| Hs688(B).T | | Gastric ca. KATO III | 0.0 |
| Melanoma* M14 | 23.0 | Colon ca. SW-948 | 0.0 |
| Melanoma* | 0.6 | Colon ca. SW480 | 0.0 |
| LOXIMVI | | Colon ca.* (SW480 | 0.0 |
| Melanoma* | 23.7 | met) SW620 | |
| SK-MEL-5 | | Colon ca. HT29 | 0.0 |
| Squamous cell | 0.0 | Colon ca. HCT-116 | 0.1 |
| carcinoma SCC-4 | | Colon ca. CaCo-2 | 0.2 |
| Testis Pool | 3.8 | Colon cancer tissue | 4.6 |
| Prostate ca.* | 1.3 | Colon ca. SW1116 | 0.0 |
| (bone met) PC-3 | | Colon ca. Colo-205 | 0.0 |
| Prostate Pool | 4.3 | Colon ca. SW-48 | 0.0 |
| Placenta | 0.2 | Colon Pool | 15.6 |
| Uterus Pool | 7.4 | Small Intestine Pool | 13.3 |
| Ovarian ca. | 0.3 | Stomach Pool | 8.5 |
| OVCAR-3 | | Bone Marrow Pool | 5.9 |
| Ovarian ca. | 3.8 | Fetal Heart | 2.0 |
| SK-OV-3 | | Heart Pool | 6.7 |
| Ovarian ca. | 0.0 | Lymph Node Pool | 12.8 |
| OVCAR-4 | | Fetal Skeletal Muscle | 2.0 |
| Ovarian ca. | 1.7 | Skeletal Muscle Pool | 5.9 |
| OVCAR-5 | | Spleen Pool | 16.6 |
| Ovarian ca. | 0.1 | Thymus Pool | 7.2 |
| IGROV-1 | | CNS cancer (glio/ | 4.7 |
| Ovarian ca. | 0.1 | astro) U87-MG | |
| OVCAR-8 | | CNS cancer (glio/ | 11.7 |
| Ovary | 5.5 | astro) U-118-MG | |
| Breast ca. MCF-7 | 0.0 | CNS cancer (neuro; | 0.6 |
| Breast ca. MDA- | 0.1 | met) SK-N-AS | |
| MB-231 | | CNS cancer (astro) | 0.1 |
| Breast ca. BT 549 | 0.0 | SF-539 | |
| Breast ca. T47D | 5.0 | CNS cancer (astro) | 0.0 |
| Breast ca. MDA-N | 4.5 | SNB-75 | |
| Breast Pool | 13.9 | CNS cancer (glio) | 0.5 |
| Trachea | 5.3 | SNB-19 | |
| Lung | 5.0 | CNS cancer (glio) | 3.1 |
| Fetal Lung | 100.0 | SF-295 | |
| Lung ca. NCI-N417 | 0.2 | Brain (Amygdala) | 4.9 |
| Lung ca. LX-1 | 0.0 | Pool | |
| Lung ca. NCI-H146 | 0.0 | Brain (cerebellum) | 1.1 |
| Lung ca. SHP-77 | 0.1 | Brain (fetal) | 2.9 |
| Lung ca. A549 | 21.3 | Brain (Hippocampus) | 6.2 |
| Lung ca. NCI-H526 | 0.0 | Pool | |
| Lung ca. NCI-H23 | 1.9 | Cerebral Cortex Pool | 12.5 |
| Lung ca. NCI-H460 | 0.7 | Brain (Substantia | 7.6 |
| Lung ca. HOP-62 | 0.4 | nigra) Pool | |
| Lung ca. NCI-H522 | 0.0 | Brain (Thalamus) Pool | 13.8 |
| Liver | 0.3 | Brain (whole) | 5.7 |
| Fetal Liver | 9.3 | Spinal Cord Pool | 6.3 |
| Liver ca. HepG2 | 0.0 | Adrenal Gland | 3.7 |
| Kidney Pool | 23.2 | Pituitary gland Pool | 2.0 |
| Fetal Kidney | 27.7 | Salivary Gland | 1.3 |
| Renal ca. 786-0 | 17.9 | Thyroid (female) | 7.7 |
| Renal ca. A498 | 4.8 | Pancreatic ca. | 0.0 |
| Renal ca. ACHN | 9.0 | CAPAN2 | |
| Renal ca. UO-31 | 4.0 | Pancreas Pool | 9.7 |

TABLE NF
Panel 1.3D

| | Rel. Exp. | | Rel. Exp. |
|---|---|---|---|
| | (%) | | (%) |
| | Ag941, | | Ag941, |
| | Run | | Run |
| Tissue Name | 167819097 | Tissue Name | 167819097 |
| Liver | 0.0 | Kidney (fetal) | 76.8 |
| adenocarcinoma | | Renal ca. 786-0 | 27.4 |
| Pancreas | 3.4 | Renal ca. A498 | 3.6 |
| Pancreatic ca. | 0.0 | Renal ca. RXF 393 | 0.0 |
| CAPAN2 | | Renal ca. ACHN | 14.8 |
| Adrenal gland | 5.6 | Renal ca. UO-31 | 3.1 |
| Thyroid | 4.0 | Renal ca. TK-10 | 6.9 |
| Salivary gland | 3.2 | Liver | 8.2 |
| Pituitary gland | 4.0 | Liver (fetal) | 3.8 |
| Brain (fetal) | 6.2 | Liver ca. | 0.0 |
| Brain (whole) | 51.4 | (hepatoblast) HepG2 | |
| Brain (amygdala) | 13.4 | Lung | 38.4 |
| Brain (cerebellum) | 23.7 | Lung (fetal) | 100.0 |
| Brain | 17.4 | Lung ca. (small cell) | 0.0 |
| (hippocampus) | | LX-1 | |
| Brain (substantia | 19.9 | Lung ca. (small cell) | 0.0 |
| nigra) | | NCI-H69 | |
| Brain (thalamus) | 14.3 | Lung ca. (s.cell var.) | 0.0 |
| Cerebral Cortex | 13.6 | SHP-77 | |
| Spinal cord | 17.8 | Lung ca. (large cell) | 0.0 |
| glio/astro U87-MG | 2.6 | NCI-H460 | |
| glio/astro | 7.3 | Lung ca. (non-sm. | 41.8 |
| U-118-MG | | cell) A549 | |
| astrocytoma SW | 0.0 | Lung ca. (non-s.cell) | 1.8 |
| 1783 | | NCI-H23 | |
| neuro*; met | 1.3 | Lung ca. (non-s.cell) | 1.5 |
| SK-N-AS | | HOP-62 | |
| astrocytoma | 0.1 | Lung ca. (non s.cl) | 0.0 |
| SF-539 | | NCI-H522 | |
| astrocytoma | 1.4 | Lung ca. (squam.) | 0.8 |
| SNB-75 | | SW 900 | |
| glioma SNB-19 | 0.1 | Lung ca. (squam.) | 0.0 |
| glioma U251 | 0.5 | NCI-H596 | |
| glioma SF-295 | 2.4 | Mammary gland | 8.2 |
| Heart (fetal) | 0.9 | Breast ca.* (pl.ef) | 0.0 |
| Heart | 7.2 | MCF-7 | |
| Skeletal muscle | 1.7 | Breast ca.* (pl.ef) | 0.0 |
| (fetal) | | MDA-MB-231 | |
| Skeletal muscle | 22.7 | Breast ca*. (pl.ef) | 23.5 |
| Bone marrow | 22.4 | T47D | |
| Thymus | 3.3 | Breast ca. BT-549 | 0.0 |
| Spleen | 11.1 | Breast ca. MDA-N | 10.9 |
| Lymph node | 12.0 | Ovary | 0.3 |
| Colorectal | 3.4 | Ovarian ca. OVCAR-3 | 1.0 |
| Stomach | 4.4 | Ovarian ca. OVCAR-4 | 0.3 |
| Small intestine | 2.6 | Ovarian ca. OVCAR-5 | 0.0 |
| Colon ca. SW480 | 0.0 | Ovarian ca. OVCAR-8 | 0.5 |
| Colon ca.* | 0.0 | Ovarian ca. IGROV-1 | 0.0 |
| SW620(SW480 | | Ovarian ca.* (ascites) | 13.6 |
| met) | | SK-OV-3 | |
| Colon ca. HT29 | 0.0 | Uterus | 10.2 |
| Colon ca. HCT-116 | 0.0 | Placenta | 1.4 |
| Colon ca. CaCo-2 | 0.0 | Prostate | 1.3 |
| Colon ca. tissue | 7.3 | Prostate ca.* (bone | 1.6 |
| (ODO3866) | | met)PC-3 | |
| Colon ca. | 0.0 | Testis | 1.2 |
| HCC-2998 | | Melanoma | 1.3 |
| Gastric ca.* (liver | 0.0 | Hs688(A).T | |
| met) NCI-N87 | | Melanoma* (met) | 0.7 |
| Bladder | 8.2 | Hs688(B).T | |
| Trachea | 2.3 | Melanoma UACC-62 | 10.6 |
| Kidney | 49.0 | Melamona M14 | 5.9 |
| | | Melamona LOX | 1.4 |
| | | IMVI | |
| | | Melanoma* (met) | 21.3 |
| | | SK-MEL-5 | |
| | | Adipose | 30.6 |

TABLE NG
Panel 4.1D

| | Rel. Exp. | | Rel. Exp. |
|---|---|---|---|
| | (%) | | (%) |
| | Ag3771, | | Ag3771, |
| | Run | | Run |
| Tissue Name | 170130259 | Tissue Name | 170130259 |
| Secondary Th1 act | 0.0 | HUVEC IL-1beta | 0.1 |
| Secondary Th2 act | 0.0 | HUVEC IFN gamma | 0.7 |
| Secondary Tr1 act | 0.0 | HUVEC TNF alpha + | 0.1 |
| Secondary Th1 rest | 0.0 | IFN gamma | |
| Secondary Th2 rest | 0.0 | HUVEC TNF alpha + | 0.0 |
| Secondary Tr1 rest | 0.0 | IL4 | |
| Primary Th1 act | 0.0 | HUVEC IL-11 | 0.2 |
| Primary Th2 act | 0.0 | Lung Microvascular | 0.0 |
| Primary Tr1 act | 0.0 | EC none | |
| Primary Th1 rest | 0.0 | Lung Microvascular | 0.0 |
| Primary Th2 rest | 0.0 | EC TNFalpha + | |
| Primary Tr1 rest | 0.0 | IL-1beta | |
| CD45RA CD4 | 0.6 | Microvascular Dermal | 0.0 |
| lymphocyte act | | EC none | |
| CD45RO CD4 | 0.2 | Microvascular Dermal | 0.0 |
| lymphocyte act | | EC TNFalpha + | |
| CD8 lymphocyte act | 0.1 | IL-1beta | |
| Secondary CD8 | 0.0 | Bronchical epithelium | 0.3 |
| lymphocyte rest | | TNFalpha + IL1beta | |
| Secondary CD8 | 0.0 | Small airway | 0.1 |
| lymphocyte act | | epithelium none | |
| CD4 lymphocyte | 0.7 | Small airway | 0.0 |
| none | | epithelium | |
| 2ry Th1/Th2/Tr1_{—} | 0.0 | TNFalpha + IL-1beta | |
| anti CD95 CH11 | | Coronery artery | 1.0 |
| LAK cells rest | 25.9 | SMC rest | |
| LAK cells IL-2 | 0.7 | Coronery artery SMC | 0.8 |
| LAK cells IL-2 + | 0.6 | TNFalpha + IL-1beta | |
| IL-12 | | Astrocytes rest | 0.1 |
| LAK cells IL-2 + | 1.3 | Astrocytes | 0.0 |
| IFN gamma | | TNFalpha + IL-1beta | |
| LAK cells IL-2 + | 0.8 | KU-812 (Basophil) | 0.0 |
| IL-18 | | rest | |
| LAK cells | 7.3 | KU-812 (Basophil) | 0.1 |
| PMA/ionomycin | | PMA/ionomycin | |
| NK Cells IL-2 rest | 0.7 | CCD1106 | 0.0 |
| Two Way MLR 3 | 23.0 | (Keratinocytes) none | |
| day | | CCD1106 | 0.0 |
| Two Way MLR 5 | 7.7 | (Keratinocytes) | |
| day | | TNFalpha + IL-1beta | |
| Two Way MLR 7 | 1.7 | Liver cirrhosis | 3.2 |
| day | | NCI-H292 none | 1.9 |
| PBMC rest | 10.0 | NCI-H292 IL-4 | 1.5 |
| PBMC PWM | 2.0 | NCI-H292 IL-9 | 2.1 |
| PBMC PHA-L | 3.0 | NCI-H292 IL-13 | 1.3 |
| Ramos (B cell) none | 0.2 | NCI-H292 IFN gamma | 2.5 |
| Ramos (B cell) | 0.1 | HPAEC none | 0.8 |
| ionomycin | | HPAEC TNF alpha + | 0.7 |
| B lymphocytes | 1.6 | IL-1 beta | |
| PWM | | Lung fibroblast none | 1.4 |
| B lymphocytes | 6.6 | Lung fibroblast | 3.9 |
| CD40L and IL-4 | | TNF alpha + IL-1 beta | |
| EOL-1 dbcAMP | 0.1 | Lung fibroblast IL-4 | 0.5 |
| EOL-1 dbcAMP | 0.0 | Lung fibroblast IL-9 | 1.2 |
| PMA/ionomycin | | Lung fibroblast IL-13 | 0.4 |
| Dendritic cells none | 11.1 | Lung fibroblast IFN | 0.9 |
| Dendritic cells LPS | 10.5 | gamma | |
| Dendritic cells | 8.1 | Dermal fibroblast | 0.5 |
| anti-CD40 | | CCD1070 rest | |
| Monocytes rest | 63.7 | Dermal fibroblast | 0.4 |
| Monocytes LPS | 3.5 | CCD1070 TNF alpha | |
| Macrophages rest | 6.1 | Dermal fibroblast | 0.7 |
| Macrophages LPS | 6.6 | CCD1070 IL-1 beta | |
| HUVEC none | 0.2 | Dermal fibroblast | 2.2 |
| HUVEC starved | 0.3 | IFN gamma | |
| | | Dermal fibroblast IL-4 | 1.6 |
| | | Dermal fibroblast rest | 2.0 |
| | | Neutrophilis TNFa + | 21.8 |
| | | LPS | |
| | | Neutrophils rest | 100.0 |
| | | Colon | 1.4 |
| | | Lung | 27.9 |
| | | Thymus | 3.1 |
| | | Kidney | 14.2 |

CNS_Neurodegeneration_v1.0 Summary:

Ag3771 This panel confirms the expression of the CG90866-01 gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_Screening_Panel_v1.4 Summary:

Ag3771 Highest expression of the CG90866-01 gene is detected in fetal lung sample (CT=27.5). Interestingly, expression of this gene is much higher in fetal (27-31) as compared to adult lung and liver (CT=32−35). Therefore, expression of this gene can be used to distinguish these fetal from adult tissues. In addition, the relative overexpression of this gene in these fetal tissues suggests that the protein product may enhance growth or development of these tissues in the fetus and thus may also act in a regenerative capacity in the adult. Therefore, therapeutic modulation of the protein kinase encoded by this gene could be useful in treatment of lung and liver related diseases.

Among tissues with metabolic or endocrine function, this gene is expressed at moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 1.3D Summary:

Ag3771 Highest expression of the CG90866-01 gene is detected in fetal lung sample (CT=29). This gene is expressed at moderate levels in all the brain region and also in tissues with metabolic or endocrine functions. Please see panel 1.4 for discussion on potential utility of this gene in CNS and metabolic disorders.

In addition, this gene is expressed at low to moderat levels in number of cancer cell lines (melanoma, ovarian, breast, lung and renal) used in this panel. Therefore, therapeutic modulation of this gene product may be useful in the treatment of these cancers.

Panel 4.1D Summary:

Ag3771 Highest expression of the CG90866-01 gene is detected in resting neutropils (CT=27.3). In addition, this gene is expressed in TNFalpha+LPS treated neutrophils. Therefore, the gene product may reduce activation of these inflammatory cells and be useful as a protein therapeutic to reduce or eliminate the symptoms in patients with Crohn's disease, ulcerative colitis, multiple sclerosis, chronic obstructive pulmonary disease, asthma, emphysema, rheumatoid arthritis, lupus erythematosus, or psoriasis. In addition, small molecule or antibody antagonists of this gene product may be effective in increasing the immune response in patients with AIDS or other immunodeficiencies.

In addition, expression of this gene is down-regulated in cytokine stimulated LAK cells and LPS-treated monocytes. Therefore, expression of this gene can be used to distinguish these stimulated versus resting cells.

In addition, low to moderate expression of this gene is also seen in B cells, dendritic cells, endothelial cells, fibroblasts and normal tissues represented by kidney, thymus, lung, and colon. Therefore, therapeutic modulation of this gene may be beneficial in the treatements of cancer, Crohn's disease, ulcerative colitis, multiple sclerosis, chronic obstructive pulmonary disease, asthma, emphysema, rheumatoid arthritis, lupus erythematosus, or psoriasis, microbial and viral infections.

O. CG93781-01: Pancreatic Hormone Peptide Domain Containing Protein

Expression of gene CG93781-01 was assessed using the primer-probe set Ag3879, described in Table OA. Results of the RTQ-PCR runs are shown in Tables OB, OC and OD.

TABLE OA

Probe Name Ag3879

| | | | | |
|---|---|---|---|---|
| | | | Start | |
| Primers | Sequences | Length | Position | SEQ ID No |

| | | | | |
|---|---|---|---|---|
| Forward | 5′-aggtgatccgctaccagaag-3′ | 20 | 1826 | 128 |
| Probe | TET-5′-cacaactacatccagatgtaccggcg-3′- | 26 | 1855 | 129 |
| | TAMRA | | | |
| Reverse | 5′-tgcagctcctgctctagct-3′ | 19 | 1889 | 130 |

TABLE OB
CNS_neurodegeneration_v1.0

| | Rel. Exp. | | Rel. Exp. |
|---|---|---|---|
| | (%) | | (%) |
| | Ag3879, | | Ag3879, |
| | Run | | Run |
| Tissue Name | 212195188 | Tissue Name | 212195188 |
| AD 1 Hippo | 81.8 | Control (Path) 3 | 17.0 |
| AD 2 Hippo | 66.9 | Temporal Ctx | |
| AD 3 Hippo | 9.6 | Control (Path) 4 | 19.1 |
| AD 4 Hippo | 18.9 | Temporal Ctx | |
| AD 5 hippo | 53.2 | AD 1 Occipital Ctx | 39.2 |
| AD 6 Hippo | 72.7 | AD 2 Occipital Ctx | 0.0 |
| Control 2 Hippo | 18.9 | (Missing) | |
| Control 4 Hippo | 44.8 | AD 3 Occipital Ctx | 12.9 |
| Control (Path) 3 | 7.5 | AD 4 Occipital Ctx | 18.0 |
| Hippo | | AD 5 Occipital Ctx | 5.4 |
| AD 1 Temporal Ctx | 36.9 | AD 6 Occipital Ctx | 36.3 |
| AD 2 Temporal Ctx | 74.7 | Control 1 Occipital | 18.0 |
| AD 3 Temporal Ctx | 22.4 | Ctx | |
| AD 4 Temporal Ctx | 37.9 | Control 2 Occipital | 47.6 |
| AD 5 Inf Temporal | 81.8 | Ctx | |
| Ctx | | Control 3 Occipital | 19.2 |
| AD 5 Sup Temporal | 83.5 | Ctx | |
| Ctx | | Control 4 Occipital | 27.4 |
| AD 6 Inf Temporal | 53.6 | Ctx | |
| Ctx | | Control (Path) 1 | 100.0 |
| AD 6 Sup Temporal | 60.7 | Occipital Ctx | |
| Ctx | | Control (Path) 2 | 16.0 |
| Control 1 Temporal | 18.7 | Occipital Ctx | |
| Ctx | | Control (Path) 3 | 9.7 |
| Control 2 Temporal | 59.5 | Occipital Ctx | |
| Ctx | | Control (Path) 4 | 26.4 |
| Control 3 Temporal | 52.9 | Occipital Ctx | |
| Ctx | | Control 1 Parietal | 18.0 |
| Control 4 Temporal | 35.4 | Ctx | |
| Ctx | | Control 2 Parietal | 62.9 |
| Control (Path) 1 | 59.9 | Ctx | |
| Temporal Ctx | | Control 3 Parietal | 26.6 |
| Control (Path) 2 | 31.9 | Ctx | |
| Temporal Ctx | | Control (Path) 1 | 79.0 |
| | | Parietal Ctx | |
| | | Control (Path) 2 | 35.8 |
| | | Parietal Ctx | |
| | | Control (Path) 3 | 6.8 |
| | | Parietal Ctx | |
| | | Control (Path) 4 | 50.3 |
| | | Parietal Ctx | |

TABLE OC
General_screening_panel_v1.4

| | Rel. Exp. | | Rel. Exp. |
|---|---|---|---|
| | (%) | | (%) |
| | Ag3879, | | Ag3879, |
| | Run | | Run |
| Tissue Name | 214145891 | Tissue Name | 214145891 |
| Adipose | 0.7 | Renal ca. TK-10 | 4.2 |
| Melanoma* | 6.2 | Bladder | 3.0 |
| Hs688(A).T | | Gastric ca. (liver met.) | 5.8 |
| Melanoma* | 5.8 | NCI-N87 | |
| Hs688(B).T | | Gastric ca. KATO III | 1.9 |
| Melanoma* M14 | 9.3 | Colon ca. SW-948 | 2.8 |
| Melanoma* | 2.8 | Colon ca. SW480 | 12.2 |
| LOXIMVI | | Colon ca.* (SW480 | 5.0 |
| Melanoma* | 3.6 | met) SW620 | |
| SK-MEL-5 | | Colon ca. HT29 | 5.0 |
| Squamous cell | 1.8 | Colon ca. HCT-116 | 11.1 |
| carcinoma SCC-4 | | Colon ca. CaCo-2 | 5.0 |
| Testis Pool | 1.3 | Colon cancer tissue | 5.6 |
| Prostate ca.* | 3.8 | Colon ca. SW1116 | 4.6 |
| (bone met) PC-3 | | Colon ca. Colo-205 | 1.8 |
| Prostate Pool | 1.0 | Colon ca. SW-48 | 3.3 |
| Placenta | 1.4 | Colon Pool | 3.7 |
| Uterus Pool | 0.7 | Small Intestine Pool | 3.8 |
| Ovarian ca. | 18.8 | Stomach Pool | 2.5 |
| OVCAR-3 | | Bone Marrow Pool | 0.5 |
| Ovarian ca. | 4.0 | Fetal Heart | 0.9 |
| SK-OV-3 | | Heart Pool | 1.8 |
| Ovarian ca. | 1.8 | Lymph Node Pool | 5.0 |
| OVCAR-4 | | Fetal Skeletal Muscle | 1.1 |
| Ovarian ca. | 11.0 | Skeletal Muscle Pool | 3.5 |
| OVCAR-5 | | Spleen Pool | 2.5 |
| Ovarian ca. | 12.1 | Thymus Pool | 2.0 |
| IGROV-1 | | CNS cancer (glio/ | 3.8 |
| Ovarian ca. | 14.8 | astro) U87-MG | |
| OVCAR-8 | | CNS cancer (glio/ | 3.2 |
| Ovary | 1.6 | astro) U-118-MG | |
| Breast ca. MCF-7 | 6.7 | CNS cancer (neuro; | 5.0 |
| Breast ca. MDA- | 15.0 | met) SK-N-AS | |
| MB-231 | | CNS cancer (astro) | 1.8 |
| Breast ca. BT 549 | 6.8 | SF-539 | |
| Breast ca. T47D | 100.0 | CNS cancer (astro) | 4.1 |
| Breast ca. MDA-N | 7.4 | SNB-75 | |
| Breast Pool | 3.8 | CNS cancer (glio) | 8.8 |
| Trachea | 1.0 | SNB-19 | |
| Lung | 1.0 | CNS cancer (glio) | 6.0 |
| Fetal Lung | 1.7 | SF-295 | |
| Lung ca. NCI-N417 | 2.1 | Brain (Amygdala) | 4.2 |
| Lung ca. LX-1 | 5.6 | Pool | |
| Lung ca. NCI-H146 | 3.2 | Brain (cerebellum) | 1.9 |
| Lung ca. SHP-77 | 3.7 | Brain (fetal) | 0.1 |
| Lung ca. A549 | 3.8 | Brain (Hippocampus) | 2.0 |
| Lung ca. NCI-H526 | 6.0 | Pool | |
| Lung ca. NCI-H23 | 6.0 | Cerebral Cortex Pool | 2.2 |
| Lung ca. NCI-H460 | 3.3 | Brain (Substantia | 5.7 |
| Lung ca. HOP-62 | 3.5 | nigra) Pool | |
| Lung ca. NCI-H522 | 6.5 | Brain (Thalamus) Pool | 4.4 |
| Liver | 0.0 | Brain (whole) | 1.0 |
| Fetal Liver | 0.6 | Spinal Cord Pool | 4.9 |
| Liver ca. HepG2 | 8.7 | Adrenal Gland | 1.8 |
| Kidney Pool | 7.7 | Pituitary gland Pool | 1.1 |
| Fetal Kidney | 0.9 | Salivary Gland | 0.6 |
| Renal ca. 786-0 | 7.0 | Thyroid (female) | 1.8 |
| Renal ca. A498 | 2.7 | Pancreatic ca. | 2.8 |
| Renal ca. ACHN | 3.8 | CAPAN2 | |
| Renal ca. UO-31 | 3.9 | Pancreas Pool | 5.4 |

TABLE OD
Panel 4.1D

| | Rel. Exp. | | Rel. Exp. |
|---|---|---|---|
| | (%) | | (%) |
| | Ag3879, | | Ag3879, |
| | Run | | Run |
| Tissue Name | 170129764 | Tissue Name | 170129764 |
| Secondary Th1 act | 2.7 | HUVEC IL-1beta | 23.7 |
| Secondary Th2 act | 7.0 | HUVEC IFN gamma | 27.5 |
| Secondary Tr1 act | 1.7 | HUVEC TNF alpha + | 21.2 |
| Secondary Th1 rest | 2.8 | IFN gamma | |
| Secondary Th2 rest | 3.0 | HUVEC TNF alpha + | 16.4 |
| Secondary Tr1 rest | 6.7 | IL4 | |
| Primary Th1 act | 4.8 | HUVEC IL-11 | 17.7 |
| Primary Th2 act | 5.4 | Lung Microvascular | 55.9 |
| Primary Tr1 act | 6.1 | EC none | |
| Primary Th1 rest | 0.9 | Lung Microvascular | 36.1 |
| Primary Th2 rest | 0.8 | EC TNFalpha + | |
| Primary Tr1 rest | 1.8 | IL-1beta | |
| CD45RA CD4 | 7.4 | Microvascular Dermal | 21.9 |
| lymphocyte act | | EC none | |
| CD45RO CD4 | 6.8 | Microvascular Dermal | 10.2 |
| lymphocyte act | | EC TNFalpha + | |
| CD8 lymphocyte act | 3.1 | IL-1beta | |
| Secondary CD8 | 3.7 | Bronchical epithelium | 20.6 |
| lymphocyte rest | | TNFalpha + IL1beta | |
| Secondary CD8 | 0.4 | Small airway | 9.8 |
| lymphocyte act | | epithelium none | |
| CD4 lymphocyte | 1.9 | Small airway | 14.7 |
| none | | epithelium | |
| 2ry Th1/Th2/Tr1_{—} | 8.1 | TNFalpha + IL-1beta | |
| anti CD95 CH11 | | Coronery artery | 13.7 |
| LAK cells rest | 0.5 | SMC rest | |
| LAK cells IL-2 | 1.6 | Coronery artery SMC | 16.5 |
| LAK cells IL-2 + | 2.7 | TNFalpha + IL-1beta | |
| IL-12 | | Astrocytes rest | 13.0 |
| LAK cells IL-2 + | 4.2 | Astrocytes | 6.7 |
| IFN gamma | | TNFalpha + IL-1beta | |
| LAK cells IL-2 + | 0.8 | KU-812 (Basophil) | 3.8 |
| IL-18 | | rest | |
| LAK cells | 1.4 | KU-812 (Basophil) | 2.9 |
| PMA/ionomycin | | PMA/ionomycin | |
| NK Cells IL-2 rest | 2.1 | CCD1106 | 22.1 |
| Two Way MLR 3 | 4.2 | (Keratinocytes) none | |
| day | | CCD1106 | 9.7 |
| Two Way MLR 5 | 3.9 | (Keratinocytes) | |
| day | | TNFalpha + IL-1beta | |
| Two Way MLR 7 | 4.1 | Liver cirrhosis | 3.7 |
| day | | NCI-H292 none | 39.5 |
| PBMC rest | 0.4 | NCI-H292 IL-4 | 60.7 |
| PBMC PWM | 2.3 | NCI-H292 IL-9 | 25.2 |
| PBMC PHA-L | 2.4 | NCI-H292 IL-13 | 62.9 |
| Ramos (B cell) none | 0.3 | NCI-H292 IFN gamma | 26.1 |
| Ramos (B cell) | 5.5 | HPAEC none | 7.5 |
| ionomycin | | HPAEC TNF alpha + | 21.8 |
| B lymphocytes | 0.7 | IL-1 beta | |
| PWM | | Lung fibroblast none | 33.4 |
| B lymphocytes | 7.0 | Lung fibroblast | 25.5 |
| CD40L and IL-4 | | TNF alpha + IL-1 beta | |
| EOL-1 dbcAMP | 6.9 | Lung fibroblast IL-4 | 64.2 |
| EOL-1 dbcAMP | 3.5 | Lung fibroblast IL-9 | 61.6 |
| PMA/ionomycin | | Lung fibroblast IL-13 | 100.0 |
| Dendritic cells none | 23.3 | Lung fibroblast IFN | 79.0 |
| Dendritic cells LPS | 11.7 | gamma | |
| Dendritic cells | 7.0 | Dermal fibroblast | 33.0 |
| anti-CD40 | | CCD1070 rest | |
| Monocytes rest | 1.4 | Dermal fibroblast | 15.8 |
| Monocytes LPS | 5.9 | CCD1070 TNF alpha | |
| Macrophages rest | 21.5 | Dermal fibroblast | 21.9 |
| Macrophages LPS | 9.6 | CCD1070 IL-1 beta | |
| HUVEC none | 30.4 | Dermal fibroblast | 42.6 |
| HUVEC starved | 33.7 | IFN gamma | |
| | | Dermal fibroblast IL-4 | 42.0 |
| | | Dermal fibroblast rest | 31.6 |
| | | Neutrophilis TNFa + | 0.0 |
| | | LPS | |
| | | Neutrophils rest | 0.9 |
| | | Colon | 8.3 |
| | | Lung | 4.9 |
| | | Thymus | 9.5 |
| | | Kidney | 16.6 |

CNS_Neurodegeneration_v1.0 Summary:

Ag3879 This panel confirms the expression of the CG93781-01 gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_Screening_Panel_v1.4 Summary:

Ag3879 Expression of of the CG93781-01 gene is ubiquitous with highest level in breast cancer T47D cell line (CT=24.3). High expression of this gene is seen in cluster of cancer cell lines (CNS, colon, renal, breast, ovarian, prostate, squamous cell carcinoma, and melanoma). Therefore, therapeutic modulation of this gene product may be beneficial in treatment of these cancers.

Among tissues with metabolic or endocrine function, this gene is expressed at high to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

Interestingly, this gene is expressed at much higher levels in fetal (CT=31.7) when compared to adult liver (CT35.9). Therefore, expression of this gene can be used to distinguish fetal from adult liver. In addition, the relative overexpression of this gene in fetal liver suggests that the protein product may enhance livergrowth or development in the fetus and thus may also act in a regenerative capacity in the adult. Therefore, therapeutic modulation of the protein encoded by this gene could be useful in treatment of liver related diseases.

In addition, this gene is expressed at high levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4.1D Summary:

Ag3879 Expression of of the CG93781-01 gene is ubiquitous with highest level in IL-13 treated lung fibroblast (CT=29.5). This gene is expressed at moderate to low levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Interestingly, expression of this gene is up-regulated in ionomycin treated Ramos B cells (CT=33) as compared to the resting cells (CT=37). Therefore, expression of this gene can be used to distinguish between the resting and stimulated Ramos B cells.

P. CG93848-02: MADD

Expression of gene CG93848-02 was assessed using the primer-probe set Ag3891, described in Table PA. Results of the RTQ-PCR runs are shown in Tables PB, and PC.

TABLE PA

Probe Name Ag3891

| | | | | |
|---|---|---|---|---|
| | | | | SEQ ID |
| Primers | Sequences | Length | Position | No |

| | | | | |
|---|---|---|---|---|
| Forward | 5′-gggatcaacctcaaattcatg-3′ | 21 | 1339 | 131 |
| Probe | TET-5′-caatcaggttttcatagagctgaatcaca-3′- | 29 | 1362 | 132 |
| Reverse | 5′-aagacgcctcgaactgtattg-3′ | 21 | 1401 | 133 |

TABLE PB
CNS_neurodegeneration_v1.0

| | Rel. Exp. | | Rel. Exp. |
|---|---|---|---|
| | (%) | | (%) |
| | Ag3891, | | Ag3891, |
| | Run | | Run |
| Tissue Name | 212195211 | Tissue Name | 212195211 |
| AD 1 Hippo | 29.9 | Control (Path) 3 | 3.0 |
| AD 2 Hippo | 31.2 | Temporal Ctx | |
| AD 3 Hippo | 9.7 | Control (Path) 4 | 38.2 |
| AD 4 Hippo | 10.8 | Temporal Ctx | |
| AD 5 hippo | 57.4 | AD 1 Occipital Ctx | 23.5 |
| AD 6 Hippo | 76.3 | AD 2 Occipital Ctx | 0.0 |
| Control 2 Hippo | 14.4 | (Missing) | |
| Control 4 Hippo | 15.6 | AD 3 Occipital Ctx | 10.5 |
| Control (Path) 3 | 11.1 | AD 4 Occipital Ctx | 20.4 |
| Hippo | | AD 5 Occipital Ctx | 11.9 |
| AD 1 Temporal Ctx | 15.3 | AD 6 Occipital Ctx | 57.8 |
| AD 2 Temporal Ctx | 46.3 | Control 1 Occipital | 6.4 |
| AD 3 Temporal Ctx | 7.9 | Ctx | |
| AD 4 Temporal Ctx | 23.0 | Control 2 Occipital | 54.7 |
| AD 5 Inf Temporal | 81.2 | Ctx | |
| Ctx | | Control 3 Occipital | 21.2 |
| AD 5 Sup Temporal | 33.0 | Ctx | |
| Ctx | | Control 4 Occipital | 7.5 |
| AD 6 Inf Temporal | 60.7 | Ctx | |
| Ctx | | Control (Path) 1 | 100.0 |
| AD 6 Sup Temporal | 51.1 | Occipital Ctx | |
| Ctx | | Control (Path) 2 | 6.0 |
| Control 1 Temporal | 7.4 | Occipital Ctx | |
| Ctx | | Control (Path) 3 | 5.5 |
| Control 2 Temporal | 65.5 | Occipital Ctx | |
| Ctx | | Control (Path) 4 | 6.8 |
| Control 3 Temporal | 11.8 | Occipital Ctx | |
| Ctx | | Control 1 Parietal | 8.9 |
| Control 4 Temporal | 11.1 | Ctx | |
| Ctx | | Control 2 Parietal | 29.1 |
| Control (Path) 1 | 26.2 | Ctx | |
| Temporal Ctx | | Control 3 Parietal | 24.8 |
| Control (Path) 2 | 42.0 | Ctx | |
| Temporal Ctx | | Control (Path) 1 | 90.1 |
| | | Parietal Ctx | |
| | | Control (Path) 2 | 16.2 |
| | | Parietal Ctx | |
| | | Control (Path) 3 | 6.5 |
| | | Parietal Ctx | |
| | | Control (Path) 4 | 21.6 |
| | | Parietal Ctx | |

TABLE PC
Panel 4.1D

| | Rel. Exp. | | Rel. Exp. |
|---|---|---|---|
| | (%) | | (%) |
| | Ag3891, | | Ag3891, |
| | Run | | Run |
| Tissue Name | 170130430 | Tissue Name | 170130430 |
| Secondary Th1 act | 41.2 | HUVEC IL-1beta | 20.4 |
| Secondary Th2 act | 55.9 | HUVEC IFN gamma | 25.5 |
| Secondary Tr1 act | 41.5 | HUVEC TNF alpha + | 10.4 |
| Secondary Th1 rest | 13.1 | IFN gamma | |
| Secondary Th2 rest | 27.5 | HUVEC TNF alpha + | 9.7 |
| Secondary Tr1 rest | 27.9 | IL4 | |
| Primary Th1 act | 17.0 | HUVEC IL-11 | 7.6 |
| Primary Th2 act | 45.4 | Lung Microvascular | 25.5 |
| Primary Tr1 act | 33.2 | EC none | |
| Primary Th1 rest | 14.8 | Lung Microvascular | 16.2 |
| Primary Th2 rest | 18.4 | EC TNFalpha + | |
| Primary Tr1 rest | 26.4 | IL-1beta | |
| CD45RA CD4 | 24.8 | Microvascular Dermal | 13.9 |
| lymphocyte act | | EC none | |
| CD45RO CD4 | 47.6 | Microvascular Dermal | 9.1 |
| lymphocyte act | | EC TNFalpha + | |
| CD8 lymphocyte act | 31.4 | IL-1beta | |
| Secondary CD8 | 31.9 | Bronchical epithelium | 7.1 |
| lymphocyte rest | | TNFalpha + IL1beta | |
| Secondary CD8 | 17.7 | Small airway | 3.5 |
| lymphocyte act | | epithelium none | |
| CD4 lymphocyte | 15.5 | Small airway | 7.5 |
| none | | epithelium | |
| 2ry Th1/Th2/Tr1_{—} | 52.1 | TNFalpha + IL-1beta | |
| anti CD95 CH11 | | Coronery artery | 5.3 |
| LAK cells rest | 38.4 | SMC rest | |
| LAK cells IL-2 | 25.0 | Coronery artery SMC | 6.4 |
| LAK cells IL-2 + | 14.6 | TNFalpha + IL-1beta | |
| IL-12 | | Astrocytes rest | 4.4 |
| LAK cells IL-2 + | 11.2 | Astrocytes | 4.1 |
| IFN gamma | | TNFalpha + IL-1beta | |
| LAK cells IL-2 + | 22.8 | KU-812 (Basophil) | 13.6 |
| IL-18 | | rest | |
| LAK cells | 27.7 | KU-812 (Basophil) | 31.9 |
| PMA/ionomycin | | PMA/ionomycin | |
| NK Cells IL-2 rest | 61.6 | CCD1106 | 12.4 |
| Two Way MLR 3 | 39.0 | (Keratinocytes) none | |
| day | | CCD1106 | 8.9 |
| Two Way MLR 5 | 22.5 | (Keratinocytes) | |
| day | | TNFalpha + IL-1beta | |
| Two Way MLR 7 | 21.3 | Liver cirrhosis | 4.6 |
| day | | NCI-H292 none | 14.9 |
| PBMC rest | 14.5 | NCI-H292 IL-4 | 19.5 |
| PBMC PWM | 26.2 | NCI-H292 IL-9 | 25.0 |
| PBMC PHA-L | 29.1 | NCI-H292 IL-13 | 19.5 |
| Ramos (B cell) none | 14.4 | NCI-H292 IFN gamma | 20.2 |
| Ramos (B cell) | 16.8 | HPAEC none | 5.4 |
| ionomycin | | HPAEC TNF alpha + | 17.7 |
| B lymphocytes | 24.1 | IL-1 beta | |
| PWM | | Lung fibroblast none | 11.0 |
| B lymphocytes | 37.1 | Lung fibroblast | 23.7 |
| CD40L and IL-4 | | TNF alpha + IL-1 beta | |
| EOL-1 dbcAMP | 27.9 | Lung fibroblast IL-4 | 10.1 |
| EOL-1 dbcAMP | 23.8 | Lung fibroblast IL-9 | 19.6 |
| PMA/ionomycin | | Lung fibroblast IL-13 | 13.0 |
| Dendritic cells none | 25.0 | Lung fibroblast IFN | 15.4 |
| Dendritic cells LPS | 28.5 | gamma | |
| Dendritic cells | 24.7 | Dermal fibroblast | 17.8 |
| anti-CD40 | | CCD1070 rest | |
| Monocytes rest | 34.4 | Dermal fibroblast | 56.3 |
| Monocytes LPS | 45.1 | CCD1070 TNF alpha | |
| Macrophages rest | 100.0 | Dermal fibroblast | 20.0 |
| Macrophages LPS | 51.4 | CCD1070 IL-1 beta | |
| HUVEC none | 9.1 | Dermal fibroblast | 10.8 |
| HUVEC starved | 13.6 | IFN gamma | |
| | | Dermal fibroblast IL-4 | 15.6 |
| | | Dermal fibroblast rest | 10.7 |
| | | Neutrophilis TNFa + | 1.8 |
| | | LPS | |
| | | Neutrophils rest | 5.8 |
| | | Colon | 5.5 |
| | | Lung | 8.7 |
| | | Thymus | 18.9 |
| | | Kidney | 14.4 |

CNS_Neurodegeneration_v1.0 Summary:

Ag3891 This panel confirms the expression of the CG93495-01 gene at low levels in the brains of an independent group of individuals. However, no differential expression of this gene was detected between Alzheimer's diseased postmortem brains and those of non-demented controls in this experiment.

The CG93495-01 gene codes for a splice variant of MAP kinase-activating death domain protein (MADD). The MADD gene is differentially expressed in neoplastic versus normal cells and the protein is a substrate for c-Jun N-terminal kinase in the human central nervous system (Ref. 1). MADD homolog from C. elegans, AEX-3, a GDP/GTP exchange proteins specific for the Rab3 subfamily members has been shown to regulate exocytosis of neurotransmitters (Ref. 2). Therefore, therapeutic modulation of the activity of this gene may prove useful in the treatment of neurological disorders. (Zhang Y, Zhou L, Miller C A. (1998) A splicing variant of a death domain protein that is regulated by a mitogen-activated kinase is a substrate for c-Jun N-terminal kinase in the human central nervous system. Proc Natl Acad Sci USA 95(5):2586-91; Iwasaki K, Staunton J, Saifee O, Nonet M, Thomas J H. (1997) aex-3 encodes a novel regulator of presynaptic activity in C. elegans. Neuron 18(4):613-22).

General_Screening_Panel_v1.4 Summary:

Ag3891 Results from one experiment with the CG93495-01 gene are not included. The amp plot indicates that there were experimental difficulties with this run.

Panel 4.1D Summary:

Ag3891 Highest expression of the CG93495-01 gene is detected in resting macrophage (CT—27). This gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Q. CG94161-01: GAR22 Protein

Expression of gene CG94161-01 was assessed using the primer-probe set Ag3906, described in Table QA. Results of the RTQ-PCR runs are shown in Tables QB, and QC.

TABLE QA

Probe Name Ag3906

| | | | | |
|---|---|---|---|---|
| | | | Start | |
| Primers | Sequences | Length | Position | SEQ ID No |

| | | | | |
|---|---|---|---|---|
| Forward | 5′-tcaaagtgtctgaggggaagta-3′ | 22 | 827 | 134 |
| Probe | TET-5′-acaccctcatcttcatcgggtacag-3′- | 26 | 866 | 135 |
| Reverse | 5′-cctacacgtaccatcacatggt-3′ | 22 | 902 | 136 |

TABLE QB
CNS_neurodegeneration_v1.0

| | Rel. Exp. | | Rel. Exp. |
|---|---|---|---|
| | (%) | | (%) |
| | Ag3906, | | Ag3906, |
| | Run | | Run |
| Tissue Name | 212248229 | Tissue Name | 212248229 |
| AD 1 Hippo | 42.9 | Control (Path) 3 | 0.0 |
| AD 2 Hippo | 32.1 | Temporal Ctx | |
| AD 3 Hippo | 0.0 | Control (Path) 4 | 20.7 |
| AD 4 Hippo | 7.9 | Temporal Ctx | |
| AD 5 Hippo | 90.8 | AD 1 Occipital Ctx | 7.6 |
| AD 6 Hippo | 41.2 | AD 2 Occipital Ctx | 0.0 |
| Control 2 Hippo | 0.0 | (Missing) | |
| Control 4 Hippo | 21.5 | AD 3 Occipital Ctx | 29.3 |
| Control (Path) 3 | 19.8 | AD 4 Occipital Ctx | 5.6 |
| Hippo | | AD 5 Occipital Ctx | 28.3 |
| AD 1 Temporal Ctx | 19.9 | AD 6 Occipital Ctx | 11.0 |
| AD 2 Temporal Ctx | 12.9 | Control 1 Occipital | 0.0 |
| AD 3 Temporal Ctx | 10.7 | Ctx | |
| AD 4 Temporal Ctx | 16.0 | Control 2 Occipital | 45.7 |
| AD 5 Inf Temporal | 82.4 | Ctx | |
| Ctx | | Control 3 Occipital | 23.7 |
| AD 5 Sup Temporal | 32.3 | Ctx | |
| Ctx | | Control 4 Occipital | 0.0 |
| AD 6 Inf Temporal | 38.4 | Ctx | |
| Ctx | | Control (Path) 1 | 74.2 |
| AD 6 Sup Temporal | 50.7 | Occipital Ctx | |
| Ctx | | Control (Path) 2 | 15.9 |
| Control 1 Temporal | 0.0 | Occipital Ctx | |
| Ctx | | Control (Path) 3 | 0.0 |
| Control 2 Temporal | 10.2 | Occipital Ctx | |
| Ctx | | Control (Path) 4 | 15.2 |
| Control 3 Temporal | 54.7 | Occipital Ctx | |
| Ctx | | Control 1 Parietal | 7.1 |
| Control 3 Temporal | 0.0 | Ctx | |
| Ctx | | Control 2 Parietal | 45.7 |
| Control (Path) 1 | 56.6 | Ctx | |
| Temporal Ctx | | Control 3 Parietal | 17.0 |
| Control (Path) 2 | 36.3 | Ctx | |
| Temporal Ctx | | Control (Path) 1 | 45.4 |
| | | Parietal Ctx | |
| | | Control (Path) 2 | 100.0 |
| | | Parietal Ctx | |
| | | Control (Path) 3 | 6.6 |
| | | Parietal Ctx | |
| | | Control (Path) 4 | 10.3 |
| | | Parietal Ctx | |

TABLE QC
General_screening_panel_v1.4

| | Rel. Exp. | | Rel. Exp. |
|---|---|---|---|
| | (%) | | (%) |
| | Ag3906, | | Ag3906, |
| | Run | | Run |
| Tissue Name | 219168275 | Tissue Name | 219168275 |
| Adipose | 1.6 | Renal ca. TK-10 | 0.8 |
| Melanoma* | 0.0 | Bladder | 1.1 |
| Hs688(A).T | | Gastric ca. (liver met.) | 2.1 |
| Melanoma* | 0.0 | NCI-N87 | |
| Hs688(B).T | | Gastric ca. KATO III | 0.0 |
| Melanoma* M14 | 0.0 | Colon ca. SW-948 | 0.0 |
| Melanoma* | 0.0 | Colon ca. SW480 | 1.0 |
| LOXIMVI | | Colon ca.* (SW480 | 0.0 |
| Melanoma* | 0.0 | met) SW620 | |
| SK-MEL-5 | | Colon ca. HT29 | 0.0 |
| Squamous cell | 1.4 | Colon ca. HCT-116 | 0.0 |
| carcinoma SCC-4 | | Colon ca. CaCo-2 | 0.0 |
| Testis Pool | 4.8 | Colon cancer tissue | 0.0 |
| Prostate ca.* | 0.0 | Colon ca. SW1116 | 0.0 |
| (bone met) PC-3 | | Colon ca. Colo-205 | 0.0 |
| Prostate Pool | 0.0 | Colon ca. SW-48 | 0.0 |
| Placenta | 0.0 | Colon Pool | 0.0 |
| Uterus Pool | 0.0 | Small Intestine Pool | 0.0 |
| Ovarian ca. | 0.0 | Stomach Pool | 2.7 |
| OVCAR-3 | | Bone Marrow Pool | 0.0 |
| Ovarian ca. | 0.0 | Fetal Heart | 0.0 |
| SK-OV-3 | | Heart Pool | 0.0 |
| Ovarian ca. | 0.0 | Lymph Node Pool | 0.0 |
| OVCAR-4 | | Fetal Skeletal Muscle | 0.9 |
| Ovarian ca. | 20.2 | Skeletal Muscle Pool | 28.5 |
| OVCAR-5 | | Spleen Pool | 0.0 |
| Ovarian ca. | 0.0 | Thymus Pool | 1.6 |
| IGROV-1 | | CNS cancer (glio/ | 0.0 |
| Ovarian ca. | 1.8 | astro) U87-MG | |
| OVCAR-8 | | CNS cancer (glio/ | 0.0 |
| Ovary | 0.0 | astro) U-118-MG | |
| Breast ca. MCF-7 | 0.0 | CNS cancer (neuro; | 0.0 |
| Breast ca. MDA- | 0.0 | met) SK-N-AS | |
| MB-231 | | CNS cancer (astro) | 0.0 |
| Breast ca. BT 549 | 0.0 | SF-539 | |
| Breast ca. T47D | 77.4 | CNS cancer (astro) | 0.0 |
| Breast ca. MDA-N | 0.0 | SNB-75 | |
| Breast Pool | 1.3 | CNS cancer (glio) | 0.0 |
| Trachea | 72.2 | SNB-19 | |
| Lung | 0.0 | CNS cancer (glio) | 4.2 |
| Fetal Lung | 100.0 | SF-295 | |
| Lung ca. NCI-N417 | 0.0 | Brain (Amygdala) | 3.0 |
| Lung ca. LX-1 | 1.2 | Pool | |
| Lung ca. NCI-H146 | 0.0 | Brain (cerebellum) | 0.9 |
| Lung ca. SHP-77 | 0.0 | Brain (fetal) | 12.9 |
| Lung ca. A549 | 0.0 | Brain (Hippocampus) | 3.8 |
| Lung ca. NCI-H526 | 0.0 | Pool | |
| Lung ca. NCI-H23 | 3.8 | Cerebral Cortex Pool | 1.9 |
| Lung ca. NCI-H460 | 0.0 | Brain (Substantia | 5.9 |
| Lung ca. HOP-62 | 0.0 | nigra) Pool | |
| Lung ca. NCI-H522 | 1.8 | Brain (Thalamus) Pool | 5.8 |
| Liver | 0.0 | Brain (whole) | 2.9 |
| Fetal Liver | 0.0 | Spinal Cord Pool | 11.7 |
| Liver ca. HepG2 | 0.0 | Adrenal Gland | 0.0 |
| Kidney Pool | 0.0 | Pituitary gland Pool | 0.0 |
| Fetal Kidney | 0.0 | Salivary Gland | 2.2 |
| Renal ca. 786-0 | 0.0 | Thyroid (female) | 0.8 |
| Renal ca. A498 | 0.0 | Pancreatic ca. | 6.7 |
| Renal ca. ACHN | 0.0 | CAPAN2 | |
| Renal ca. UO-31 | 0.0 | Pancreas Pool | 3.4 |

CNS_Neurodegeneration_v1.0 Summary:

Ag3906 Expression of the CG94161-01 gene is low/undetectable (CTs>35) across all of the samples on this panel (data not shown).

General_Screening_Panel_v1.4 Summary:

Ag3906 Highest expression of the CG94161-01 gene is detected in fetal lung (CT=32.3). Similar expression of this gene is also seen in trachea and a breast cancer cell line T47D (Cts=32.7). Therefore expression of this gene can be used to distinguish these samples from other samples used in the panel. Low but significant expression of this gene is also detected in a ovarian cancer cell line. Therefore, therapeutic modulation of this gene product may be useful in treatment of ovarian and breast cancer.

Interestingly, this gene is expressed at much higher levels in fetal (CT=32.3) when compared to adult lung (CT=40). This observation suggests that expression of this gene can be used to distinguish fetal from adult lung. In addition, the relative overexpression of this gene in fetal lung suggests that the protein product may enhance lung growth or development in the fetus and thus may also act in a regenerative capacity in the adult. Therefore, therapeutic modulation of the protein encoded by this gene could be useful in treatment of lung related diseases.

In addition, significant expression is also detected in adult skeletal muscle. Interestingly, this gene is expressed at much higher levels in adult (CT=34) when compared to fetal skeletal muscle (CT=39). Therefore, expression of this gene can be used to distinguish fetal from adult skeletal muscle.

Panel 4.1D Summary:

Ag3906 Expression of the CG94161-01 gene is low/undetectable (CTs>35) across all of the samples on this panel (data not shown).

R. CG94346-01: High Sulfur Keratin

Expression of gene CG94346-01 was assessed using the primer-probe set Ag3914, described in Table RA. Results of the RTQ-PCR runs are shown in Tables RB, and RC.

TABLE RA

Probe Name Ag3914

| | | | | |
|---|---|---|---|---|
| | | | Start | |
| Primers | Sequences | Length | Position | SEQ ID No |

| | | | | |
|---|---|---|---|---|
| Forward | 5′-cttagggccagaactaggaaga-3′ | 22 | 271 | 134 |
| Probe | TET-5′-ctggcttccagagactgaatcagcaa-3′- | 26 | 314 | 135 |
| | TAMRA | | | |
| Reverse | 5′-cacctcggtcttgagaatatga-3′ | 22 | 341 | 136 |

TABLE RB
CNS_neurodegeneration_v1.0

| | Rel. Exp. | | Rel. Exp. |
|---|---|---|---|
| | (%) | | (%) |
| | Ag3914, | | Ag3914, |
| | Run | | Run |
| Tissue Name | 212248457 | Tissue Name | 212248457 |
| AD 1 Hippo | 6.8 | Control (Path) 3 | 4.1 |
| AD 2 Hippo | 57.0 | Temporal Ctx | |
| AD 3 Hippo | 20.6 | Control (Path) 4 | 55.9 |
| AD 4 Hippo | 8.4 | Temporal Ctx | |
| AD 5 hippo | 20.9 | AD 1 Occipital Ctx | 14.6 |
| AD 6 Hippo | 88.9 | AD 2 Occipital Ctx | 0.0 |
| Control 2 Hippo | 31.9 | (Missing) | |
| Control 4 Hippo | 29.7 | AD 3 Occipital Ctx | 0.0 |
| Control (Path) 3 | 11.6 | AD 4 Occipital Ctx | 14.8 |
| Hippo | | AD 5 Occipital Ctx | 14.1 |
| AD 1 Temporal Ctx | 12.5 | AD 6 Occipital Ctx | 68.8 |
| AD 2 Temporal Ctx | 48.3 | Control 1 Occipital | 12.1 |
| AD 3 Temporal Ctx | 0.0 | Ctx | |
| AD 4 Temporal Ctx | 12.1 | Control 2 Occipital | 26.4 |
| AD 5 Inf Temporal | 14.0 | Ctx | |
| Ctx | | Control 3 Occipital | 30.6 |
| AD 5 Sup Temporal | 43.8 | Ctx | |
| Ctx | | Control 4 Occipital | 39.2 |
| AD 6 Inf Temporal | 90.1 | Ctx | |
| Ctx | | Control (Path) 1 | 100.0 |
| AD 6 Sup Temporal | 76.8 | Occipital Ctx | |
| Ctx | | Control (Path) 2 | 0.0 |
| Control 1 Temporal | 23.3 | Occipital Ctx | |
| Ctx | | Control (Path) 3 | 7.5 |
| Control 2 Temporal | 39.2 | Occipital Ctx | |
| Ctx | | Control (Path) 4 | 20.4 |
| Control 3 Temporal | 8.5 | Occipital Ctx | |
| Ctx | | Control 1 Parietal | 8.8 |
| Control 4 Temporal | 17.6 | Ctx | |
| Ctx | | Control 2 Parietal | 39.8 |
| Control (Path) 1 | 82.4 | Ctx | |
| Temporal Ctx | | Control 3 Parietal | 0.0 |
| Control (Path) 2 | 24.7 | Ctx | |
| Temporal Ctx | | Control (Path) 1 | 20.4 |
| | | Parietal Ctx | |
| | | Control (Path) 2 | 13.4 |
| | | Parietal Ctx | |
| | | Control (Path) 3 | 0.0 |
| | | Parietal Ctx | |
| | | Control (Path) 4 | 78.5 |
| | | Parietal Ctx | |

TABLE RC
Panel 4.1D

| | Rel. Exp. | | Rel. Exp. |
|---|---|---|---|
| | (%) | | (%) |
| | Ag3914, | | Ag3914, |
| | Run | | Run |
| Tissue Name | 170701766 | Tissue Name | 170701766 |
| Secondary Th1 act | 0.0 | HUVEC IL-1beta | 1.0 |
| Secondary Th2 act | 2.1 | HUVEC IFN gamma | 0.0 |
| Secondary Tr1 act | 0.0 | HUVEC TNF alpha + | 1.4 |
| Secondary Th1 rest | 0.0 | IFN gamma | |
| Secondary Th2 rest | 0.0 | HUVEC TNF alpha + | 1.4 |
| Secondary Tr1 rest | 0.0 | IL4 | |
| Primary Th1 act | 0.0 | HUVEC IL-11 | 1.4 |
| Primary Th2 act | 0.0 | Lung Microvascular | 3.8 |
| Primary Tr1 act | 0.0 | EC none | |
| Primary Th1 rest | 0.0 | Lung Microvascular | 4.0 |
| Primary Th2 rest | 0.0 | EC TNFalpha + | |
| Primary Tr1 rest | 0.0 | IL-1beta | |
| CD45RA CD4 | 0.0 | Microvascular Dermal | 1.9 |
| lymphocyte act | | EC none | |
| CD45RO CD4 | 0.0 | Microvascular Dermal | 0.0 |
| lymphocyte act | | EC TNFalpha + | |
| CD8 lymphocyte act | 0.0 | IL-1beta | |
| Secondary CD8 | 0.0 | Bronchical epithelium | 1.9 |
| lymphocyte rest | | TNFalpha + IL1beta | |
| Secondary CD8 | 0.0 | Small airway | 0.0 |
| lymphocyte act | | epithelium none | |
| CD4 lymphocyte | 0.0 | Small airway | 3.6 |
| none | | epithelium | |
| 2ry Th1/Th2/Tr1_{—} | 1.4 | TNFalpha + IL-1beta | |
| anti CD95 CH11 | | Coronery artery | 0.9 |
| LAK cells rest | 1.0 | SMC rest | |
| LAK cells IL-2 | 0.7 | Coronery artery SMC | 0.0 |
| LAK cells IL-2 + | 0.0 | TNFalpha + IL-1beta | |
| IL-12 | | Astrocytes rest | 3.0 |
| LAK cells IL-2 + | 1.1 | Astrocytes | 1.5 |
| IFN gamma | | TNFalpha + IL-1beta | |
| LAK cells IL-2 + | 0.0 | KU-812 (Basophil) | 0.6 |
| IL-18 | | rest | |
| LAK cells | 3.8 | KU-812 (Basophil) | 0.7 |
| PMA/ionomycin | | PMA/ionomycin | |
| NK Cells IL-2 rest | 0.0 | CCD1106 | 2.8 |
| Two Way MLR 3 | 1.0 | (Keratinocytes) none | |
| day | | CCD1106 | 2.0 |
| Two Way MLR 5 | 0.0 | (Keratinocytes) | |
| day | | TNFalpha + IL-1beta | |
| Two Way MLR 7 | 1.7 | Liver cirrhosis | 0.0 |
| day | | NCI-H292 none | 3.2 |
| PBMC rest | 0.0 | NCI-H292 IL-4 | 4.5 |
| PBMC PWM | 2.1 | NCI-H292 IL-9 | 4.2 |
| PBMC PHA-L | 1.6 | NCI-H292 IL-13 | 5.0 |
| Ramos (B cell) none | 17.4 | NCI-H292 IFN gamma | 2.3 |
| Ramos (B cell) | 12.2 | HPAEC none | 0.0 |
| ionomycin | | HPAEC TNF alpha + | 0.0 |
| B lymphocytes | 0.0 | IL-1 beta | |
| PWM | | Lung fibroblast none | 2.5 |
| B lymphocytes | 8.7 | Lung fibroblast | 0.9 |
| CD40L and IL-4 | | TNF alpha + IL-1 beta | |
| EOL-1 dbcAMP | 0.0 | Lung fibroblast IL-4 | 1.1 |
| EOL-1 dbcAMP | 3.0 | Lung fibroblast IL-9 | 2.3 |
| PMA/ionomycin | | Lung fibroblast IL-13 | 7.1 |
| Dendritic cells none | 1.1 | Lung fibroblast IFN | 1.4 |
| Dendritic cells LPS | 0.0 | gamma | |
| Dendritic cells | 0.0 | Dermal fibroblast | 2.0 |
| anti-CD40 | | CCD1070 rest | |
| Monocytes rest | 1.3 | Dermal fibroblast | 8.2 |
| Monocytes LPS | 1.8 | CCD1070 TNF alpha | |
| Macrophages rest | 0.0 | Dermal fibroblast | 2.7 |
| Macrophages LPS | 0.0 | CCD1070 IL-1 beta | |
| HUVEC none | 0.0 | Dermal fibroblast | 1.0 |
| HUVEC starved | 1.0 | IFN gamma | |
| | | Dermal fibroblast IL-4 | 4.5 |
| | | Dermal fibroblast rest | 0.0 |
| | | Neutrophilis TNFa + | 1.3 |
| | | LPS | |
| | | Neutrophils rest | 0.0 |
| | | Colon | 5.0 |
| | | Lung | 17.8 |
| | | Thymus | 27.5 |
| | | Kidney | 100.0 |

CNS_Neurodegeneration_v1.0 Summary:

Ag3914 This panel does not show differential expression of the CG94346-01 gene in Alzheimer's disease. However, this expression profile shows that this gene is expressed at low levels in the CNS. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

General_Screening_Panel_v1.4 Summary:

Ag3914 Expression of the CG94346-01 gene is low/undetectable in all samples on this panel (CTs>35). (Data not shown.)

Panel 4.1D Summary:

Ag3914 Expression of the CG94346-01 gene is highest in the kidney (CT=30.5). Low levels of expression are also seen in the B cell line Ramos (treated and non-treated), B lymphocytes treated with CD40L and IL-4, IL-13 treated lung fibroblasts and NCI-H292 cells, TNF-alpha activated dermal fibroblasts and lung and thymus. Expression of this gene in the kidney and other cells involved in the immune response suggests that this gene product may be involved in the homeostasis of this organ. Therapeutic modulation of the expression or function of this gene product may be useful in restoring or maintaining function of the kidney during inflammation and in the treatment of asthma, allergies, chronic obstructive pulmonary disease, emphysema, Crohn's disease, ulcerative colitis, rheumatoid arthritis, psoriasis, osteoarthritis, systemic lupus erythematosus and other autoimmune disorders.

S. CG94600-01: Ring Finger-Like Protein

Expression of gene CG94600-01 was assessed using the primer-probe set Ag5869, described in Table SA. Results of the RTQ-PCR runs are shown in Tables SB, SC and SD.

TABLE SA

Probe Name Ag5869

| | | | | |
|---|---|---|---|---|
| | | | | SEQ ID |
| Primers | Sequences | Length | Start Position | No |

| | | | | |
|---|---|---|---|---|
| Forward | 5′-atgcagactgttagataaactttggta-3′ | 27 | 1358 | 137 |
| Probe | TET-5′-tggttttctgaagcctctctatctgtt-3′- | 27 | 1331 | 138 |
| Reverse | 5′-tttcaaccaacacatcataacct-3′ | 23 | 1285 | 139 |

TABLE SB
CNS_neurodegeneration_v1.0

| | Rel. Exp. | | Rel. Exp. |
|---|---|---|---|
| | (%) | | (%) |
| | Ag5869, | | Ag5869, |
| | Run | | Run |
| Tissue Name | 248162678 | Tissue Name | 248162678 |
| AD 1 Hippo | 1.7 | Control (Path) 3 | 0.7 |
| AD 2 Hippo | 20.3 | Temporal Ctx | |
| AD 3 Hippo | 2.2 | Control (Path) 4 | 5.8 |
| AD 4 Hippo | 4.1 | Temporal Ctx | |
| AD 5 Hippo | 23.0 | AD 1 Occipital Ctx | 16.5 |
| AD 6 Hippo | 68.3 | AD 2 Occipital Ctx | 0.0 |
| Control 2 Hippo | 21.6 | (Missing) | |
| Control 4 Hippo | 3.7 | AD 3 Occipital Ctx | 4.7 |
| Control (Path) 3 | 1.7 | AD 4 Occipital Ctx | 9.7 |
| Hippo | | AD 5 Occipital Ctx | 15.7 |
| AD 1 Temporal Ctx | 22.4 | AD 6 Occipital Ctx | 9.9 |
| AD 2 Temporal Ctx | 20.9 | Control 1 Occipital | 3.6 |
| AD 3 Temporal Ctx | 3.0 | Ctx | |
| AD 4 Temporal Ctx | 2.0 | Control 2 Occipital | 22.1 |
| AD 5 Inf Temporal | 95.9 | Ctx | |
| Ctx | | Control 3 Occipital | 12.8 |
| AD 5 Sup Temporal | 56.3 | Ctx | |
| Ctx | | Control 4 Occipital | 5.8 |
| AD 6 Inf Temporal | 87.7 | Ctx | |
| Ctx | | Control (Path) 1 | 100.0 |
| AD 6 Sup Temporal | 24.7 | Occipital Ctx | |
| Ctx | | Control (Path) 2 | 0.7 |
| Control 1 Temporal | 2.5 | Occipital Ctx | |
| Ctx | | Control (Path) 3 | 2.2 |
| Control 2 Temporal | 20.2 | Occipital Ctx | |
| Ctx | | Control (Path) 4 | 7.9 |
| Control 3 Temporal | 6.1 | Occipital Ctx | |
| Ctx | | Control 1 Parietal | 3.5 |
| Control 3 Temporal | 3.0 | Ctx | |
| Ctx | | Control 2 Parietal | 18.6 |
| Control (Path) 1 | 20.9 | Ctx | |
| Temporal Ctx | | Control 3 Parietal | 8.0 |
| Control (Path) 2 | 5.3 | Ctx | |
| Temporal Ctx | | Control (Path) 1 | 38.7 |
| | | Parietal Ctx | |
| | | Control (Path) 2 | 3.1 |
| | | Parietal Ctx | |
| | | Control (Path) 3 | 0.0 |
| | | Parietal Ctx | |
| | | Control (Path) 4 | 20.3 |
| | | Parietal Ctx | |

TABLE SC
General_screening_panel_v1.5

| | Rel. Exp. | | Rel. Exp. |
|---|---|---|---|
| | (%) | | (%) |
| | Ag5869, | | Ag5869, |
| | Run | | Run |
| Tissue Name | 247945097 | Tissue Name | 247945097 |
| Adipose | 0.9 | Renal ca. TK-10 | 2.6 |
| Melanoma* | 59.0 | Bladder | 17.6 |
| Hs688(A).T | | Gastric ca. (liver met.) | 33.0 |
| Melanoma* | 53.2 | NCI-N87 | |
| Hs688(B).T | | Gastric ca. KATO III | 69.3 |
| Melanoma* M14 | 10.7 | Colon ca. SW-948 | 9.7 |
| Melanoma* | 31.6 | Colon ca. SW480 | 48.0 |
| LOXIMVI | | Colon ca.* (SW480 | 13.3 |
| Melanoma* | 16.6 | met) SW620 | |
| SK-MEL-5 | | Colon ca. HT29 | 10.8 |
| Squamous cell | 5.7 | Colon ca. HCT-116 | 100.0 |
| carcinoma SCC-4 | | Colon ca. CaCo-2 | 5.1 |
| Testis Pool | 3.0 | Colon cancer tissue | 6.4 |
| Prostate ca.* | 30.1 | Colon ca. SW1116 | 3.0 |
| (bone met) PC-3 | | Colon ca. Colo-205 | 7.6 |
| Prostate Pool | 2.3 | Colon ca. SW-48 | 6.5 |
| Placenta | 0.1 | Colon Pool | 5.6 |
| Uterus Pool | 0.6 | Small Intestine Pool | 2.9 |
| Ovarian ca. | 47.3 | Stomach Pool | 2.5 |
| OVCAR-3 | | Bone Marrow Pool | 1.5 |
| Ovarian ca. | 92.0 | Fetal Heart | 4.1 |
| SK-OV-3 | | Heart Pool | 1.0 |
| Ovarian ca. | 5.1 | Lymph Node Pool | 4.2 |
| OVCAR-4 | | Fetal Skeletal Muscle | 1.6 |
| Ovarian ca. | 38.7 | Skeletal Muscle Pool | 0.6 |
| OVCAR-5 | | Spleen Pool | 4.8 |
| Ovarian ca. | 9.2 | Thymus Pool | 4.1 |
| IGROV-1 | | CNS cancer (glio/ | 5.4 |
| Ovarian ca. | 21.3 | astro) U87-MG | |
| OVCAR-8 | | CNS cancer (glio/ | 12.6 |
| Ovary | 1.9 | astro) U-118-MG | |
| Breast ca. MCF-7 | 44.8 | CNS cancer (neuro; | 9.9 |
| Breast ca. MDA- | 27.5 | met) SK-N-AS | |
| MB-231 | | CNS cancer (astro) | 18.7 |
| Breast ca. BT 549 | 2.5 | SF-539 | |
| Breast ca. T47D | 12.5 | CNS cancer (astro) | 7.9 |
| Breast ca. MDA-N | 2.1 | SNB-75 | |
| Breast Pool | 4.0 | CNS cancer (glio) | 7.1 |
| Trachea | 1.9 | SNB-19 | |
| Lung | 0.5 | CNS cancer (glio) | 15.9 |
| Fetal Lung | 6.8 | SF-295 | |
| Lung ca. NCI-N417 | 0.1 | Brain (Amygdala) | 12.6 |
| Lung ca. LX-1 | 21.6 | Pool | |
| Lung ca. NCI-H146 | 1.5 | Brain (cerebellum) | 1.4 |
| Lung ca. SHP-77 | 0.8 | Brain (fetal) | 2.9 |
| Lung ca. A549 | 26.6 | Brain (Hippocampus) | 4.1 |
| Lung ca. NCI-H526 | 5.4 | Pool | |
| Lung ca. NCI-H23 | 18.4 | Cerebral Cortex Pool | 2.0 |
| Lung ca. NCI-H460 | 8.1 | Brain (Substantia | 1.6 |
| Lung ca. HOP-62 | 6.7 | nigra) Pool | |
| Lung ca. NCI-H522 | 24.7 | Brain (Thalamus) Pool | 3.1 |
| Liver | 0.1 | Brain (whole) | 0.7 |
| Fetal Liver | 48.3 | Spinal Cord Pool | 7.3 |
| Liver ca. HepG2 | 1.8 | Adrenal Gland | 0.3 |
| Kidney Pool | 5.6 | Pituitary gland Pool | 0.2 |
| Fetal Kidney | 11.0 | Salivary Gland | 0.5 |
| Renal ca. 786-0 | 44.1 | Thyroid (female) | 1.8 |
| Renal ca. A498 | 3.1 | Pancreatic ca. | 62.4 |
| Renal ca. ACHN | 37.9 | CAPAN2 | |
| Renal ca. UO-31 | 36.9 | Pancreas Pool | 4.4 |

TABLE SD
Panel 4.1D

| | Rel. Exp. | | Rel. Exp. |
|---|---|---|---|
| | (%) | | (%) |
| | Ag5869, | | Ag5869, |
| | Run | | Run |
| Tissue Name | 247683517 | Tissue Name | 247683517 |
| Secondary Th1 act | 25.3 | HUVEC IL-1beta | 19.8 |
| Secondary Th2 act | 42.0 | HUVEC IFN gamma | 15.2 |
| Secondary Tr1 act | 11.7 | HUVEC TNF alpha + | 5.0 |
| Secondary Th1 rest | 2.4 | IFN gamma | |
| Secondary Th2 rest | 3.4 | HUVEC TNF alpha + | 7.2 |
| Secondary Tr1 rest | 2.3 | IL4 | |
| Primary Th1 act | 3.8 | HUVEC IL-11 | 7.6 |
| Primary Th2 act | 14.7 | Lung Microvascular | 11.8 |
| Primary Tr1 act | 22.2 | EC none | |
| Primary Th1 rest | 1.2 | Lung Microvascular | 2.5 |
| Primary Th2 rest | 5.0 | EC TNFalpha + | |
| Primary Tr1 rest | 1.0 | IL-1beta | |
| CD45RA CD4 | 37.1 | Microvascular Dermal | 4.2 |
| lymphocyte act | | EC none | |
| CD45RO CD4 | 40.9 | Microvascular Dermal | 3.4 |
| lymphocyte act | | EC TNFalpha + | |
| CD8 lymphocyte act | 14.3 | IL-1beta | |
| Secondary CD8 | 12.3 | Bronchical epithelium | 0.4 |
| lymphocyte rest | | TNFalpha + IL1beta | |
| Secondary CD8 | 4.5 | Small airway | 1.2 |
| lymphocyte act | | epithelium none | |
| CD4 lymphocyte | 1.5 | Small airway | 4.4 |
| none | | epithelium | |
| 2ry Th1/Th2/Tr1_{—} | 9.1 | TNFalpha + IL-1beta | |
| anti CD95 CH11 | | Coronery artery | 1.4 |
| LAK cells rest | 3.3 | SMC rest | |
| LAK cells IL-2 | 14.7 | Coronery artery SMC | 2.8 |
| LAK cells IL-2 + | 3.1 | TNFalpha + IL-1beta | |
| IL-12 | | Astrocytes rest | 2.0 |
| LAK cells IL-2 + | 3.3 | Astrocytes | 0.7 |
| IFN gamma | | TNFalpha + IL-1beta | |
| LAK cells IL-2 + | 6.1 | KU-812 (Basophil) | 3.0 |
| IL-18 | | rest | |
| LAK cells | 3.8 | KU-812 (Basophil) | 6.9 |
| PMA/ionomycin | | PMA/ionomycin | |
| NK Cells IL-2 rest | 31.6 | CCD1106 | 18.8 |
| Two Way MLR 3 | 1.0 | (Keratinocytes) none | |
| day | | CCD1106 | 4.7 |
| Two Way MLR 5 | 1.7 | (Keratinocytes) | |
| day | | TNFalpha + IL-1beta | |
| Two Way MLR 7 | 3.1 | Liver cirrhosis | 0.0 |
| day | | NCI-H292 none | 4.8 |
| PBMC rest | 0.9 | NCI-H292 IL-4 | 6.7 |
| PBMC PWM | 3.5 | NCI-H292 IL-9 | 17.6 |
| PBMC PHA-L | 3.3 | NCI-H292 IL-13 | 15.4 |
| Ramos (B cell) none | 15.3 | NCI-H292 IFN gamma | 9.4 |
| Ramos (B cell) | 25.2 | HPAEC none | 6.1 |
| ionomycin | | HPAEC TNF alpha + | 11.0 |
| B lymphocytes | 9.2 | IL-1 beta | |
| PWM | | Lung fibroblast none | 5.1 |
| B lymphocytes | 16.6 | Lung fibroblast | 2.4 |
| CD40L and IL-4 | | TNF alpha + IL-1 beta | |
| EOL-1 dbcAMP | 3.4 | Lung fibroblast IL-4 | 1.6 |
| EOL-1 dbcAMP | 1.0 | Lung fibroblast IL-9 | 3.3 |
| PMA/ionomycin | | Lung fibroblast IL-13 | 0.6 |
| Dendritic cells none | 0.9 | Lung fibroblast IFN | 2.5 |
| Dendritic cells LPS | 0.2 | gamma | |
| Dendritic cells | 0.0 | Dermal fibroblast | 33.2 |
| anti-CD40 | | CCD1070 rest | |
| Monocytes rest | 0.3 | Dermal fibroblast | 100.0 |
| Monocytes LPS | 0.7 | CCD1070 TNF alpha | |
| Macrophages rest | 0.5 | Dermal fibroblast | 32.1 |
| Macrophages LPS | 0.3 | CCD1070 IL-1 beta | |
| HUVEC none | 8.9 | Dermal fibroblast | 10.8 |
| HUVEC starved | 20.6 | IFN gamma | |
| | | Dermal fibroblast IL-4 | 11.1 |
| | | Dermal fibroblast rest | 9.0 |
| | | Neutrophilis TNFa + | 0.0 |
| | | LPS | |
| | | Neutrophils rest | 0.6 |
| | | Colon | 0.0 |
| | | Lung | 0.0 |
| | | Thymus | 2.1 |
| | | Kidney | 0.5 |

CNS_Neurodegeneration_v1.0 Summary:

Ag5869 This panel does not show differential expression of the CG94600-01 gene in Alzheimer's disease. However, this expression profile confirms the presence of this gene in the brain. Please see Panel 1.5 for discussion of utility of this gene in the central nervous system.

General_Screening_Panel_v1.5 Summary:

Ag5869 The CG94600-01 gene is widely expressed in this panel, with highest expression in a colon cancer cell line (CT=29.1). Significant levels of expression are also seen in samples derived from pancreatic, gastric, lung, breast, ovarian, melanoma, and renal cancers. Thus, expression of this gene could be used to differentiate between the colon cancer sample and other samples on this panel and as a marker to detect the presence of these cancers. The CG94600-01 gene codes for a ring finger protein similar to Ret finger protein 2. Ret finger protein is a member of the B-box zinc finger gene family many of which may function in growth regulation and in the appropriate context become oncogenic (Ref.1). Therefore, therapeutic modulation of the expression or function of the CG94600-01 gene may be effective in the treatment of pancreatic, gastric, lung, colon, breast, ovarian, melanoma, and renal cancers.

Among tissues with metabolic function, this gene is expressed at low but significant levels in pancreas, thyroid, and fetal heart and liver. This expression among these tissues suggests that this gene product may play a role in normal neuroendocrine and metabolic and that disregulated expression of this gene may contribute to neuroendocrine disorders or metabolic diseases, such as obesity and diabetes.

This gene is also expressed at low levels in the CNS, including the thalamus, amygdala, and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

In addition, this gene is expressed at much higher levels in fetal liver tissue (CT=30) when compared to expression in the adult counterpart (CT=39.5). Thus, expression of this gene may be used to differentiate between the fetal and adult source of this tissue. (Cao T, Duprez E, Borden K L, Freemont P S, Etkin L D. (1998) Ret finger protein is a normal component of PML nuclear bodies and interacts directly with PML. J Cell Sci 111 (Pt 10): 1319-29).

Panel 4.1D Summary:

Ag5869 The CG94600-01 gene is widely expressed in this panel, with highest expression in TNF alpha treated dermal fibroblasts (CT=29.6). Thus, that this gene product may be involved in skin disorders, including psoriasis. Low but significant levels of expression are also seen in activated T and B cells. Non-activated CD4 cells do not express the transcript, however T cells induced with specific activators (CD3/CD28 regardless of the presence of polarizing cytokines) (i.e. CD45RA/CD45RO) or mitogens such as phytohemaglutinin (PHA) express the transcript. Likewise, no expression of the transcript is seen in PBMC that contain normal B cells, but the transcript is induced when PBMC are treated with the B cell selective pokeweed mitogen. In addition, the transcript is seen in the B cell lymphoma Ramos regardless of stimulation. Therefore, the putative protein encoded by this gene could potentially be used diagnostically to identify activated B or T cells. In addition, the gene product could also potentially be used therapeutically in the treatment of asthma, emphysema, IBD, lupus or arthritis and in other diseases in which T cells and B cells are activated.

T. CG94820-02: Probable Cation-Transporting ATPase

Expression of gene CG94820-02 was assessed using the primer-probe sets Ag1417, Ag3604 and Ag3956, described in Tables TA, TB and TC. Results of the RTQ-PCR runs are shown in Tables TD, TE, TF and TG.

TABLE TA

Probe Name Ag1417

| | | | | |
|---|---|---|---|---|
| | | | Start | |
| Primers | Sequences | Length | Position | SEQ ID No |

| | | | | |
|---|---|---|---|---|
| Forward | 5′-ataggaaaatggacgcctacat-3′ | 22 | 1276 | 140 |
| Probe | TET-5′-ccattgccggtctctgtaaaacctgaa-3′- | 26 | 1315 | 141 |
| | TAMRA | | | |
| Reverse | 5′-ttttgaaaatcgacaggaactg | 22 | 1342 | 142 |

TABLE TB

Probe Name Ag3604

| | | | | |
|---|---|---|---|---|
| | | | Start | |
| Primers | Sequences | Length | Position | SEQ ID No |

| | | | | |
|---|---|---|---|---|
| Forward | 5′-gcaattgagaacaacatggatt-3′ | 22 | 1470 | 143 |
| Probe | TET-5′-caaattaaagcaagaaacccctgcag-3′- | 26 | 1517 | 144 |
| | TAMRA | | | |
| Reverse | 5′-tgttggctttatgcaaatcttc-3′ | 22 | 1548 | 145 |

TABLE TC

Probe Name Ag3956

| | | | | |
|---|---|---|---|---|
| | | | Start | SEQ ID |
| Primers | Sequences | Length | Position | No |

| | | | | |
|---|---|---|---|---|
| Forward | 5′-cagcttgttcgttccatattgt-3′ | 22 | 531 | 146 |
| Probe | TET-5′-tcccaaaccaactgattttaaactctaca-3′- | 29 | 554 | 147 |
| | TAMRA | | | |
| Reverse | 5′-agcaactgccacaagacatagt-3′ | 22 | 602 | 69 |

TABLE TD
CNS_neurodegeneration_v1.0

| | Rel. Exp. (%) | Rel. Exp. (%) | | Rel. Exp. (%) | Rel. Exp. (%) |
|---|---|---|---|---|---|
| | Ag3604, Run | Ag3956, Run | Tissue | Ag3604, Run | Ag3956, Run |
| Tissue Name | 210997046 | 212347080 | Name | 210997046 | 212347080 |
| AD 1 Hippo | 8.8 | 9.9 | Control | 9.1 | 7.4 |
| | | | (Path) 3 | | |
| | | | Temporal | | |
| | | | Ctx | | |
| AD 2 Hippo | 26.8 | 25.0 | Control | 39.8 | 25.0 |
| | | | (path) 4 | | |
| | | | Temporal | | |
| | | | Ctx | | |
| AD 3 Hippo | 7.3 | 6.6 | AD 1 | 14.3 | 9.5 |
| | | | Occipital | | |
| | | | Ctx | | |
| AD 4 Hippo | 10.7 | 4.5 | AD 2 | 0.0 | 0.0 |
| | | | Occipital | | |
| | | | Ctx | | |
| | | | (Missing) | | |
| AD 5 hippo | 97.9 | 52.5 | AD 3 | 5.0 | 4.3 |
| | | | Occipital | | |
| | | | Ctx | | |
| AD 6 Hippo | 87.7 | 74.7 | AD 4 | 23.3 | 15.6 |
| | | | Occipital | | |
| | | | Ctx | | |
| Control 2 Hippo | 28.9 | 16.4 | AD 5 | 47.3 | 43.5 |
| | | | Occipital | | |
| | | | Ctx | | |
| Control 4 Hippo | 18.9 | 13.8 | AD 6 | 48.6 | 56.3 |
| | | | Occipital | | |
| | | | Ctx | | |
| Control (Path) 3 | 11.3 | 8.8 | Control 1 | 5.8 | 9.3 |
| Hippo | | | Occipital | | |
| | | | Ctx | | |
| AD 1 Temporal | 15.5 | 14.2 | Control 2 | 74.7 | 70.2 |
| Ctx | | | Occipital | | |
| | | | Ctx | | |
| AD 2 Temporal | 35.4 | 33.9 | Control 3 | 26.6 | 9.5 |
| Ctx | | | Occipital | | |
| | | | Ctx | | |
| AD 3 Temporal | 6.0 | 4.0 | Control 4 | 6.8 | 8.0 |
| Ctx | | | Occipital | | |
| | | | Ctx | | |
| AD 4 Temporal | 23.8 | 21.3 | Control | 100.0 | 82.9 |
| Ctx | | | (Path) 1 | | |
| | | | Occipital Ctx | | |
| AD 5 Inf | 94.0 | 100.0 | Control | 13.9 | 7.9 |
| Temporal Ctx | | | (Path) 2 | | |
| | | | Occipital | | |
| | | | Ctx | | |
| AD 5 | 55.1 | 52.9 | Control | 5.0 | 6.2 |
| SupTemporal | | | (Path) 3 | | |
| Ctx | | | Occipital | | |
| | | | Ctx | | |
| AD 6 Inf | 65.5 | 69.7 | Control | 30.8 | 11.0 |
| Temporal Ctx | | | (Path) 4 | | |
| | | | Occipital | | |
| | | | Ctx | | |
| AD 6 Sup | 66.0 | 57.0 | Control 1 | 10.7 | 6.7 |
| Temporal Ctx | | | Parietal Ctx | | |
| Control 1 | 9.3 | 7.1 | Control 2 | 46.7 | 32.3 |
| Temporal Ctx | | | Parietal Ctx | | |
| Control 2 | 42.3 | 40.1 | Control 3 | 16.5 | 15.7 |
| Temporal Ctx | | | Parietal Ctx | | |
| Control 3 | 15.6 | 13.0 | Control | 88.9 | 73.7 |
| Temporal Ctx | | | (Path) 1 | | |
| | | | Parietal Ctx | | |
| Control 4 | 12.8 | 8.0 | Control | 25.7 | 25.7 |
| Temporal Ctx | | | (Path) 2 | | |
| | | | Parietal Ctx | | |
| Control (Path) 1 | 52.9 | 58.6 | Control | 6.3 | 7.1 |
| Temporal Ctx | | | (Path) 3 | | |
| | | | Parietal Ctx | | |
| Control (Path) 2 | 48.3 | 29.3 | Control | 52.5 | 34.6 |
| Temporal Ctx | | | (Path) 4 | | |
| | | | Parietal Ctx | | |

TABLE TE
General_screening_panel_v1.4

| | Rel. Exp. (%) | Rel. Exp. (%) | | Rel. Exp. (%) | Rel. Exp. (%) |
|---|---|---|---|---|---|
| | Ag3604, Run | Ag3956, Run | | Ag3604, Run | Ag3956, Run |
| Tissue Name | 217674539 | 213856332 | Tissue Name | 217674539 | 213856332 |
| Adipose | 5.6 | 9.2 | Renal ca. TK-10 | 17.9 | 28.5 |
| Melanoma* | 17.9 | 29.1 | Bladder | 10.9 | 14.4 |
| Hs688(A).T | | | | | |
| Melanoma* | 24.0 | 37.1 | Gastric ca. (liver | 17.0 | 22.4 |
| Hs688(B).T | | | met.) NCI-N87 | | |
| Melanoma* | 12.3 | 21.9 | Gastric ca. KATO | 38.7 | 55.9 |
| M14 | | | III | | |
| Melanoma* | 13.4 | 22.1 | Colon ca. SW- | 4.4 | 6.9 |
| LOXIMVI | | | 948 | | |
| Melanoma* | 17.8 | 24.1 | Colon ca. SW480 | 31.9 | 46.3 |
| SK-MEL-5 | | | | | |
| Squamous cell | 11.9 | 21.0 | Colon ca.* | 17.0 | 25.3 |
| carcinoma | | | (SW480 met) | | |
| SCC-4 | | | SW620 | | |
| Testis Pool | 1.3 | 2.1 | Colon ca. HT29 | 9.1 | 14.1 |
| Prostate ca.* | 15.5 | 22.8 | Colon ca. HCT- | 27.9 | 45.1 |
| (bone met) | | | 116 | | |
| PC-3 | | | | | |
| Prostate Pool | 1.4 | 2.1 | Colon ca.CaCo-2 | 14.8 | 22.8 |
| Placenta | 0.9 | 1.0 | Colon cancer | 10.2 | 13.6 |
| | | | tissue | | |
| Uterus Pool | 1.4 | 3.2 | Colon ca. | 1.5 | 1.7 |
| | | | SW1116 | | |
| Ovarian ca. | 12.4 | 20.9 | Colon ca. Colo- | 4.1 | 6.7 |
| OVCAR-3 | | | 205 | | |
| Ovarian ca. | 24.3 | 35.6 | Colon ca. SW-48 | 5.8 | 4.3 |
| SK-OV-3 | | | | | |
| Ovarian ca. | 10.8 | 17.7 | Colon Pool | 4.0 | 7.7 |
| OVCAR-4 | | | | | |
| Ovarian ca | 50.3 | 52.1 | Small Intestine | 2.5 | 4.3 |
| OVCAR-5 | | | Pool | | |
| Ovarian ca. | 9.0 | 11.4 | Stomach Pool | 3.0 | 5.2 |
| IGROV-1 | | | | | |
| Ovarian ca | 5.4 | 5.8 | Bone Marrow | 1.2 | 2.7 |
| OVCAR-8 | | | Pool | | |
| Ovary | 2.1 | 4.9 | Fetal Heart | 5.6 | 7.3 |
| Breast ca. | 12.0 | 16.2 | Heart Pool | 2.1 | 2.8 |
| MCF-7 | | | | | |
| Breast ca. | 15.3 | 23.2 | Lymph Node | 4.7 | 7.5 |
| MDA-MB- | | | Pool | | |
| 231 | | | | | |
| Breast ca. BT | 9.2 | 14.7 | Fetal Skeletal | 0.6 | 1.0 |
| 549 | | | Muscle | | |
| Breast ca. | 100.0 | 100.0 | Skeletal Muscle | 1.7 | 2.4 |
| T47D | | | Pool | | |
| Breast ca. | 15.2 | 16.6 | Spleen Pool | 4.8 | 4.8 |
| MDA-N | | | | | |
| Breast Pool | 3.9 | 7.9 | Thymus Pool | 2.9 | 5.4 |
| Trachea | 3.0 | 6.4 | CNS cancer | 84.7 | 98.6 |
| | | | (glio/astro) U87- | | |
| | | | MG | | |
| Lung | 0.5 | 0.8 | CNS cancer | 30.8 | 51.4 |
| | | | (glio/astro)U- | | |
| | | | 118-MG | | |
| Fetal Lung | 8.0 | 10.6 | CNS cancer | 14.5 | 22.1 |
| | | | (neuro;met) SK- | | |
| | | | N-AS | | |
| Lung ca. NCI- | 1.5 | 1.9 | CNS cancer | 13.1 | 18.6 |
| N417 | | | (astro) SF-539 | | |
| Lung ca. LX-1 | 10.9 | 15.3 | CNS cancer | 39.8 | 50.0 |
| | | | (astro) SNB-75 | | |
| Lung ca. NCI- | 11.7 | 20.0 | CNS cancer (glio) | 9.8 | 9.5 |
| H146 | | | SNB-19 | | |
| Lung ca. SHP- | 5.3 | 8.1 | CNS cancer (glio) | 30.6 | 43.8 |
| 77 | | | SF-295 | | |
| Lung ca. A549 | 9.6 | 15.3 | Brain (Amygdala) | 1.9 | 2.7 |
| | | | Pool | | |
| Lung ca. NCI- | 4.5 | 5.3 | Brain | 1.4 | 1.8 |
| H526 | | | (cerebellum) | | |
| Lung ca. NCI- | 25.7 | 40.6 | Brain (fetal) | 4.4 | 7.4 |
| H23 | | | | | |
| Lung ca. NCI- | 5.9 | 7.2 | Brain | 2.1 | 2.9 |
| H460 | | | (Hippocampus) | | |
| | | | Pool | | |
| Lung ca. | 5.8 | 7.0 | Cerebral Cortex | 2.7 | 3.8 |
| HOP-62 | | | Pool | | |
| Lung ca. NCI- | 8.8 | 13.3 | Brain (Substantia | 1.9 | 2.4 |
| H522 | | | nigra) Pool | | |
| Liver | 0.6 | 0.9 | Brain (Thalamus) | 2.8 | 3.8 |
| | | | Pool | | |
| Fetal Liver | 11.1 | 14.5 | Brain (whole) | 2.4 | 3.4 |
| Liver ca. | 6.2 | 10.5 | Spinal Cord Pool | 1.9 | 2.1 |
| HepG2 | | | | | |
| Kidney Pool | 5.2 | 10.8 | Adrenal Gland | 2.5 | 3.8 |
| Fetal Kidney | 4.2 | 6.4 | Pituitary gland | 0.7 | 0.9 |
| | | | Pool | | |
| Renal ca. 786-0 | 44.1 | 56.3 | Salivary Gland | 0.8 | 1.1 |
| Renal ca. | 10.2 | 13.3 | Thyroid (female) | 5.0 | 7.5 |
| A498 | | | | | |
| Renal ca. | 6.4 | 11.4 | Pancreatic ca. | 12.0 | 18.4 |
| ACHN | | | CAPAN2 | | |
| Renal ca. UO-31 | 37.9 | 49.0 | Pancreas Pool | 5.6 | 7.8 |

TABLE TF
Panel 2.1

| | Rel. Exp. | | Rel. Exp. |
|---|---|---|---|
| | (%) | | (%) |
| | Ag3956, | | Ag3956, |
| | Run | | Run |
| Tissue Name | 170720927 | Tissue Name | 170720927 |
| Normal Colon | 18.2 | Kidney Cancer | 9.6 |
| Colon caner | 30.4 | 9010320 | |
| (OD06064) | | Kidney margin | 43.2 |
| Colon cancer margin | 14.0 | 9010321 | |
| (OD06064) | | Kidney Cancer | 4.5 |
| Colon cancer | 4.8 | 8120607 | |
| (OD06159) | | Kidney margin | 3.4 |
| Colon cancer margin | 5.8 | 8120608 | |
| (OD06159) | | Normal Uterus | 31.9 |
| Colon cancer | 6.7 | Uterus Cancer | 18.0 |
| (OD06298-08) | | Normal Thyroid | 2.5 |
| Colon cancer margin | 5.6 | Thyroid Cancer | 19.2 |
| (OD06298-018) | | Thyroid Cancer | 6.7 |
| Colon Cancer Gr.2 | 11.2 | A302152 | |
| ascend colon | | Thyroid margin | 22.7 |
| (ODO3921) | | A302153 | |
| Colon Cancer | 12.3 | Normal Breast | 25.7 |
| margin (ODO3921) | | Breast Cancer | 0.0 |
| Colon cancer | 12.9 | Breast Cancer | 2.2 |
| metastasis | | Breast Cancer | 0.0 |
| (OD06104) | | (OD04590-01) | |
| Lung margin | 34.4 | Breast Cancer Mets | 13.7 |
| (OD06104) | | (OD04590-03) | |
| Colon mets to lung | 7.3 | Breast Cancer | 39.2 |
| (OD04451-01) | | Metastasis | |
| Lung margin | 18.3 | Breast Cancer | 2.1 |
| (OD04451-02) | | Breast Cancer | 6.5 |
| Normal Prostate | 0.6 | 9100266 | |
| Prostate Cancer | 3.8 | Breast Cancer | 14.2 |
| (OD04410) | | 9100265 | |
| Prostate margin | 10.7 | Breast Cancer | 4.1 |
| (OD04410) | | A209073 | |
| Normal Lung | 37.9 | Breast margin | 12.0 |
| Invasive poor diff. | 13.9 | A2090734 | |
| lung adeno 1 | | Normal Liver | 38.4 |
| (ODO4945-01) | | Liver Cancer 1026 | 2.8 |
| Lung margin | 59.0 | Liver Cancer 1025 | 10.3 |
| (ODO4945-03) | | Liver Cancer | 9.0 |
| Lung Malignant | 6.9 | 6004-T | |
| Cancer (OD03126) | | Liver Tissue 6004-N | 1.1 |
| Lung margin | 14.2 | Liver Cancer | 11.7 |
| (OD03126) | | 6005-T | |
| Lung Cancer | 23.8 | Liver Cancer | 8.0 |
| (OD05014A) | | 6005-N | |
| Lung margin | 12.7 | Liver Cancer | 7.0 |
| (OD05014B) | | Normal Bladder | 34.9 |
| Lung Cancer | 33.9 | Bladder Cancer | 1.3 |
| (OD04237-01) | | Bladder Cancer | 7.7 |
| Lung margin | 40.3 | Normal Ovary | 1.7 |
| (OD04237-02) | | Ovarian Cancer | 9.4 |
| Ocular Mel Met to | 31.4 | Ovarian cancer | 3.2 |
| Liver (ODO4310) | | (OD06145) | |
| Liver margin | 41.5 | Ovarian cancer | 14.4 |
| (ODO4310) | | margin (OD06145) | |
| Melanoma Mets to | 31.0 | Normal Stomach | 20.0 |
| Lung (OD04321) | | Gastric Cancer | 5.2 |
| Lung margin | 26.6 | 9060397 | |
| (OD04321) | | Stomach margin | 1.2 |
| Normal Kidney | 15.6 | 9060396 | |
| Kidney Ca, Nuclear | 34.4 | Gastric Cancer | 30.1 |
| grade 2 (OD04338) | | 9060395 | |
| Kidney margin | 24.3 | Stomach margin | 12.4 |
| (OD04338) | 9060394 | | |
| Kidney Ca Nuclear | 7.7 | Gastric Cancer | 18.7 |
| grade 1/2 (OD04339) | | 064005 | |
| Kidney margin | 11.0 | | |
| (OD04339) | | | |
| Kidney Ca, Clear | 19.2 | | |
| cell type (OD04340) | | | |
| Kidney margin | 26.4 | | |
| (OD04340) | | | |
| Kidney Ca, Nuclear | 10.2 | | |
| grade 3 (OD04348) | | | |
| Kidney margin | 12.2 | | |
| (OD04348) | | | |
| Kidney Cancer | 100.0 | | |
| (OD04450-01) | | | |
| Kidney margin | 18.3 | | |
| (OD04450-03) | | | |
| Kidney Cancer | 0.7 | | |
| 8120613 | | | |
| Kidney margin | 1.4 | | |
| 8120614 | | | |

TABLE TG
Panel 4.1D

| | Rel. | Rel. | | Rel. | Rel. |
|---|---|---|---|---|---|
| | Exp. (%) | Exp. (%) | | Exp. (%) | Exp. (%) |
| | Ag3604, | Ag3956, | | Ag3604, | Ag3956, |
| | Run | Run | | Run | Run |
| Tissue Name | 169910577 | 170729090 | Tissue Name | 169910577 | 170729090 |
| Secondary Th1 act | 14.2 | 11.5 | HUVEC IL-1 beta | 8.5 | 5.0 |
| Secondary Th2 act | 18.0 | 13.5 | HUVEC IFN | 5.2 | 4.1 |
| | | | gamma | | |
| Secondary Tr1 act | 17.9 | 10.2 | HUVEC TNF | 7.4 | 4.6 |
| | | | alpha + IFN | | |
| | | | gamma | | |
| Secondary Th1 rest | 1.6 | 1.1 | HUVEC TNF | 11.3 | 6.8 |
| | | | alpha + IL4 | | |
| Secondary Th2 rest | 3.8 | 2.7 | HUVEC IL-11 | 1.8 | 1.5 |
| Secondary Tr1 rest | 2.5 | 1.8 | Lung | 8.0 | 5.8 |
| | | | Microvascular | | |
| | | | none | | |
| Primary Th1 act | 11.8 | 9.0 | Lung | 24.1 | 17.0 |
| | | | Microvascular EC | | |
| | | | TNF alpha + IL- | | |
| | | | 1 beta | | |
| Primary Th2 act | 13.6 | 10.2 | Microvascular | 4.1 | 2.6 |
| | | | Dermal EC none | | |
| Primary Tr1 act | 12.1 | 8.8 | Microsvasular | 12.2 | 6.7 |
| | | | Dermal EC | | |
| | | | TNF alpha + IL- | | |
| | | | 1 beta | | |
| Primary Th1 rest | 3.6 | 2.0 | Bronchial | 11.7 | 7.7 |
| | | | epithelium | | |
| | | | TNF alpha + | | |
| | | | IL1 beta | | |
| Primary Th2 rest | 3.4 | 1.2 | Small airway | 4.2 | 2.5 |
| | | | epithelium none | | |
| Primary Tr1 rest | 3.4 | 3.0 | Small airway | 13.6 | 9.3 |
| | | | epithelium | | |
| | | | TNF alpha + IL- | | |
| | | | 1 beta | | |
| CD45RA CD4 | 13.5 | 9.2 | Coronery artery | 37.1 | 24.7 |
| lymphocyte act | | | SMC rest | | |
| CD45RO CD4 | 14.8 | 10.4 | Coronery artery | 48.6 | 31.6 |
| lymphocyte act | | | SMC TNF alpha + | | |
| | | | IL-1 beta | | |
| CD8 lymphocyte | 14.1 | 8.7 | Astrocytes rest | 6.7 | 3.7 |
| act | | | | | |
| Secondary CD8 | 11.9 | 9.3 | Astrocytes | 15.1 | 7.9 |
| lymphocyte rest | | | TNF alpha + IL- | | |
| | | | 1 beta | | |
| Secondary CD8 | 7.2 | 5.1 | KU-812 (Basophil) | 9.3 | 6.5 |
| lymphocyte act | | | rest | | |
| CD4 lymphocyte | 1.6 | 1.2 | KU-812 (Basophil) | 23.0 | 17.1 |
| none | | | PMA/ionomycin | | |
| 2ry | 2.8 | 2.5 | CCD1106 | 10.6 | 7.6 |
| Th1/Th2/Tr1_anti- | | | (Keratinocytes) | | |
| CD95 CH11 | | | none | | |
| LAK cells rest | 15.7 | 15.3 | CCD1106 | 16.2 | 10.1 |
| | | | (Keratinocytes) | | |
| | | | TNF alpha + IL- | | |
| | | | 1 beta | | |
| LAK cells IL-2 | 6.7 | 5.3 | Liver cirrhosis | 3.5 | 1.8 |
| LAK cells IL-2 + | 7.2 | 4.5 | NCI-H292 none | 6.0 | 4.0 |
| IL-12 | | | | | |
| LAK cells IL- | 10.4 | 4.3 | NCI-H292 IL-4 | 13.3 | 7.4 |
| 2 + IFN gamma | | | | | |
| LAK cells IL-2 + | 9.4 | 4.9 | NCI-H292 IL-9 | 13.6 | 8.3 |
| IL-18 | | | | | |
| LAK cells | 60.7 | 34.2 | NCI-H292 IL-13 | 12.5 | 8.6 |
| PMA/ionomycin | | | | | |
| NK Cells IL-2 rest | 7.2 | 5.0 | NCI-H292 IFN | 13.7 | 8.1 |
| | | | gamma | | |
| Two Way MLR 3 | 15.1 | 7.0 | HPAEC none | 5.3 | 6.9 |
| day | | | | | |
| Two Way MLR 5 | 13.1 | 8.5 | HPAEC TNF | 54.7 | 38.7 |
| day | | | alpha + IL-1 beta | | |
| Two Way MLR 7 | 8.7 | 6.3 | Lung fibroblast | 11.1 | 9.4 |
| day | | | none | | |
| PBMC rest | 1.6 | 1.2 | Lung fibroblast | 7.4 | 7.5 |
| | | | TNF alpha + IL-1 | | |
| | | | beta | | |
| PBMC PWM | 12.8 | 7.5 | Lung fibroblast IL-4 | 18.6 | 10.2 |
| PBMC PHA-L | 10.1 | 6.1 | Lung fibroblast IL-9 | 24.7 | 19.1 |
| Ramos (B cell) | 10.0 | 5.0 | Lung fibroblast IL- | 13.8 | 10.2 |
| none | | | 13 | | |
| Ramos (B cell) | 8.4 | 5.1 | Lung fibroblast | 20.4 | 14.6 |
| ionomycin | | | IFN gamma | | |
| B lymphocytes | 9.7 | 6.5 | Dermal fibroblast | 11.8 | 10.6 |
| PWM | | | CCD1070 rest | | |
| B lymphocytes | 6.7 | 3.8 | Dermal fibroblast | 23.2 | 16.7 |
| CD40L and IL-4 | | | CCD1070 TNF | | |
| | | | alpha | | |
| EOL-1 dbcAMP | 7.9 | 5.1 | Dermal fibroblast | 25.7 | 13.3 |
| | | | CCD1070 IL-1 | | |
| | | | beta | | |
| EOL-1 dbcAMP | 24.0 | 16.0 | Dermal fibroblast | 12.2 | 8.4 |
| PMA/ionomycin | | | IFN gamma | | |
| Dendritic cells none | 23.3 | 13.4 | Dermal fibroblast | 12.6 | 8.5 |
| | | | IL-4 | | |
| Dendritic cells LPS | 28.7 | 20.7 | Dermal Fibroblasts | 8.7 | 8.6 |
| | | | rest | | |
| Dendritic cells anti- | 18.6 | 12.9 | Neutrophils | 7.5 | 6.4 |
| CD40 | | | TNFa + LPS | | |
| Monocytes rest | 2.8 | 1.8 | Neutrophils rest | 0.6 | 0.7 |
| Monocytes LPS | 100.0 | 100.0 | Colon | 1.6 | 1.0 |
| Macrophages rest | 27.7 | 27.4 | Lung | 3.7 | 3.3 |
| Macrophages LPS | 24.8 | 12.5 | Thymus | 5.7 | 3.5 |
| HUVEC none | 3.5 | 2.3 | Kidney | 6.6 | 4.6 |
| HUVEC starved | 4.2 | 2.8 | | | |

CNS_Neurodegeneration_v1.0 Summary:

Ag3604/Ag3956 Two experiments with two different probe and primer sets produce results that are in excellent agreement. This panel does not show differential expression of the CG94820-02 gene in Alzheimer's disease. However, this expression profile confirms the presence of this gene in the brain, with highest expression in the cortex (CTs=28.5). Please see Panel 1.4 for discussion of utility of this gene in the central nervous system.

General_Screening_Panel_v1.4 Summary:

Ag3604/Ag3956 Two experiments with two different probe and primer sets produce results that are in excellent agreement. Highest expression of the CG94820-02 gene is seen in a breast cancer cell line (CTs=24-25). High levels of expression are also seen in all the cell lines on this panel. In addition, higher levels of expression are seen in the fetal tissue samples. Expression in fetal liver and lung (CTs=27) is significantly higher than in the adult liver and lung (CTs=31.5). Therefore, expression of this gene could be used to differentiate between the adult and fetal sources of these tissues. Furthermore, this expression profile suggests a role for this gene product in cell growth and proliferation.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

This gene is also expressed at moderate levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

The CG94820-02 gene codes for a cation-transporting ATPase A, P type. A P-type cation transporting ATPase has been implicated in Menkes disease, a disorder of copper transport characterized by progressive neurological degeneration and death in early childhood (Ref. 1). Thus, the CG94820-02 gene product may play a role in this disease. Therefore, therapeutic modulation of this gene may be useful in the treatment of Menkes disease. (Harrison M D, Dameron C T. (1999) Molecular mechanisms of copper metabolism and the role of the Menkes disease protein. J Biochem Mol Toxicol 1999;13(2):93-106).

Panel 2.1 Summary:

Ag3956 Highest expression of the CG94820-02 gene is seen in a kidney cancer (CT=28.8). Thus, expression of this gene could be used to differentiate between this sample and other samples on this panel and as a marker to detect the presence of kidney cancer. Furthermore, therapeutic modulation of the expression or function of this gene may be effective in the treatment of kidney cancer.

Panel 4.1D Summary:

Ag3604/Ag3956 Two experiments with two different probe and primer sets produce results that are in excellent agreement. Highest expression of the CG94820-02 gene is seen in LPS stimulated monocytes (CTs=25-26). The protein encoded by this gene may therefore be involved in the activation of monocytes in their function as antigen-presenting cells. This suggests that therapeutics that block the function of this membrane protein may be useful as anti-inflammatory therapeutics for the treatment of autoimmune and inflammatory diseases. Furthermore, antibodies or small molecule therapeutics that stimulate the function of this protein may be useful therapeutics for the treatment of immunosupressed individuals.

This gene is also expressed at moderate to low levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_Panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

### Example D. Identification of Single Nucleotide Polymorphisms in NOVX Nucleic Acid Sequences

Variant sequences are also included within the scope of this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process are selected and extended using the following criteria. Genomic clones having regions with 98% identity to all or part of the initial or extended sequence are identified by BLASTN searches using the relevant sequence to query human genomic databases. The genomic clones that resulted are selected for further analysis because this identity indicates that these clones contain the genomic locus for these SeqCalling assemblies. These sequences are analyzed for putative coding regions as well as for similarity to the known DNA and protein sequences. Programs used for these analyses include Grail, Genscan, BLAST, HMMER, FASTA, Hybrid and other relevant programs.

Some additional genomic regions may also be identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified is manually assembled and then may be extended using one or more additional sequences taken from CuraGen Corporation's human SeqCalling database. SeqCalling fragments suitable for inclusion are identified by the CuraTools™ program SeqExtend or by identifying SeqCalling fragments mapping to the appropriate regions of the genomic clones analyzed.

The regions defined by the procedures described above are then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones is reiterated to derive the full length sequence (Alderborn et al., Determination of Single Nucleotide Polymorphisms by Real-time Pyrophosphate DNA Sequencing. Genome Research. 10 (8) 1249-1265, 2000).

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

The claims presented are representative of the inventions disclosed herein. Other, unclaimed inventions are also contemplated. Applicants reserve the right to pursue such inventions in later claims.

What is claimed is:

1. An isolated polypeptide comprising an amino acid sequence selected from the group consisting of:
a) a mature form of the amino acid sequence selected from the group consisting of SEQ ID NO: 2n, wherein n is an integer between 1 and 34;
b) a variant of a mature form of the amino acid sequence selected from the group consisting of SEQ ID NO: 2n, wherein n is an integer between 1 and 34, wherein any amino acid in the mature form is changed to a different amino acid, provided that no more than 15% of the amino acid residues in the sequence of the mature form are so changed;
c) the amino acid sequence selected from the group consisting of SEQ ID NO: 2n, wherein n is an integer between 1 and 34;
d) a variant of the amino acid sequence selected from the group consisting of SEQ ID NO: 2n, wherein n is an integer between 1 and 34, wherein any amino acid specified in the chosen sequence is changed to a different amino acid, provided that no more than 15% of the amino acid residues in the sequence are so changed; and
e) a fragment of any of a) through d).

2. The polypeptide of claim 1 that is a naturally occurring allelic variant of the sequence selected from the group consisting of SEQ ID NO: 2n, wherein n is an integer between 1 and 34.

3. The polypeptide of claim 2, wherein the allelic variant comprises an amino acid sequence that is the translation of a nucleic acid sequence differing by a single nucleotide from a nucleic acid sequence selected from the group consisting of SEQ ID NOS: 2n, wherein n is an integer between 1 and 34.

4. The polypeptide of claim 1 that is a variant polypeptide described therein, wherein any amino acid specified in the chosen sequence is changed to provide a conservative substitution.

5. A pharmaceutical composition comprising the polypeptide of claim 1 and a pharmaceutically acceptable carrier.

6. A kit comprising in one or more containers, the pharmaceutical composition of claim 5.

7. The use of a therapeutic in the manufacture of a medicament for treating a syndrome associated with a human disease, the disease selected from a pathology associated with the polypeptide of claim 1, wherein the therapeutic is the polypeptide of claim 1.

8. A method for determining the presence or amount of the polypeptide of claim 1 in a sample, the method comprising:
(a) providing the sample;
(b) introducing the sample to an antibody that binds immunospecifically to the polypeptide; and
(c) determining the presence or amount of antibody bound to the polypeptide, thereby determining the presence or amount of polypeptide in the sample.

9. A method for determining the presence of or predisposition to a disease associated with altered levels of the polypeptide of claim 1 in a first mammalian subject, the method comprising:
a) measuring the level of expression of the polypeptide in a sample from the first mammalian subject; and
b) comparing the amount of the polypeptide in the sample of step (a) to the amount of the polypeptide present in a control sample from a second mammalian subject known not to have, or not to be predisposed to, the disease,
wherein an alteration in the expression level of the polypeptide in the first subject as compared to the control sample indicates the presence of or predisposition to the disease.

10. A method of identifying an agent that binds to the polypeptide of claim 1, the method comprising:
(a) introducing the polypeptide to the agent; and
(b) determining whether the agent binds to the polypeptide.

11. The method of claim 10 wherein the agent is a cellular receptor or a downstream effector.

12. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition devoid of the substance, the substance is identified as a potential therapeutic agent.

13. A method for screening for a modulator of activity or of latency or predisposition to a pathology associated with the polypeptide of claim 1, the method comprising:
a) administering a test compound to a test animal at increased risk for a pathology associated with the polypeptide of claim 1, wherein the test animal recombinantly expresses the polypeptide of claim 1;
b) measuring the activity of the polypeptide in the test animal after administering the compound of step (a); and
c) comparing the activity of the protein in the test animal with the activity of the polypeptide in a control animal not administered the polypeptide, wherein a change in the activity of the polypeptide in the test animal relative to the control animal indicates the test compound is a modulator of latency of, or predisposition to, a pathology associated with the polypeptide of claim 1.

14. The method of claim 13, wherein the test animal is a recombinant test animal that expresses a test protein transgene or expresses the transgene under the control of a promoter at an increased level relative to a wild-type test animal, and wherein the promoter is not the native gene promoter of the transgene.

15. A method for modulating the activity of the polypeptide of claim 1, the method comprising introducing a cell sample expressing the polypeptide of the claim with a compound that binds to the polypeptide in an amount sufficient to modulate the activity of the polypeptide.

16. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

17. The method of claim 16, wherein the subject is a human.

18. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO: 2n, wherein n is an integer between 1 and 34, or a biologically active fragment thereof.

19. An isolated nucleic acid molecule comprising a nucleic acid sequence encoding a polypeptide comprising an amino acid sequence selected from the group consisting of:
a) a mature form of the amino acid sequence given SEQ ID NO: 2n, wherein n is an integer between 1 and 34;
b) a variant of a mature form of the amino acid sequence selected from the group consisting of SEQ ID NO: 2n, wherein n is an integer between 1 and 34, wherein any amino acid in the mature form of the chosen sequence is changed to a different amino acid, provided that no more than 15% of the amino acid residues in the sequence of the mature form are so changed;
c) the amino acid sequence selected from the group consisting of SEQ ID NO: 2n, wherein n is an integer between 1 and 34;
d) a variant of the amino acid sequence selected from the group consisting of SEQ ID NO: 2n, wherein n is an integer between 1 and 34, in which any amino acid specified in the chosen sequence is changed to a different amino acid, provided that no more than 15% of the amino acid residues in the sequence are so changed;
e) a nucleic acid fragment encoding at least a portion of a polypeptide comprising the amino acid sequence selected from the group consisting of SEQ ID NO: 2n, wherein n is an integer between 1 and 34, or any variant of the polypeptide wherein any amino acid of the chosen sequence is changed to a different amino acid, provided that no more than 10% of the amino acid residues in the sequence are so changed; and
f) the complement of any of the nucleic acid molecules.

20. The nucleic acid molecule of claim 19, wherein the nucleic acid molecule comprises the nucleotide sequence of a naturally occurring allelic nucleic acid variant.

21. The nucleic acid molecule of claim 19 that encodes a variant polypeptide, wherein the variant polypeptide has the polypeptide sequence of a naturally occurring polypeptide variant.

22. The nucleic acid molecule of claim 19, wherein the nucleic acid molecule differs by a single nucleotide from a nucleic acid sequence selected from the group consisting of SEQ ID NOS: 2n-1, wherein n is an integer between 1 and 34.

23. The nucleic acid molecule of claim 19, wherein the nucleic acid molecule comprises a nucleotide sequence selected from the group consisting of
a) the nucleotide sequence selected from the group consisting of SEQ ID NO: 2n-1, wherein n is an integer between 1 and 34;
b) a nucleotide sequence wherein one or more nucleotides in the nucleotide sequence selected from the group consisting of SEQ ID NO: 2n-1, wherein n is an integer between 1 and 34, is changed from that selected from the group consisting of the chosen sequence to a different nucleotide provided that no more than 15% of the nucleotides are so changed;
c) a nucleic acid fragment of the sequence selected from the group consisting of SEQ ID NO: 2n-1, wherein n is an integer between 1 and 34; and
d) a nucleic acid fragment wherein one or more nucleotides in the nucleotide sequence selected from the group consisting of SEQ ID NO: 2n-1, wherein n is an integer between 1 and 34, is changed from that selected from the group consisting of the chosen sequence to a different nucleotide provided that no more than 15% of the nucleotides are so changed.

24. The nucleic acid molecule of claim 19, wherein the nucleic acid molecule hybridizes under stringent conditions to the nucleotide sequence selected from the group consisting of SEQ ID NO: 2n-1, wherein n is an integer between 1 and 34, or a complement of the nucleotide sequence.

25. The nucleic acid molecule of claim 19, wherein the nucleic acid molecule comprises a nucleotide sequence in which any nucleotide specified in the coding sequence of the chosen nucleotide sequence is changed from that selected from the group consisting of the chosen sequence to a different nucleotide provided that no more than 15% of the nucleotides in the chosen coding sequence are so changed, an isolated second polynucleotide that is a complement of the first polynucleotide, or a fragment of any of them.

26. A vector comprising the nucleic acid molecule of claim 19.

27. The vector of claim 26, further comprising a promoter operably linked to the nucleic acid molecule.

28. A cell comprising the vector of claim 27.

29. A method for determining the presence or amount of the nucleic acid molecule of claim 19 in a sample, the method comprising:
(a) providing the sample;
(b) introducing the sample to a probe that binds to the nucleic acid molecule; and
(c) determining the presence or amount of the probe bound to the nucleic acid molecule,
thereby determining the presence or amount of the nucleic acid molecule in the sample.

30. The method of claim 29 wherein presence or amount of the nucleic acid molecule is used as a marker for cell or tissue type.

31. The method of claim 30 wherein the cell or tissue type is cancerous.

32. A method for determining the presence of or predisposition to a disease associated with altered levels of the nucleic acid molecule of claim 19 in a first mammalian subject, the method comprising:
a) measuring the amount of the nucleic acid in a sample from the first mammalian subject; and
b) comparing the amount of the nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.
